(19) United States
(12) Patent Application Publication
(10) Pub. No.: US 2007/0071681 A1
(43) Pub. Date: Mar. 29, 2007

(54) Apparatus and method for computer modeling type 1 diabetes

(75) Inventors: Kapil Gadkar, Foster City, CA (US); Huub Kreuwel, San Francisco, CA (US); Thomas Paterson, Redondo Beach, CA (US); David Polidori, Rancho Santa Fe, CA (US); Saroja Ramanujan, Daly City, CA (US); Lisl Katharine Mie Shoda, Menlo Park, CA (US); Chan D. Whiting, San Jose, CA (US); Daniel L. Young, San Francisco, CA (US); Yanan Zheng, Foster City, CA (US)

Correspondence Address: ENTELOS, INC.;c/o FOLEY & LARDNER LLP, 1530 PAGE MILL RD., PALO ALTO, CA, 94304, US

(73) Assignee: Entelos, Inc.

(21) Appl. No.: 11/377,614

(22) Filed: Mar. 15, 2006

Related U.S. Application Data
(60) Provisional application No. 60/662,494, filed on Mar. 15, 2005.
(60) Provisional application No. 60/691,473, filed on Jun. 16, 2005.

(51) Int. Cl. A61K 49/00; G06F 19/00; G06Q 50/00

(52) U.S. Cl. 424/9.2; 702/19; 705/3

(57) ABSTRACT

The invention encompasses novel methods for developing a computer model of type 1 diabetes in a mammal. In particular, the models can include representations of biological processes associated with a pancreatic lymph node and one or more pancreatic islets. Alternatively, the models can include representations of biological processes associated with at least two conditions selected from the group consisting of autoreactive T cell production, autoreactive T cell priming, insulitis and hyperglycemia. The invention also provides methods for developing a computer model of a non-insulin replacement treatment of type 1 diabetes. The invention also encompasses computer models of type 1 diabetes, methods of simulating type 1 diabetes and computer systems for simulating type 1 diabetes and the uses thereof.

### CROSS-REFERENCE TO RELATED APPLICATIONS

This application claims benefit of U.S. Application Ser. No. 60/662,494, filed 15 Mar. 2005, and of U.S. Application Ser. No. 60/691,473, filed 16 Jun. 2005, each incorporated herein by reference in its entirety.

### FIELD OF THE INVENTION

The present invention relates generally to the field of simulating type 1 diabetes in mammals.

### BACKGROUND OF THE INVENTION

Type 1 diabetes is a multifactorial autoimmune disease that affects approximately one million people in the United States alone. As disease onset often occurs early in life, primary disease and associated complications pose significant social and financial costs. The disease arises from the autoimmune destruction of islet β cells in the pancreas and the subsequent loss of glucose control. However, the understanding of type 1 diabetes pathogenesis and efforts to prevent, halt, or reverse the disease are significantly impaired by the inherent difficulties associated with studying these processes in prediabetic and diabetic humans. These difficulties include the challenge of identifying individuals that will develop type 1 diabetes, as well as practical considerations in studying the involved tissues.

Since the study of type 1 diabetes pathogenesis in humans is difficult, much of the current understanding regarding disease progression and pathogenesis is derived from rodent models. The non-obese diabetic (NOD) mouse is a particularly well studied model in which the majority of females spontaneously develop diabetes. In these mice, the importance of numerous immune components to disease development has been experimentally established, and numerous therapies have been shown to inhibit the development of type 1 diabetes. Despite multiple successes in protecting the NOD mouse from disease, however, success in advancing therapies from the NOD mouse to human patients has been limited. Currently, no preventative or curative treatments are available for human type 1 diabetes.

Due to the complexity of the biological processes in type 1 diabetes, mathematical and computer models can be used to help better understand the interactions between the tissue compartments, cell populations, mediators, and other factors involved in autoimmune pancreatic disease and healthy homeostasis. Several researchers have constructed simple models of beta cells, insulin production, and glucose control (e.g., Toffolo et al. Diabetes 29:979-990 (1980); Walton et al. Am J Physiol 262:E755-E762 (1992); Sweet and Matschinsky Am J Physiol 268:E775-E788 (1995); Andersen and Hojbjerre Stat Med 24:2381-2400 (2005)). Other researchers have constructed simple models of beta cells and a limited number of immune cell types (Freiesleben et al. Diabetes 48:1677-1685 (1999); Trudeau et al. Diabetes 49:1-7 (2000); Maree et al. J Theor Biol 233:533-551 (2005)). Still others have constructed simple to complex models designed to improve disease management or determine economic costs of the disease (e.g., (Lehmann et al. Med Inform (Lond) 18:83-101 (1993); Cavan et al. J Telemed Telecare 9 Suppl 1:S50-2.:S50-S52 (2003); Palmer et al. Curr Med Res Opin 20 Suppl 1:S5-26.:S5-26 (2004); Warren et al. Health Technol Assess 8:iii, 1-iii,57 (2004)). However, these models have not focused on autoimmune pathogenesis of type 1 diabetes; wherein, the specific elements of both target tissues and immune components are dynamically and mechanistically represented over time. Further, these models have not included contributions of events in the pancreatic lymph nodes to the development and progression of type 1 diabetes. Hence, there is a need for a computer or mathematical model which includes biological compartments and the interactions between compartments required for the representation of type 1 diabetes autoimmune disease progression.

### SUMMARY OF THE INVENTION

One aspect of the invention provides methods for developing a model of type 1 diabetes said method comprising identifying one or more biological processes associated with a pancreatic lymph node; identifying one or more biological processes associated with one or more pancreatic islets; mathematically representing each biological process to generate one or more representations of a biological process associated with the pancreatic lymph node and one or more representations of a biological process associated with the one or more pancreatic islets; and combining the representations of biological processes to form a model of type 1 diabetes. The methods further can comprise identifying one or more biological processes associated with gut and/or gut associated lymphoid tissue; and mathematically representing each biological process associated with the gut and/or gut associated lymphoid tissue. In certain implementations the one or more pancreatic islets comprise at least two pancreatic islets. In another preferred implementation, at least one of the one or more biological processes associated with a pancreatic lymph node is a biological process related to a balance of effector and regulatory cell populations. In yet another preferred implementation, at least one of the one or more biological processes associated with a one or more pancreatic islets is a biological process related to a balance of effector and regulatory cell populations. Preferably, the regulatory cell population comprises cells of lymphoid lineage. More preferably, the regulatory cell population comprises regulatory T cells. The regulatory T cells preferably do not express intrinsic effector cell activity.

One aspect of the invention provides computer-readable media having computer-readable instructions stored thereon that, upon execution by a processor, cause the processor to simulate type 1 diabetes, and further wherein the instructions comprise: a) mathematically representing one or more biological processes associated with a pancreatic lymph node; b) mathematically representing one or more biological processes associated with one or more pancreatic islets; c) defining a set of mathematical relationships between the representations of biological processes to form a model of type 1 diabetes. The instructions can further comprise mathematically representing one or more biological processes associated with gut and/or gut associated lymphoid tissue. Alternatively, the instructions can further comprise accepting user input specifying one or more parameters associated with one or more of the mathematical representations. In another implementation, the instructions further comprise accepting user input specifying one or more variables associated with one or more of the mathematical representations. In yet another implementation, the instructions further comprise applying a virtual protocol to the model of type 1 diabetes. In yet another implementation, the instructions further comprise defining one or more virtual patients.

Yet another aspect of the invention provides systems comprising a) a processor including computer-readable instructions stored thereon that, upon execution by a processor, cause the processor to simulate type 1 diabetes in a mammal; b) a first user terminal, the first user terminal operable to receive a user input specifying one or more parameters associated with one or more mathematical representations defined by the computer readable instructions; and c) a second user terminal, the second user terminal operable to provide the set of outputs to a second user. The computer readable instructions preferably comprises: i) mathematically representing one or more biological processes associated with a pancreatic lymph node; ii) mathematically representing one or more biological processes associated with one or more pancreatic islets; iii) defining a set of mathematical relationships between the representations of biological processes associated with the pancreatic lymph node and representations of biological processes associated with the one or more pancreatic islets; and iv) applying a virtual protocol to the set of mathematical relationships to generate a set of outputs. In one implementation, the first and second users are the same user. In another implementation, the first and second users are different users.

Another aspect of the invention provides methods for developing a model of progression of type 1 diabetes, said method comprising: identifying one or more biological processes associated with each of at least two conditions selected from the group consisting of autoreactive T cell production, autoreactive T cell priming; insulitis and hyperglycemia; mathematically representing each biological process to generate one or more representations of a biological process associated with each of the at least two conditions; and combining the representations of biological processes to form a model of progression of type 1 diabetes. Preferably, the at least two conditions comprise insulitis and hyperglycemia. In an alternate implementation, the at least two conditions comprise autoreactive T cell priming. The biological processes can be associated with the onset, existence, progression or transition from any of the conditions. The methods for developing a model of progression of type 1 diabetes can further comprise identifying one or more biological processes associated with inflammatory dendritic cells and one or more biological processes associated with suppressive dendritic cells; and mathematically representing each biological process to generate one or more representations of a biological process associated with the inflammatory dendritic cells and one or more representations of a biological process associated with the suppressive dendritic cells.

Yet another aspect of the invention provides computer-readable media having computer-readable instructions stored thereon that, upon execution by a processor, cause the processor to simulate progression of type 1 diabetes, and further wherein the instructions comprise: a) mathematically representing one or more biological processes associated with each of at least two conditions selected from the group consisting of autoreactive T cell production, autoreactive T cell priming; insulitis and hyperglycemia; b) defining a set of mathematical relationships between the representations of biological processes to form a model of progression of type 1 diabetes. The instructions can further comprise mathematically representing one or more biological processes associated with gut and/or gut associated lymphoid tissue. Alternatively, the instructions can further comprise accepting user input specifying one or more parameters associated with one or more of the mathematical representations. In another implementation, the instructions further comprise accepting user input specifying one or more variables associated with one or more of the mathematical representations. In yet another implementation, the instructions further comprise applying a virtual protocol to the model of type 1 diabetes. In yet another implementation, the instructions further comprise defining one or more virtual patients.

Another aspect of the invention provides systems comprising a) a processor including computer-readable instructions stored thereon that, upon execution by a processor, cause the processor to simulate progression of type 1 diabetes in a mammal; b) a first user terminal, the first user terminal operable to receive a user input specifying one or more parameters associated with one or more mathematical representations defined by the computer readable instructions; and c) a second user terminal, the second user terminal operable to provide the set of outputs to a second user. The computer readable instructions comprise: i) mathematically representing one or more biological processes associated with development of each of at least two conditions selected from the group consisting of autoreactive T cell production, autoreactive T cell priming; insulitis and hyperglycemia; ii) defining a set of mathematical relationships between the representations of biological processes associated with the at least two conditions; and iii) applying a virtual protocol to the set of mathematical relationships to generate a set of outputs. In one implementation, the first and second users are the same user. In another implementation, the first and second users are different users.

Yet another aspect of the invention provides methods for developing a model of a non-insulin replacement treatment of type 1 diabetes said method comprising: identifying one or more biological processes associated with a β cell population in at least one of one or more pancreatic islets; identifying one or more biological processes associated with an effect of a non-insulin replacement treatment of type 1 diabetes; mathematically representing each biological process to generate one or more representations of a biological process associated with the β cell population and one or more representations of a biological process associated with an effect of the non-insulin replacement treatment of type 1 diabetes; and combining the representations of the biological processes to form the model of a non-insulin replacement treatment of type 1 diabetes. The method further can comprise the steps of identifying one or more biological processes associated with a pancreatic lymph node; and mathematically representing each biological process to generate one or more representations of a biological process associated with the pancreatic lymph node. In a preferred implementations the one or more biological processes associated with the β cell population comprises a biological process associated with an autoimmune response against β cells. In another implementation, the one or more biological processes associated with the β cell population comprises a biological process associated with resistance of β cells to death. In yet another implementation, the one or more biological processes associated with the β cell population comprises a biological process associated with β cell proliferation. In another implementation, the one or more biological processes associated with the β cell population comprises a biological process associated with β cell neogenesis. In another preferred implementation, at least one of the one or more biological processes associated with the β cell population is a biological process related to a balance of effector and regulatory cell populations. The balance of effector and regulatory cell populations can include a balance of cell numbers as well as a balance of cell functions. Preferably, the regulatory cell population comprises cells of lymphoid lineage. More preferably, the regulatory cell population comprises regulatory T cells. The regulatory T cells of the regulatory cell population preferably do not express intrinsic effector cell activity.

One aspect of the invention provides computer-readable media having computer-readable instructions stored thereon that, upon execution by a processor, cause the processor to simulate a non-insulin replacement treatment of type 1 diabetes, and further wherein the instructions comprise: a) mathematically representing one or more biological processes associated with a β cell population in at least one of one or more pancreatic islets; b) mathematically representing one or more biological processes associated with an effect of the non-insulin replacement treatment of type 1 diabetes; c) defining a set of mathematical relationships between the representations of biological processes to form a model of the non-insulin replacement treatment of type 1 diabetes. The instructions can further comprise mathematically representing one or more biological processes associated with a pancreatic lymph node. Alternatively or in addition, the instructions can further comprise mathematically representing one or more biological processes associated with gut and/or gut associated lymphoid tissue. The instructions also can further comprise accepting user input specifying one or more parameters associated with one or more of the mathematical representations. In another implementation, the instructions further comprise accepting user input specifying one or more variables associated with one or more of the mathematical representations. In yet another implementation, the instructions further comprise applying a virtual protocol to the model of type 1 diabetes. In yet another implementation, the instructions further comprise defining one or more virtual patients.

Another aspect of the invention provides systems comprising a) a processor including computer-readable instructions stored thereon that, upon execution by a processor, cause the processor to simulate a non-insulin replacement treatment of type 1 diabetes in a mammal; b) a first user terminal, the first user terminal operable to receive a user input specifying one or more parameters associated with one or more mathematical representations defined by the computer readable instructions; and c) a second user terminal, the second user terminal operable to provide the set of outputs to a second user. The computer readable instructions comprise: i) mathematically representing one or more biological processes associated with one or more pancreatic islets; ii) mathematically representing one or more biological processes associated with a β cell population in at least one of the one or more pancreatic islets; iii) mathematically representing one or more biological processes associated with an effect of a non-insulin replacement treatment of type 1 diabetes; iv) defining a set of mathematical relationships between the representations of biological processes associated with the one or more pancreatic islets and the representations of biological processes associated with the β cell population and the representations associated with an effect of the non-insulin replacement treatment of type 1 diabetes; and v) applying a virtual protocol to the set of mathematical relationships to generate a set of outputs. In one implementation, the first and second users are the same user. In another implementation, the first and second users are different users.

One aspect of the invention provides a computer-based mathematical model of a biological system comprising a representation of a tissue, wherein the tissue comprises a plurality of distinct distributed sites and the representation of the tissue comprises a plurality of representations, wherein each of the plurality of representations associated with one of the plurality of distinct distributed sites. Preferably the tissue is selected from the group consisting of lung, brain, liver, joints, intestine and pancreas. In one implementation of the invention, the distinct distributed sites describe spatial heterogeneity within the tissue. Another implementation provides models wherein the distinct distributed sites describe temporal heterogeneity within the tissue. In another implementation, the distinct distributed sites describe distinct stages in progression of a disorder within the tissue.

Yet another aspect of the invention provides a computer-based mathematical model of a T lymphocyte response comprising a representation of one or more biological processes associated with inflammatory dendritic cells and one or more biological processes associated with suppressive dendritic cells. Preferably the T cell response includes both effector and regulatory T cells. In one implementation of the invention, the balance of inflammatory vs. suppressive dendritic cells drives the relative expansion of effector vs. regulatory T cells. Another implementation provides models wherein inflammatory vs. suppressive dendritic cells characterize a lack of immune response (i.e., little to no effector T cell expansion) to a particular antigen or set of antigens.

One aspect of the invention provides methods of simulating type 1 diabetes, said method comprising executing a computer model of the invention, as described herein. In certain implementations, the method of simulating type 1 diabetes further comprises applying a virtual protocol to the computer model to generate set of outputs representing a phenotype of type 1 diabetes. Another preferred implementation includes methods, wherein the virtual protocol comprises a therapeutic regimen, a diagnostic procedure, passage of time, exposure to environmental toxins, or physical exercise. Yet another implementation of the invention provides methods further comprising accepting user input specifying one or more parameters or variables associated with one or more mathematical representations prior to executing the computer model. Preferably, the user input comprises a definition of a virtual patient.

It will be appreciated by one of skill in the art that the embodiments summarized above may be used together in any suitable combination to generate additional embodiments not expressly recited above, and that such embodiments are considered to be part of the present invention

### BRIEF DESCRIPTION OF THE DRAWINGS

An overview of the methods used to develop computer models of type 1 is illustrated in FIG. 1.

FIG. 2 illustrates exemplary Summary Diagram that links modules relating to pancreatic lymph nodes, islets and other related biological processes.

FIG. 3 illustrates islet heterogeneity, as reproduced through the explicit modeling of distinct distributed sites.

FIG. 4 illustrates a series of distinct events, also called checkpoints, characterizing pathogenesis of type 1 diabetes in NOD mice.

FIG. 5 demonstrates that a virtual NOD mouse, as simulated by a model of type 1 diabetes, becomes diabetic at approximately 20 weeks of age, in agreement with actual data collected from live NOD mice. Blood glucose data from diabetic (squares) NOD mice and non-diabetic (circles) NOD mice are overlaid by simulation data from the virtual NOD mouse. The virtual NOD mouse becomes hyperglycemic within the same timeframe as actual NOD mice.

FIG. 6 illustrates the relative contributions from each distinct distributed site, combined to yield the average infiltrate across the total pancreas. FIG. 6A illustrates the progressive infiltration of distinct distributed sites (islet bins) over the course of disease progression in a virtual NOD mouse. FIG. 6B illustrates how the events occurring in distinct distributed are combined to yield a reflection of the average infiltration across all pancreatic islets in a virtual NOD mouse.

FIG. 7 provides an exemplary Effect Diagram describing conventional CD4+ T cell recruitment and life cycle within a pancreatic lymph node.

FIG. 8 demonstrates that depletion of innate regulatory T cells mechanistically causes exacerbation of disease processes leading to earlier disease onset within the model of type 1 diabetes. FIG. 8A illustrates the virtual NOD mouse without innate regulatory T cells exhibits a stronger and more rapid expansion of PLN CD4+ Th1 cells (upper solid line), relative to the reference virtual NOD mouse (upper dashed line). The number of innate T regulatory cells is quantified in the lower two lines (dashed is the reference virtual NOD mouse, solid is the virtual NOD mouse without innate T regulatory cells). FIG. 8B illustrates that the virtual NOD mouse without innate regulatory T cells develops disease, as defined by hyperglycemia, at an earlier time than the reference mouse.

FIG. 9 shows an example of a module diagram for the CD8+ T cell life cycle in the islet.

FIG. 10 shows an exemplary Effect Diagram describing conventional CD4+ T cell priming in pancreatic lymph nodes.

FIG. 11 shows an exemplary Effect Diagram describing CD4+ and innate regulatory T cell life cycle regulation in pancreatic lymph nodes.

FIG. 12 shows an exemplary Effect Diagram describing conventional CD4+ T cell differentiation in pancreatic lymph nodes.

FIG. 13 shows an exemplary Effect Diagram describing innate regulatory T cell recruitment and life cycle in pancreatic lymph nodes.

FIG. 14 shows an exemplary Effect Diagram describing innate regulatory T cell activation in pancreatic lymph nodes.

FIG. 15 shows an exemplary Effect Diagram describing CD4+ T cell and innate regulatory T cell mediator synthesis in pancreatic lymph nodes.

FIG. 16 shows an exemplary Effect Diagram describing T cell calculation in pancreatic lymph nodes.

FIG. 17 shows an exemplary Effect Diagram describing CD8+ T cell recruitment and life cycle in pancreatic lymph nodes.

FIG. 18 shows an exemplary Effect Diagram describing CD8+ T cell activation in pancreatic lymph nodes.

FIG. 19 shows an exemplary Effect Diagram describing CD8+ life cycle regulation and mediator synthesis in pancreatic lymph nodes.

FIG. 20 shows an exemplary Effect Diagram describing CD8+ T cell calculations in pancreatic lymph nodes.

FIG. 21 shows an exemplary Effect Diagram describing B lymphocyte recruitment and life cycle in pancreatic lymph nodes.

FIG. 22 shows an exemplary Effect Diagram describing B lymphocyte activation in pancreatic lymph nodes.

FIG. 23 shows an exemplary Effect Diagram describing B lymphocyte life cycle regulation, mediator synthesis, and antibody production in pancreatic lymph nodes.

FIG. 24 shows an exemplary Effect Diagram describing B lymphocyte calculations in pancreatic lymph nodes.

FIG. 25 shows an exemplary Effect Diagram describing natural killer (NK) cell recruitment and life cycle in pancreatic lymph nodes.

FIG. 26 shows an exemplary Effect Diagram describing NK cell activation in pancreatic lymph nodes.

FIG. 27 shows an exemplary Effect Diagram describing NK cell life cycle regulation and mediator synthesis in pancreatic lymph nodes.

FIG. 28 shows an exemplary Effect Diagram describing NK cell calculations in pancreatic lymph nodes.

FIG. 29 shows an exemplary Effect Diagram describing dendritic cell and macrophage trafficking and life cycle in pancreatic lymph nodes.

FIG. 30 shows an exemplary Effect Diagram describing macrophage activation in pancreatic lymph nodes.

FIG. 31 shows an exemplary Effect Diagram describing macrophage mediator synthesis and dendritic cell/macrophage:T cell contact in pancreatic lymph nodes.

FIG. 32 shows an exemplary Effect Diagram describing dendritic cell and macrophage calculations in pancreatic lymph nodes.

FIG. 33 shows an exemplary Effect Diagram describing antigen presenting cells in pancreatic lymph nodes.

FIG. 34 shows an exemplary Effect Diagram describing dendritic cell activation and surface molecule expression in pancreatic lymph nodes.

FIG. 35 shows an exemplary Effect Diagram describing dendritic cell mediator synthesis in pancreatic lymph nodes.

FIG. 36 shows an exemplary Effect Diagram describing tissue composition and volume calculations in pancreatic lymph nodes.

FIG. 37 shows an exemplary Effect Diagram describing T cell surface molecule expression and cell contact in pancreatic lymph nodes.

FIGS. 38 and 39 show an exemplary Effect Diagram describing total mediator production in pancreatic lymph nodes.

FIG. 40 shows an exemplary Effect Diagram describing CD4+ T cell and innate regulatory T cell calculations in pancreatic lymph nodes.

FIG. 41 shows an exemplary Effect Diagram describing CD4+ T cell and innate regulatory T cell recruitment and life cycle in pancreatic islets.

FIG. 42 shows an exemplary Effect Diagram describing CD4+ T cell activation in pancreatic islets.

FIG. 43 shows an exemplary Effect Diagram describing innate regulatory T cell activation in pancreatic islets.

FIG. 44 shows an exemplary Effect Diagram describing CD4+ T cell and innate regulatory T cell life cycle regulation in pancreatic islets.

FIG. 45 shows an exemplary Effect Diagram describing CD8+ T cell activation in pancreatic islets.

FIG. 46 shows an exemplary Effect Diagram describing CD8+ T cell life cycle regulation in pancreatic islets.

FIG. 47 shows an exemplary Effect Diagram describing T cell mediator synthesis and innate regulatory T cell contact in pancreatic islets.

FIG. 48 shows an exemplary Effect Diagram describing B lymphocyte recruitment and life cycle in pancreatic islets.

FIG. 49 shows an exemplary Effect Diagram describing B lymphocyte activation, mediator synthesis and antibody production in pancreatic islets.

FIG. 50 shows an exemplary Effect Diagram describing B lymphocyte life cycle regulation in pancreatic islets.

FIG. 51 shows an exemplary Effect Diagram describing B lymphocyte antigen loading in pancreatic islets.

FIG. 52 shows an exemplary Effect Diagram describing NK cell recruitment and life cycle in pancreatic islets.

FIG. 53 shows an exemplary Effect Diagram describing NK cell activation in pancreatic islets.

FIG. 54 shows an exemplary Effect Diagram describing NK cell life cycle regulation in pancreatic islets.

FIG. 55 shows an exemplary Effect Diagram describing NK mediator synthesis and effector functions in pancreatic islets.

FIG. 56 shows an exemplary Effect Diagram describing dendritic cell and macrophage recruitment and life cycle in pancreatic islets.

FIG. 57 shows an exemplary Effect Diagram describing dendritic cell and macrophage activation and surface molecule expression in pancreatic islets.

FIG. 58 shows an exemplary Effect Diagram describing dendritic cell and macrophage mediator synthesis in pancreatic islets.

FIGS. 59 and 60 show exemplary Effect Diagrams describing dendritic cell and macrophage antigen loading in pancreatic islets.

FIG. 61 shows an exemplary Effect Diagram describing dendritic cell surface molecule in β islet and antigen traffic to pancreatic lymph node.

FIG. 62 shows an exemplary Effect Diagram describing dendritic cell and macrophage maturation and activation calculations in pancreatic islets.

FIG. 63 shows an exemplary Effect Diagram describing dendritic cell and macrophage calculations in pancreatic islets.

FIGS. 64 and 65 show exemplary Effect Diagrams describing tissue composition in pancreatic islets.

FIG. 66 shows an exemplary Effect Diagram describing surface molecule expression and cell contact in pancreatic islets.

FIGS. 67 and 68 show exemplary Effect Diagrams describing total mediator production in pancreatic islets.

FIG. 69 shows an exemplary Effect Diagram describing β cell life cycle.

FIG. 70 shows an exemplary Effect Diagram describing β cell surface molecule expression and mediator synthesis in pancreatic islets.

FIG. 71 shows an exemplary Effect Diagram describing glucose regulation and β cell function.

FIG. 72 shows an exemplary Effect Diagram describing β cell mass and exhaustion.

FIG. 73 shows an exemplary Effect Diagram describing islet involvement calculations.

FIG. 74 shows an exemplary Effect Diagram describing islet involvement rate calculations.

FIG. 75 shows an exemplary Effect Diagram describing disease induced destruction.

FIG. 76 shows an exemplary Effect Diagram describing age dependent characteristics and data.

FIG. 77 shows an exemplary Effect Diagram describing innate regulatory T cell mediator calculations in pancreatic islets.

FIG. 78 shows an exemplary Effect Diagram describing T cell calculations in the blood compartment.

FIG. 79 shows an exemplary Effect Diagram describing average involved total CD4+ T cells across multiple distinct pancreatic islets.

FIG. 80 shows an exemplary Effect Diagram describing average involved CD4+ T cell activation across multiple distinct pancreatic islets.

FIG. 81 shows an exemplary Effect Diagram describing average involved apoptotic CD4+ T cells across multiple distinct pancreatic islets.

FIG. 82 shows an exemplary Effect Diagram describing average involved Th1 cells across multiple distinct pancreatic islets.

FIG. 83 shows an exemplary Effect Diagram describing average involved Th1 cell proliferation across multiple distinct pancreatic islets.

FIG. 84 shows an exemplary Effect Diagram describing average involved Th2 cells across multiple distinct pancreatic islets.

FIG. 85 shows an exemplary Effect Diagram describing average involved Th2 cell proliferation across multiple distinct pancreatic islets.

FIG. 86 shows an exemplary Effect Diagram describing average involved adaptive regulatory T cells across multiple distinct pancreatic islets.

FIG. 87 shows an exemplary Effect Diagram describing average involved adaptive regulatory T cell proliferation across multiple distinct pancreatic islets.

FIG. 88 shows an exemplary Effect Diagram describing average involved innate regulatory T cells across multiple distinct pancreatic islets.

FIG. 89 shows an exemplary Effect Diagram describing average involved innate regulatory T cell activation across multiple distinct pancreatic islets.

FIG. 90 shows an exemplary Effect Diagram describing average involved innate regulatory T cell proliferation across multiple distinct pancreatic islets.

FIG. 91 shows an exemplary Effect Diagram describing average involved apoptotic innate regulatory T cells across multiple distinct pancreatic islets.

FIG. 92 shows an exemplary Effect Diagram describing average involved total CD8+ T cells across multiple distinct pancreatic islets.

FIG. 93 shows an exemplary Effect Diagram describing average involved CD8+ T cell activation across multiple distinct pancreatic islets.

FIG. 94 shows an exemplary Effect Diagram describing average involved CD8+ T cell proliferation across multiple distinct pancreatic islets.

FIG. 95 shows an exemplary Effect Diagram describing average involved apoptotic CD8+ T cells across multiple distinct pancreatic islets.

FIG. 96 shows an exemplary Effect Diagram describing average involved B lymphocytes across multiple distinct pancreatic islets.

FIG. 97 shows an exemplary Effect Diagram describing average involved B lymphocyte activation across multiple distinct pancreatic islets.

FIG. 98 shows an exemplary Effect Diagram describing average involved B lymphocyte proliferation across multiple distinct pancreatic islets.

FIG. 99 shows an exemplary Effect Diagram describing average involved apoptotic B lymphocytes across multiple distinct pancreatic islets.

FIG. 100 shows an exemplary Effect Diagram describing average involved NK cells across multiple distinct pancreatic islets.

FIG. 101 shows an exemplary Effect Diagram describing average involved NK cell activation across multiple distinct pancreatic islets.

FIG. 102 shows an exemplary Effect Diagram describing average involved NK cell proliferative fraction across multiple distinct pancreatic islets.

FIG. 103 shows an exemplary Effect Diagram describing average involved apoptotic NK cells across multiple distinct pancreatic islets.

FIG. 104 shows an exemplary Effect Diagram describing average involved viable macrophages across multiple distinct pancreatic islets.

FIG. 105 shows an exemplary Effect Diagram describing average involved macrophage activation across multiple distinct pancreatic islets.

FIG. 106 shows an exemplary Effect Diagram describing average involved apoptotic macrophages across multiple distinct pancreatic islets.

FIG. 107 shows an exemplary Effect Diagram describing average involved inflammatory dendritic cells across multiple distinct pancreatic islets.

FIG. 108 shows an exemplary Effect Diagram describing average involved inflammatory dendritic cell activation across multiple distinct pancreatic islets.

FIG. 109 shows an exemplary Effect Diagram describing average involved apoptotic dendritic cells across multiple distinct pancreatic islets.

FIG. 110 shows an exemplary Effect Diagram describing average involved suppressive dendritic cells across multiple distinct pancreatic islets.

FIG. 111 shows an exemplary Effect Diagram describing average involved suppressive dendritic cell activation across multiple distinct pancreatic islets.

FIG. 112 shows an exemplary Effect Diagram describing average involved dendritic cell phenotype skewing across multiple distinct pancreatic islets.

FIG. 113 shows an exemplary Effect Diagram describing average involved conventional CD4+ T cells across multiple distinct pancreatic islets.

FIG. 114 shows an exemplary Effect Diagram describing average calculations of ROS/RNS across multiple distinct pancreatic islets.

FIG. 115 shows an exemplary Effect Diagram describing calculations relating to B lymphocytes averaged across multiple distinct pancreatic islets.

FIG. 116 shows an exemplary Effect Diagram describing calculations relating to soluble antigen averaged across multiple distinct pancreatic islets.

FIG. 117 shows an exemplary Effect Diagram describing calculations relating to β cells averaged across multiple distinct pancreatic islets.

FIG. 118 shows an exemplary Effect Diagram describing calculations relating to insulin synthesis averaged across multiple distinct pancreatic islets.

FIG. 119 shows an exemplary Effect Diagram describing calculations relating to β cell fractions averaged across multiple distinct pancreatic islets.

FIG. 120 shows an exemplary Effect Diagram describing onset of diabetic treatment.

FIG. 121 shows an exemplary Effect Diagram describing exogenous IL-10 treatment.

FIG. 122 shows an exemplary Effect Diagram describing anti-CD8 treatment.

FIGS. 123-125 show exemplary Effect Diagrams describing anti-B7.1 and anti-B7.2 treatment.

FIG. 126 shows an exemplary Effect Diagram describing Liposome-encapsulated dichloromethylene diphosphonate treatment.

FIG. 127 shows an exemplary Effect Diagram describing the effects of anti-CD3 treatment on diabetes-specific and non-specific T cells in the plasma.

FIGS. 128-132 show exemplary Effect Diagrams describing the effects of anti-CD3 treatment on diabetes-specific and non-specific T cells in pancreatic islets.

FIG. 133 shows an exemplary Effect Diagram describing the effects of anti-CD3 treatment on diabetes-specific and non-specific T cells and endothelial cells in pancreatic islets.

FIG. 134 shows an exemplary Effect Diagram describing TGF-β treatment.

FIGS. 135-137 show exemplary Effect Diagrams describing anti-CD40L treatment.

FIG. 138 shows an exemplary Effect Diagram describing exendin-4 treatment.

FIG. 139 shows an exemplary Effect Diagram describing rapamycin pharmacokinetics and effects.

FIG. 140 shows an exemplary Effect Diagram describing effects of rapamycin treatment on lymphocytes.

FIG. 141 shows an exemplary Effect Diagram describing effects of rapamycin treatment on non-lymphocytes.

FIG. 142 shows an exemplary Effect Diagram describing oral insulin dosing and flow to all tissues.

FIG. 143 shows an exemplary Effect Diagram describing dendritic cell recruitment and life cycle in gut.

FIG. 144 shows an exemplary Effect Diagram describing dendritic cell activation and surface molecule expression in gut.

FIGS. 145 and 146 show exemplary Effect Diagrams describing dendritic cell and macrophage antigen loading in gut.

FIG. 147 shows an exemplary Effect Diagram describing dendritic cell surface molecule expression and antigen trafficking to gut associated lymphoid tissue (GALT).

FIG. 148 shows an exemplary Effect Diagram describing dendritic cell surface molecule expression in gut and antigen trafficking to pancreatic lymph nodes.

FIG. 149 shows an exemplary Effect Diagram describing dendritic cell and macrophage calculations in gut.

FIG. 150 shows an exemplary Effect Diagram describing conventional CD4+ T cell recruitment and life cycle in gut associated lymphoid tissue.

FIG. 151 shows an exemplary Effect Diagram describing conventional CD4+ T cell priming in gut associated lymphoid tissue.

FIG. 152 shows an exemplary Effect Diagram describing CD4+ T cell and innate regulatory T cell life cycle regulation in gut associated lymphoid tissue.

FIG. 153 shows an exemplary Effect Diagram describing conventional CD4+ T cell differentiation calculations in gut associated lymphoid tissue.

FIG. 154 shows an exemplary Effect Diagram describing innate regulatory T cell recruitment and life cycle in gut associated lymphoid tissue.

FIG. 155 shows an exemplary Effect Diagram describing innate regulatory T cell activation in gut associated lymphoid tissue.

FIG. 156 shows an exemplary Effect Diagram describing CD4+ T cell and innate regulatory T cell mediator synthesis in gut associated lymphoid tissue.

FIG. 157 shows an exemplary Effect Diagram describing T cell calculations in gut associated lymphoid tissue.

FIG. 158 shows an exemplary Effect Diagram describing CD8+ T cell recruitment and life cycle in gut associated lymphoid tissue.

FIG. 159 shows an exemplary Effect Diagram describing CD8+ T cell activation in gut associated lymphoid tissue.

FIG. 160 shows an exemplary Effect Diagram describing CD8+ T cell life cycle regulation and mediator synthesis in gut associated lymphoid tissue.

FIG. 161 shows an exemplary Effect Diagram describing CD8+ T cell calculations in gut associated lymphoid tissue.

FIG. 162 shows an exemplary Effect Diagram describing B lymphocyte recruitment and life cycle in gut associated lymphoid tissue.

FIG. 163 shows an exemplary Effect Diagram describing B lymphocyte activation in gut associated lymphoid tissue.

FIG. 164 shows an exemplary Effect Diagram describing B lymphocyte life cycle regulation, mediator synthesis and antibody production in gut associated lymphoid tissue.

FIG. 165 shows an exemplary Effect Diagram describing B lymphocyte calculations in gut associated lymphoid tissue.

FIG. 166 shows an exemplary Effect Diagram describing NK cell recruitment and life cycle in gut associated lymphoid tissue.

FIG. 167 shows an exemplary Effect Diagram describing NK cell activation in gut associated lymphoid tissue.

FIG. 168 shows an exemplary Effect Diagram describing NK cell life cycle regulation in gut associated lymphoid tissue.

FIG. 169 shows an exemplary Effect Diagram describing NK cell mediator synthesis in gut associated lymphoid tissue.

FIG. 170 shows an exemplary Effect Diagram describing dendritic cell and macrophage trafficking and life cycle in gut associated lymphoid tissue.

FIG. 171 shows an exemplary Effect Diagram describing dendritic cell activation and surface molecule expression in gut associated lymphoid tissue.

FIG. 172 shows an exemplary Effect Diagram describing dendritic cell mediator synthesis in gut associated lymphoid tissue.

FIG. 173 shows an exemplary Effect Diagram describing dendritic cell and macrophage calculations in gut associated lymphoid tissue.

FIG. 174 shows an exemplary Effect Diagram describing antigen presenting cells in gut associated lymphoid tissue.

FIG. 175 shows an exemplary Effect Diagram describing macrophage activation in gut associated lymphoid tissue.

FIG. 176 shows an exemplary Effect Diagram describing macrophage mediator synthesis and dendritic cell/macrophage:T cell contact in gut associated lymphoid tissue.

FIG. 177 shows an exemplary Effect Diagram describing Tissue composition and volume calculations in gut associated lymphoid tissue.

FIG. 178 shows an exemplary Effect Diagram describing T cell surface molecule expression and cell contact in gut associated lymphoid tissue.

FIGS. 179 and 180 show exemplary Effect Diagrams describing total mediator production in gut associated lymphoid tissue.

FIG. 181 shows an exemplary Effect Diagram describing CD4+ T cell and innate regulatory T cell calculations in gut associated lymphoid tissue.

FIG. 182 shows an exemplary Effect Diagram describing oral insulin peptide antigen transfer to antigen presenting cells in pancreatic lymph nodes.

FIG. 183 shows an exemplary Effect Diagram describing oral insulin peptide antigen presentation in pancreatic lymph nodes.

FIGS. 184 and 185 show exemplary Effect Diagrams describing apoptosis at high activation in pancreatic lymph nodes.

FIG. 186 shows an exemplary Effect Diagram describing oral insulin peptide antigen transfer to antigen presenting cells in gut associated lymphoid tissue.

FIG. 187 shows an exemplary Effect Diagram describing oral insulin peptide antigen presentation in gut associated lymphoid tissue.

FIGS. 188 and 189 show exemplary Effect Diagrams describing apoptosis at high activation in gut associated lymphoid tissue.

FIG. 190 shows an exemplary Effect Diagram describing oral insulin peptide antigen transfer to antigen presenting cells in pancreatic islets.

FIG. 191 shows an exemplary Effect Diagram describing oral insulin peptide antigen presentation in pancreatic islets.

FIGS. 192 and 193 show exemplary Effect Diagrams describing apoptosis at high activation in pancreatic islets.

FIGS. 194 and 195 show exemplary Effect Diagrams describing progressive loss of immune regulation with disease development.

### DETAILED DESCRIPTION

A. Overview

The invention encompasses novel methods for developing a computer model of type 1 diabetes in a mammal. In particular, the models can include representations of biological processes associated with a pancreatic lymph node and one or more pancreatic islets. Alternatively, the models can include representations of biological processes associated with at least two conditions selected from the group consisting of autoreactive T cell production, autoreactive T cell priming, insulitis and hyperglycemia. The invention also provides methods for developing a computer model of a non-insulin replacement treatment of type 1 diabetes. The invention also encompasses computer models of type 1 diabetes, methods of simulating type 1 diabetes and computer systems for simulating type 1 diabetes and the uses thereof.

B. Definitions

A “biological system” can include, for example, an individual cell, a collection of cells such as a cell culture, an organ, a tissue, a multi-cellular organism such as an individual human patient, a subset of cells of a multi-cellular organism, or a population of multi-cellular organisms such as a group of human patients or the general human population as a whole. A biological system can also include, for example, a multi-tissue system such as the nervous system, immune system, or cardio-vascular system.

The term “biological component” refers to a portion of a biological system. A biological component that is part of a biological system can include, for example, an extra-cellular constituent, a cellular constituent, an intra-cellular constituent, or a combination of them. Examples of suitable biological components, include, but are not limited to, metabolites, DNA, RNA, proteins, surface and intracellular receptors, enzymes, lipid molecules (i.e., free cholesterol, cholesterol ester, triglycerides, and phospholipid), hormones, cells, organs, tissues, portions of cells, tissues, or organs, subcellular organelles, chemically reactive molecules like H⁺, superoxides, ATP, as well as, combinations or aggregate representations of these types of biological variables. In addition, biological components can include therapeutic agents such as an anti-CD3 antibody (e.g., 145.2C11), an anti-CD8 antibody, liposomal dichloromethylene diphosphonate, exogenous IL-10, an anti-B7.1 antibody, an anti-B7.2 antibody, oral insulin, exogenous TGF-β, exendin-4, an anti-CD40L antibody, rapamycin or an anti-IL-2 antibody

The term “biological process” is used herein to mean an interaction or series of interactions between biological components. Examples of suitable biological processes, include, but are not limited to, activation, apoptosis or recruitment of certain cells, such as macrophages, mucus secretion, vascular permeability, mediator production, and the like. The term “biological process” can also include a process comprising one or more therapeutic agents, for example the process of binding a therapeutic agent to a cellular mediator. Each biological variable of the biological process can be influenced, for example, by at least one other biological variable in the biological process by some biological mechanism, which need not be specified or even understood.

The term “parameter” is used herein to mean a value that characterizes the interaction between two or more biological components. Examples of parameters include affinity constants, Kₘ, K_{d}, k_{cat}, half life, or net flux of cells, such macrophages or dendritic cells, into particular tissues.

The term “variable,” as used herein refers to a value that characterizes a biological component. Examples of variables include the total number of T cells, the number of active or inactive macrophages, and the concentration of a mediator, such as soluble mediators (e.g., TNF-α, IFN-γ, insulin, or ROS/RNS) or cell surface molecules (e.g., MHC class I, Fas).

The term “phenotype” is used herein to mean the result of the occurrence of a series of biological processes. As the biological processes change relative to each other, the phenotype also undergoes changes. One measurement of a phenotype is the level of activity of variables, parameters, and/or biological processes at a specified time and under specified experimental or environmental conditions.

A phenotype can include, for example, the state of an individual cell, an organ, a tissue, and/or a multi-cellular organism. Organisms useful in the methods and models disclosed herein include animals. The term “animal” as used herein includes mammals, such as humans. A phenotype can also include, but is not limited to, behavior of the system as a whole, as measured by blood glucose concentration, autoantibody levels, inflammatory/regulatory mediator levels, or inflammatory/regulatory cell populations. The conditions defined by a phenotype can be imposed experimentally, or can be conditions present in a patient type.

The term “disease state” is used herein to mean a phenotype where one or more biological processes are related to the cause or the clinical signs of the disease. For example, a disease state can be the state of a diseased cell, a diseased organ, a diseased tissue, or a diseased multi-cellular organism. A diseased multi-cellular organism can be, for example, a NOD mouse or an individual human patient. A diseased state can also include, for example, a defective enzyme or the overproduction of an immune mediator.

The term “simulation” is used herein to mean the numerical or analytical integration of a mathematical model. For example, simulation can mean the numerical integration of the mathematical model of the phenotype defined by an equation, such as dx/dt=f(x, p, t).

The term “biological characteristic” is used herein to refer to a trait, quality, or property of a particular phenotype of a biological system. For example, biological characteristics of a particular disease state include clinical signs and diagnostic criteria associated with the disease. The biological characteristics of a biological system can be measurements of biological variables, parameters, and/or processes. Suitable examples of biological characteristics associated with a type 1 diabetic state include, but are not limited to, measurements of immune cell populations, insulitis, beta cell mass, insulin production, and plasma glucose concentrations.

The term “computer-readable medium” is used herein to include any medium which is capable of storing or encoding a sequence of instructions for performing the methods described herein and can include, but not limited to, optical and/or magnetic storage devices and/or disks, and carrier wave signals.

C. Methods of Developing Models of Type 1 Diabetes

A computer model can be designed to model one or more biological processes or functions. The computer model can be built using a “top-down” approach that begins by defining a general set of behaviors indicative of a biological condition, e.g. a disease. The behaviors are then used as constraints on the system and a set of nested subsystems are developed to define the next level of underlying detail. For example, given a behavior such as beta cell destruction in type 1 diabetes, the specific mechanisms inducing the behavior are each be modeled in turn, yielding a set of subsystems, which can themselves be deconstructed and modeled in detail. The control and context of these subsystems is, therefore, already defined by the behaviors that characterize the dynamics of the system as a whole. The deconstruction process continues modeling more and more biology, from the top down, until there is enough detail to replicate a given biological behavior. Specifically, the model is capable of modeling biological processes that can be manipulated by a drug or other therapeutic agent.

An overview of the methods used to develop computer models of type 1 diabetes is illustrated in FIG. 1. The methods typically begin by identifying one or more biological processes associated with a pancreatic lymph node and one or more biological processes associated with one or more β islets. The identification of biological processes associated with pancreatic lymph nodes or pancreatic islets can be informed by data relating to the metabolic system, the pancreas or any portion thereof. Optionally, the method can also comprise the step of identifying one or biological processes associated with gut and/or gut associated lymphoid tissue. The method next comprises the step of mathematically representing each identified biological process. The biological processes can be mathematically represented in any of a variety of manners. Typically, the biological process is defined by equations having the form, dx/dt=f(x, p, t), as described below. The representations of biological processes associated with a pancreatic lymph node and with one or more pancreatic islets are combined, thus forming predictive models of type 1 diabetes. The methods may further include the steps of identifying and mathematically representing one or more biological processes associated with gut and/or gut associated lymphoid tissue

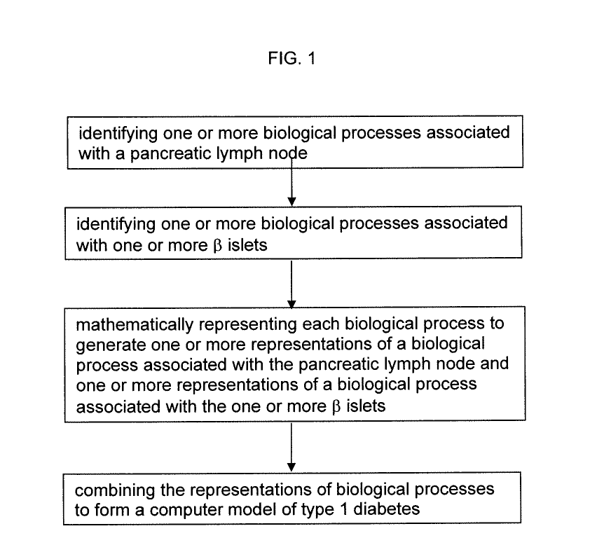

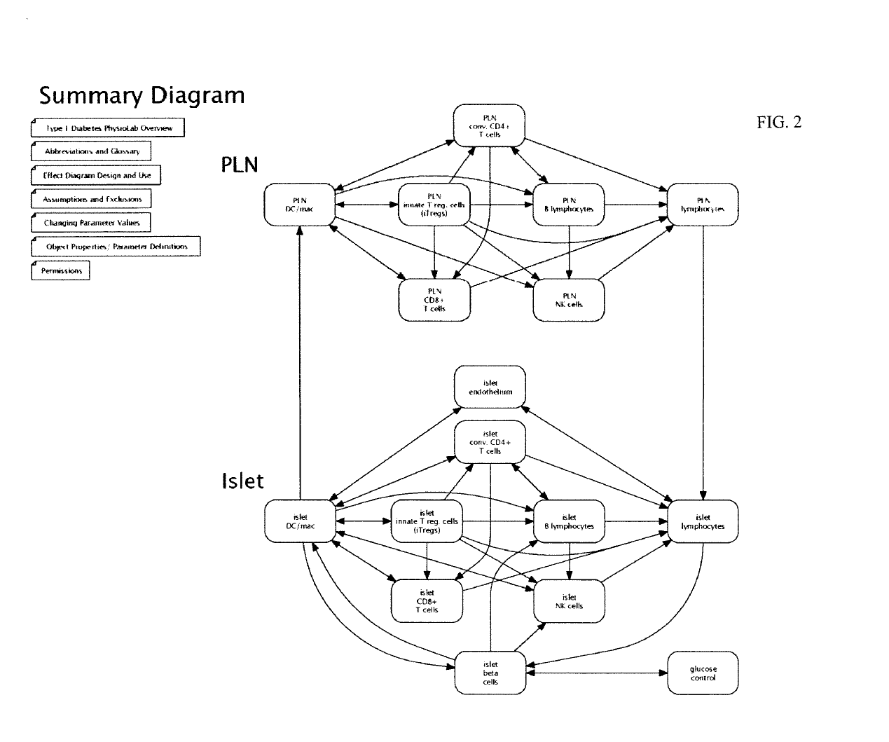

FIG. 2 illustrates an exemplary Summary Diagram that links modules relating to biological processes associated with pancreatic lymph nodes, islets and other biological compartments. In one implementation of the invention, a primary measure of the progression or severity of type 1 diabetes is glucose control, as exemplified by plasma glucose concentrations. Two primary biological compartments affect the autoimmune response targeting beta cells and thereby glucose control: the pancreatic lymph node and the pancreatic islets. Each of these compartments is dynamically responsive to changes in the environment and the phenotype of a subject.

In a preferred implementation of the invention, identifying a biological process associated with a pancreatic lymph node comprises identifying a biological process related to pancreatic lymph node leukocytes. Preferably the pancreatic lymph node leukocytes include at least one of the group consisting of antigen presenting cells (e.g., dendritic cells, macrophages and B lymphocytes), CD4+ T cells, CD8+ T cells, B lymphocytes, innate regulatory T cells and NK cells. Similarly, identifying a biological process associated with one more pancreatic islets can comprise identifying a biological process related to islet leukocytes, islet β cells, or islet endothelium. Preferably the islet leukocytes include at least one of the group consisting of antigen presenting cells (e.g., dendritic cells, macrophages and B lymphocytes), CD4+ T cells, CD8+ T cells, B lymphocytes, innate regulatory T cells and NK cells.

Once one or more biological processes are identified in the context of the methods of the invention, each biological process is mathematically represented. For example, the computer model can represent a first biological process using a first mathematical relation and a second biological process using a second mathematical relation. A mathematical relation typically includes one or more variables, the behavior (e.g., time evolution) of which can be simulated by the computer model. More particularly, mathematical relations of the computer model can define interactions among variables describing levels or activities of various biological components of the biological system as well as levels or activities of combinations or aggregate representations of the various biological components. In addition, variables can represent various stimuli that can be applied to the physiological system. The mathematical model(s) of the computer-executable software code represents the dynamic biological processes related to biological function. The form of the mathematical equations employed may include, for example partial differential equations, stochastic differential equations, differential algebraic equations, difference equations, cellular automata, coupled maps, equations of networks of Boolean or fuzzy logical networks, etc.

In some embodiments, the mathematical equations used in the model are ordinary differential equations of the form:
dx/dt=f(x,p,t)
where x is an N dimensional vector whose elements represent the biological variables of the system, t is time, dx/dt is the rate of change of x, p is an M dimensional set of system parameters, and f is a function that represents the complex interactions among biological variables. In one implementation, the parameters are used to represent intrinsic characteristics (e.g., genetic factors) as well as external characteristics (e.g., environmental factors) of a biological system.

In some embodiments, the phenotype can be mathematically defined by the values of x and p at a given time. Once a phenotype of the model is mathematically specified, numerical integration of the above equation using a computer determines, for example, the time evolution of the biological variables x(t) and hence the evolution of the phenotype over time.

The representation of the biological processes are combined to generate a model of type 1 diabetes, a model of progression of type 1 diabetes, or a model of a non-insulin replacement treatment of diabetes. Generation of models of biological systems are described, for example, in U.S. Pat. Nos. 5,657,255 and 5,808,918, entitled “Hierarchical Biological Modeling System and Method”; U.S. Pat. No. 5,914,891, entitled “System and Method for Simulating Operation of Biochemical Systems”; U.S. Pat. No. 5,930,154, entitled “Computer-based System and Methods for Information Storage, Modeling and Simulation of Complex Systems Organized in Discrete Compartments in Time and Space”; U.S. Pat. No. 6,051,029, entitled “Method of Generating a Display for a Dynamic Simulation Model Utilizing Node and Link Representations”; U.S. Pat. No. 6,069,629, entitled “Method of Providing Access to Object Parameters Within a Simulation Model”; U.S. Pat. No. 6,078,739, entitled “A Method of Managing Objects and Parameter Values Associated With the Objects Within a Simulation Model”; U.S. Pat. No. 6,539,347, entitled “Method of Generating a Display For a Dynamic Simulation Model Utilizing Node and Link Representations”; U.S. Application Publication No. 20010032068, entitled “Method and Apparatus for Conducting Linked Simulation Operations Utilizing a Computer-Based System Model”; and PCT publication WO 99/27443, entitled “A Method of Monitoring Values within a Simulation Model”.

The methods can further comprise methods for validating the computer models described herein. For example, the methods can include generating a simulated biological characteristic associated with type 1 diabetes in an animal, and comparing the simulated biological characteristic with a corresponding reference biological characteristic measured in a normal or diseased animal. The result of this comparison in combination with known dynamic constraints may confirm some part of the model, or may point the user to a change of a mathematical relationship within the model, which improves the overall fidelity of the model. Methods for validating the various models described herein are taught in U.S. Patent Publication 2002-0193979, entitled “Apparatus And Method For Validating A Computer Model, and in U.S. Pat. No. 6,862,561, entitled “Method and Apparatus for Computer Modeling a Joint”, the disclosures of which are incorporated herein by reference.

One application of a NOD mouse type 1 diabetes model is to improve understanding of type 1 diabetes pathogenesis in the NOD mouse. Model development therefore focused on islet β cell autoimmunity and tolerance, as well as related mechanisms and interventions. To provide for general immunologic function and not only progression to diabetes, the development effort included representation of several NOD mouse phenotypes. The first phenotype was an average diabetic virtual mouse. This virtual mouse exhibited characteristic insulitis and develops diabetes at some time between twelve and thirty-five weeks of age if untreated. Typically, a simulation of the average virtual mouse concludes shortly after diabetes diagnosis, which is consistent with post-diabetic sacrifice of NOD mice in the laboratory. This virtual mouse also reflected the average phenotype for female NOD mice that progress to diabetes within a colony exhibiting a high incidence (>60%) of diabetes among untreated females. Additional virtual mice, each with different clinical behaviors and/or underlying pathophysiologies, have also been generated. Similarly, development of modals of type 1 diabetes in other mammals, such as humans, can include the development of various virtual patients representing normal animals or animals having varying degrees of severity or progression of type 1 diabetes.

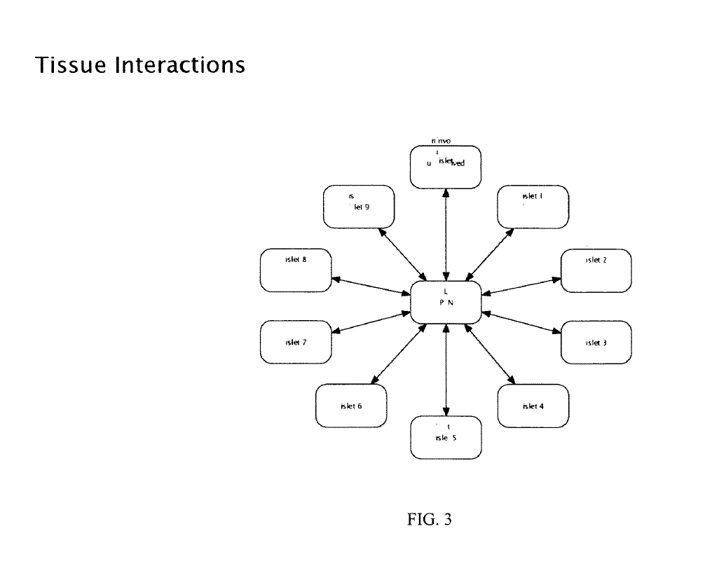

In type 1 diabetes, the diseased pancreas is characterized by extensive pathological heterogeneity. One critical aspect of this heterogeneity is the simultaneous presence of islets at different stages in the disease process (e.g., no infiltration, minimal infiltration, extensive infiltration, complete destruction). In order to accurately represent disease progression and treatment of type 1 diabetes, the model included the representation of distinct distributed sites (i.e., islets) within the pancreas that can demonstrate varying degrees of involvement (leukocyte infiltration) over the course of disease progression (FIG. 3).

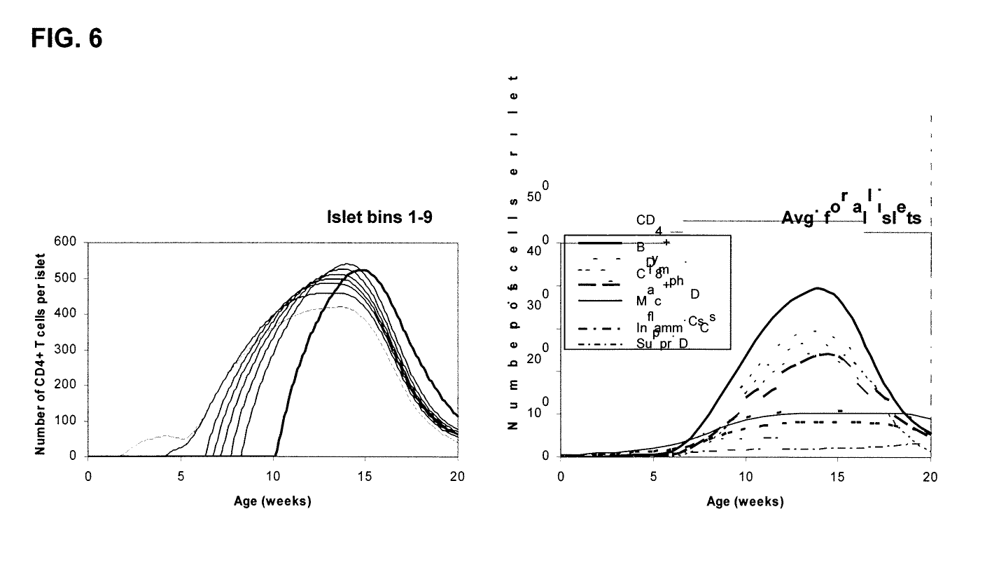

As shown in FIG. 6, more distinct distributed sites become involved over the course of disease progression, and at particular times (e.g., 10 weeks of age), some sites may be characterized by heavy infiltration, while others are yet uninvolved. The relative contributions from each site are combined to yield the average infiltrate across the total pancreas.

Because diabetes is typically assessed by measuring blood or urinary glucose, blood glucose levels were represented in the model's clinical read-out. Hyperglycemia (a diabetes indicator) was defined as a glucose level in excess of a commonly used standard (e.g., 200, 250, 300, 350 mg/dl). Glucose control was regulated based on a mathematical relationship describing interactions between blood insulin and glucose concentrations.

Selected interventions tested in the NOD mouse were also represented. These interventions were used to calibrate or validate the representation of diabetes pathogenesis in the NOD mouse, and include interventions that target several different pathways of the disease, including, for example: cytokine levels, costimulatory molecules, T cell populations, and antigen presenting cell (APC) populations.

In the untreated condition, the average diabetic virtual mouse was required to exhibit a set of behaviors that are representative of female NOD mice in colonies with a high incidence (>60%) of diabetes. These include (i) dynamics of diabetes onset, (ii) dynamics of pancreatic lymph node (PLN) lymphocyte activation and expansion, and (iii) dynamics of islet infiltrate.

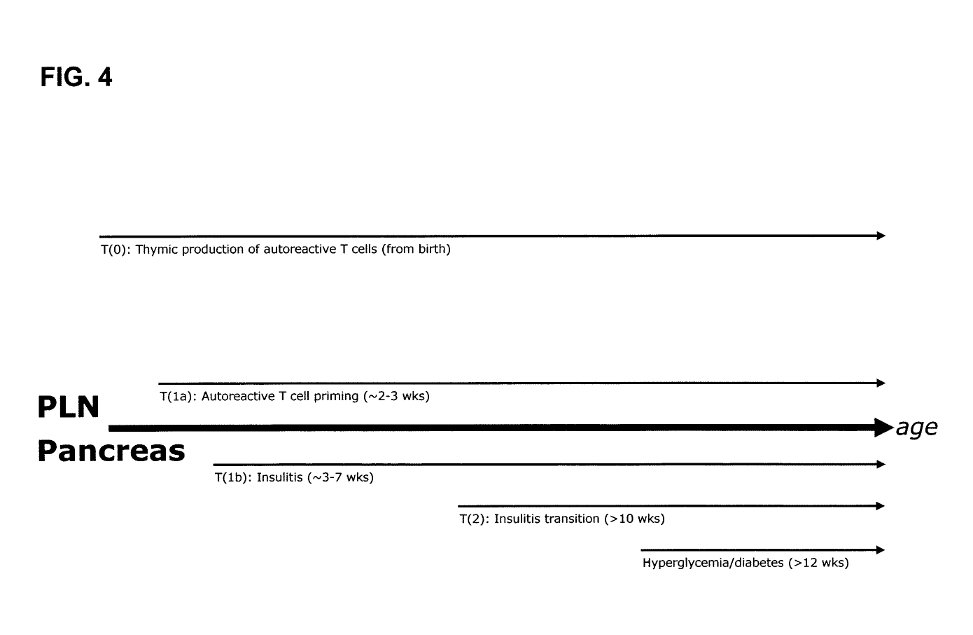

Pathogenesis of type 1 diabetes in NOD mice can be characterized by a series of distinct events, also called checkpoints (FIG. 4). An early requirement is thymic generation of auto-reactive T lymphocytes (Checkpoint 0). At an early age, these naïve auto-reactive T lymphocytes become activated in the pancreatic lymph nodes (Checkpoint 1a). T lymphocytes are activated by cross presentation of autoantigen by professional antigen-presenting cells and then undergo clonal expansion. This priming event occurs at approximately two to three weeks of age and may be driven in part by developmental remodeling in the pancreas or developmental maturation of antigen-presenting cells. T lymphocyte priming leads to an influx of T lymphocytes and other inflammatory cells into the pancreas at approximately three to seven weeks of age (Checkpoint 1b). This influx initially creates a non-destructive insulitis, in which the inflammatory infiltrate accumulates in and around the islets, but does not result in detectable net β cell loss. At some time after approximately ten weeks of age, inflammatory cells become more pervasive in the islets, and a net loss of β cells becomes detectable (Checkpoint 2). This cellular destruction eventually leads to a loss of insulin secretion and glucose control, leading to hyperglycemia usually after twelve weeks of age. The diabetic virtual mouse reproduces this general pattern of disease progression.

Accordingly, one aspect of the invention provides methods of developing computer models of the progression of type 1 diabetes, the methods comprising: identifying one or more biological processes associated with development of each of at least two conditions selected from the group consisting of autoreactive T cell production, autoreactive T cell priming, insulitis and hyperglycemia; mathematically representing each biological process to generate one or more representations of a biological process associated with each of the at least two conditions; and combining the representations of biological processes to form a model of progression of type 1 diabetes. The model can include a representation of the onset, existence, progression or transition from any of these conditions.

The first stage of developing a computer model of type 1 diabetes included an extensive review of the public literature regarding the disease and its underlying biology. This information was used to define the scope of the diabetes model, which is based on β cell autoimmunity and tolerance in type 1 diabetic non-obese diabetic (NOD) mice. The strategy focused on the development of a core immunology model that would reproduce natural disease progression and facilitate research on type 1 diabetes pathogenesis in the NOD mouse. Information developed from the NOD mouse type 1 diabetes model could then be translated to develop a human type 1 diabetes model.

Activities undertaken in constructing the model include (i) identification of the biological areas and behaviors that are important to type 1 diabetes pathogenesis in the NOD mouse; (ii) development of the NOD mouse type 1 diabetes model and integration of the knowledge of disease mechanisms into a coherent and unifying context of disease pathogenesis; (iii) development of scientific insights about the disease identified through the development of and research using the NOD mouse type 1 diabetes model, and (iv) determination of a strategy likely to successfully and rapidly translate insights gained through the NOD mouse type 1 diabetes model to human type 1 diabetes patients.

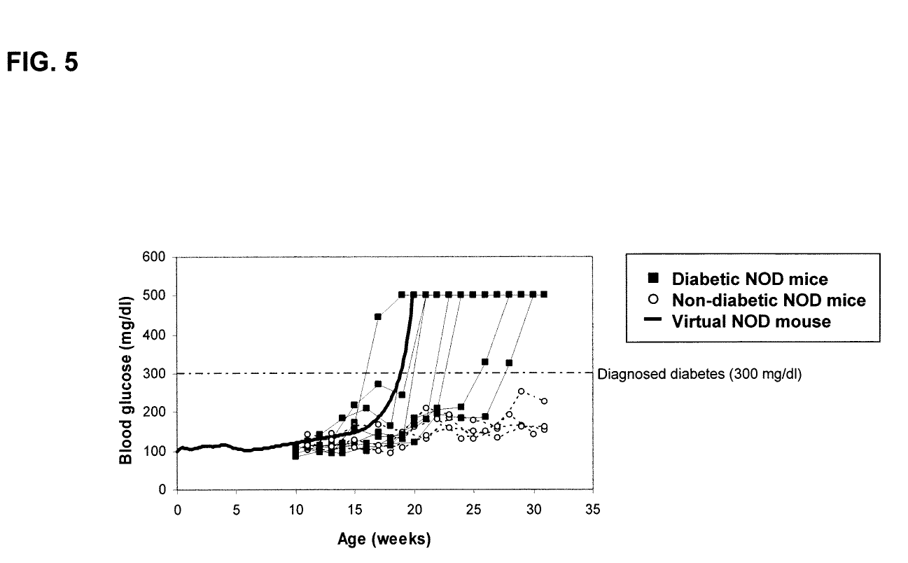

In an exemplary embodiment of a type 1 diabetes model, the average diabetic virtual NOD mouse exhibited characteristic insulitis and developed diabetes between twelve and thirty-five weeks of age if untreated, a time frame that is consistent with laboratory female NOD mice in a colony with reasonably high diabetes incidence (>60%; FIG. 5). Overall, the model is consistent with the existing data on PLN leukocyte expansion, for all leukocytes represented, and with leukocytic infiltration of the islets (data not shown). Islet leukocytic infiltration reflects the combined contributions from the distinct distributed sites (islet bins) throughout disease progression.

In a preferred embodiment of the invention, the methods of developing a computer model includes representing and integrating professional antigen-presenting cells (macrophages, dendritic cells and B lymphocytes), NK cells, and CD4+ and CD8+ T lymphocytes in the pancreatic lymph nodes and islets. These cell types are thought to be important for the initiation and progression of the autoimmune response, as well as for the effector stage of β cell destruction. Following integration of these modules, the computer model can simulate certain behaviors analogous to in vitro behaviors, including the uptake of exogenous antigen by macrophages, dendritic cells, and B lymphocytes, antigen presentation to CD4+ and CD8+ T lymphocytes, regulated lymphocyte differentiation and expansion, acquisition of diabetogenic effector activity, the dynamics of antigen presenting cell (APC) trafficking from the pancreas to pancreatic lymph nodes, and pancreatic infiltration by immune cells.

Developing a computer model of type 1 diabetes can further comprise representing and integrating antigen generation, tissue destruction, and glucose control. This can involve representing islet β cells, which both supply autoantigen and serve as the target of the autoimmune attack. The model can also represent the role of antigen presenting cells in immune priming and inflammation, T lymphocyte response and contribution to infiltration into the pancreas, and how multiple immune cell types can interact with and destroy β cells. Representation of the blood glucose concentration, which is regulated by β cell-derived insulin, provides an experimental output. Since all elements important for simulating β cell destruction are present, the integration of these permits an initial simulation of disease progression.

In certain implementations, developing a computer model of type 1 diabetes can further comprise representing endothelial adhesion molecules and an innate regulatory T cell population (iTregs). Endothelial cells regulate the entry of inflammatory cells into the pancreas, and some potential therapeutics have targeted leukocyte-endothelial cell interactions. One hypothesized driver of type 1 diabetes is a deficiency in innate regulatory T cells. These cells may modulate the rate of disease progression in natural pathogenesis, limit progression in resistant phenotypes, and mediate the efficacy of some therapeutics. When diabetogenic and regulatory elements within the model scope were included, integration of these modules resulted in a full representation of natural disease progression in the average diabetic animal, including explicit representation of the timing and magnitude of key events in disease progression and reproduction of the diabetic phenotype.

Developing the model additionally can comprise calibrating and validating the model. For example, calibration of a NOD mouse type 1 diabetes model can include representation of natural disease progression as well as appropriate responses to selected interventions. Accordingly, developing the model can comprise reproducing natural disease progression for the average diabetic phenotype, as well as the appropriate responses to interventions. In addition, other virtual mice representing different phenotypes (e.g., early development of frank diabetes) or different hypotheses on the underlying pathophysiology can be created.

Successful treatment of type 1 diabetes in rodents is assisted by a wealth of rodent immunology data. Unfortunately, the dearth of human disease and immunology data makes predicting human responses to these same treatments highly uncertain. To address these challenges, the inventors have devised a two part component approach: (1) representation and investigation of type 1 diabetes immunology and pathogenesis as manifested in the NOD mouse, and (2) representation and investigation of human type 1 diabetes immunology and pathogenesis using the original modeled mouse biology as a basis for the human condition.

A goal of the first component, NOD mouse type 1 diabetes model, is to integrate and expand the current understanding of pathways involved in β cell autoimmunity and tolerance into the unified context of type 1 diabetes pathogenesis in the NOD mouse. The development and use of a NOD mouse type 1 diabetes model provides scientific insights into type 1 diabetes pathogenesis, as well as recommendations for experimental validation of those insights. An increased understanding of pathogenesis in the NOD mouse is expected to reveal novel treatment strategies and to improve the predictive value of this rodent model in developing human treatments.

The second component focuses on human type 1 diabetes. Here, the goal is to appropriately modify the representation of type 1 diabetes in the NOD mouse to represent the human disease. The ability to apply the understanding gained through development of the NOD mouse type 1 diabetes model to represent the human disease in a manner consistent with its clinical manifestations has significant potential for human type 1 diabetes research, where pathogenesis is poorly understood and difficult to study. A human type 1 diabetes model could be used to assist in (i) the identification of mechanisms underlying human type 1 diabetes pathogenesis, (ii) the development of novel therapeutic strategies, and (iii) the initial prediction of human response to novel or preclinical therapies.

Where the known human biology is found to differ from the NOD mouse, the parameter values and, potentially, structure of the model can be modified to represent the human biology. In addition, since human clinical trials commonly measure fasting or mixed meal C-peptide levels, the representation of glucose and insulin dynamics in the model can be modified to enable the calculation of these measures. The platform will also be expanded to represent exogenous insulin administration because this treatment typically commences immediately upon diagnosis of diabetes in humans.

After making these modifications, the initial human representation can be evaluated for its consistency with both clinical and subclinical human data. Clinical data include studies reporting the age range for type 1 diabetes onset, as well as the progressive decline in C-peptide levels in diagnosed patients. Subclinically, there are cross-sectional data on the character of the inflammatory infiltrate and the level of β cell destruction.

Although a number of differences between human and mouse biology can be implemented in the model, these differences may be insufficient to account for the observed differences in disease manifestation and therapeutic response. In this case, hypotheses can be formulated to reconcile the remaining discrepancies. Formulation of hypotheses can be based on the literature, on experimental or clinical data, on the key disease-modulating pathways identified in the NOD representation, on variations in pathogenesis that resulted in disease outcomes in mice that are similar to what is needed in the human, or on insights into pathogenesis and dynamics from execution of the model of diabetes in NOD mice. For example, the identification of key disease-modulating pathways can determine which pathways most significantly affect the timing of disease onset in the NOD mouse. If necessary, hypotheses accounting for the delay in human onset can be formulated that impact these pathways and tested to determine the degree of difference required to achieve human timing of disease onset.

In recent years, the successful anti-CD3 treatment of diabetic mice has motivated clinical trials in recent onset T1D patients. Anti-CD3 studies in the NOD mouse reveal complex dependencies on timing of administration and dose. A single high dose (200-250 μg) injection of anti-CD3 antibody 145-2C11 led to extensive T cell depletion and significant protection in animals treated at 1 day or 1 week, but not 3 weeks of age (Hayward & Shriber, J Autoimmun. 5, 59-67 (1992); Hayward & Shreiber, J Immunol 143, 1555-1559 (1989)). The same antibody administered in low doses (5 μg/day for 5 days), afforded no protection in 4 and 8 week old mice, slightly delayed onset in 12 week old mice, but induced remission in 50 100% of diabetic mice (Chatenoud, et al. C. R. Acad Sci III 315, 225-228 (1992); Chatenoud, et al. J Immunol 158, 2947-2954 (1997); Chatenoud, et al. Proc Natl Acad Sci USA 91, 123-127 (1994)). Thus, the high (depleting) dose appears effective in neonatal but not pre-diabetic mice; whereas the lower dose appears most effective in diabetic mice. In remission studies, low dose 145-2C11 induced only partial (52%) depletion of peripheral T cells (Chatenoud, et al. J Immunol 158, 2947-2954 (1997)). The proposed mechanism of remission was Treg stimulation, supported by the finding that co administration of either anti-CTLA 4 or anti-TGF-β with anti-CD3 prevented remission (Belghith, et al. Nat Med 9, 1202-1208 (2003)), although secondary effects of hyperglycemia itself might contribute to anti-CD3 efficacy in diabetic animals (Kojima, et al. Proc Natl Acad Sci USA 101, 2458-2463 (2004)). Thus, time and dose dependent variations in outcome may be partly explained by different mechanisms of action.

Further studies on diabetic mice provide additional insight into the proposed anti-CD3 mechanisms. Using 145-2C11 (5 μg/day, 5 days), several groups reported 64-81% remission coincident with partial T cell depletion (Chatenoud, et al. C. R. Acad Sci III 315, 225-228 (1992); Chatenoud, et al. J Immunol 158, 2947-2954 (1997); Chatenoud, et al. Proc Natl Acad Sci USA 91, 123-127 (1994)); whereas, Mottram et al (Transpl. Immunol 10, 63-72 (2002)) observed lower remission rates (14.3%) with no depletion. Additional data suggest that 145-2C11 selectively depletes effector cells relative to regulatory cells (Yang, et al. J Immunol 173, 4407-4416 (2004)). Finally, a different anti-CD3 antibody (KT3) induced remission in diabetic NOD mice administered low, non depleting doses; whereas high, fully depleting doses failed to induce remission (Mottram, et al. Transpl. Immunol 10, 63-72 (2002)). Thus in diabetic mice, partial depletion may also contribute to efficacy through selective effector cell depletion and regulatory cell enrichment, while complete CD3 depletion, which eliminates effector and regulatory cells, may prevent the re-balancing of the immune response towards a more regulatory state.

Unlike the unmodified, mitogenic 145-2C11 antibody commonly used in NOD mice, the hOKT3γ1 (Ala-Ala) antibody used in human clinical trials is a modified, dimeric non-FcR binding anti CD3 antibody. Significant differences between these agents preclude detailed comparative analysis of mechanisms of action and dosing. However, anti-CD3 clinical trials were conducted on recent onset diabetic patients, whose inclusion is supported by the time dependent efficacy observed in NOD mice, and the human results so far are promising (Herold, et al. N. Engl. J Med 346, 1692-1698 (2002)). Thus, the limited comparison possible indicates concordance between human and NOD protocols and results.

Similar protocol discrepancies were observed in comparative analyses of other agents tested in human clinical trials. Azathioprine, Bacille Calmette-Guerin (BCG), pentoxyfilline, and linomide were only tested in pre-diabetic mice, while corresponding clinical trials were conducted on diabetic patients. Furthermore, nicotinamide, BCG, and linomide were administered at much lower doses in humans than in mice on a body weight basis.

Protocol discrepancies do not account for all unexpected clinical results. For therapies such as nicotinamide and oral insulin, differential efficacy was observed in preclinical models, with protection in pre-diabetic NOD mice but not in BB rats (Yamada, et al. Diabetes 31, 749-753 (1982); Zhang, et al. Proc Natl Acad Sci USA 88, 10252-10256 (1991); Hermitte, et al. Autoimmunity 5, 79-86 (1989); Mordes, et al. Ann N.Y. Acad Sci 778:418-21, 418-421 (1996)). For future clinical trials, efforts to understand the agent mediated mechanisms accounting for different outcomes may help determine which, if either finding is more relevant to humans. For example, efforts to characterize human vs. NOD mouse vs. BB rat β cell sensitivity to nicotinamide may have suggested that human β cells were more similar to rat than mouse. Finally, species differences in β cell susceptibility to apoptosis (Eizirik, et al. Proc. Natl. Acad. Sci. U.S A 91, 9253-9256 (1994)), expression of immunogenic proteins (Kim, et al. Diabetes 42, 1799-1808 (1993)), and other immune system differences (Mestas & Hughes, J Immunol 172, 2731-2738 (2004)), could influence trial outcomes. Models of type 1 diabetes in humans can be developed to reflect most or all of these intraspecies differences, thereby providing reliable simulated predictions of the development and progression of type 1 diabetes in humans.

Because timing, dose, and species specific biology can critically impact therapeutic efficacy, measures to identify and minimize such dependencies may improve future efforts in clinical translation. Prior to human clinical trials, the relationship between therapeutic efficacy and disease stage could be thoroughly characterized. Analysis of dose-dependency in preclinical models should help identify potential dose sensitivities, and thorough characterization of drug PK/PD should be used to guide selection of human dosing strategies. In addition, therapies should be tested in multiple animal models. Consistent findings would increase translational confidence, while inconsistencies should encourage further analysis or research to determine which model results might be more applicable to humans. Finally, mathematical models, including the T1D model driving this analysis effort, may be used to improve our understanding of disease complexity and thereby, disease modulation.

Further analysis of the human representation can include the implementation of therapies that have been tested in the NOD mouse and in human clinical trials, including cyclosporin A, insulin tolerization, and the anti-CD3 antibody hOKT3γ1 (Ala-Ala). The mechanism of action of these therapies, as well as the tested clinical protocols (including time of administration and dosing) can be implemented as was done for therapies with the reference NOD mouse. Therapeutic protocols can be run and evaluated in the reference mouse and the reference human patient. Comparison of the reference patient outcome to reported human outcomes can provide further guidance on the pathogenesis representation necessary to achieve consistent results with untreated and treated human clinical behavior.

D. Computer Models of Type 1 Diabetes

The invention provides computer models of type 1 diabetes comprising one or more mathematical representations of a biological process associated with a pancreatic lymph node; one or more mathematical representations of a biological process associated with one more pancreatic islets; and a set of mathematical relationships between the representations of biological processes to form the model. Optionally, the computer model can also comprise one or mathematical representations of a biological process associated with gut and/or gut associated lymphoid tissue.

The methods of developing models of type 1 diabetes described above may be used to generate a simulation model of type 1 diabetes in a mammal. In such a case, the simulation model may include hundreds or even thousands of objects, each of which may include a number of parameters. In order to perform effective “what-if” analyses using a simulation model, it is useful to access and observe the input values of certain key parameters prior to performance of a simulation operation, and also possibly to observe output values for these key parameters at the conclusion of such an operation. As many parameters are included in the expression of, and are affected by, a relationship between two objects, a modeler may also need to examine certain parameters at either end of such a relationship. For example, a modeler may wish to examine parameters that specify the effects a specific object has on a number of other objects, and also parameters that specify the effects of these other objects upon the specific object. Complex models are also often broken down into a system of sub-models, either using software features or merely by the modeler's convention. It is accordingly often useful for the modeler simultaneously to view selected parameters contained within a specific sub-model. The satisfaction of this need is complicated by the fact that the boundaries of a sub-model may not be mutually exclusive with respect to parameters, i.e., a single parameter may appear in many sub-models. Further, the boundaries of sub-models often change as the model evolves.

The computer-based mathematical model of type 1 diabetes described herein synthesizes the vast but disaggregate knowledge on the pathophysiology of type 1 diabetes into a single coherent framework. The model allows for in silico research to (a) test alternate hypotheses with respect to disease progression, ultimately facilitating laboratory research through the identification or optimization of discriminating laboratory experiments, (b) reconcile apparently conflicting data on therapeutic efficacy for agents reported in the public literature, (c) optimize therapeutic agents validated in the NOD mouse for translation to human clinical trials, and (d) identify new therapeutic modalities/strategies for type 1 diabetes.

The NOD mouse type 1 diabetes model and the subsequent research using this model may be used to clarify how interactions between multiple immune components affect glucose control and the onset of diabetes in the NOD mouse. The representation enables investigation of critical pathways contributing to and regulating autoimmunity and β cell destruction, thereby providing insights into approaches to evaluate and halt or reverse disease progression. In addition to representing already characterized processes and behaviors, the development process may be used to identify data gaps and explicitly represent hypothesized mechanisms that account for incompletely characterized behaviors in pathogenesis and therapeutic responses.

The NOD mouse type 1 diabetes model can be used, for example, to further research into specific scientific questions related to the pathogenesis and treatment of type 1 diabetes in the NOD mouse, and to provide a foundation for the representation and investigation of type 1 diabetes in humans. The model may therefore improve (i) the scientific understanding of type 1 diabetes pathogenesis through the use of predictive mathematical models; and (ii) the rationale for advancing potential therapies for type 1 diabetes into human clinical trials.

The model (and development thereof) may be used to generate scientific insights into the pathogenesis of type 1 diabetes. These insights may clarify and characterize major research questions surrounding disease progression. This NOD mouse type 1 diabetes model also enables in silico research into NOD type 1 diabetes pathogenesis and intervention and supports translational efforts, including the development and application of a human type 1 diabetes model.

The created computer model represents biological processes at multiple levels and then evaluates the effect of the biological processes on biological processes across all levels. Thus, the created computer model provides a multi-variable view of a biological system. The created computer model also provides cross-disciplinary observations through synthesis of information from two or more disciplines into a single computer model or through linking two computer models that represent different disciplines.

An exemplary, computer model reflects a particular biological system, e.g., the pancreas, and anatomical factors relevant to issues to be explored by the computer model. The level of detail incorporated into the model is often dictated by a particular intended use of the computer model. For example, biological components being evaluated often operate at a subcellular level; therefore, the subcellular level can occupy the lowest level of detail represented in the model. The subcellular level includes, for example, biological components such as DNA, mRNA, proteins, chemically reactive molecules, and subcellular organelles. Similarly, the model can be evaluated at the multicellular level or even at the level of a whole organism. Because an individual biological system, i.e. a single human or mouse, is a common entity of interest with respect to the ultimate effect of the biological components, the individual biological system (e.g., represented in the form of clinical outcomes) is the highest level represented in the system. Disease processes and therapeutic interventions are introduced into the model through changes in parameters at lower levels, with clinical outcomes being changed as a result of those lower level changes, as opposed to representing disease effects by directly changing the clinical outcome variables.

The level of detail reported to a user can vary depending on the level of sophistication of the target user. For a healthcare setting, especially for use by members of the public, it may be desirable to include a higher level of abstraction on top of a computer model. This higher level of abstraction can show, for example, major physiological subsystems and their interconnections, but need not report certain detailed elements of the computer model—at least not without the user explicitly deciding to view the detailed elements. This higher level of abstraction can provide a description of the virtual patient's phenotype and underlying physiological characteristics, but need not include certain parametric settings used to create that virtual patient in the computer model. When representing a therapy, this higher level of abstraction can describe what the therapy does but need not include certain parametric settings used to simulate that therapy in the computer model. A subset of outputs of the computer model that is particularly relevant for subjects and doctors can be made readily accessible.

In one implementation, the computer model is configured to allow visual representation of mathematical relations as well as interrelationships between variables, parameters, and biological processes. This visual representation includes multiple modules or functional areas that, when grouped together, represent a large complex model of a biological system.

In one implementation, simulation modeling software is used to provide a computer model, e.g., as described in U.S. Pat. No. 5,657,255, issued Aug. 12, 1997, titled “Hierarchical Biological Modeling System and Method”; U.S. Pat. No. 5,808,918, issued Sep. 15, 1998, titled “Hierarchical Biological Modeling System and Method”; U.S. Pat. No. 6,051,029, issued Apr. 18, 2000, titled “Method of Generating a Display for a Dynamic Simulation Model Utilizing Node and Link Representations”; U.S. Pat. No. 6,539,347, issued Mar. 25, 2003, titled “Method of Generating a Display For a Dynamic Simulation Model Utilizing Node and Link Representations”; U.S. Pat. No. 6,078,739, issued Jan. 25, 2000, titled “A Method of Managing Objects and Parameter Values Associated With the Objects Within a Simulation Model”; and U.S. Pat. No. 6,069,629, issued May 30, 2000, titled “Method of Providing Access to Object Parameters Within a Simulation Model”. An example of simulation modeling software is found in U.S. Pat. No. 6,078,739.

Various Diagrams can be used to illustrate the dynamic relationships among the elements of the model of type 1 diabetes. Examples of suitable diagrams include Effect and Summary Diagrams.

A Summary Diagram can provide an overview of the various pathways and modules modeled in the methods and models described herein. For example, the Summary Diagram illustrated in FIG. 2 provides an overview of pathways and modules that can affect glucose control. The Summary Diagram can also provide links to individual modules of the model. The modules model the relevant components of the phenotype through the use of “state” and “function” nodes whose relations are defined through the use of diagrammatic arrow symbols. Thus, the complex and dynamic mathematical relationships for the various elements of the phenotype are easily represented in a user-friendly manner.

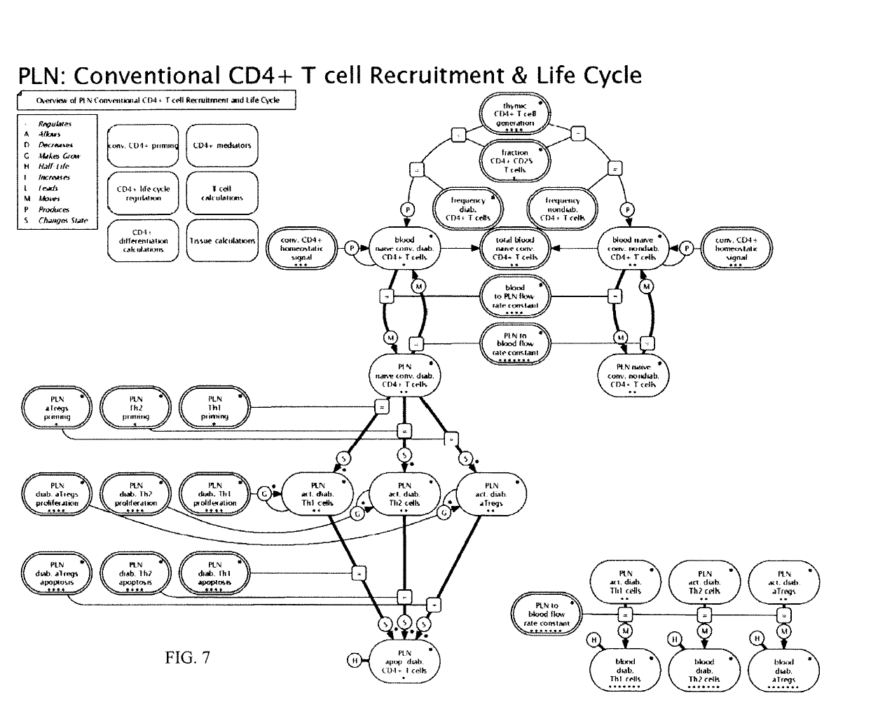

An Effect Diagram can be a visual representation of the model equations and illustrate the dynamic relationships among the elements of the model. FIG. 7 illustrates an example of an Effect Diagram, in which conventional CD4+ T cell recruitment and life cycles within the pancreatic lymph nodes are described. The Effect Diagram is organized into modules, or functional areas, which when grouped together represent the large complex physiology of the phenotype being modeled.

State and function nodes show the names of the variables they represent and their location in the model. The arrows and modifiers show the relationship of the state and function nodes to other nodes within the model. State and function nodes also contain the parameters and equations that are used to compute the values of the variables the represent in simulated experiments. In some embodiments, the state and function nodes are represented according to the method described in U.S. Pat. No. 6,051,029, entitled “Method of Generating a Display for a Dynamic Simulation Model Utilizing Node and Link Representations,” incorporated herein by reference. Further examples of state and function nodes are further discussed below.

State nodes are represented by single-border ovals and represent variables in the system, the values of which are determined by the cumulative effects of inputs over time. “Input” refers to any parameter that can affect the variable being modeled by the state node. For example, input for a state node representing tissue inactive macrophage can be macrophage recruitment or circulating inactive monocytes. State node values are defined by differential equations. The predefined parameters for a state node include its initial value (S₀) and its status. In some embodiments, state nodes can have a half-life. In these embodiments, a circle containing an “H” is attached to the node that has a half-life.

Function nodes are represented by double-border ovals and represent variables in the system, the values of which, at any point in time, are determined by inputs at the same point in time. Function nodes are defined by algebraic functions of their inputs. The predefined parameters for a function node include its initial value (F₀) and its status. Setting the status of a node effects how the value of the node is determined. The status of a state or function node can be: 1) Computed, i.e., the value is calculated as a result of its inputs; 2) Specified-Locked, i.e., the value is held constant over time; or 3) Specified Data, i.e., the value varies with time according to predefined data points.

State and function nodes can appear more than once in the module diagram as alias nodes. Alias nodes are indicated by one or more dots (see, e.g., state node “PLN act.diab. Th1 cells” in FIG. 7). State and Function nodes are also defined by their position, with respect to arrows and other nodes, as being either source nodes (S) or target nodes (T). Source nodes are located at the tails of arrows and target nodes are located at the heads of arrows. Nodes can be active or inactive.

Arrows link source nodes to target nodes and represent the mathematical relationship between the nodes. Arrows can be labeled with circles that indicate the activity of the arrow. A key to the annotations in the circles is located in the upper left corner of each module Diagrams. If an arrowhead is solid, the effect is positive. If the arrowhead is hollow, the effect is negative. For further description of arrow types, arrow characteristics, and arrow equations, see, e.g., U.S. Pat. No. 6,051,029, U.S. Pat. No. 6,069,629, U.S. Pat. No. 6,078,739, and U.S. Pat. No. 6,539,347.

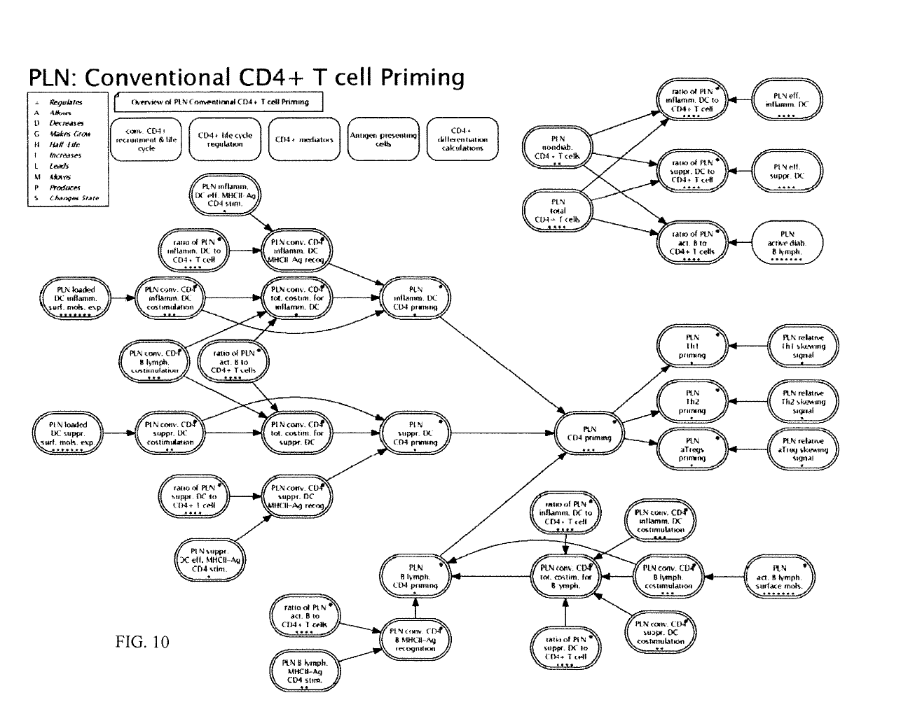

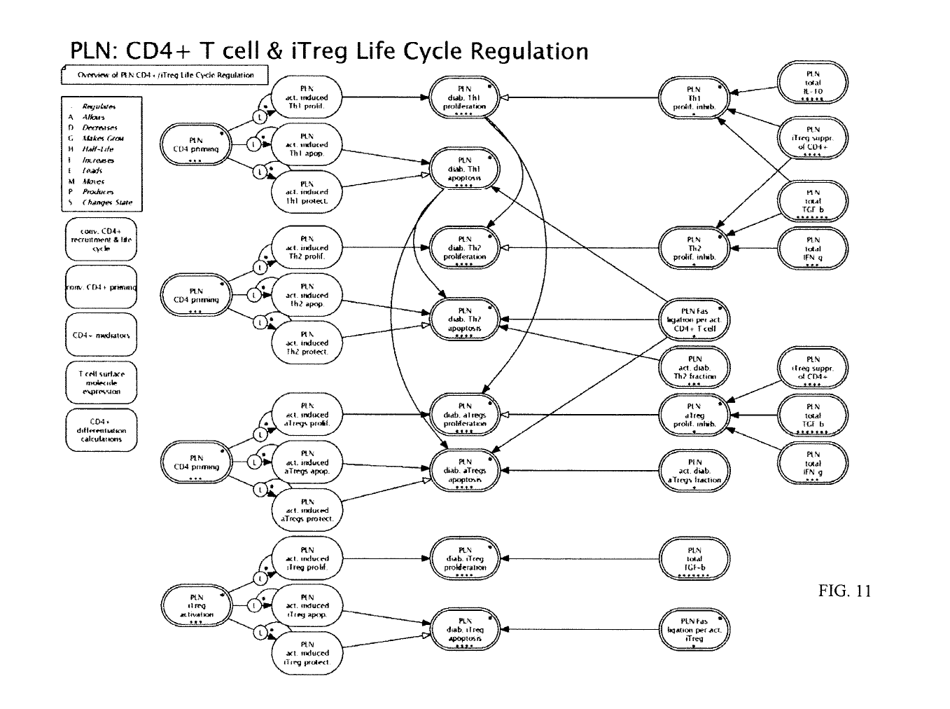

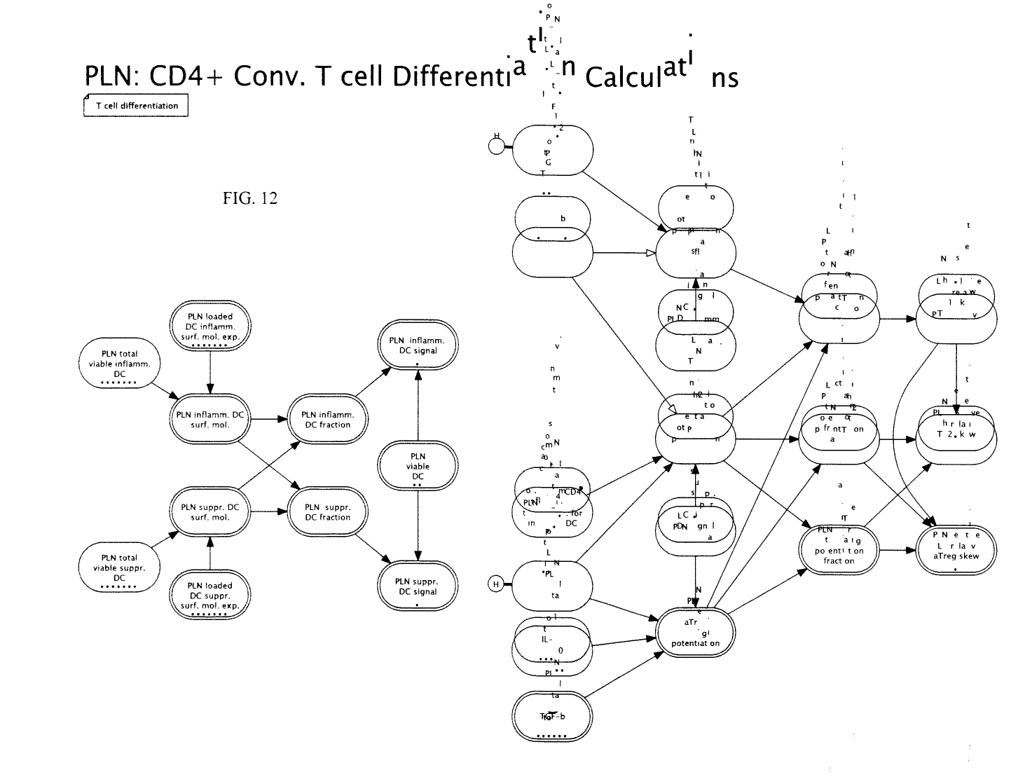

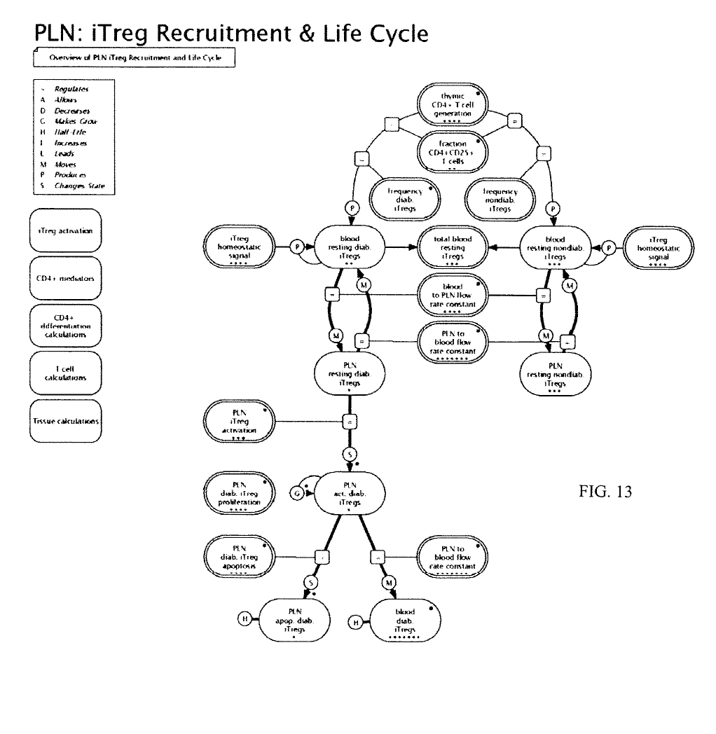

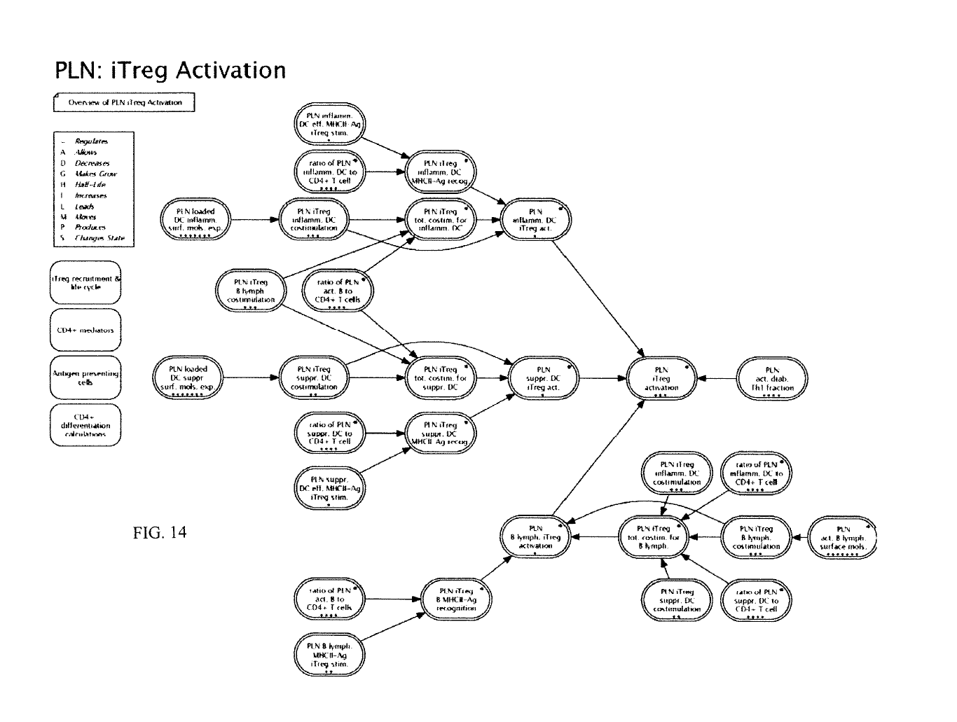

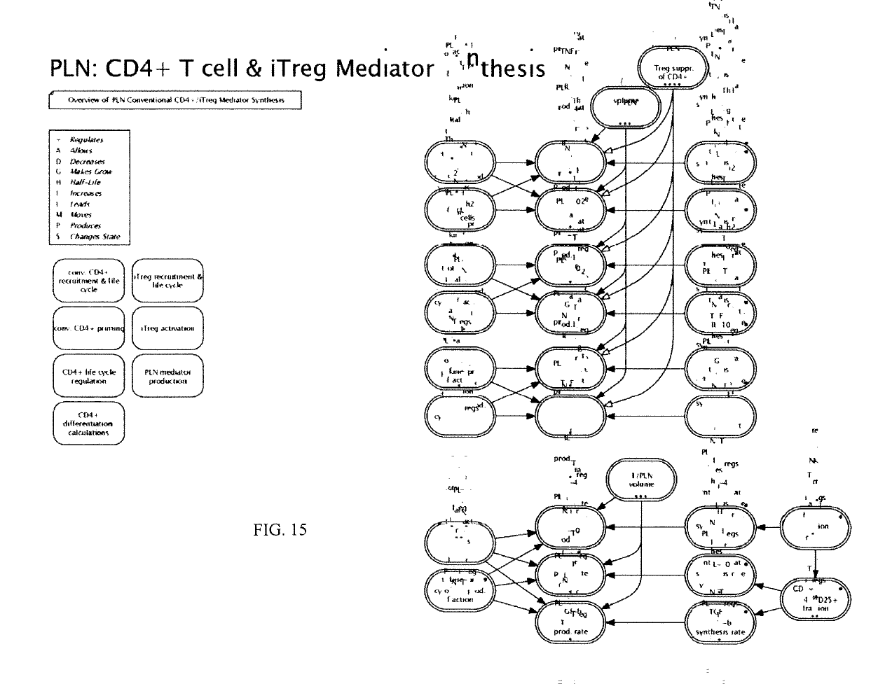

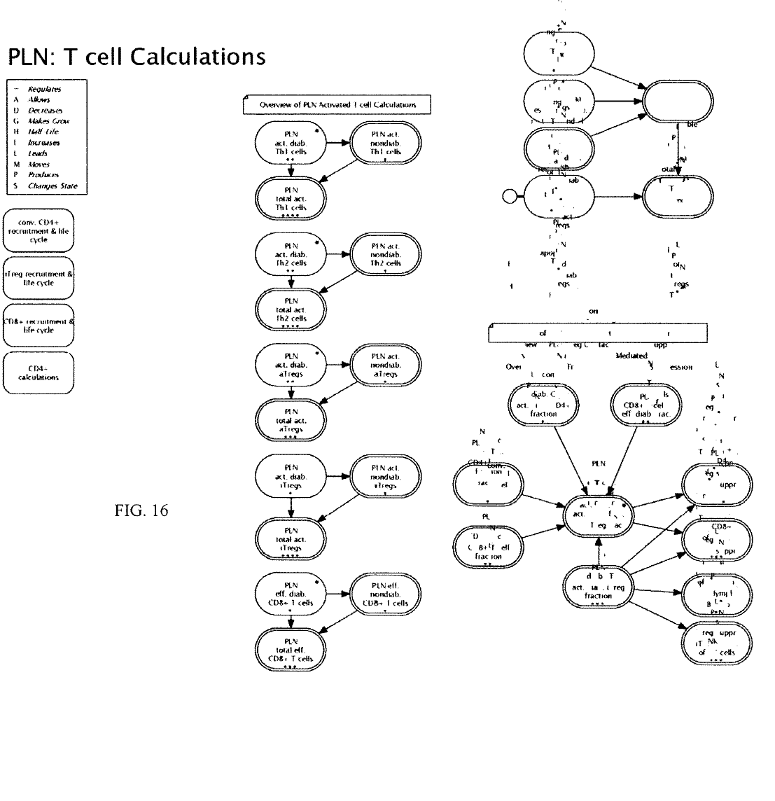

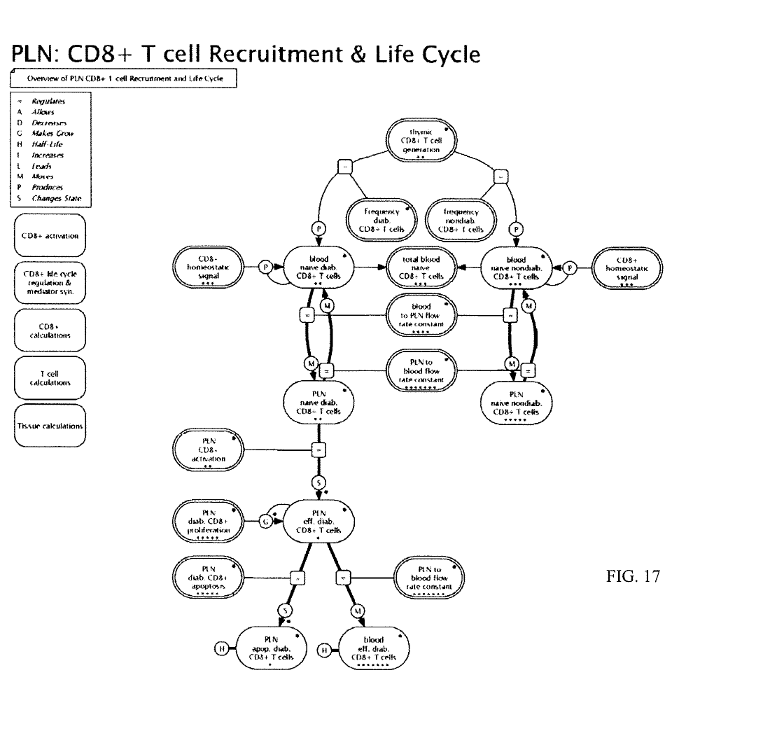

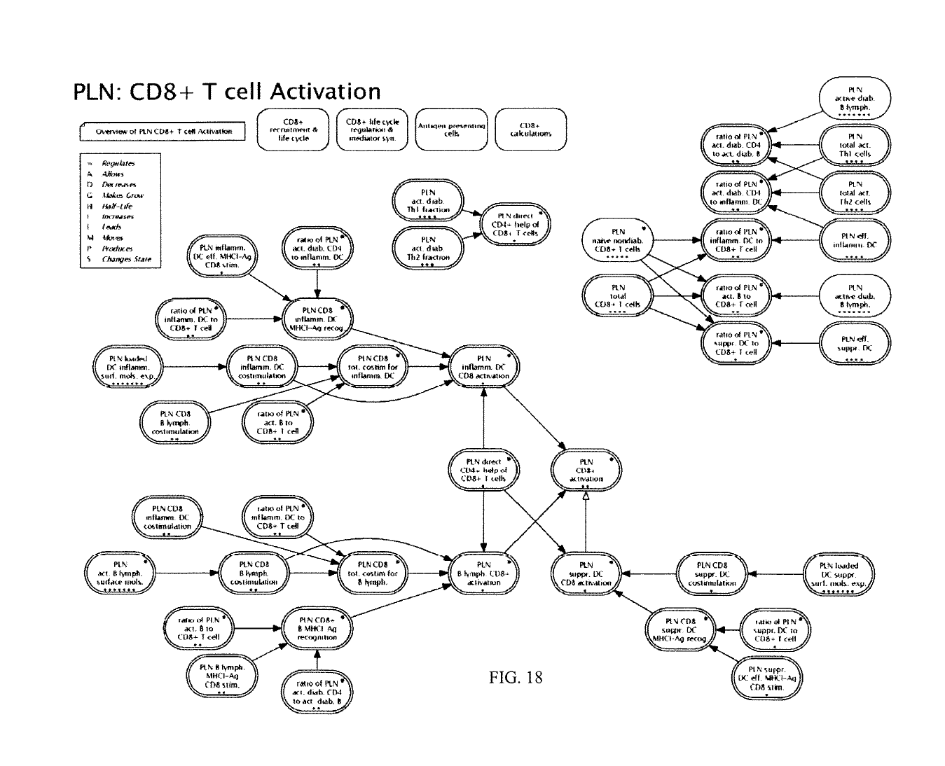

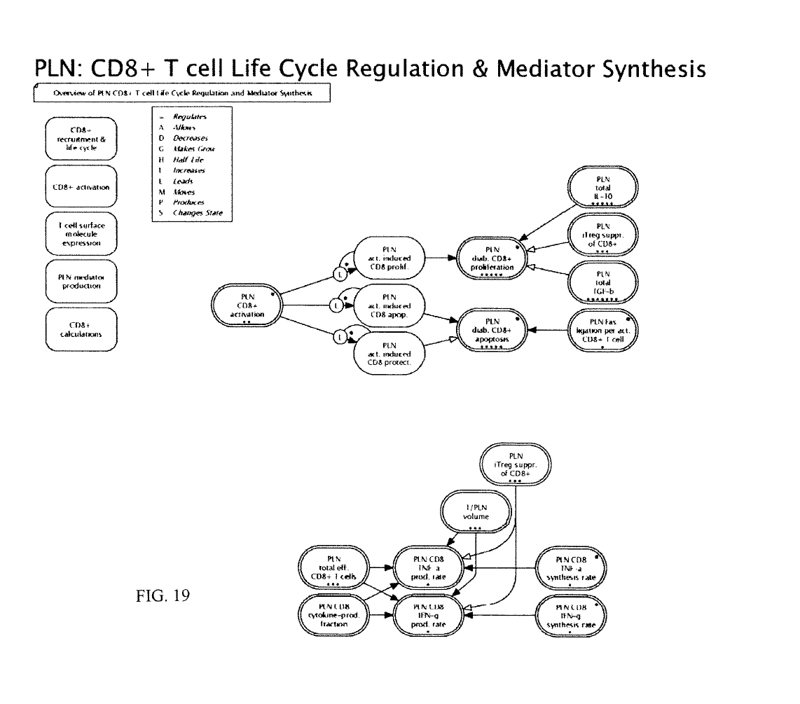

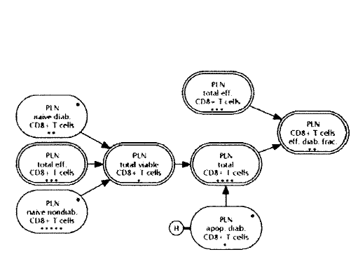

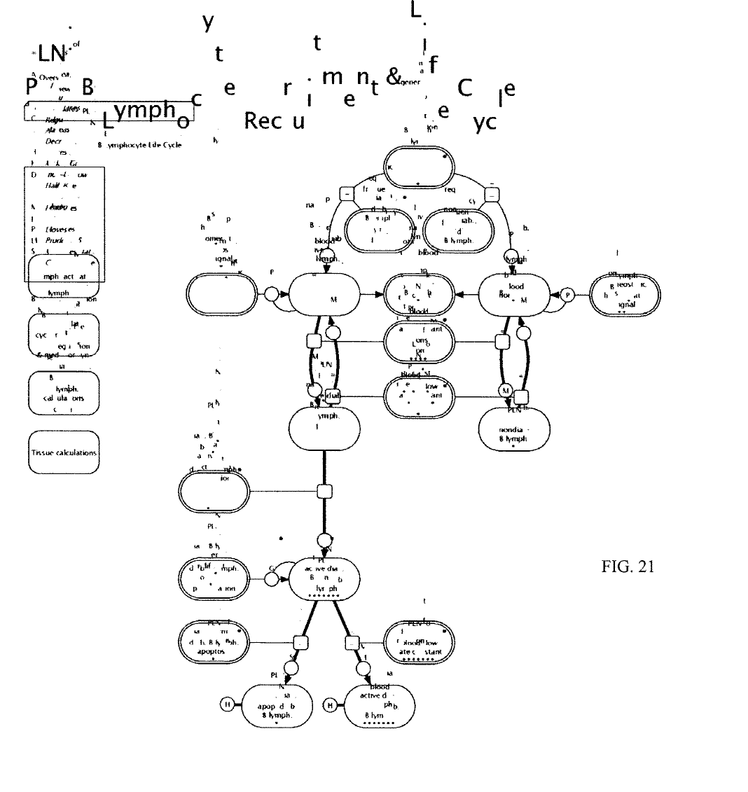

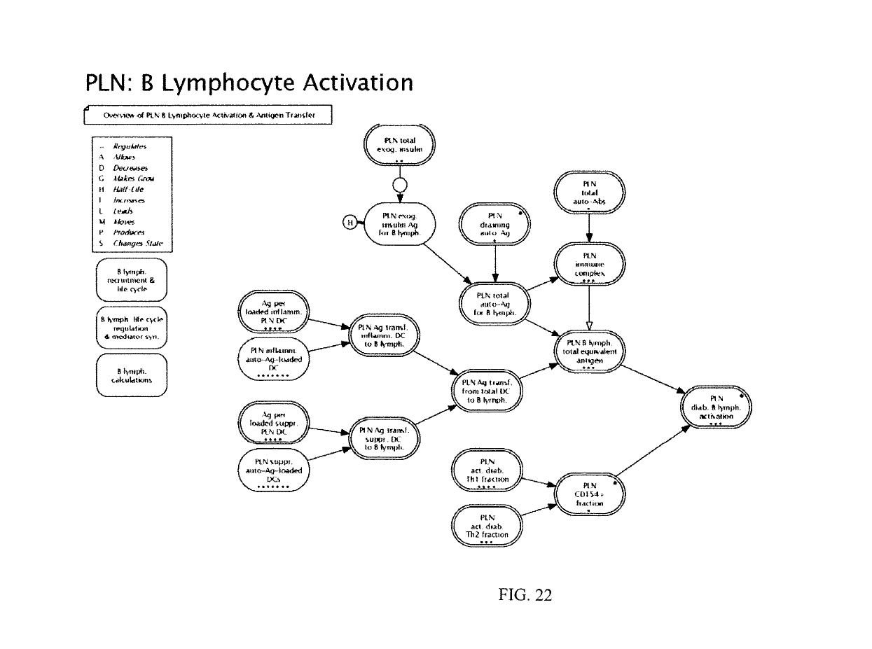

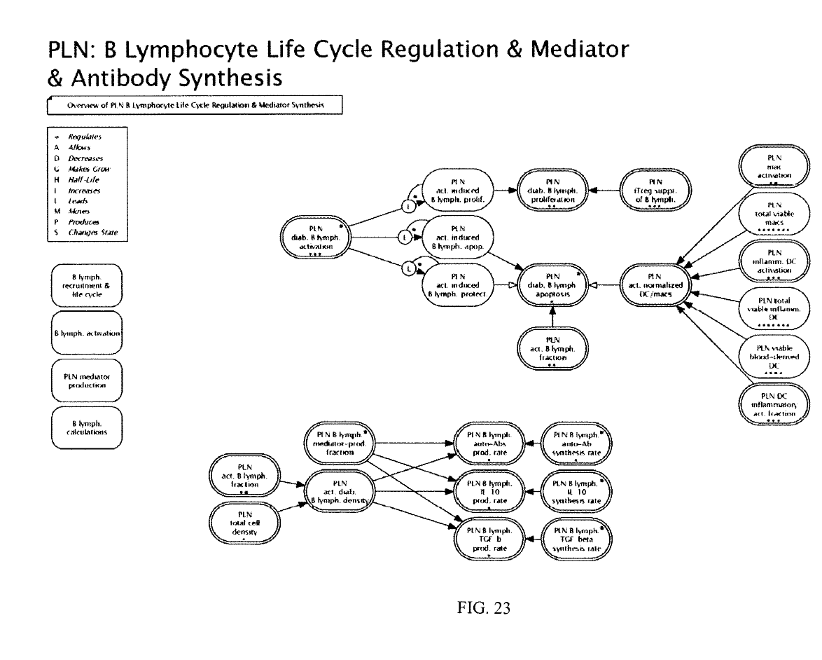

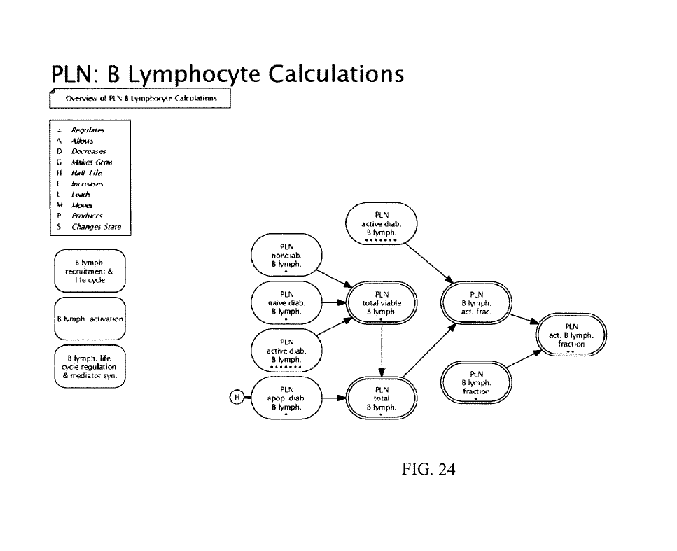

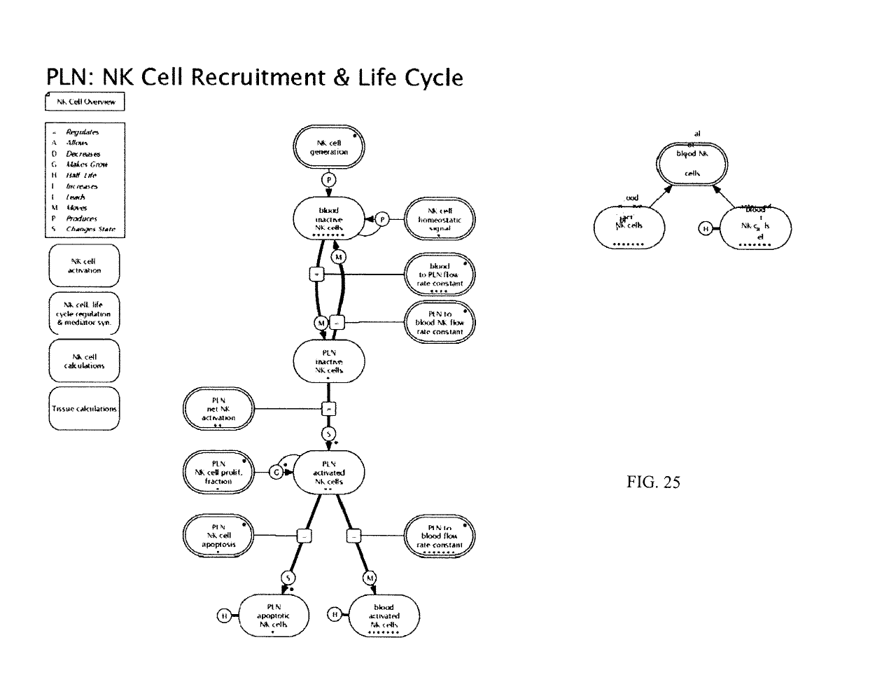

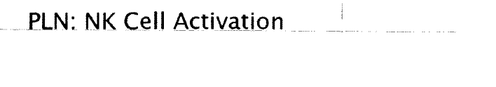

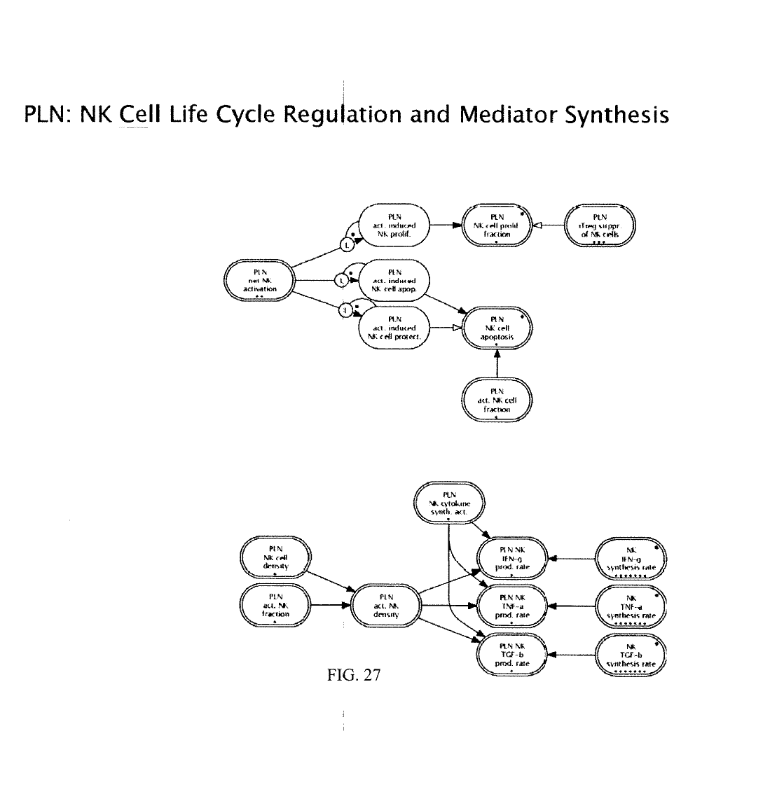

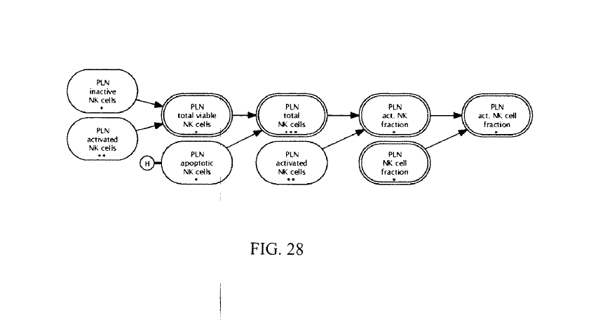

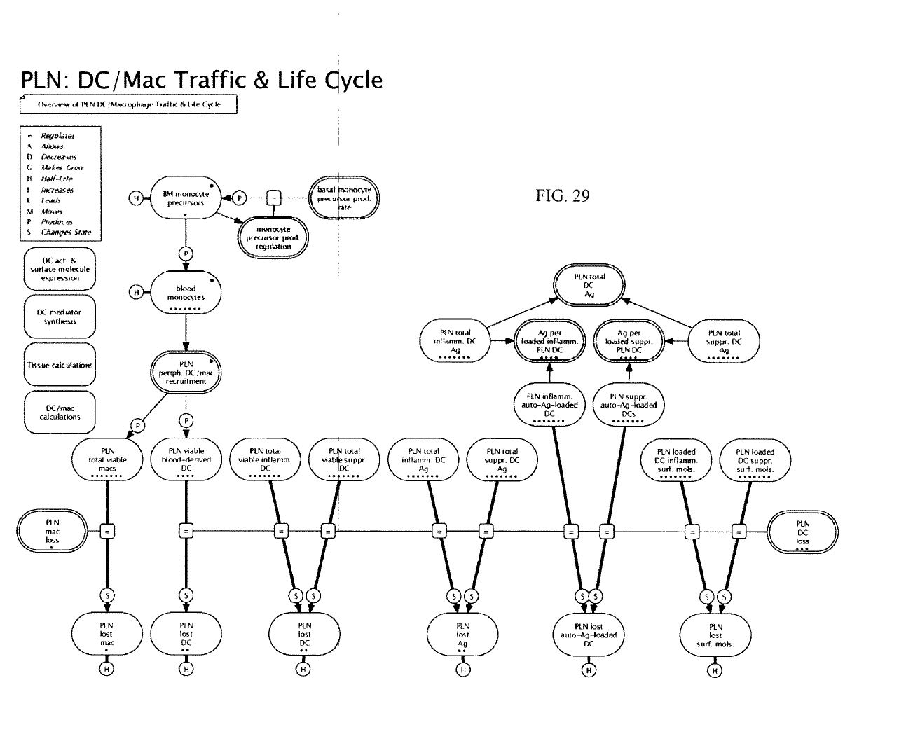

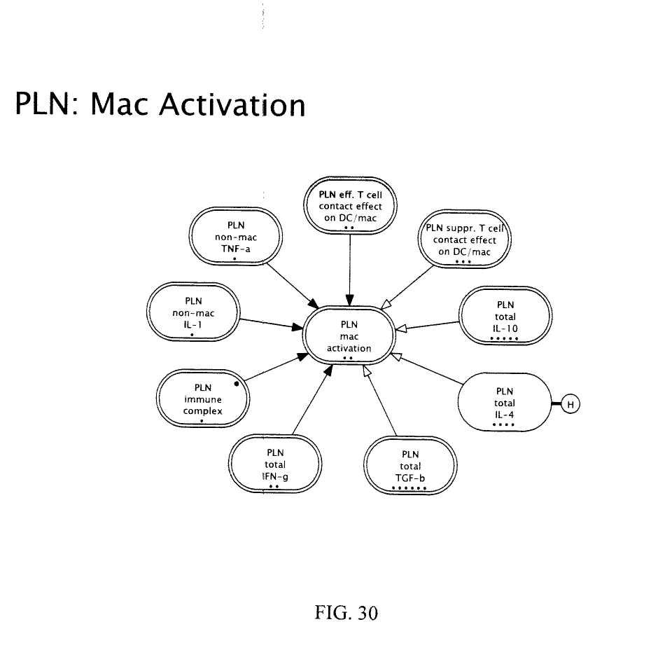

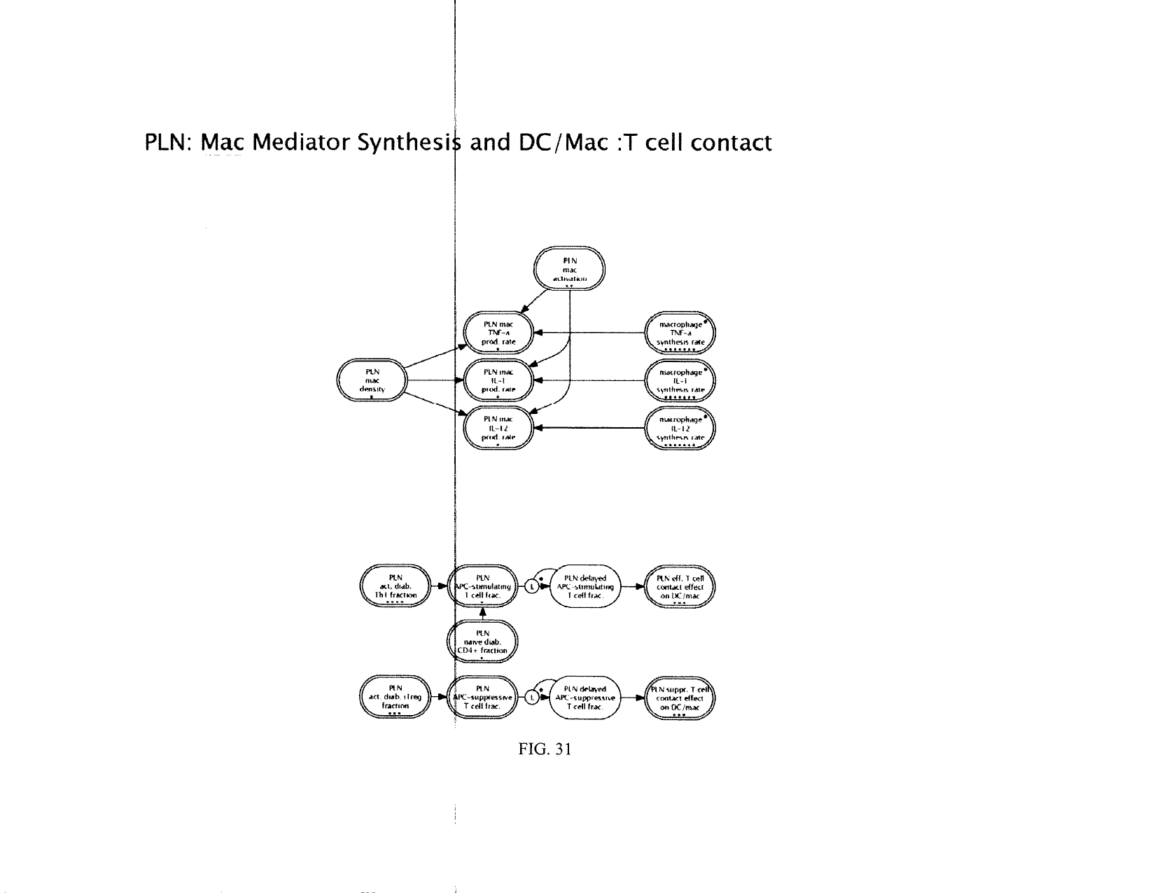

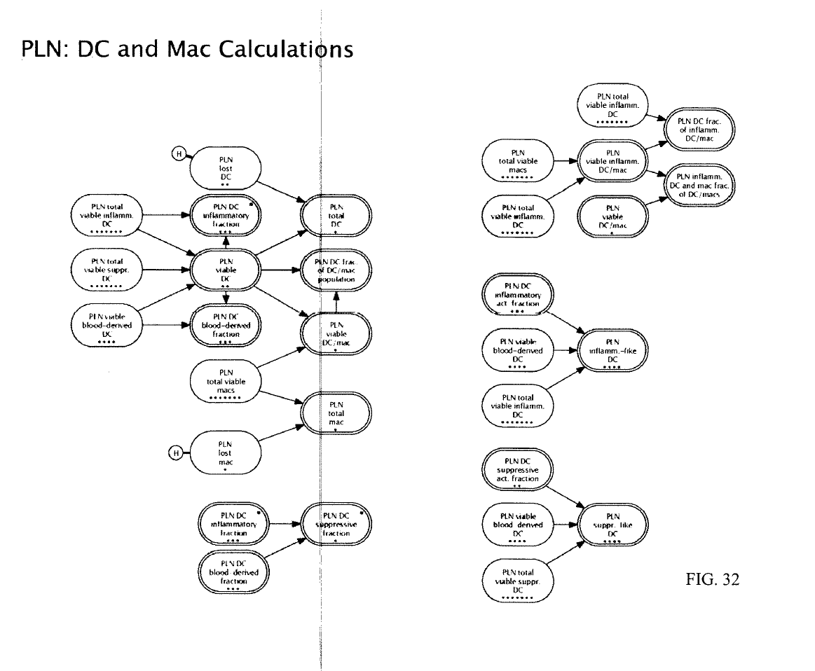

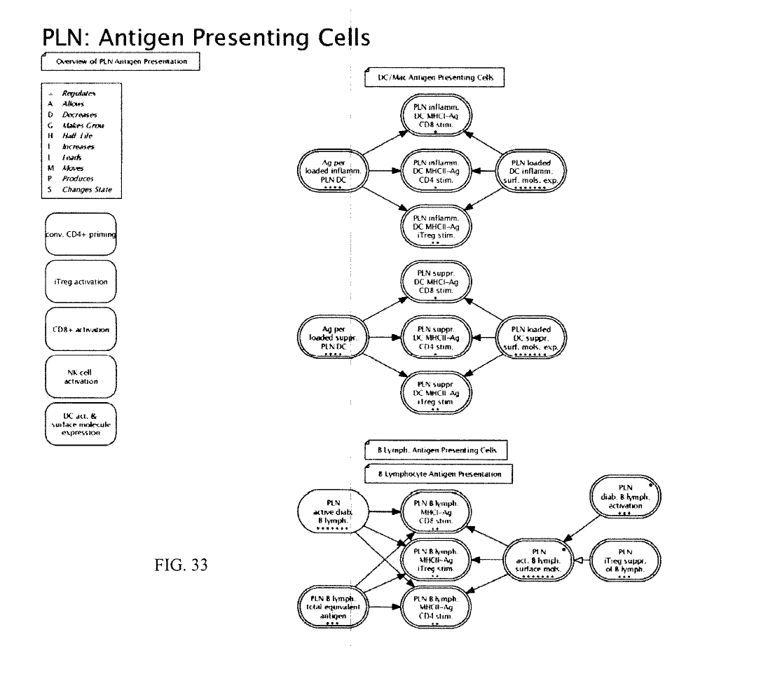

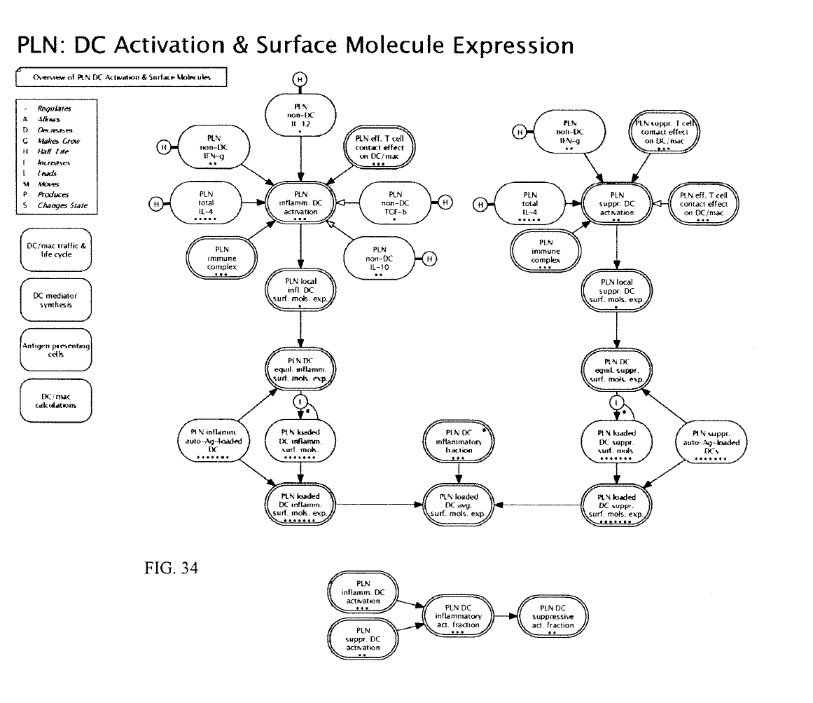

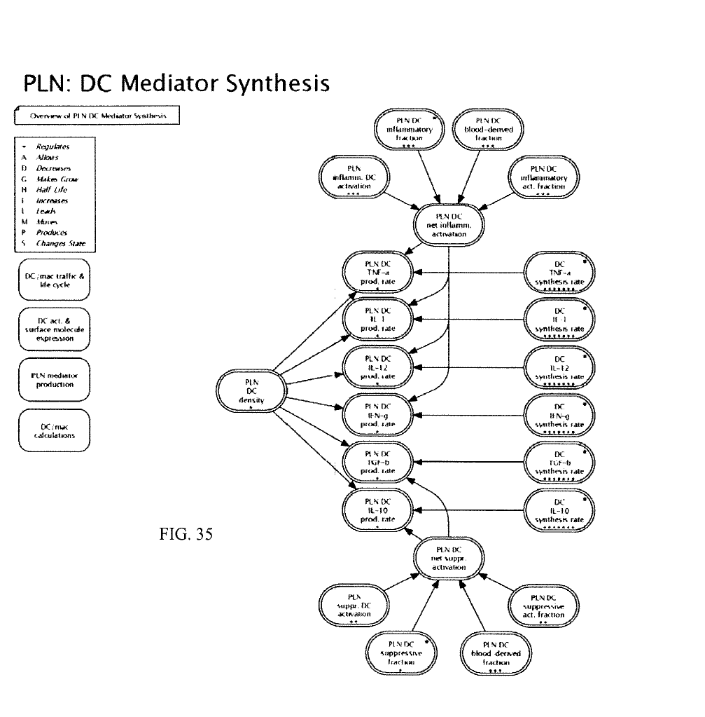

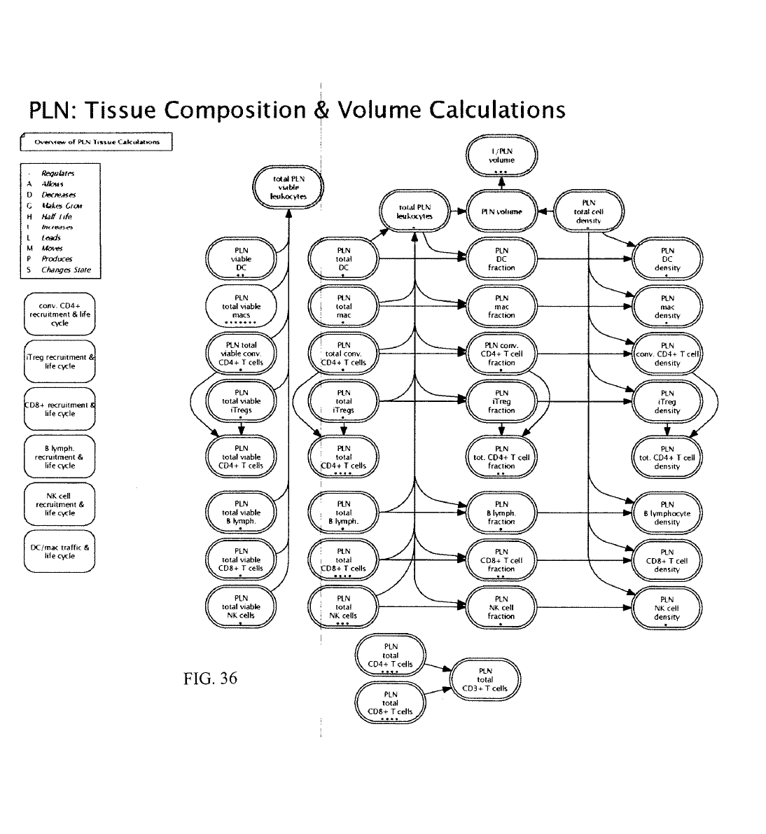

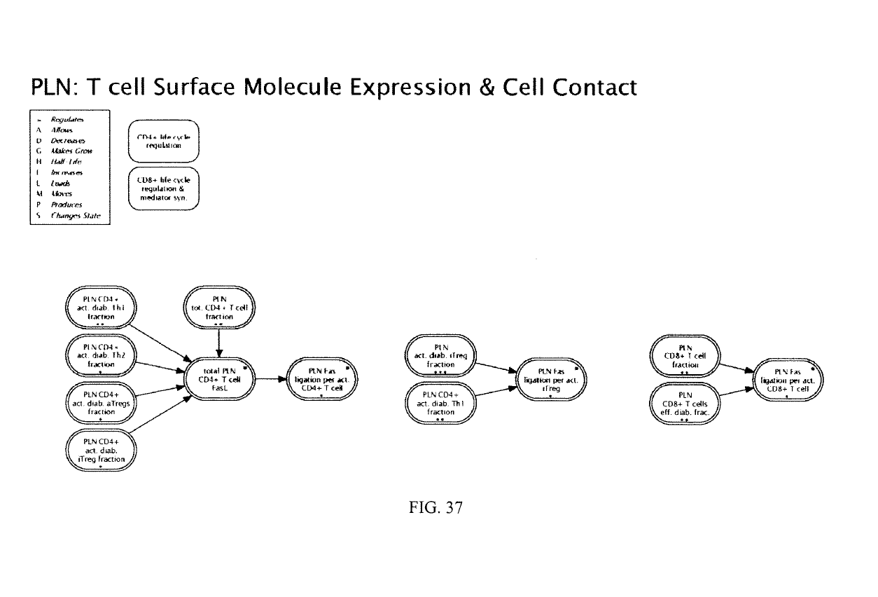

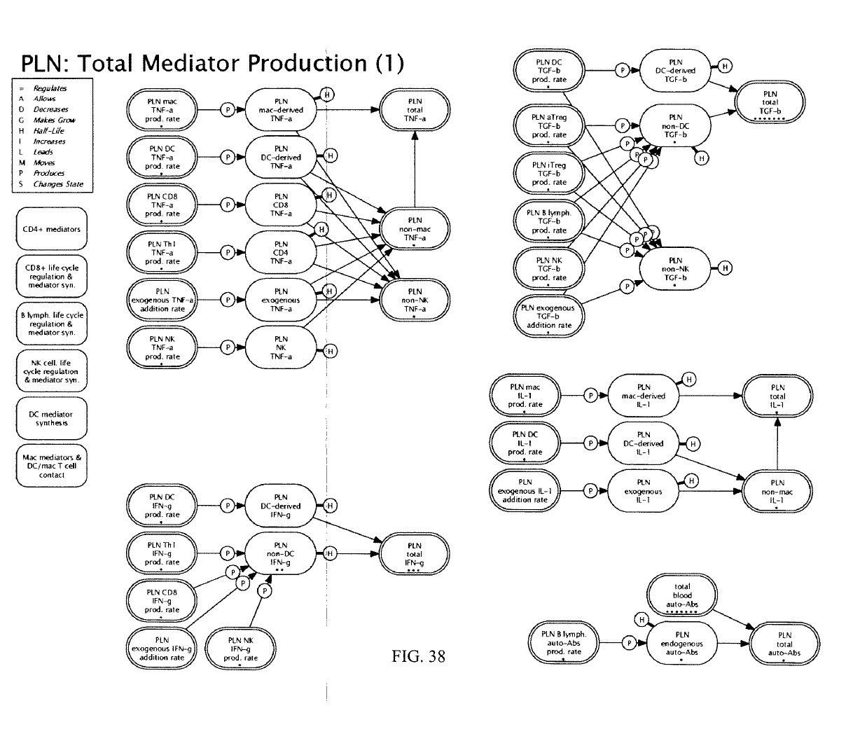

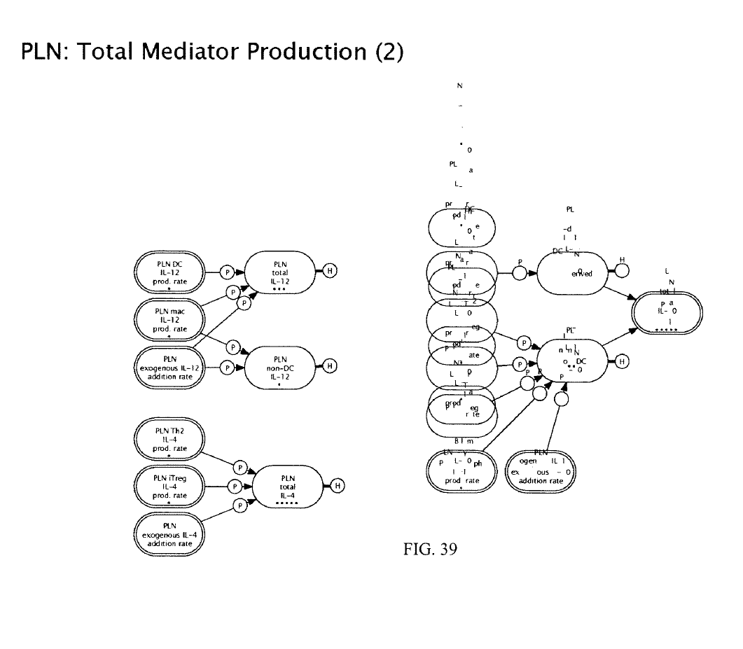

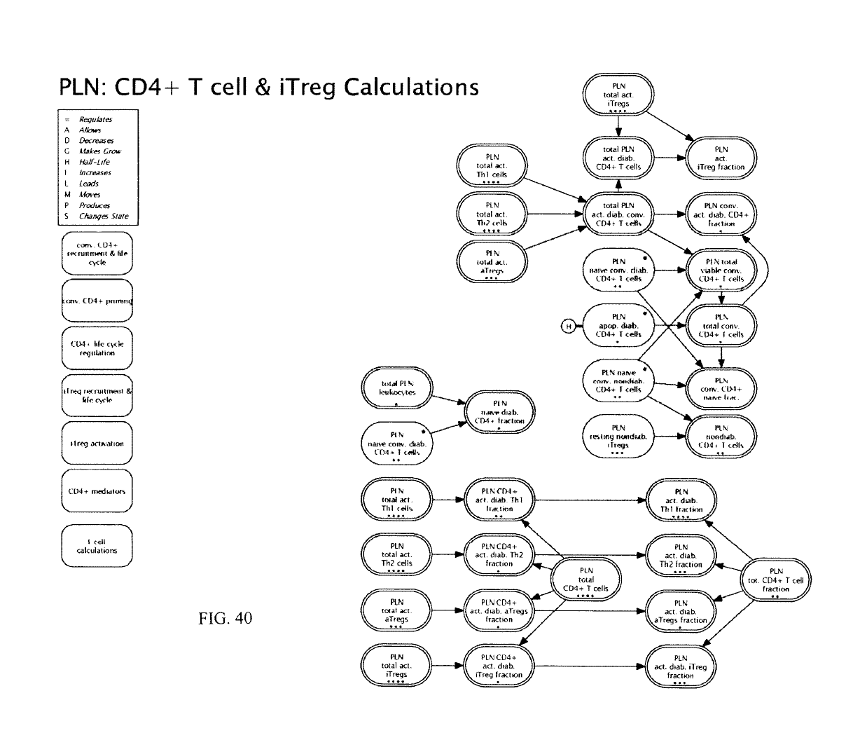

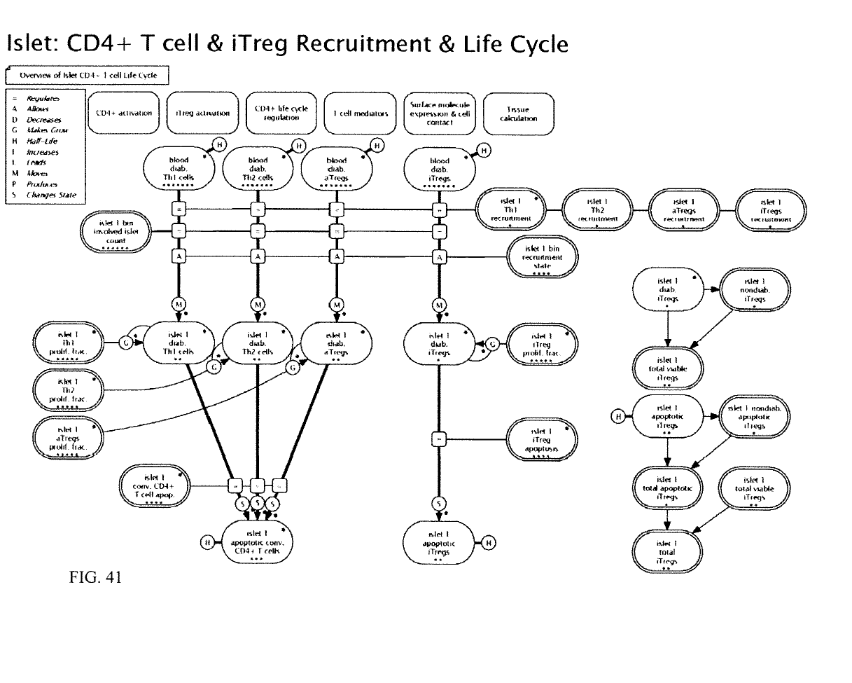

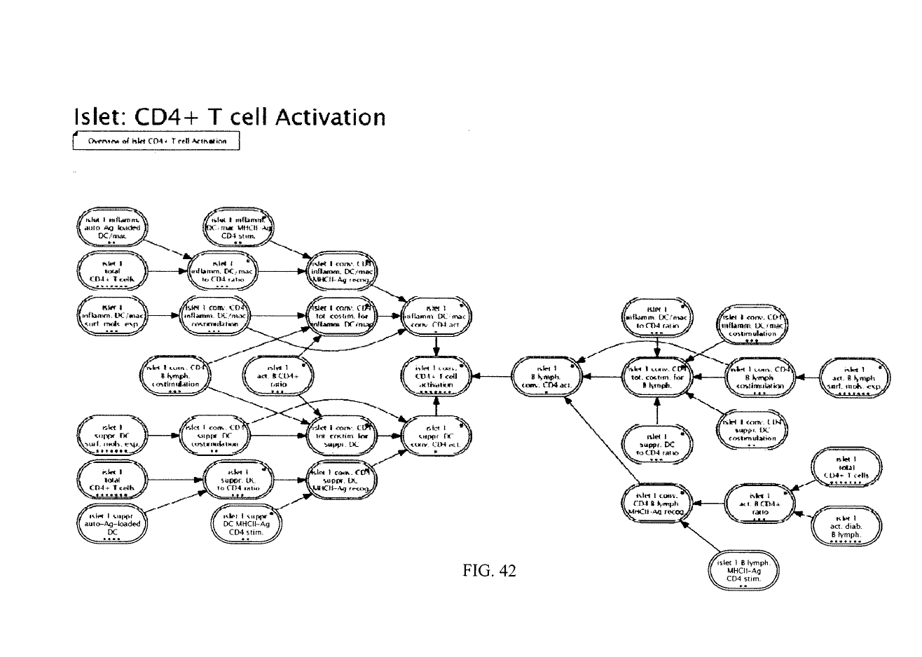

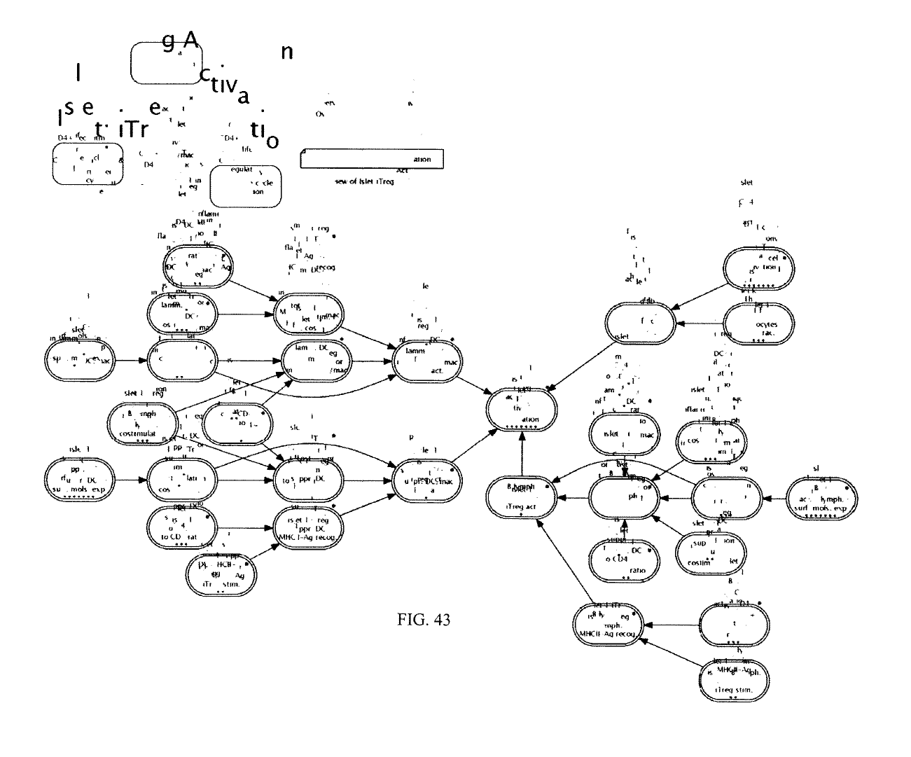

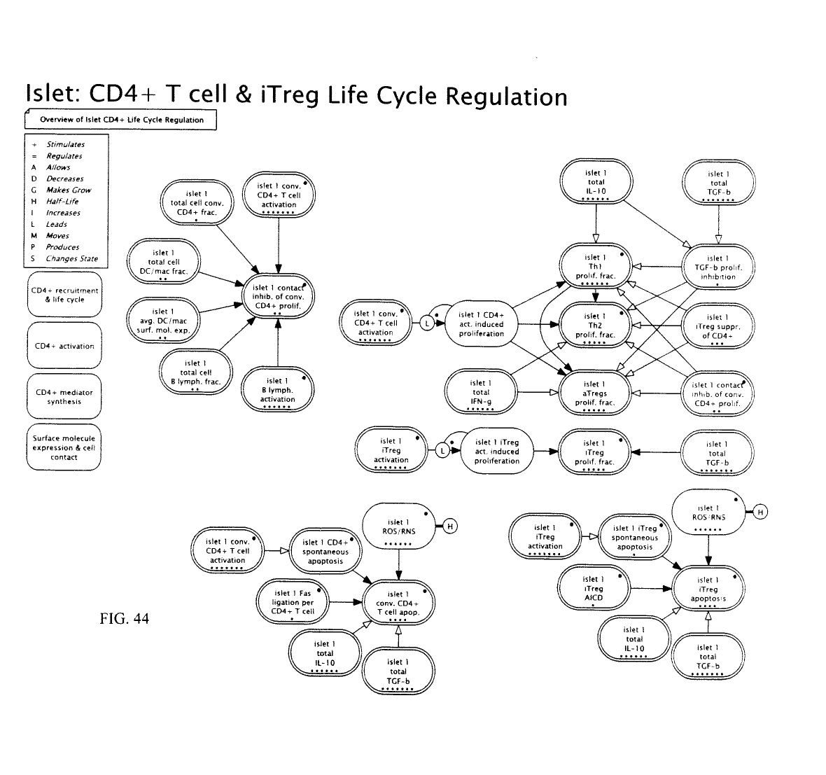

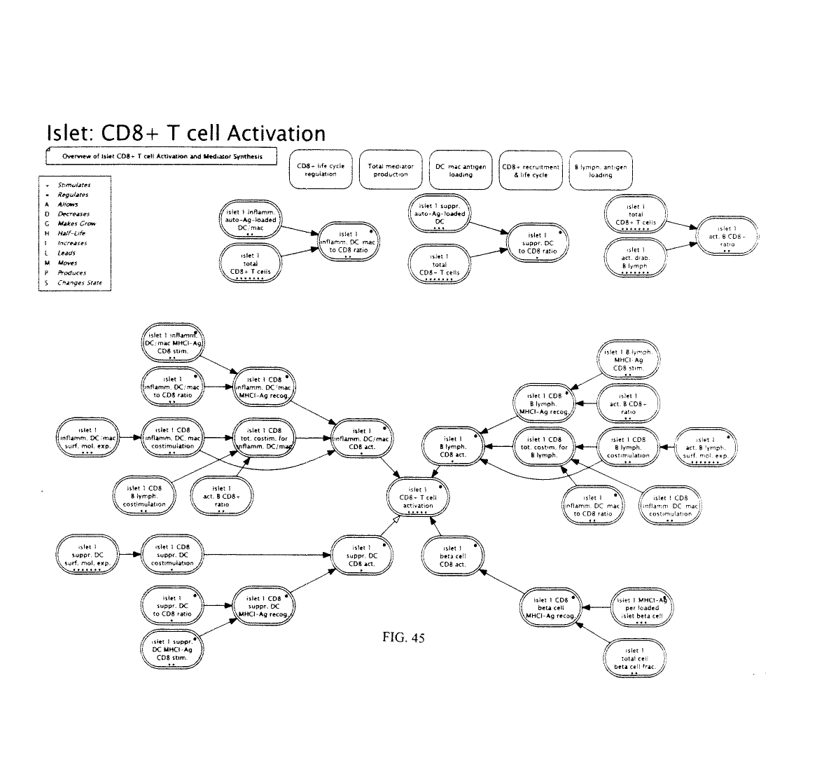

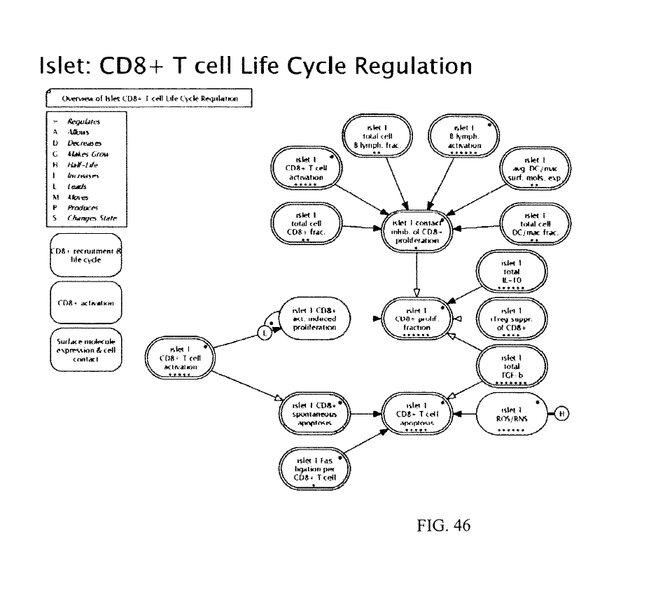

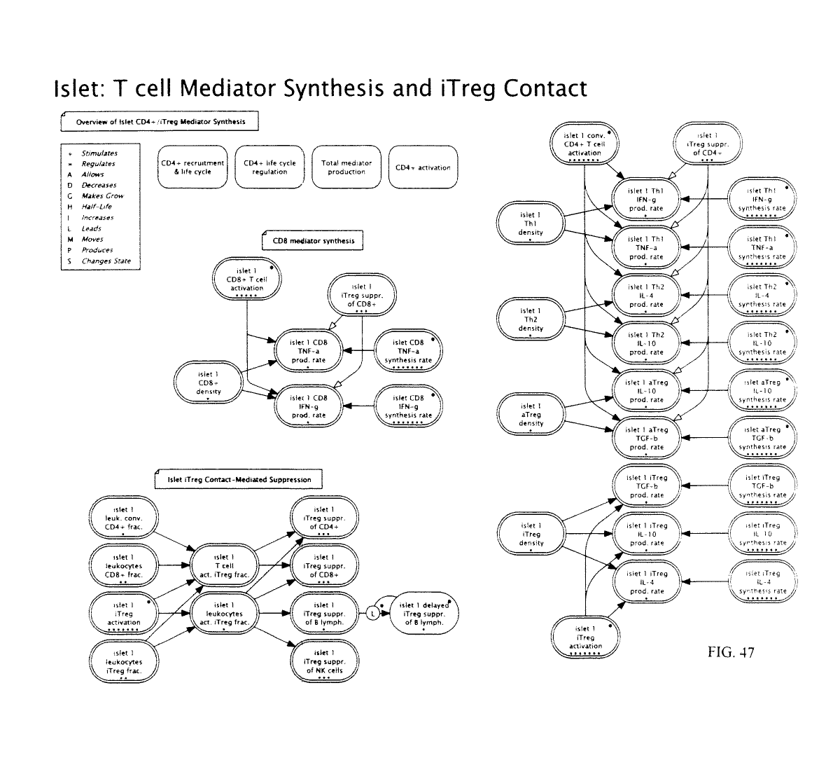

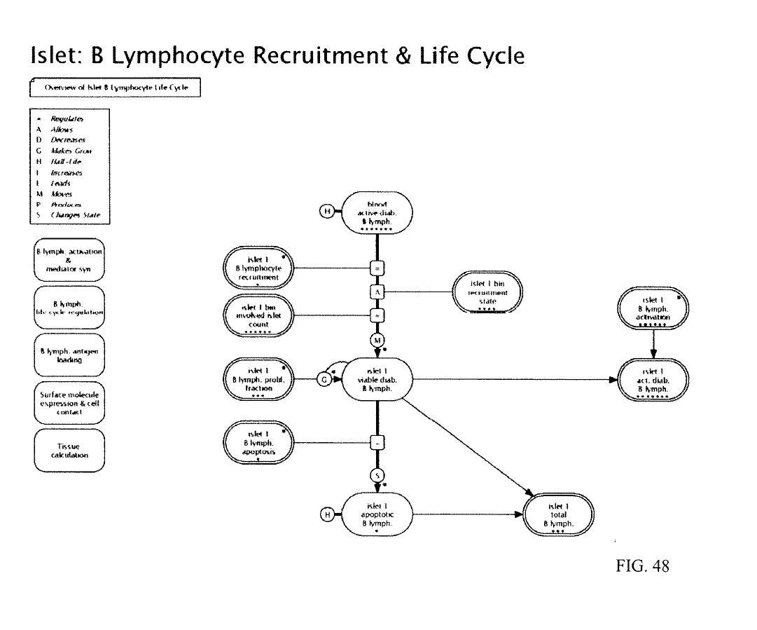

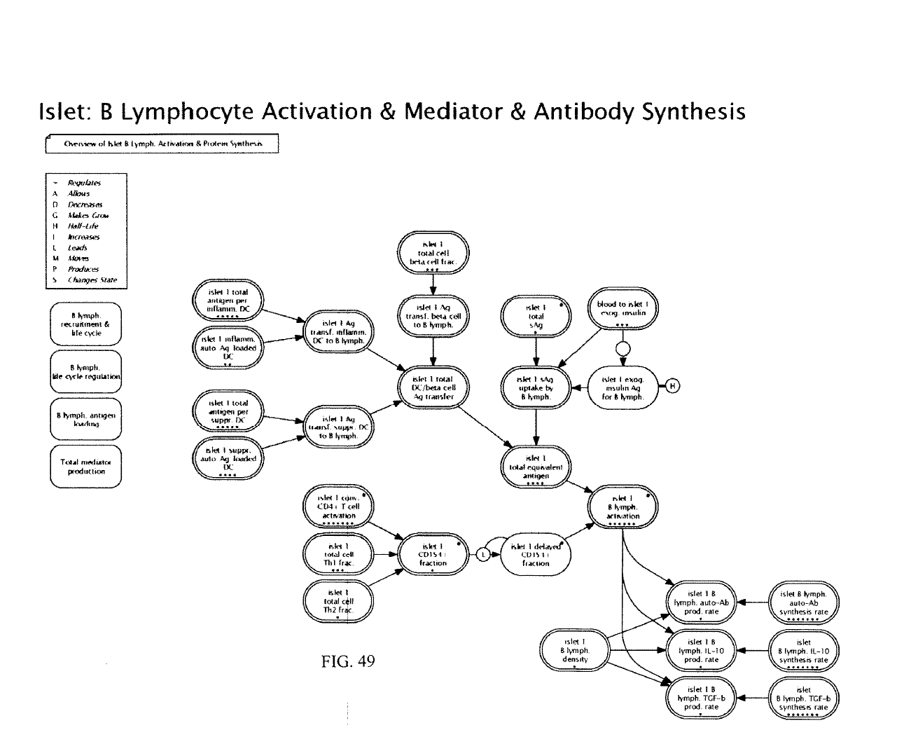

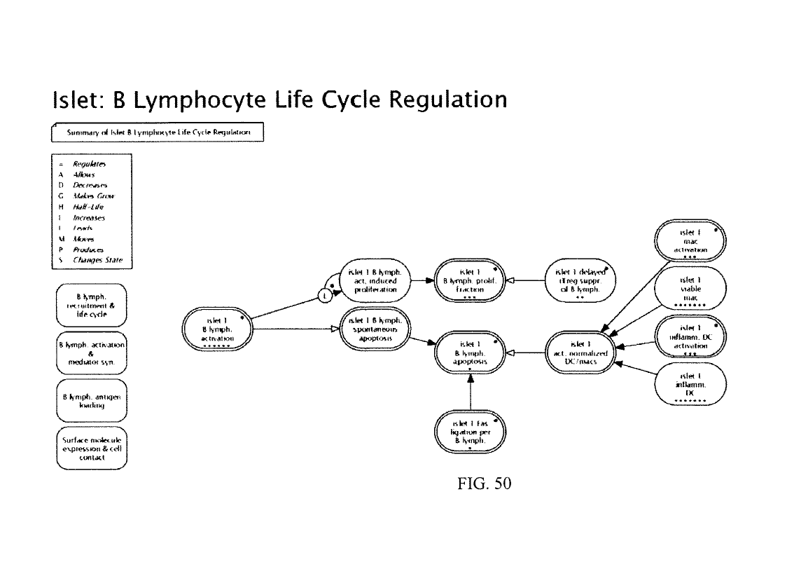

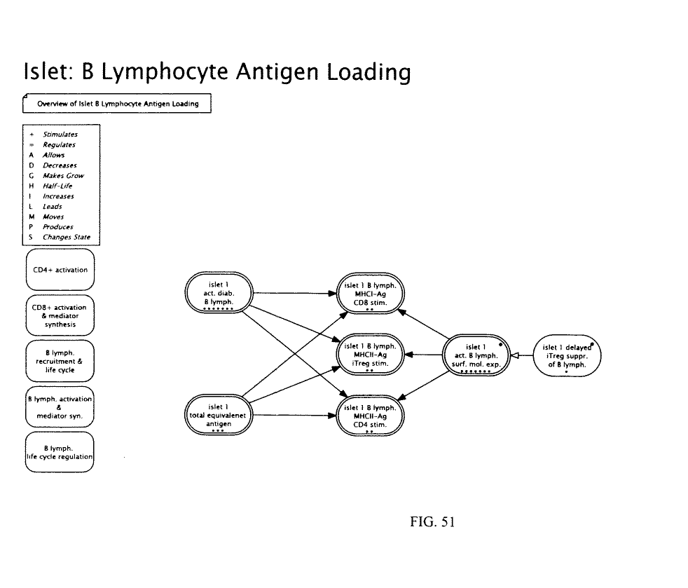

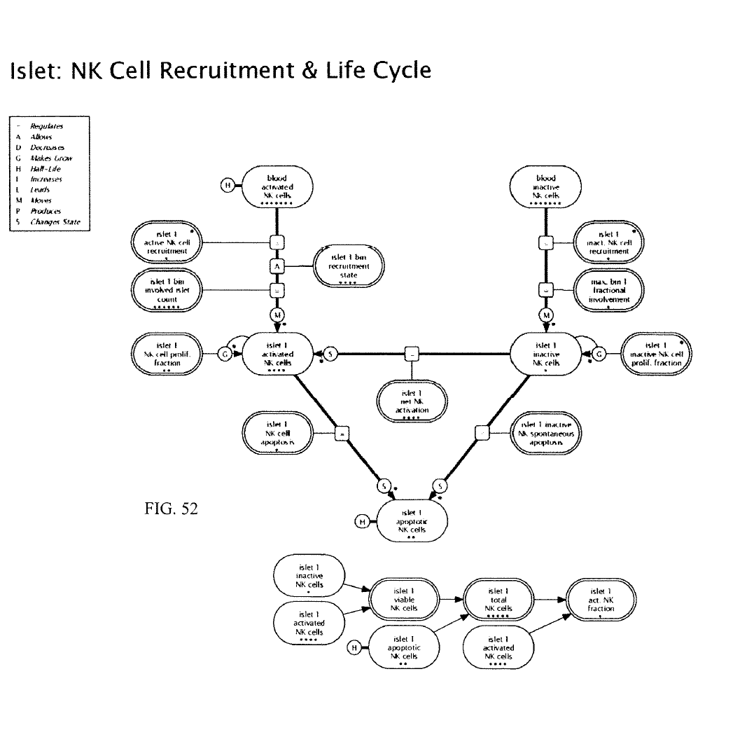

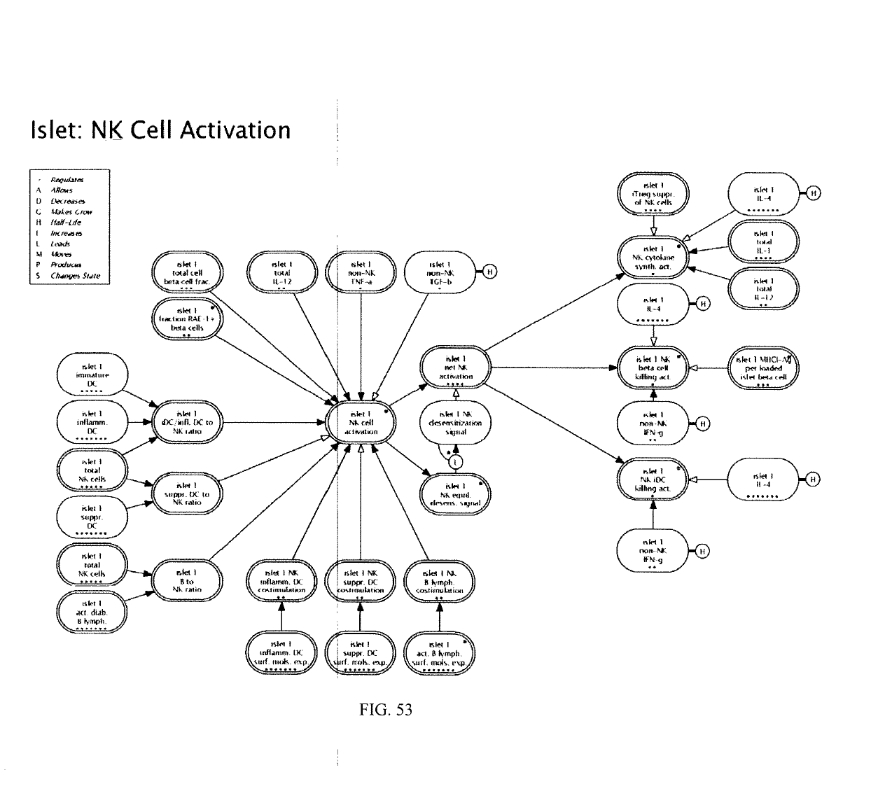

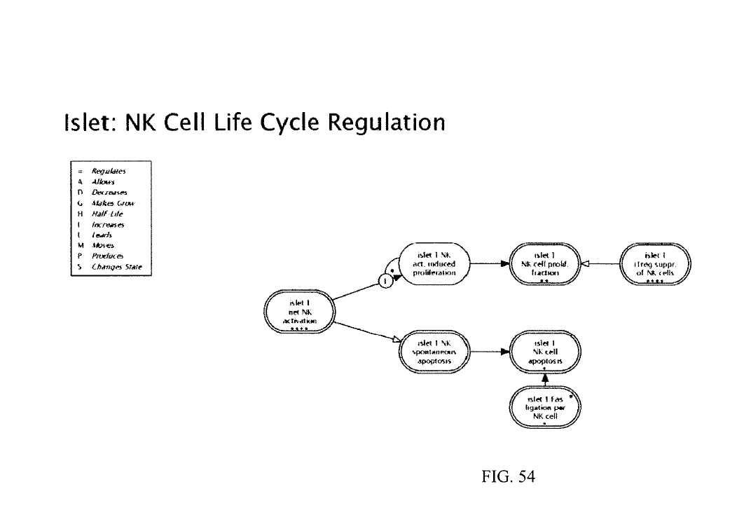

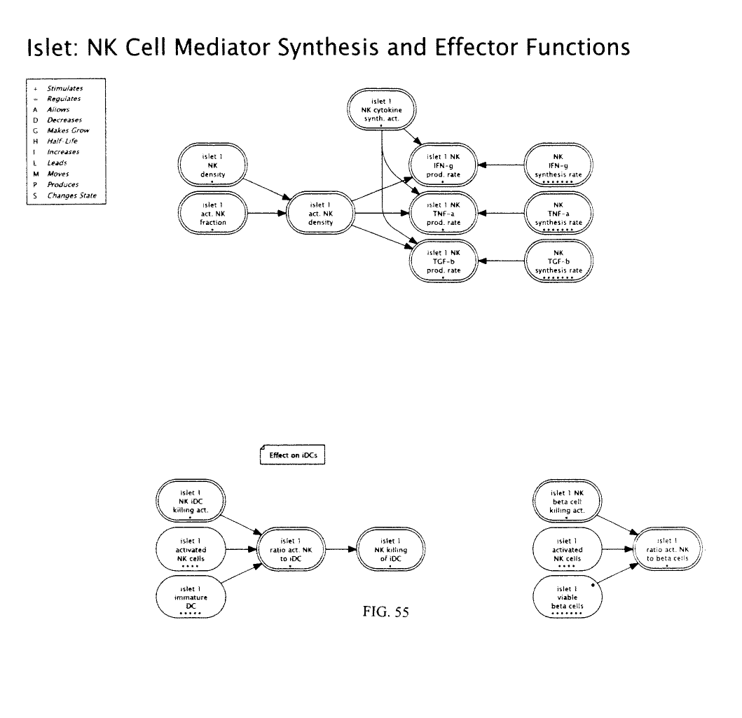

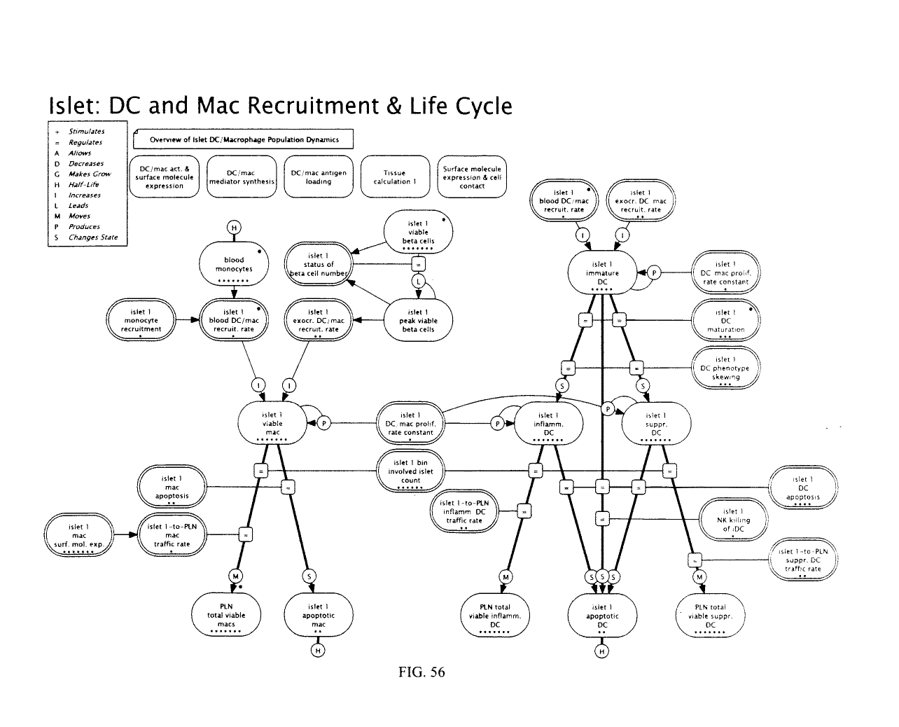

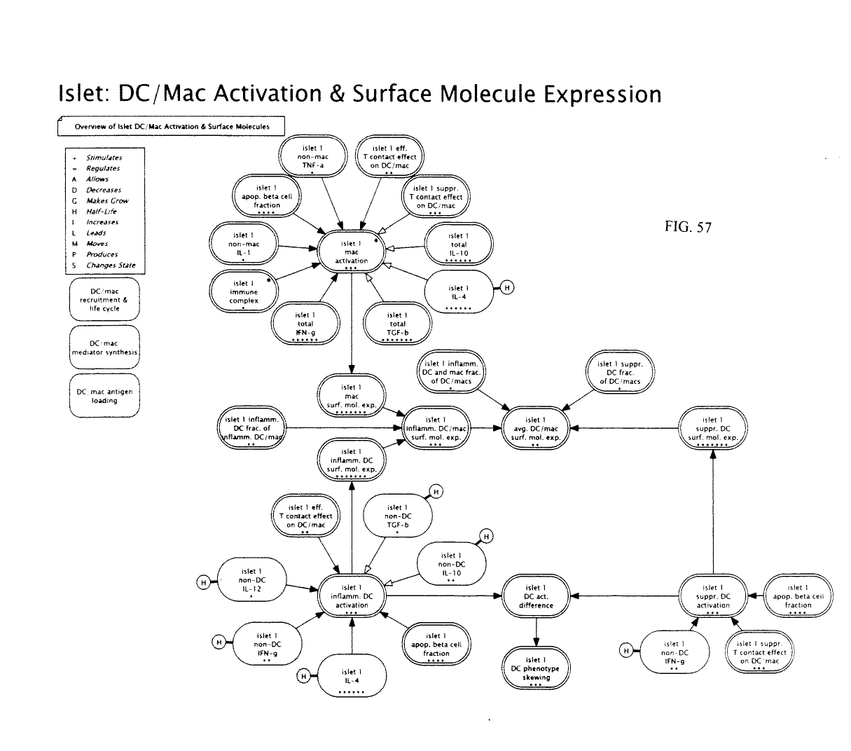

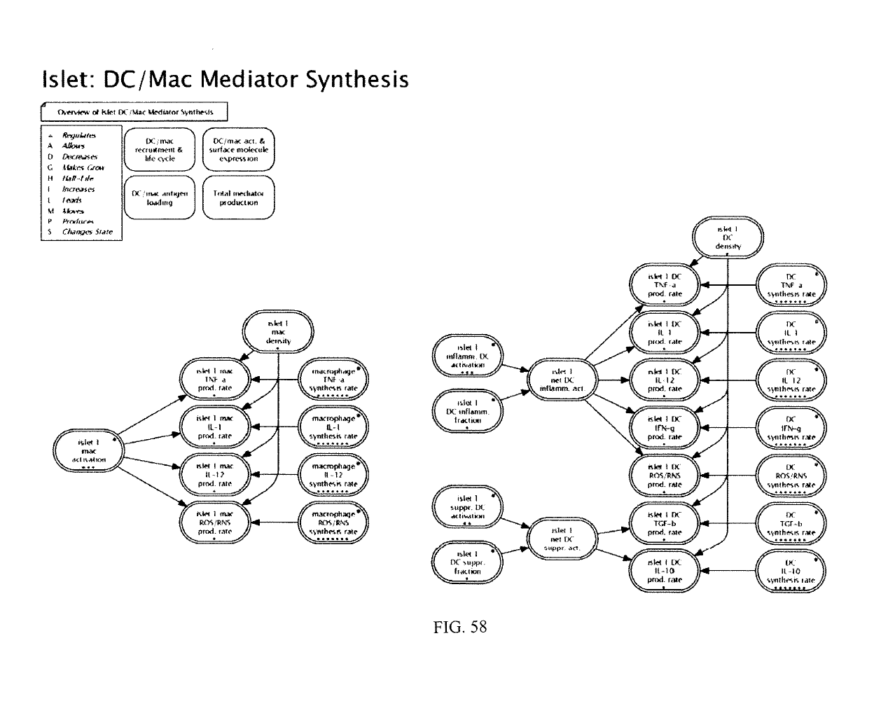

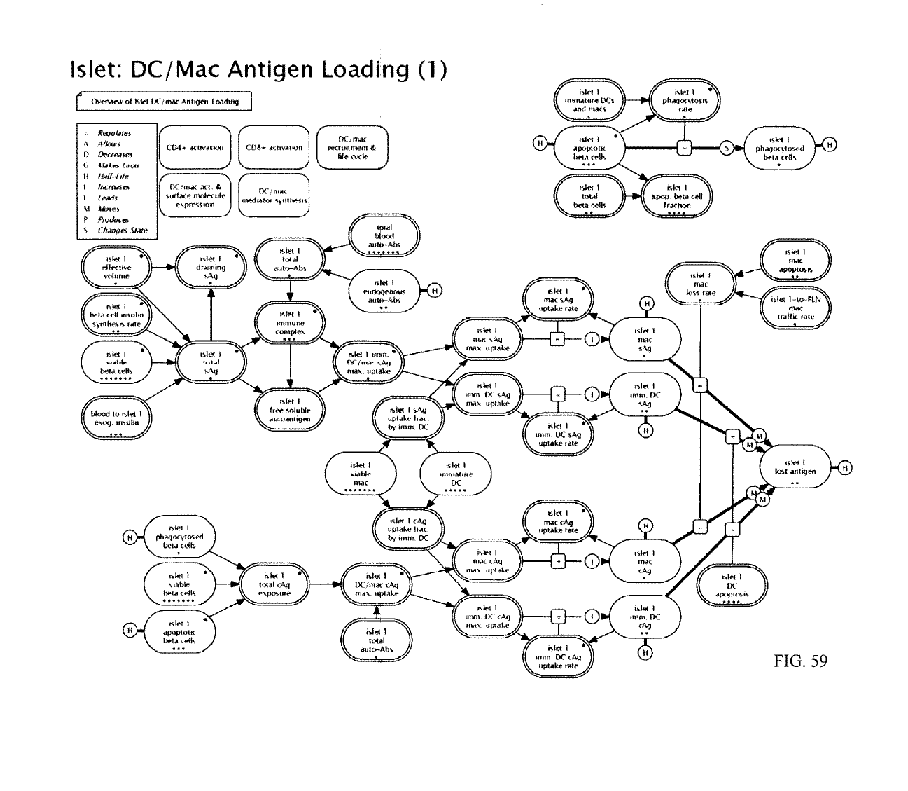

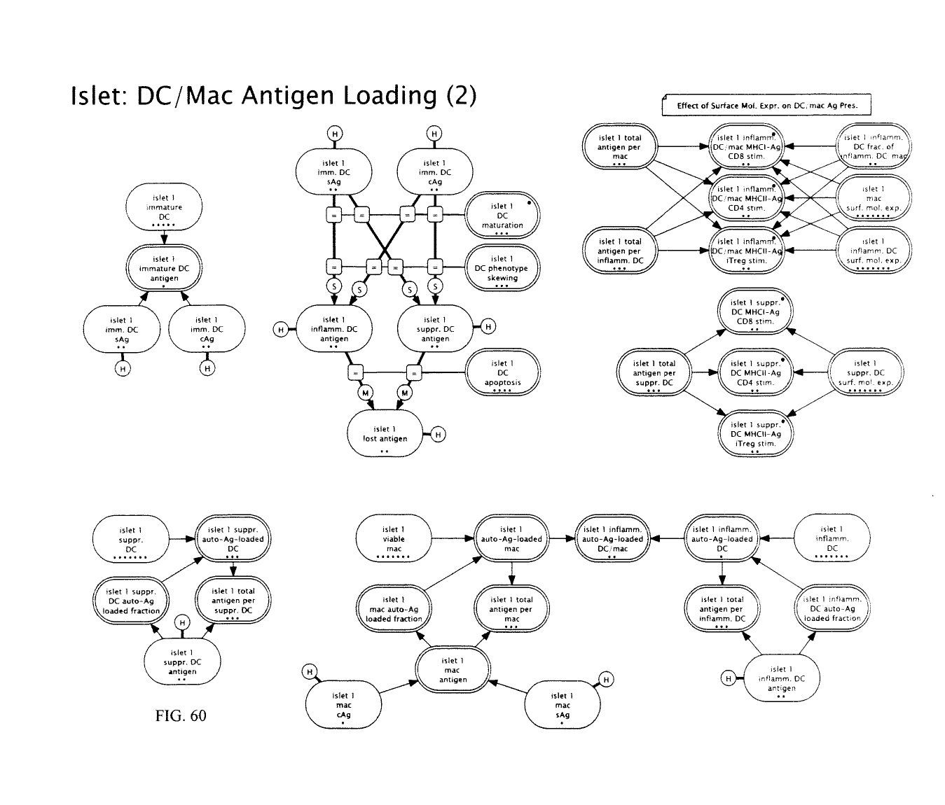

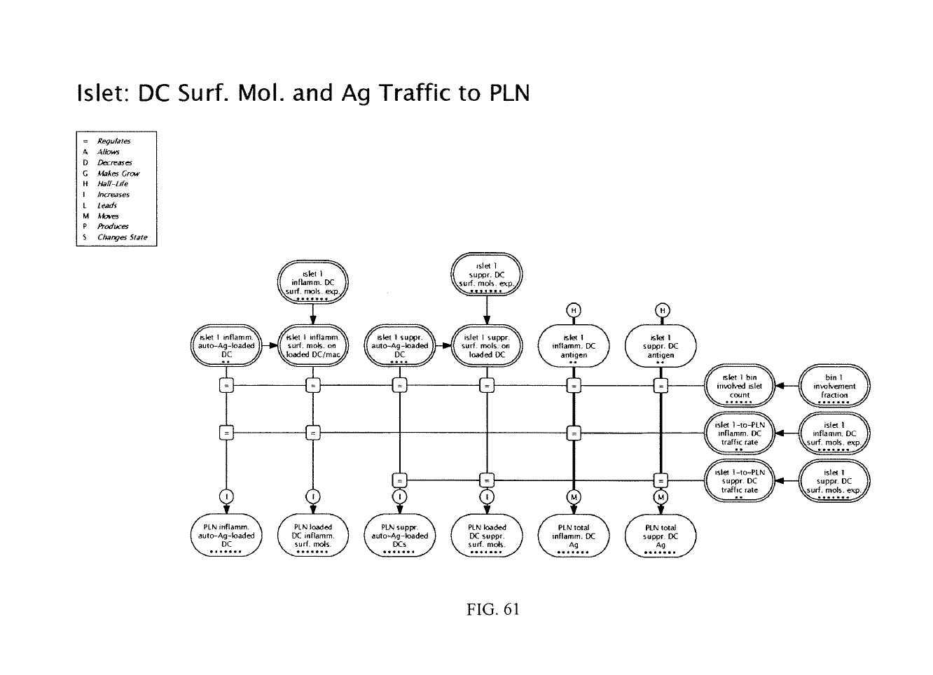

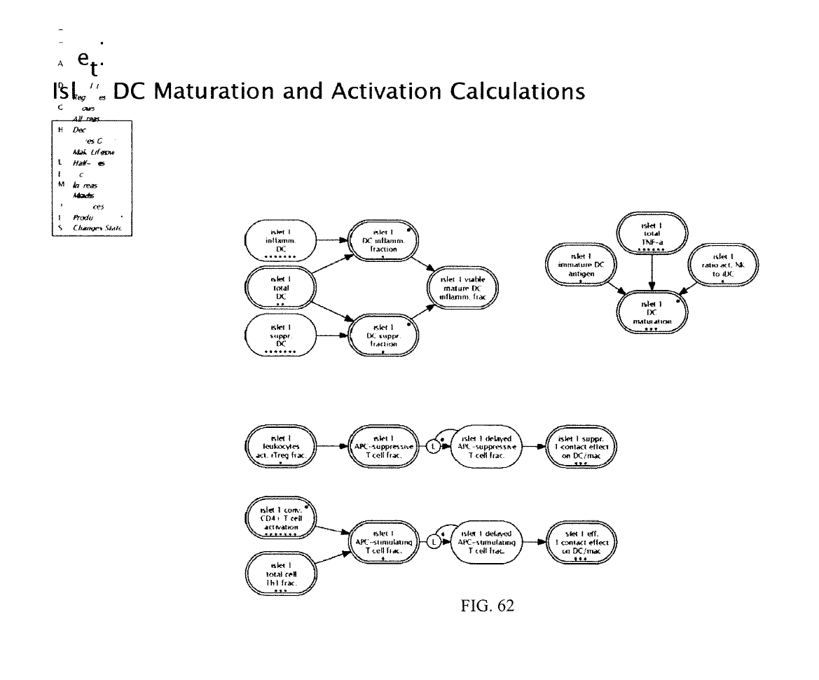

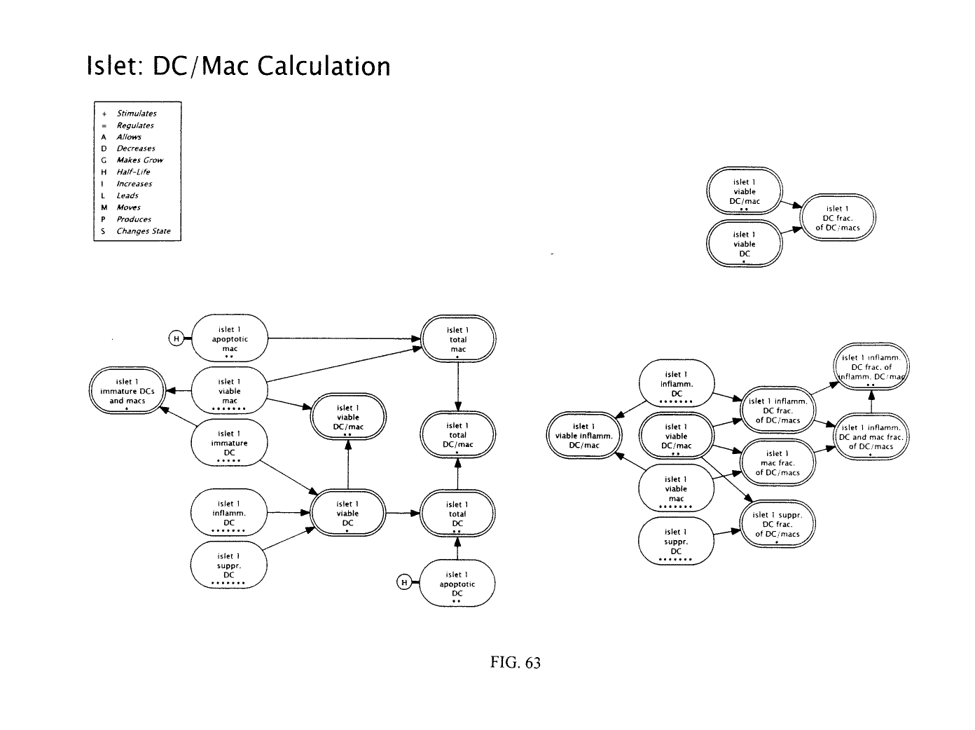

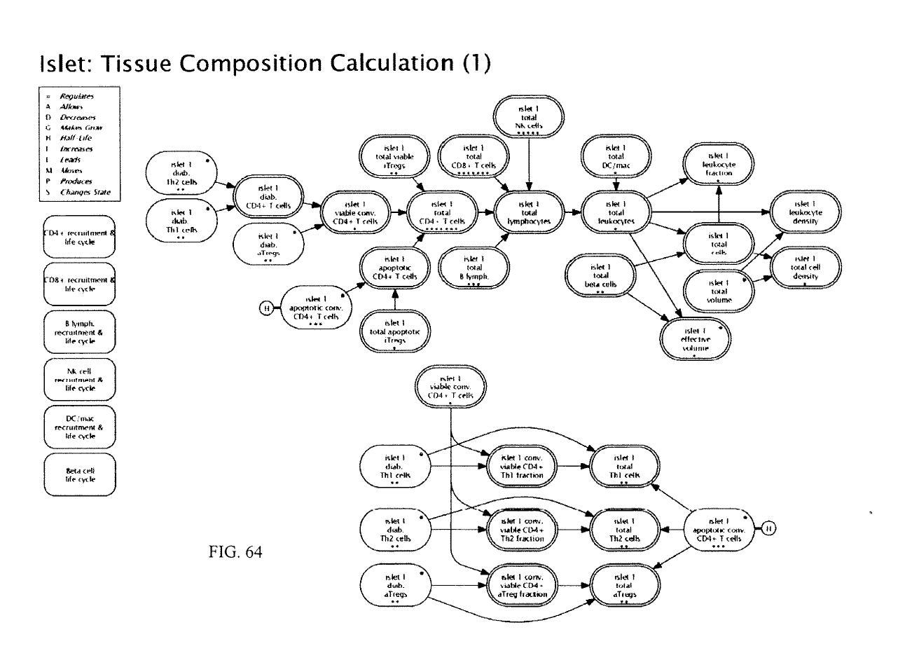

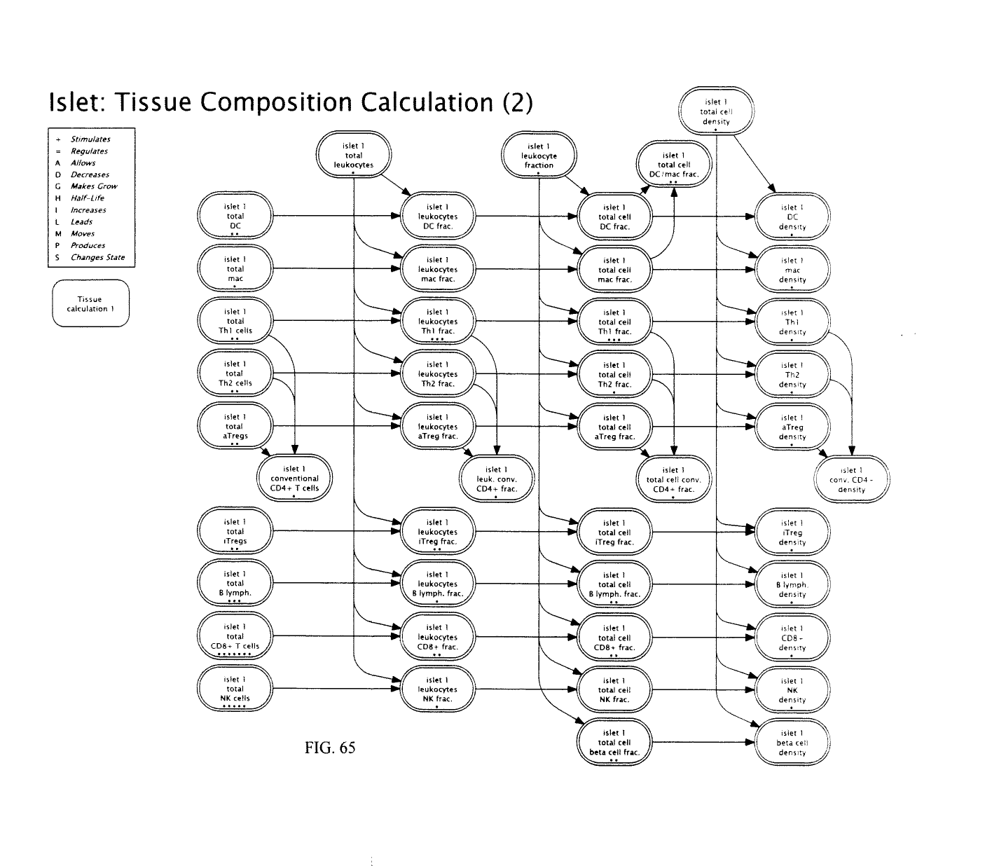

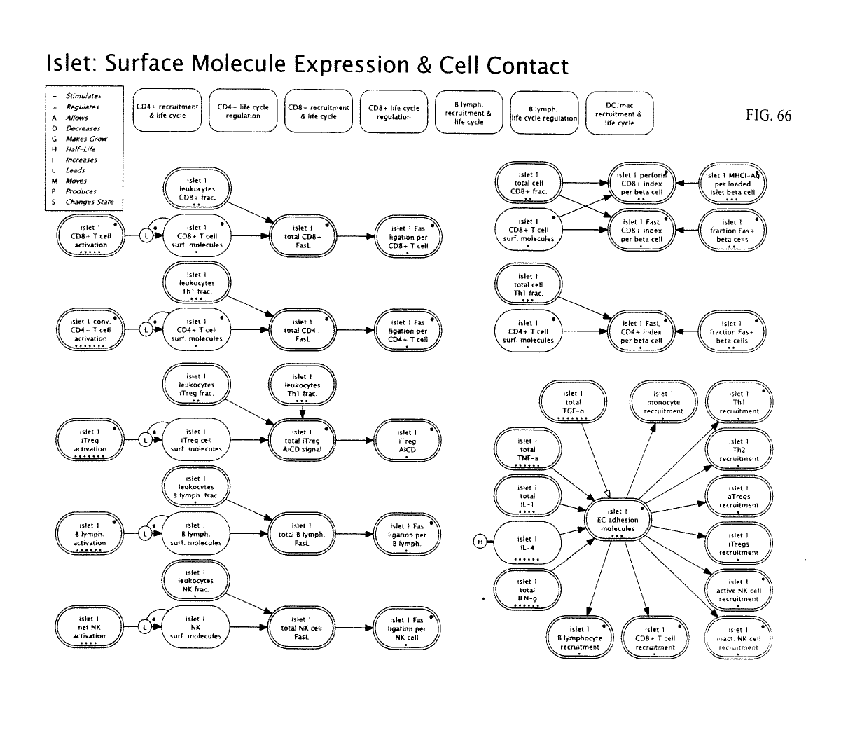

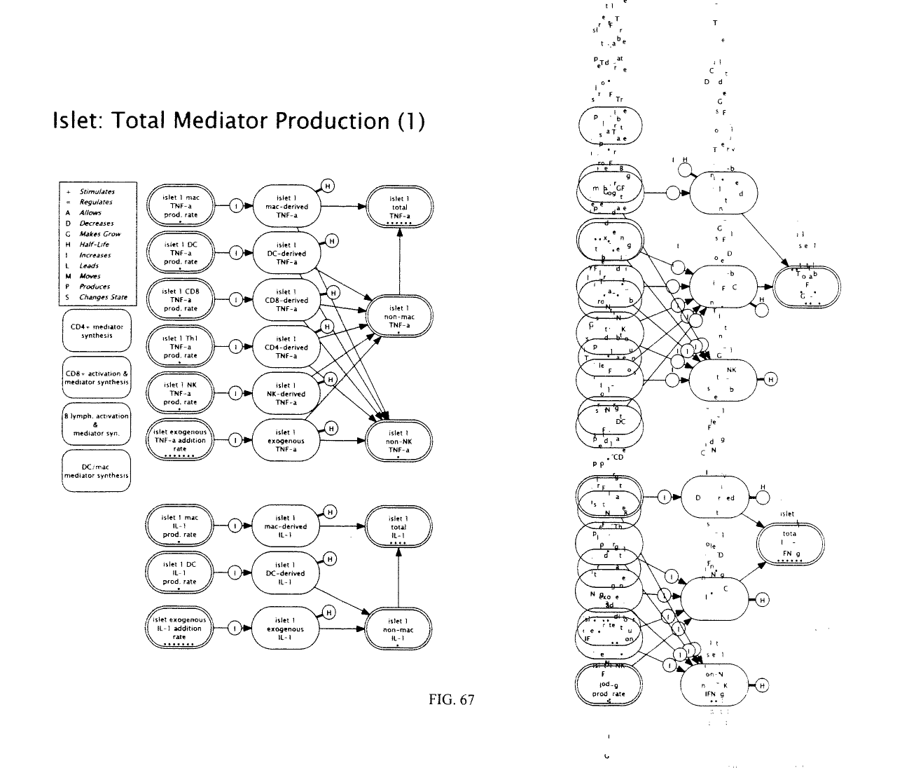

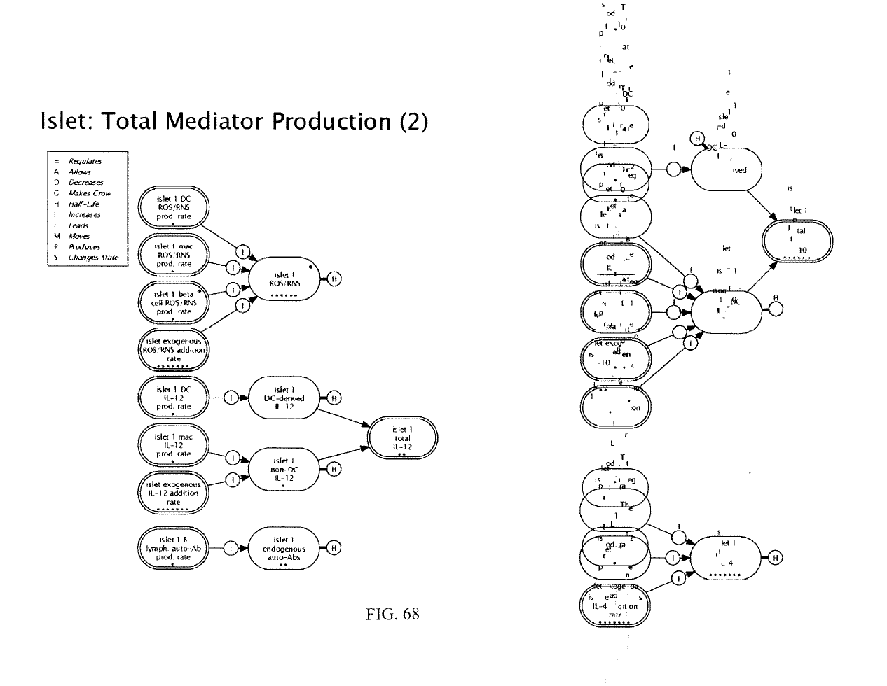

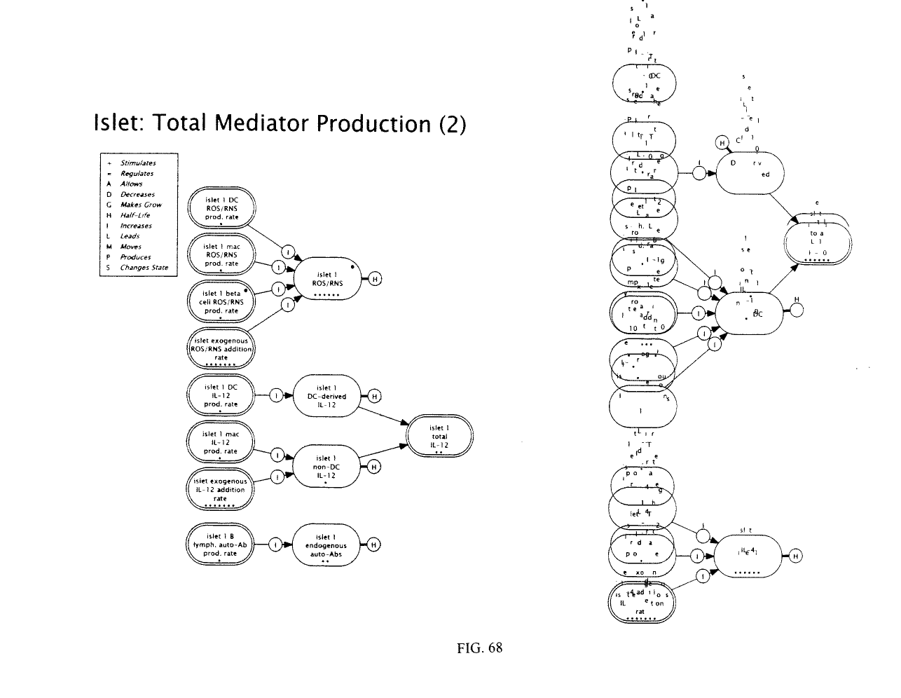

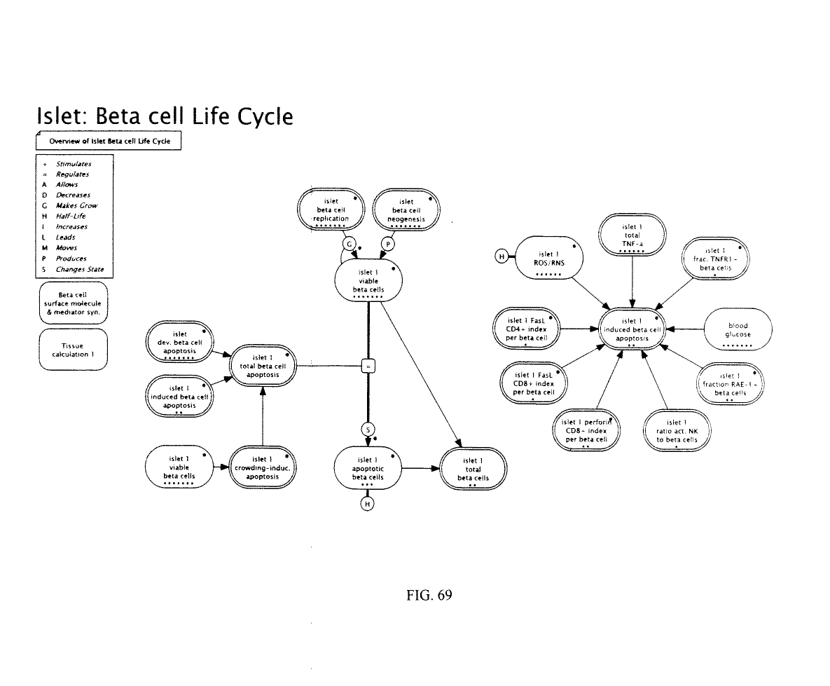

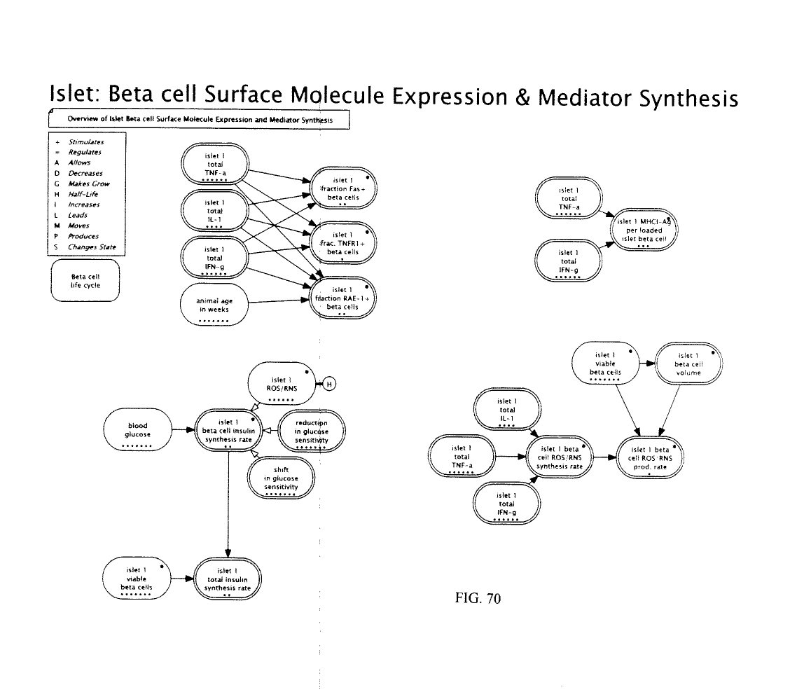

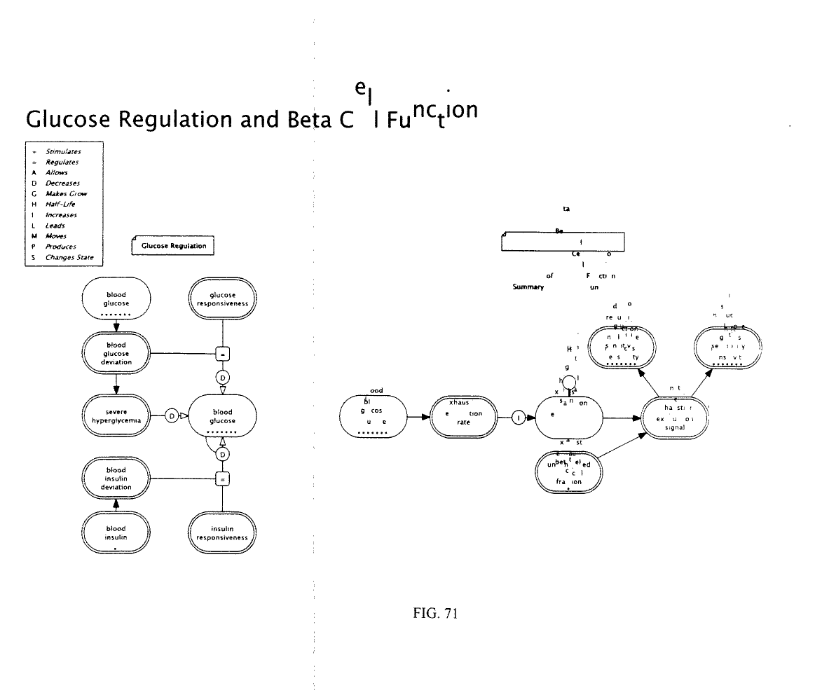

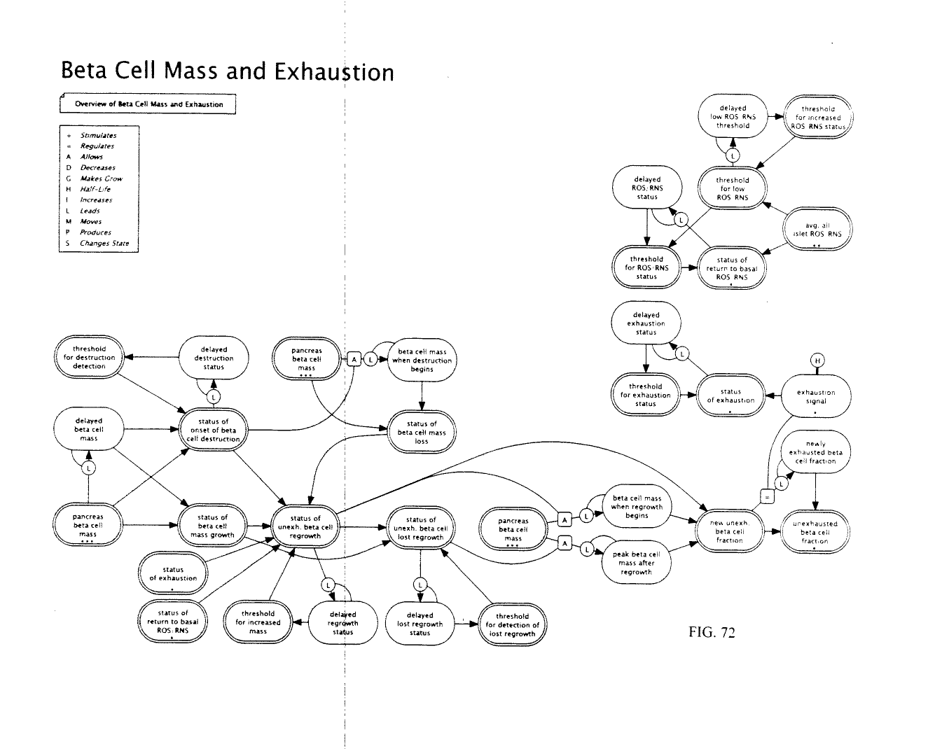

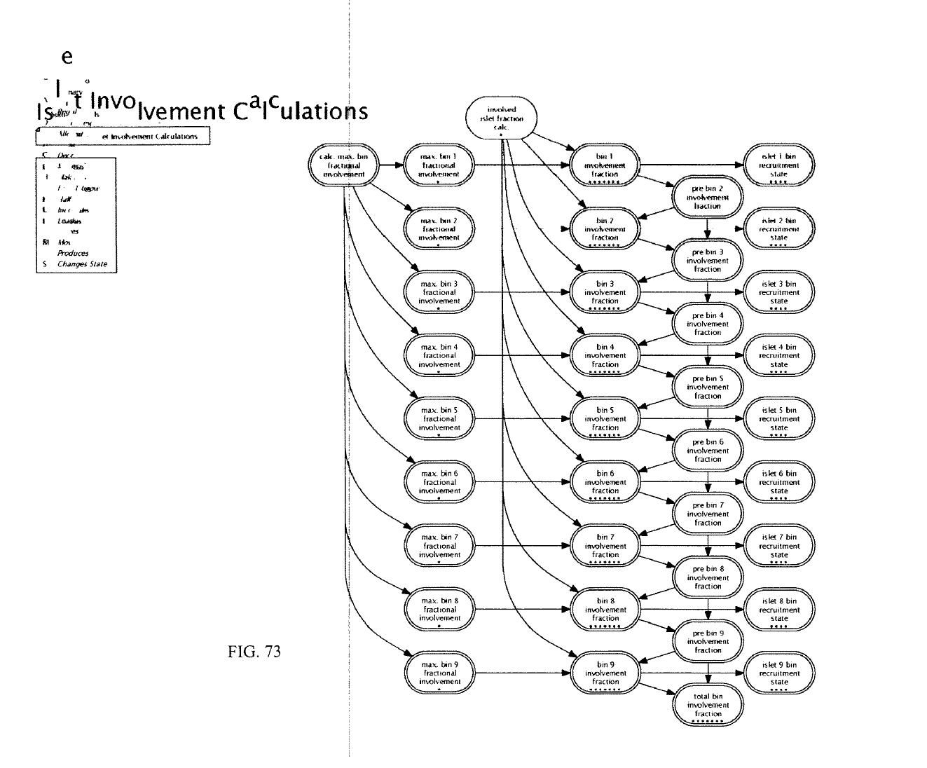

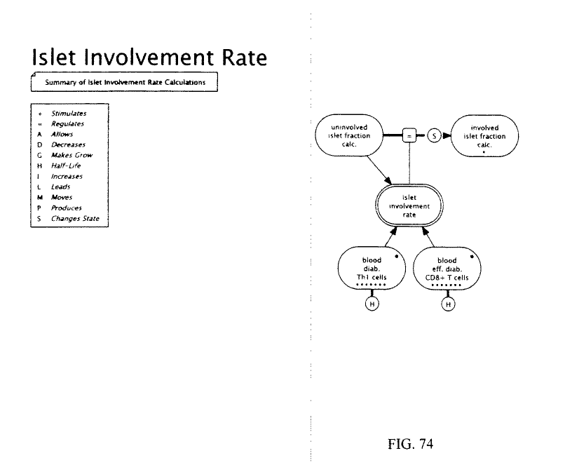

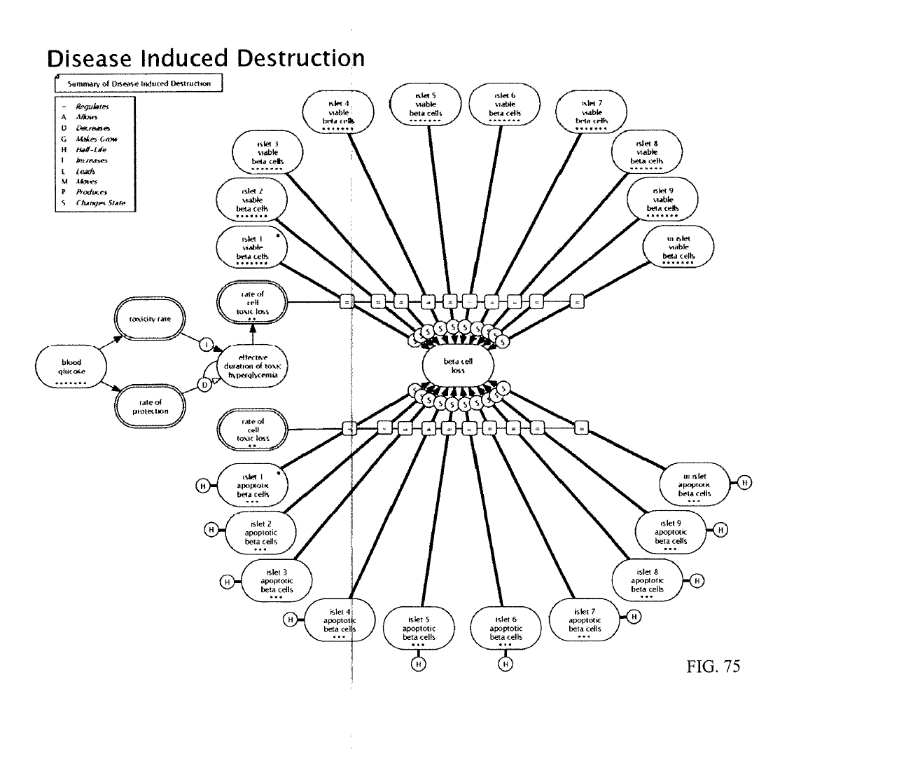

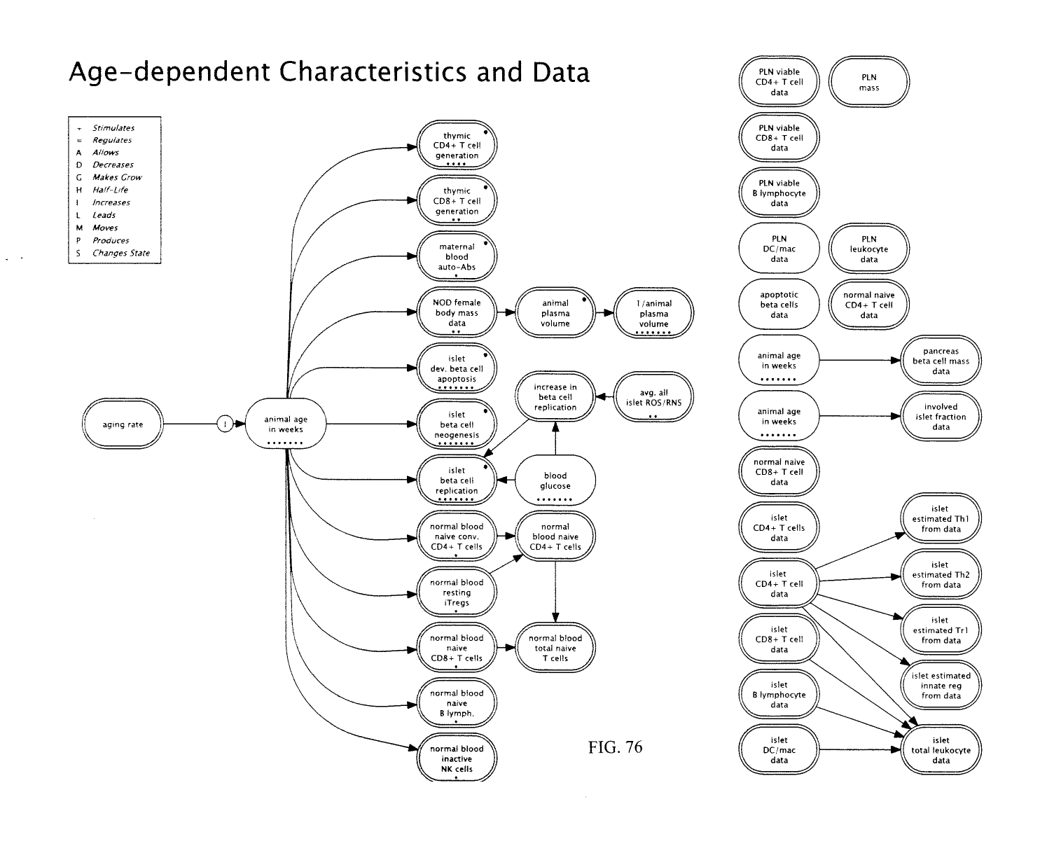

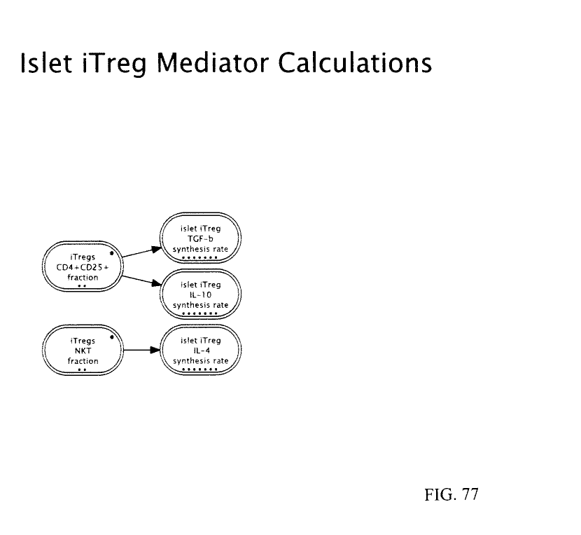

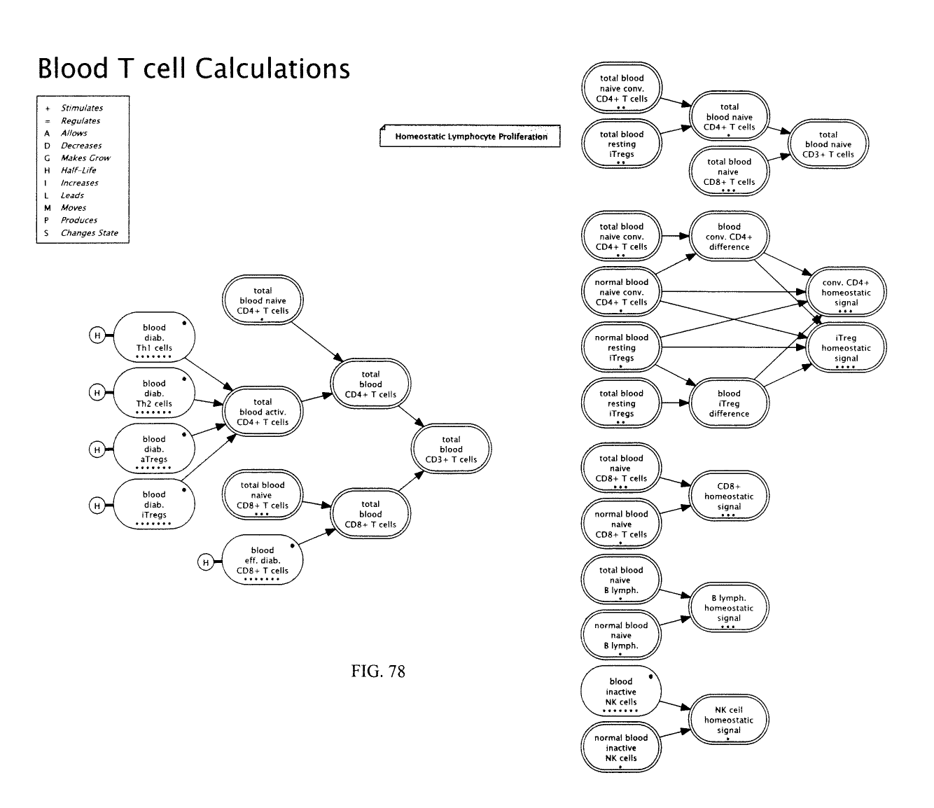

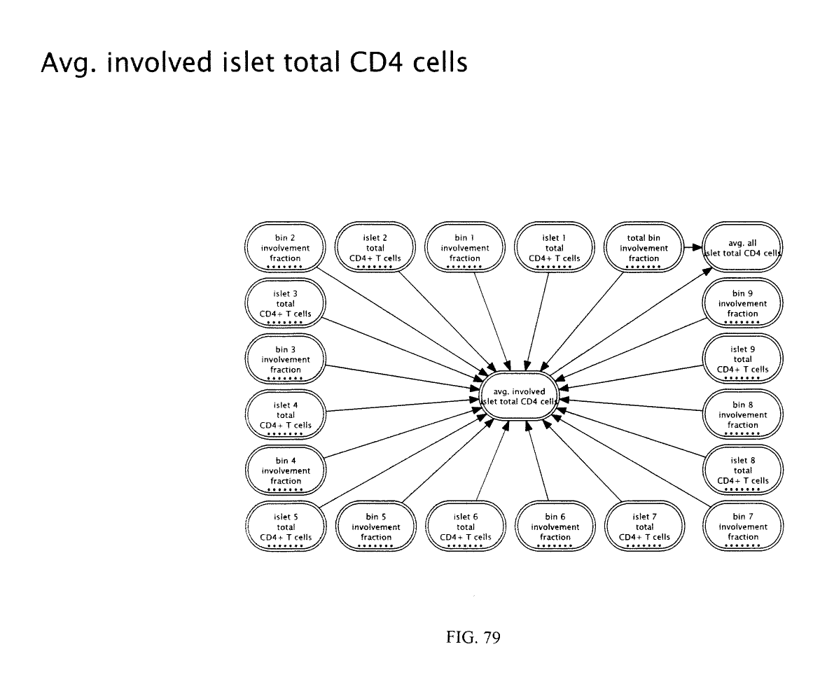

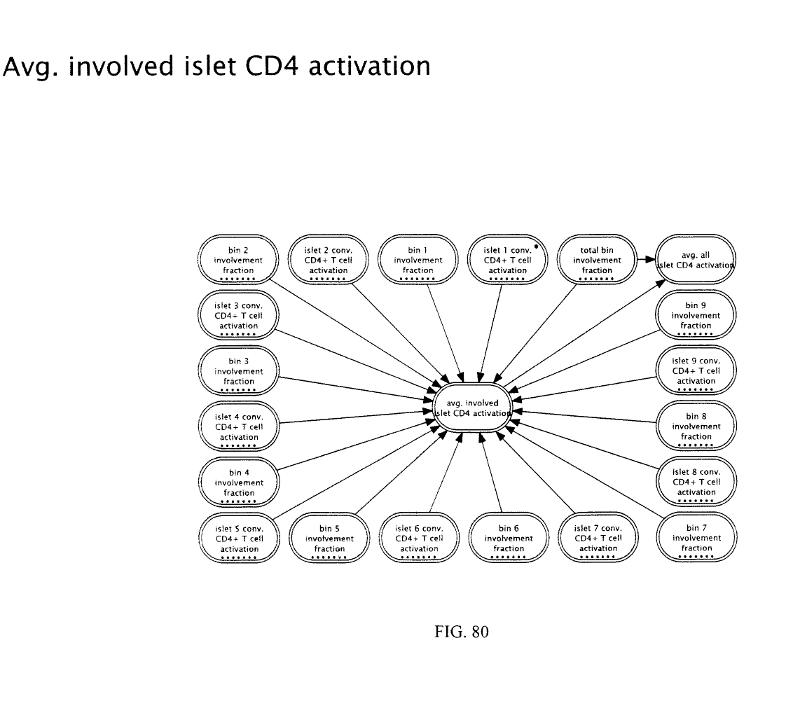

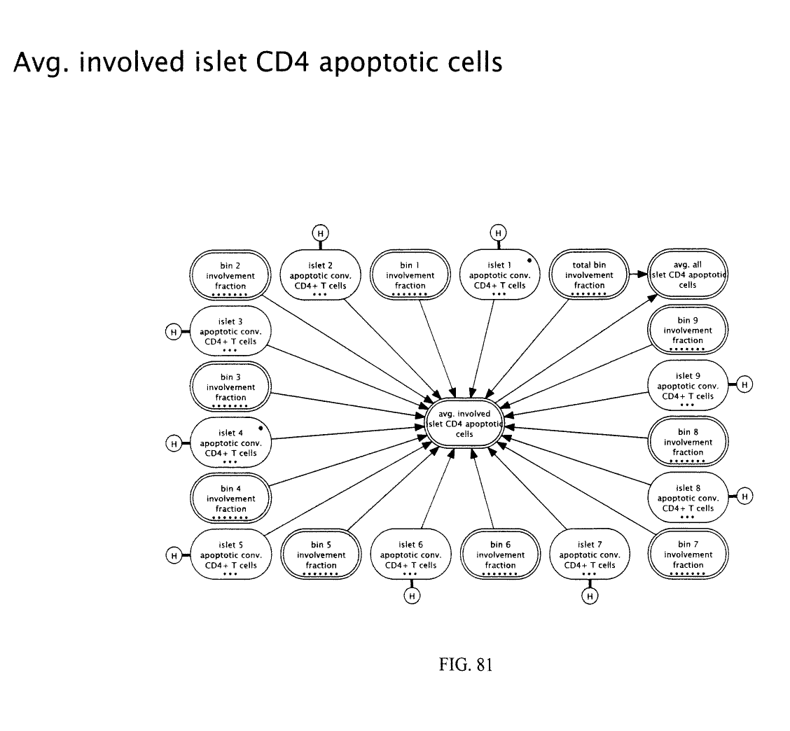

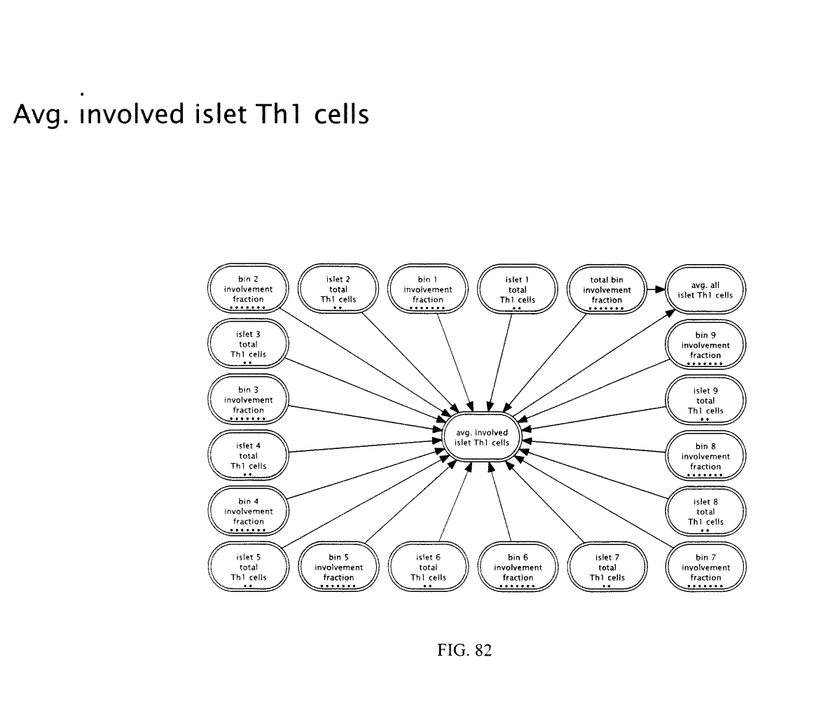

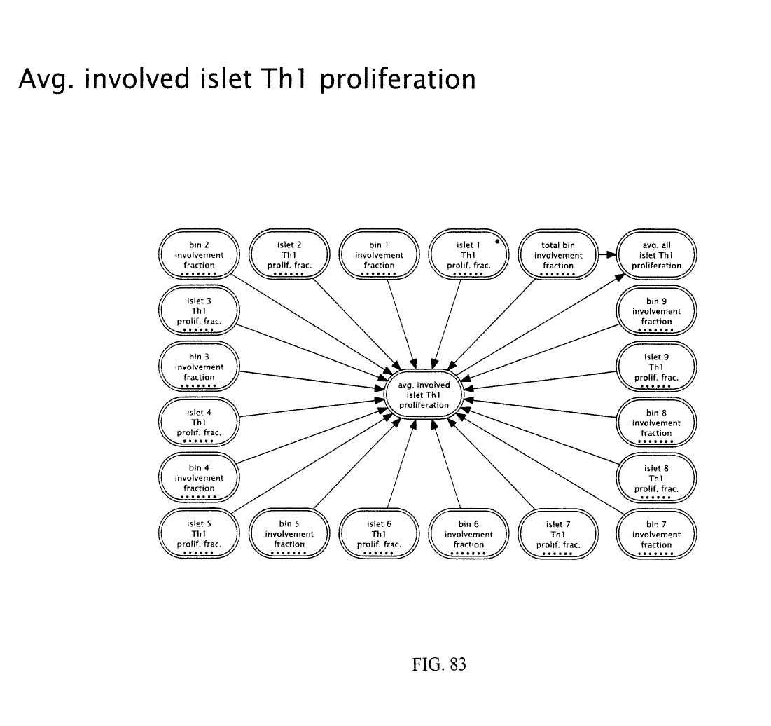

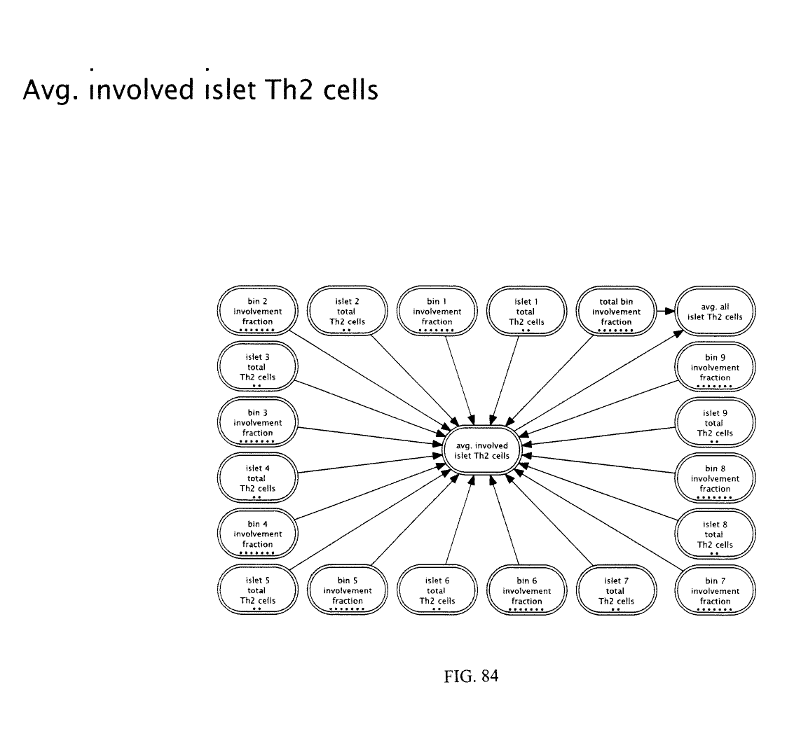

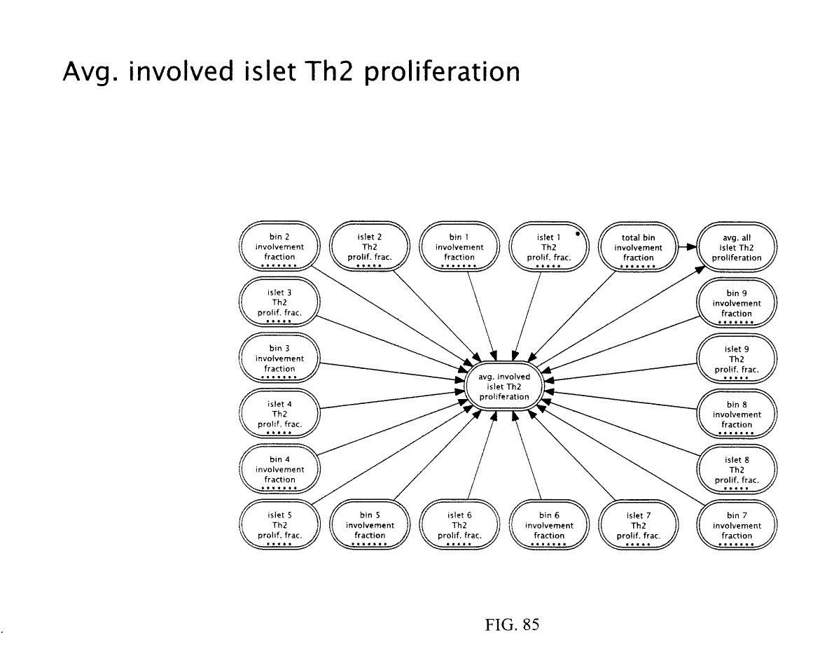

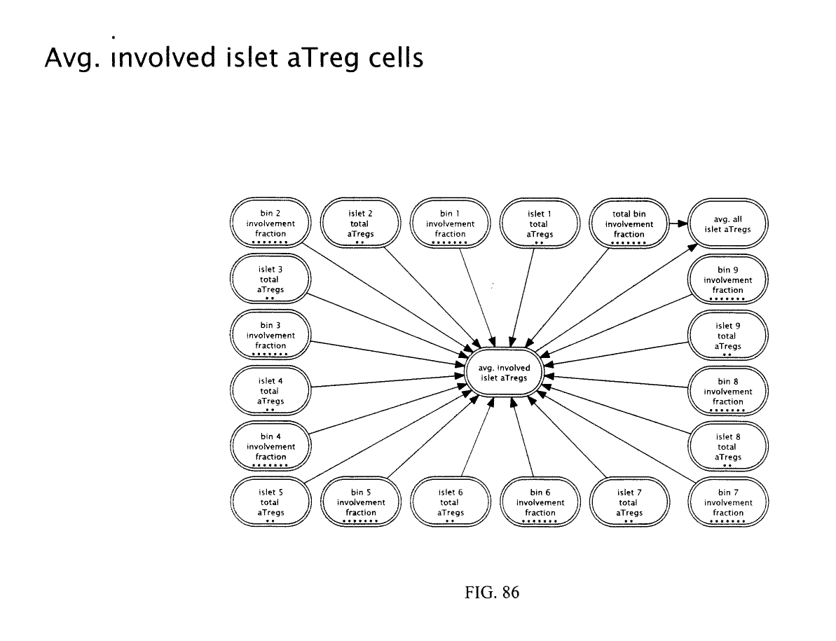

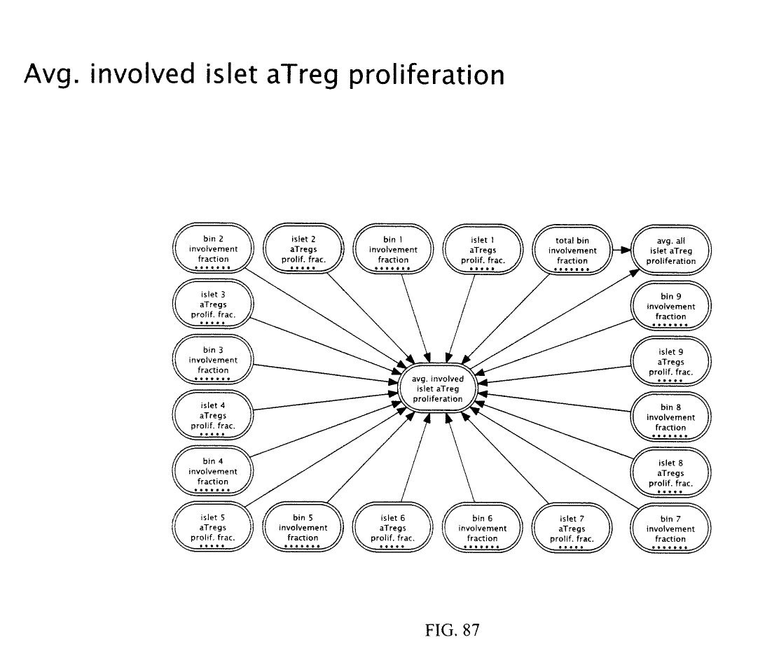

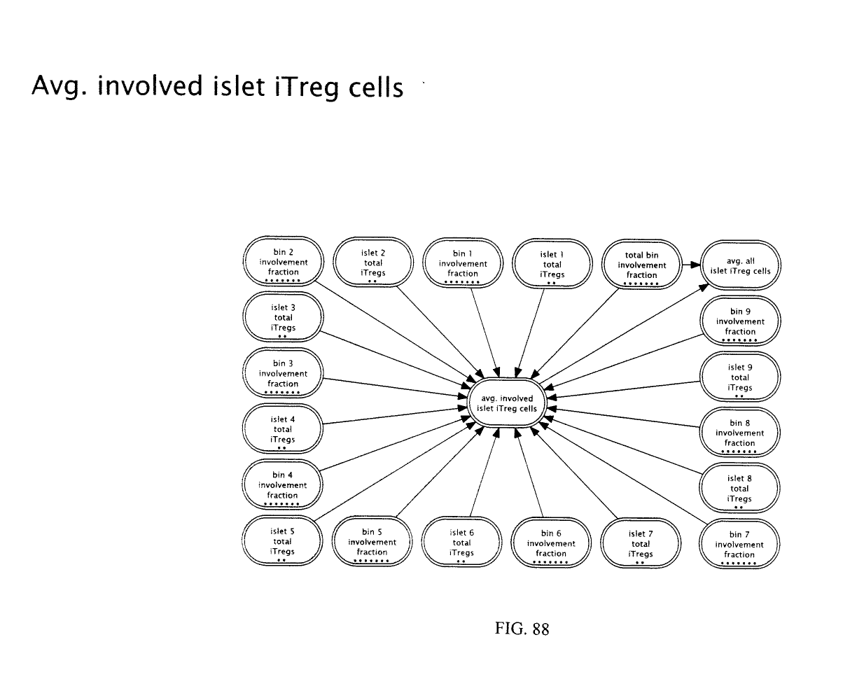

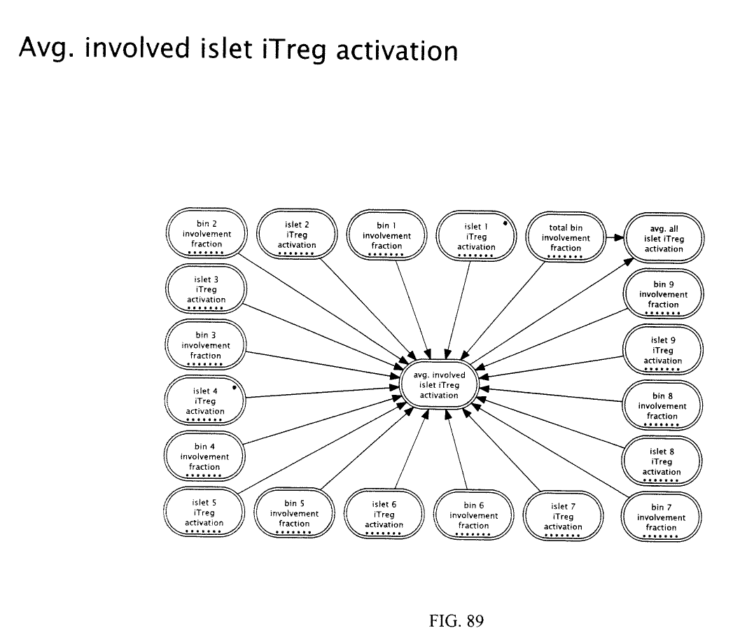

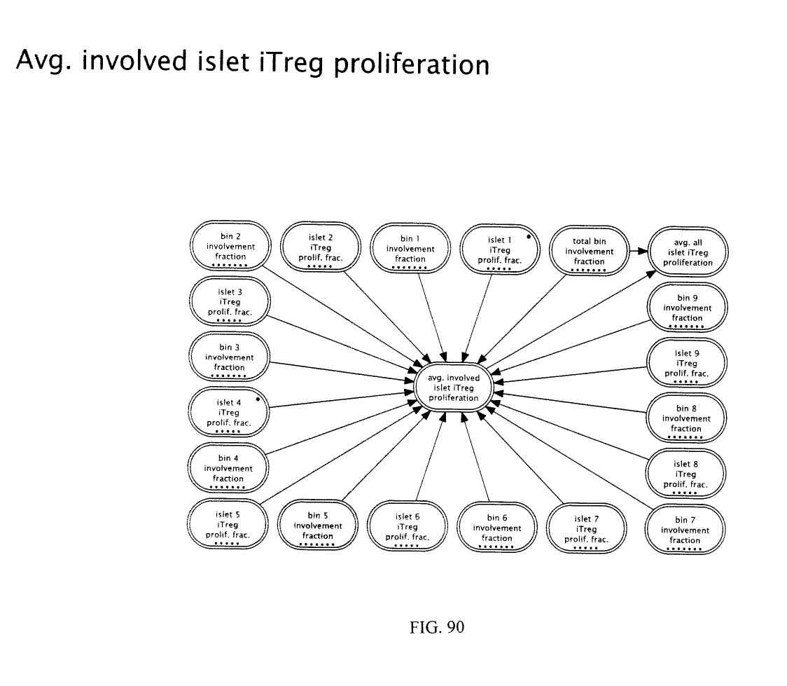

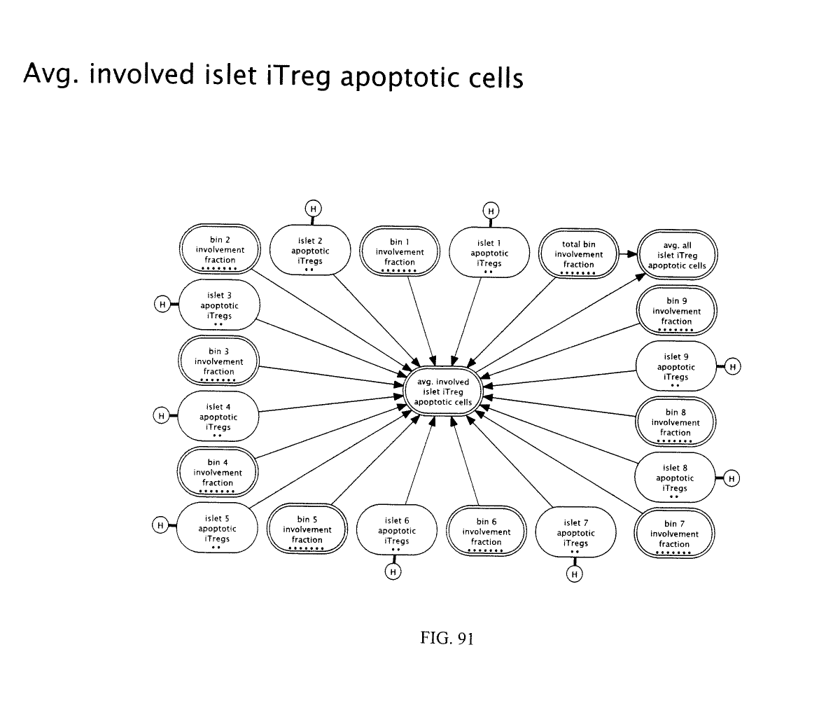

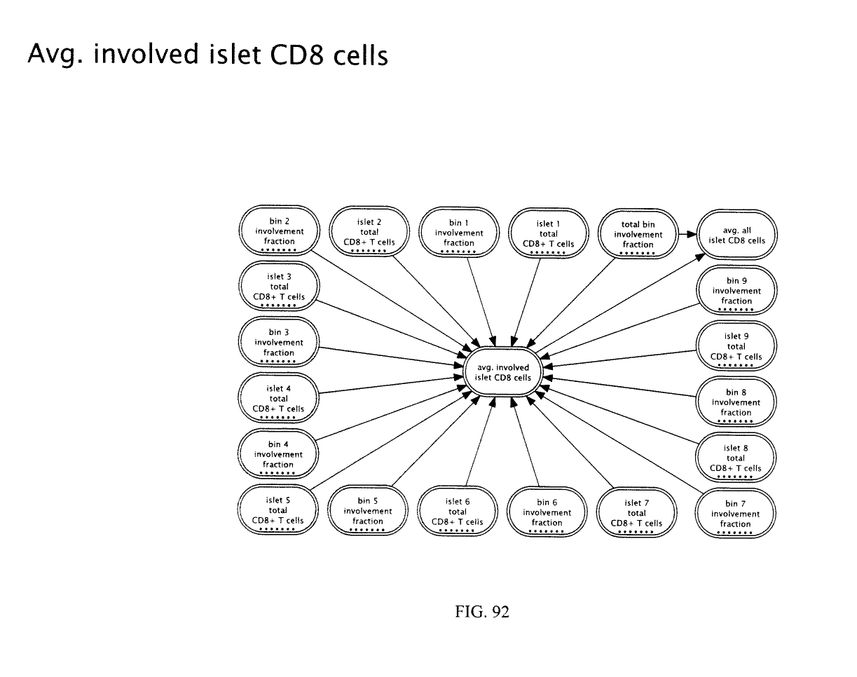

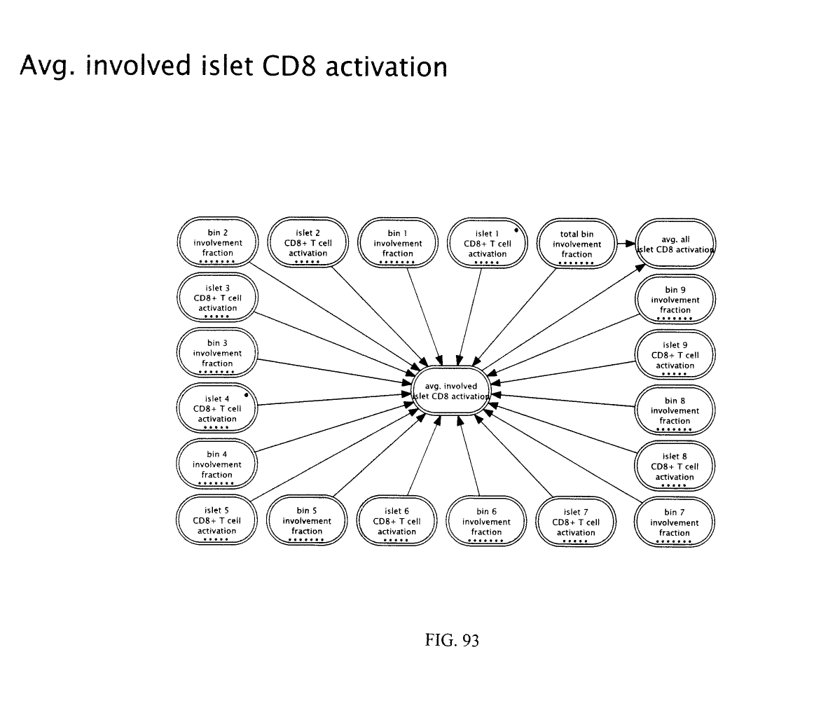

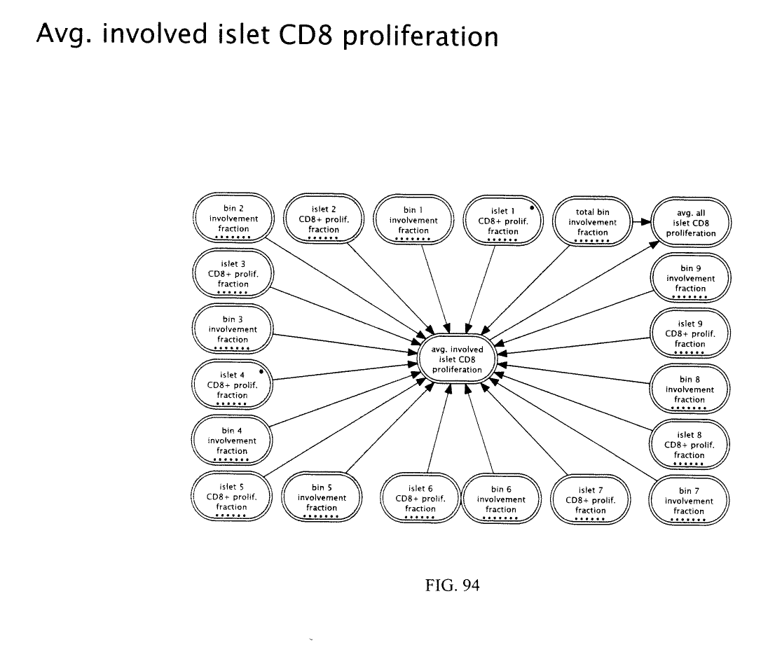

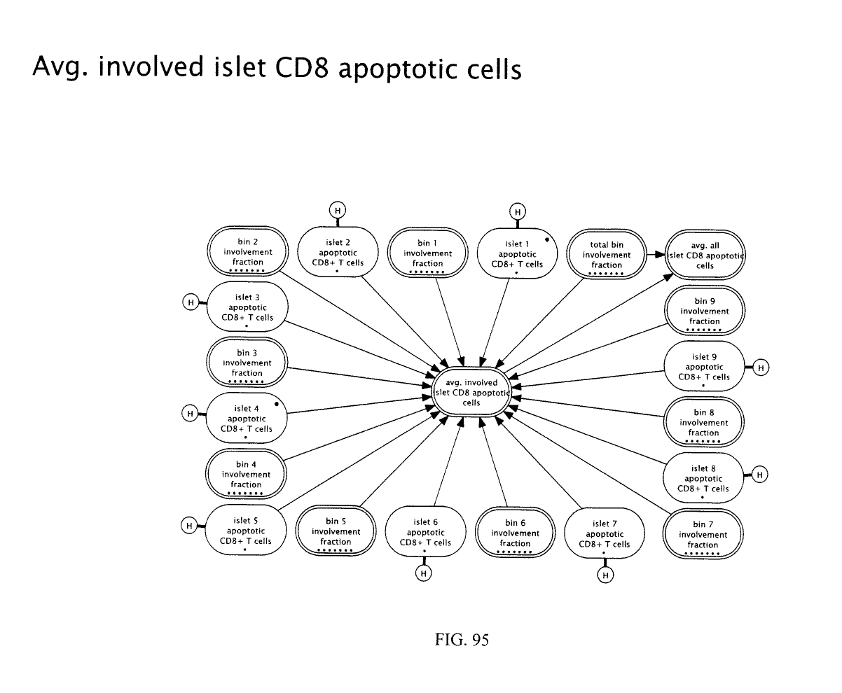

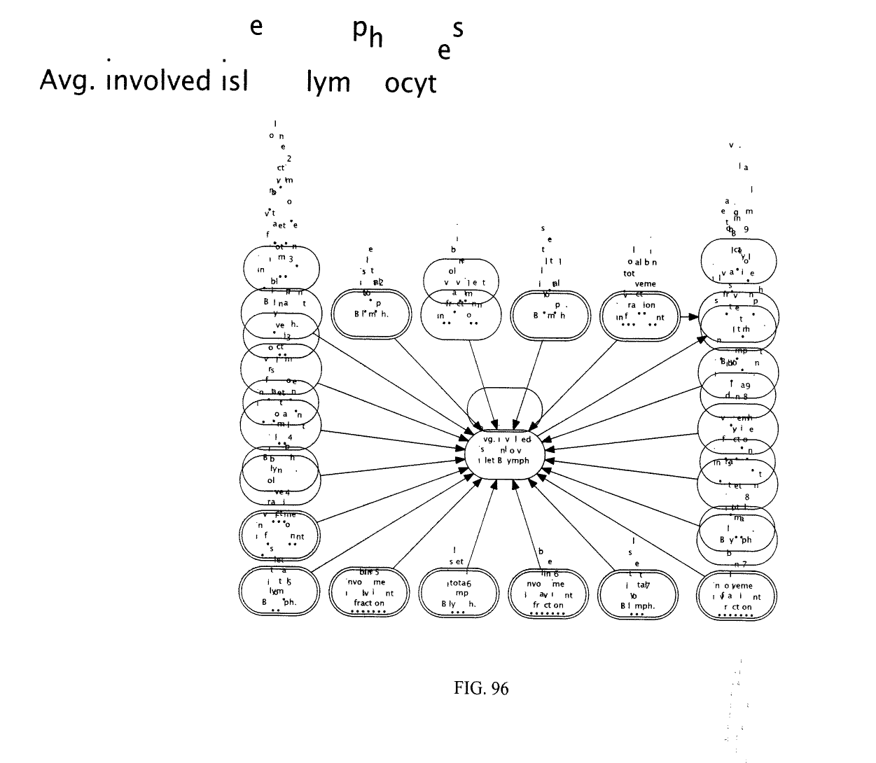

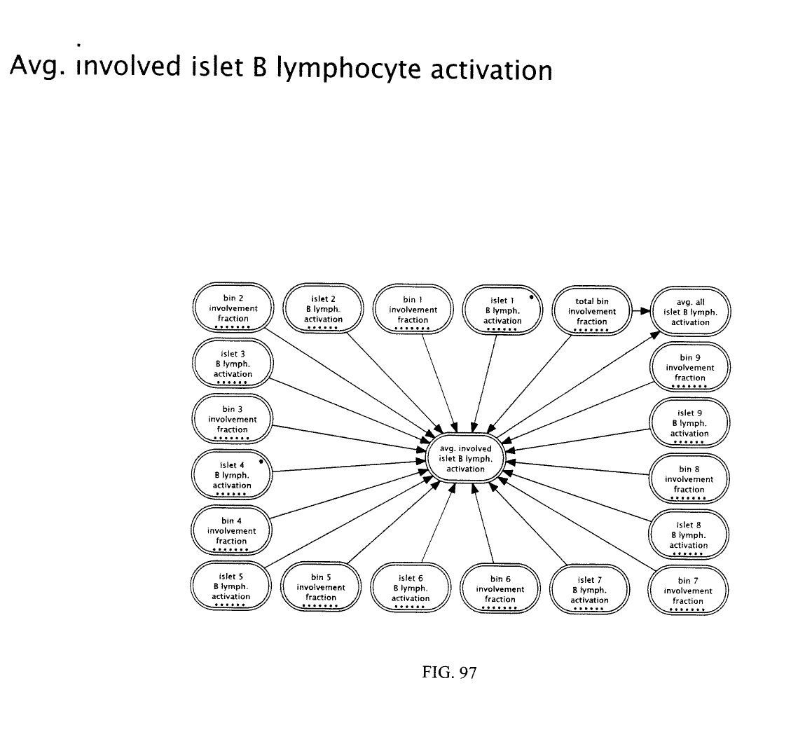

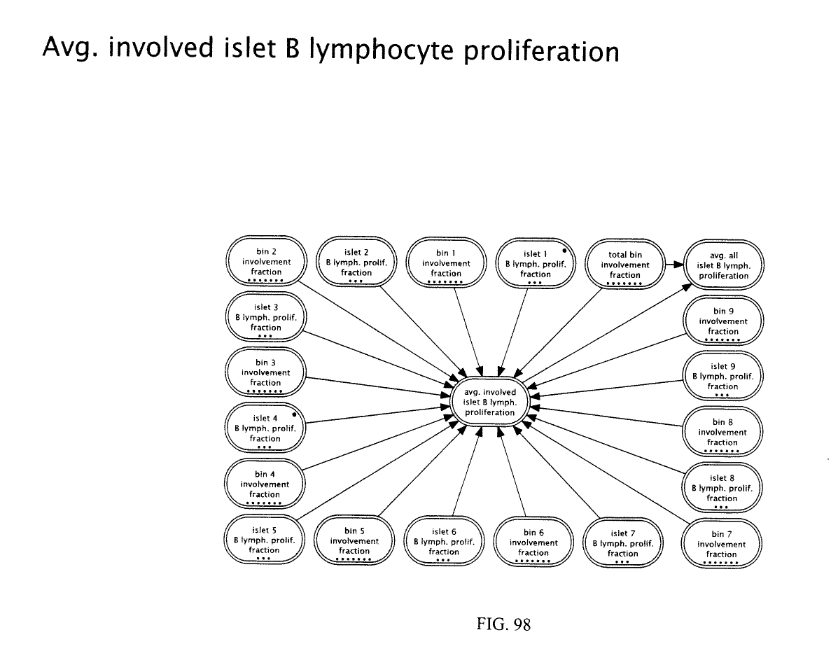

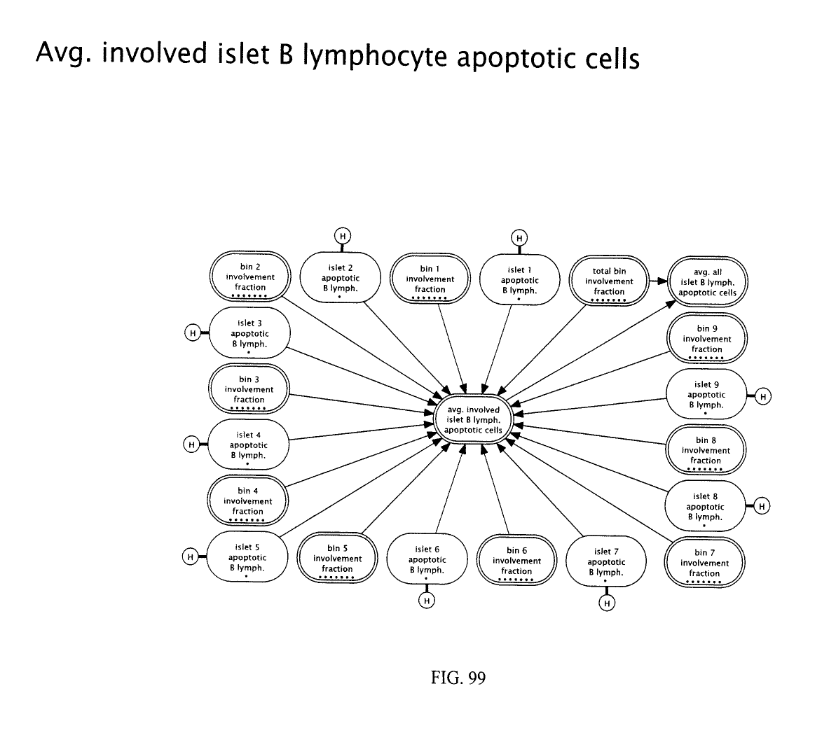

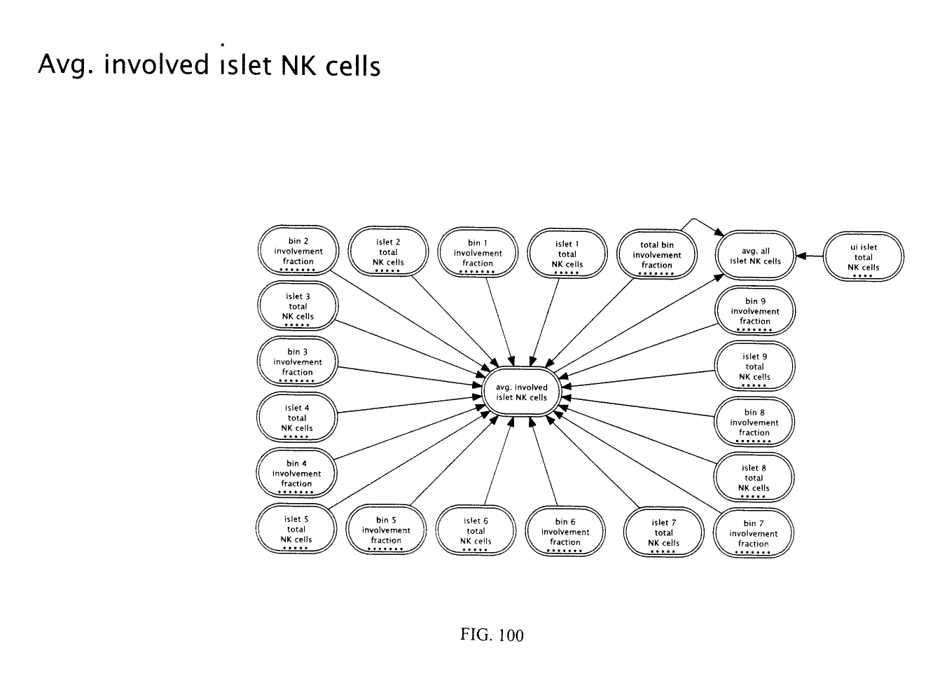

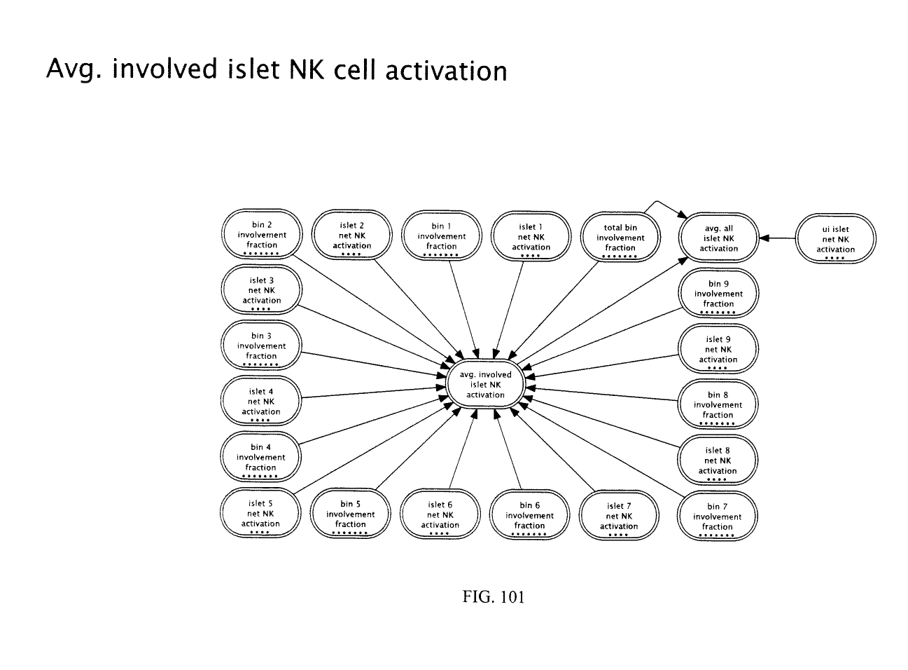

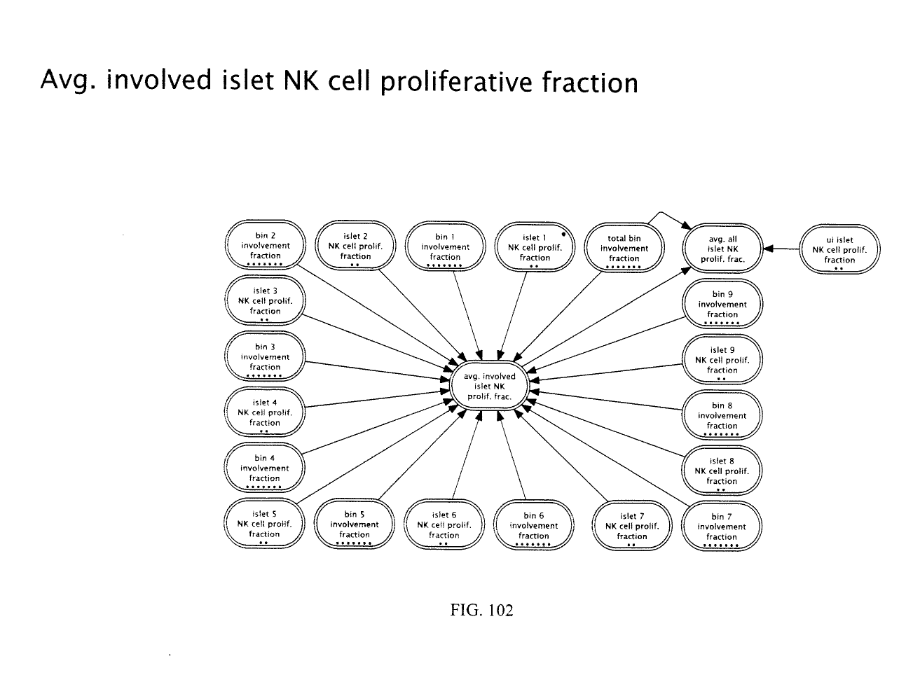

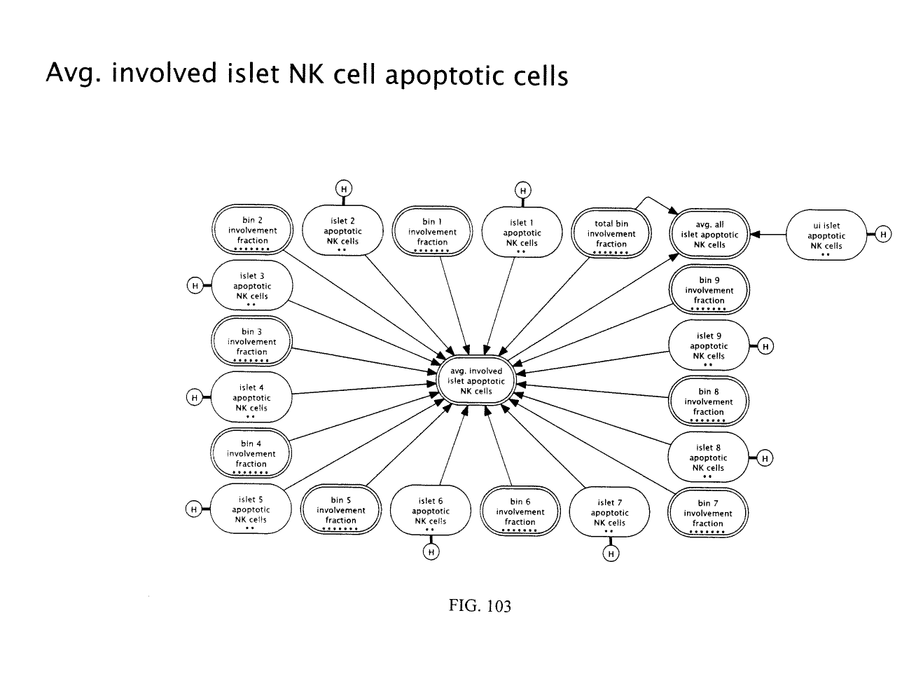

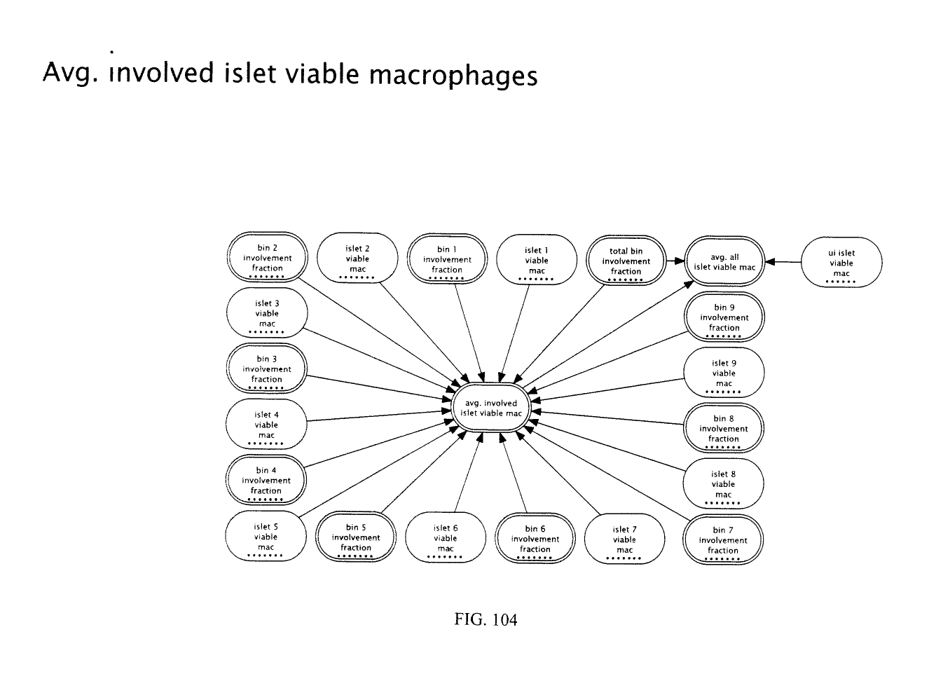

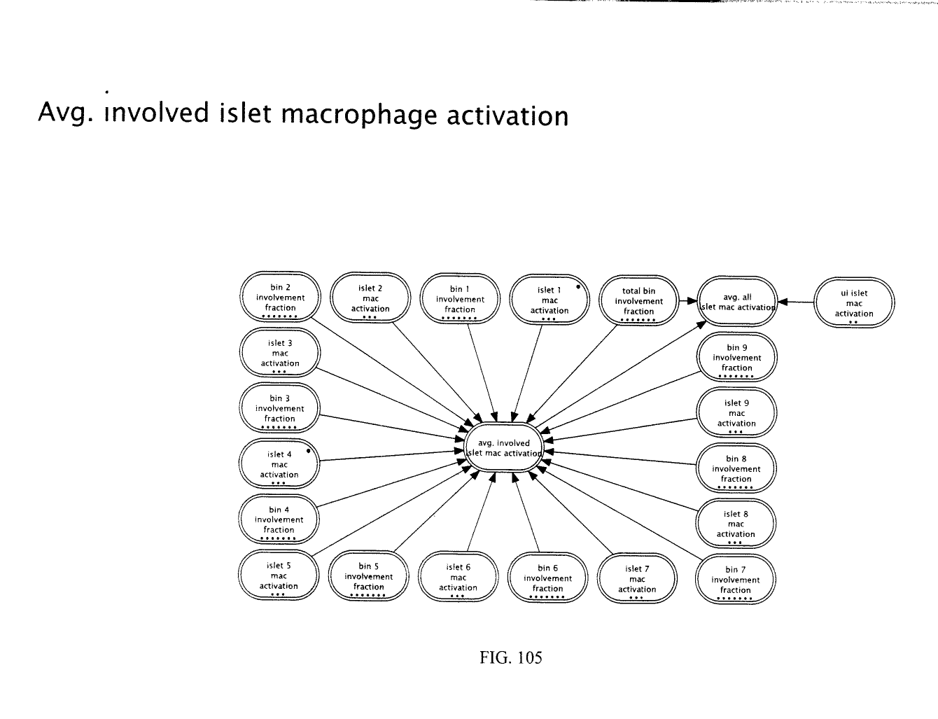

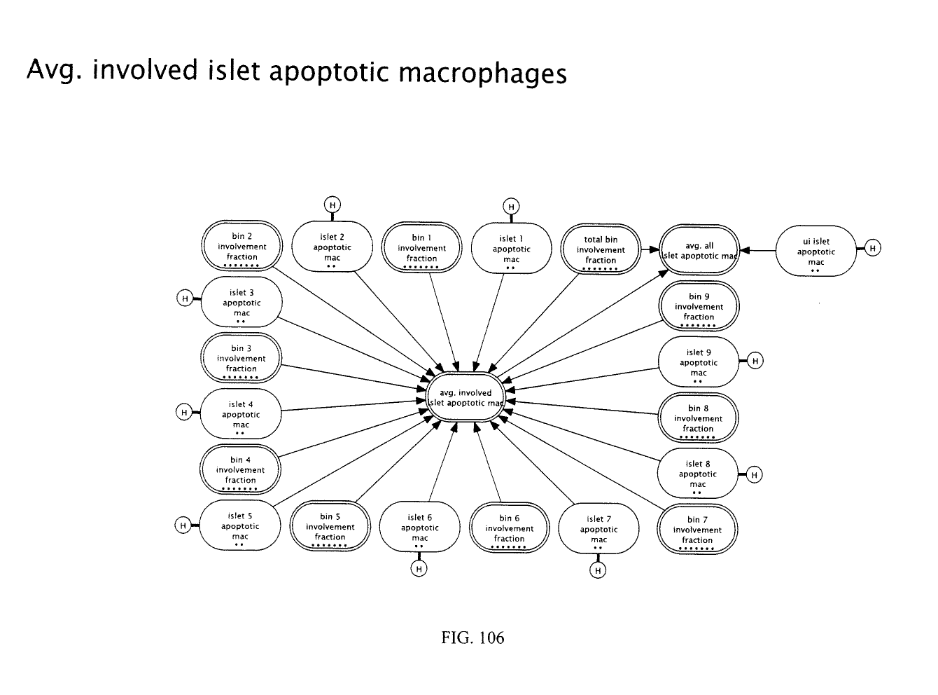

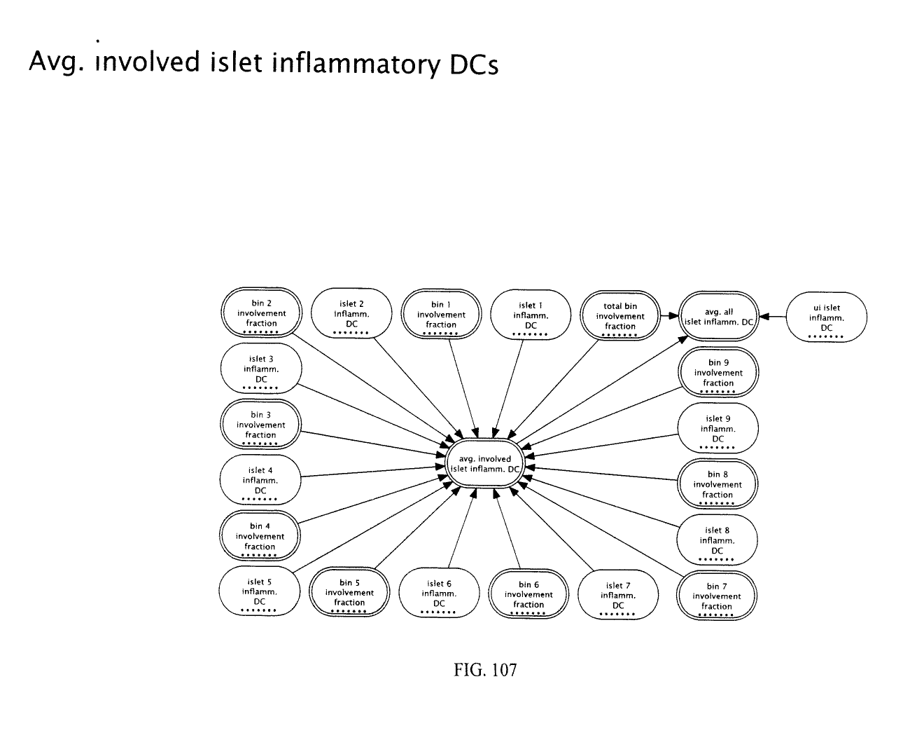

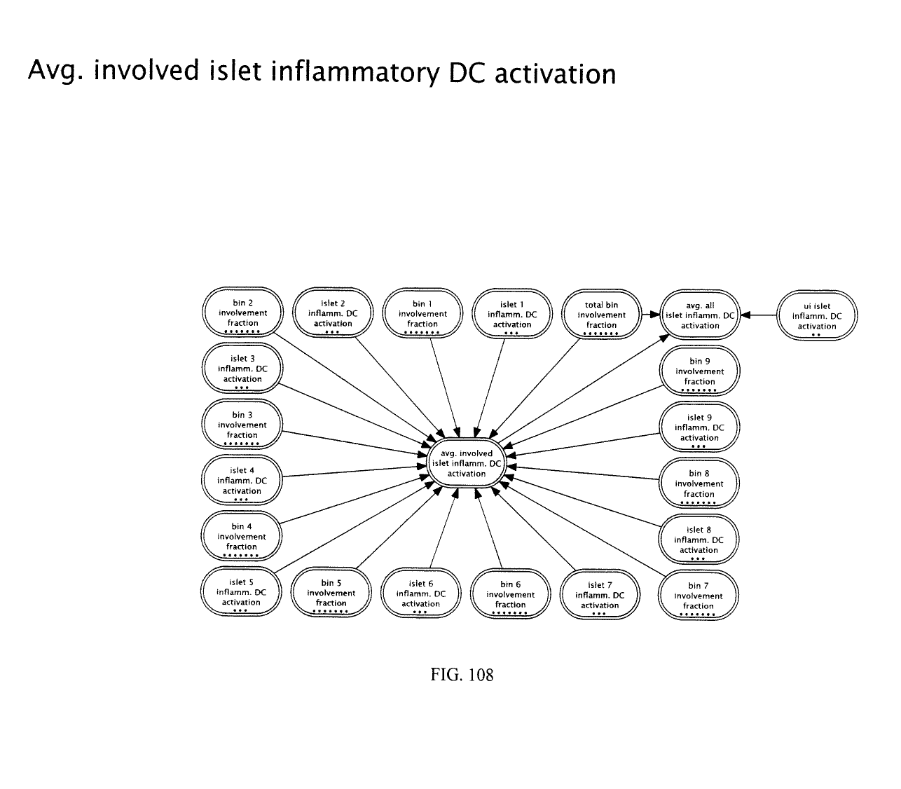

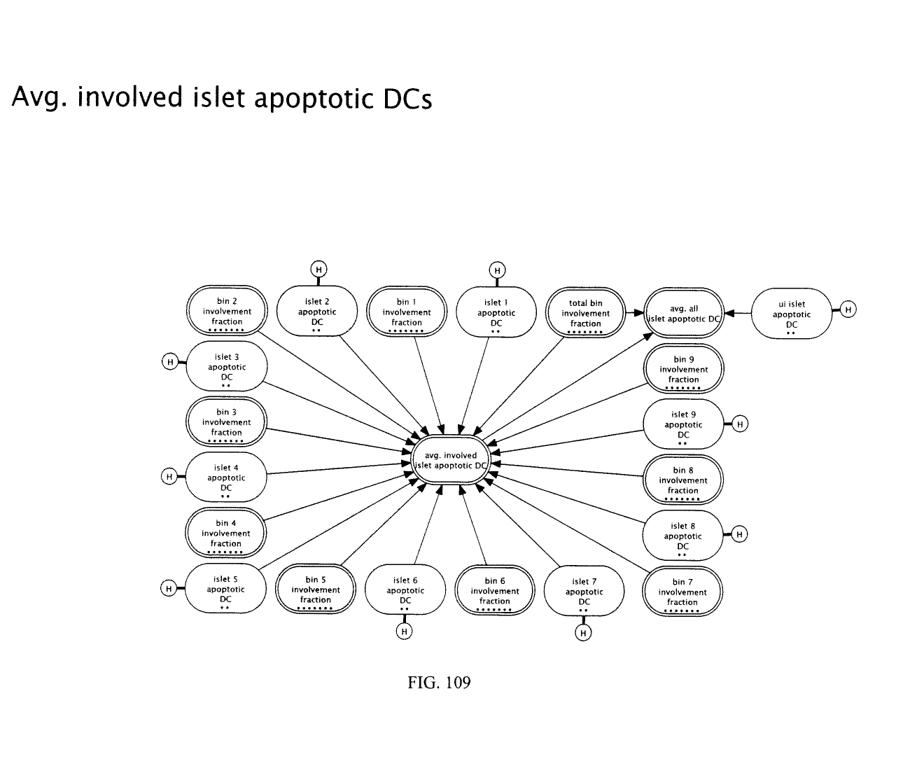

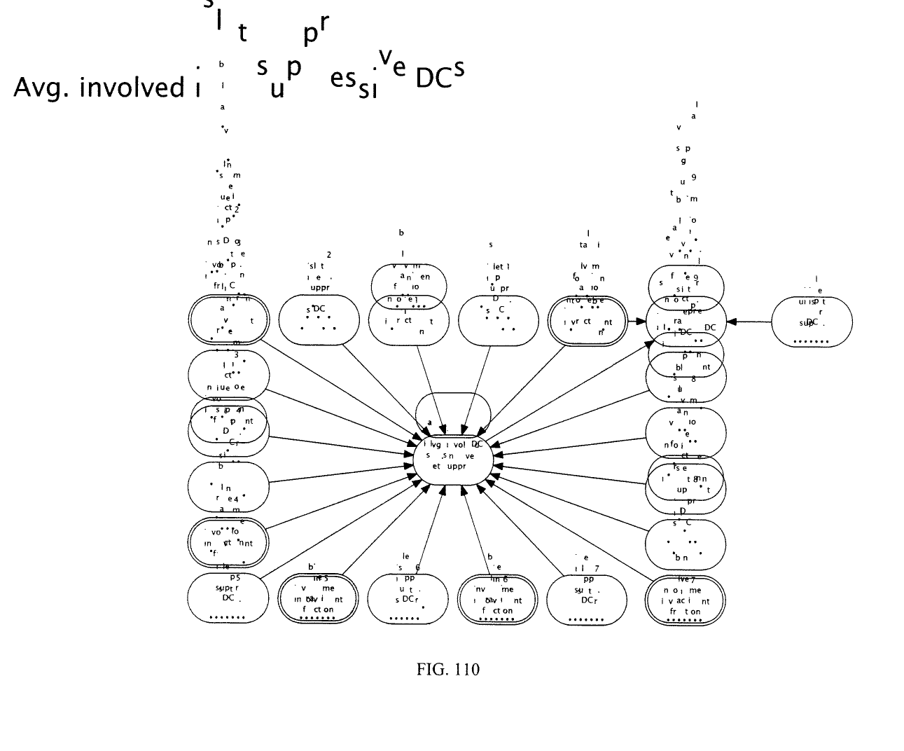

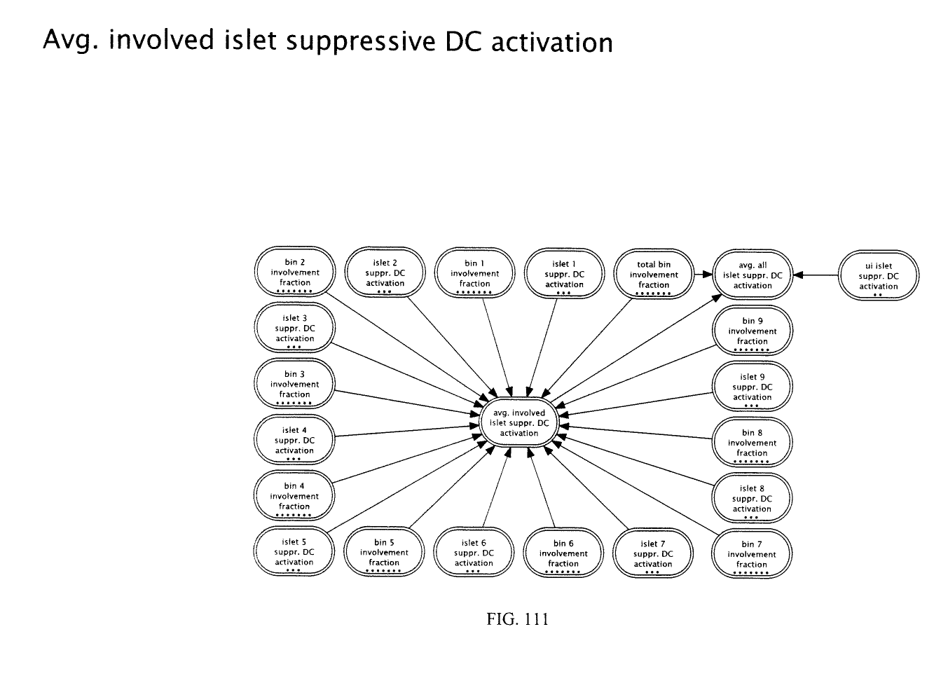

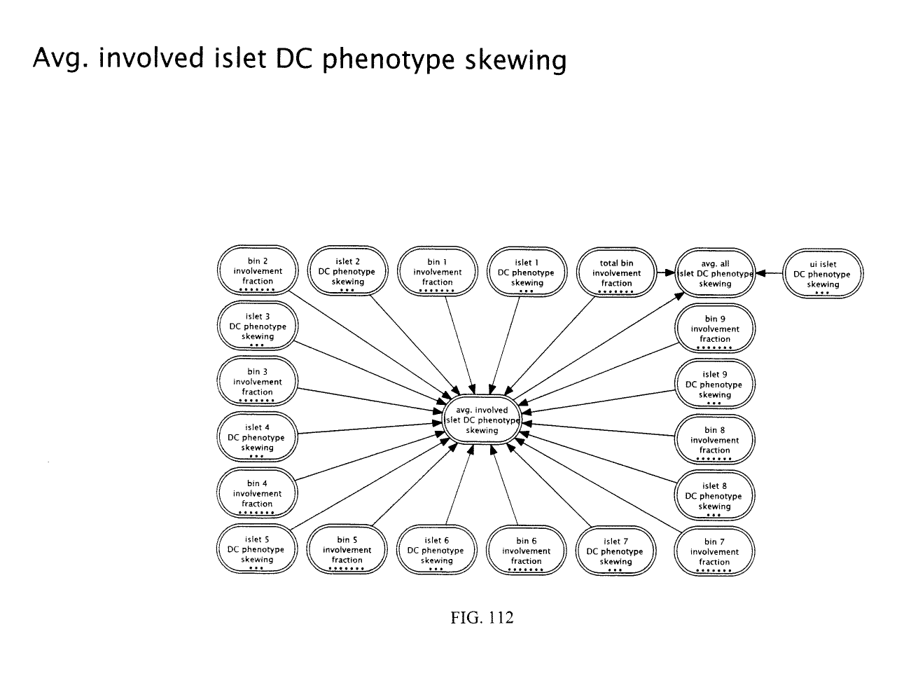

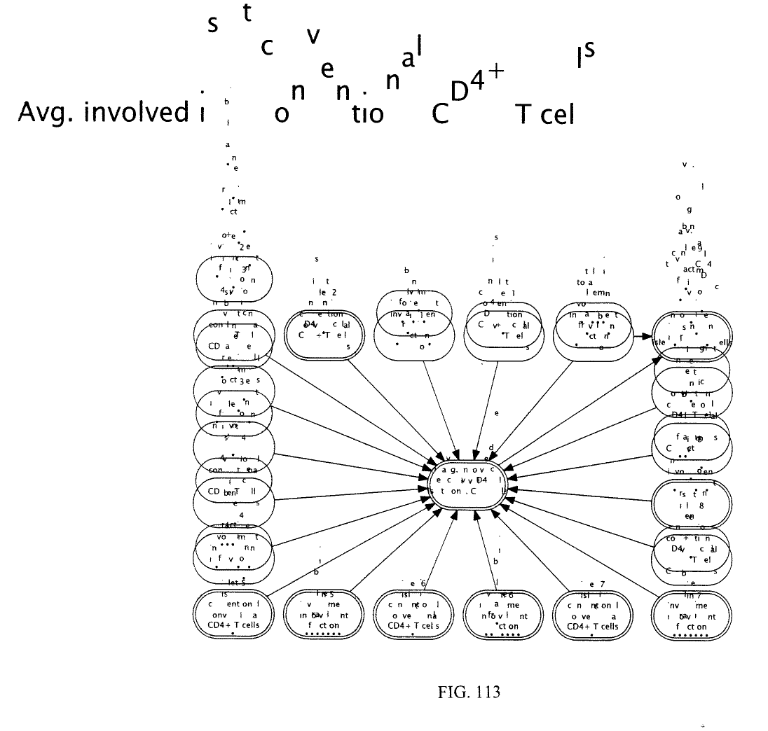

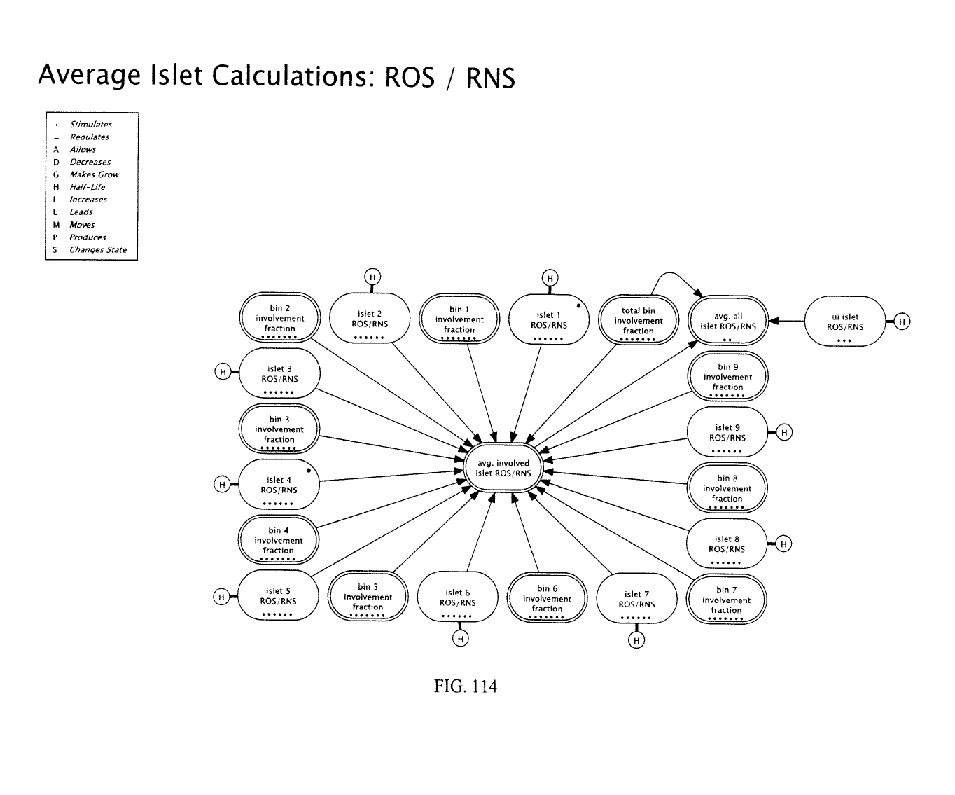

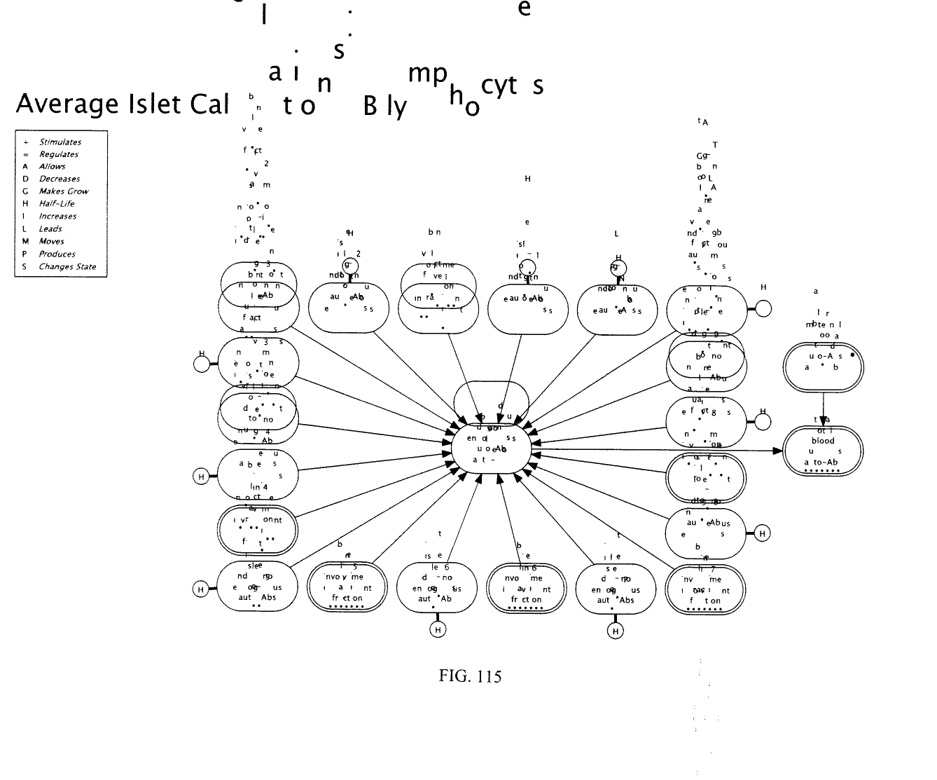

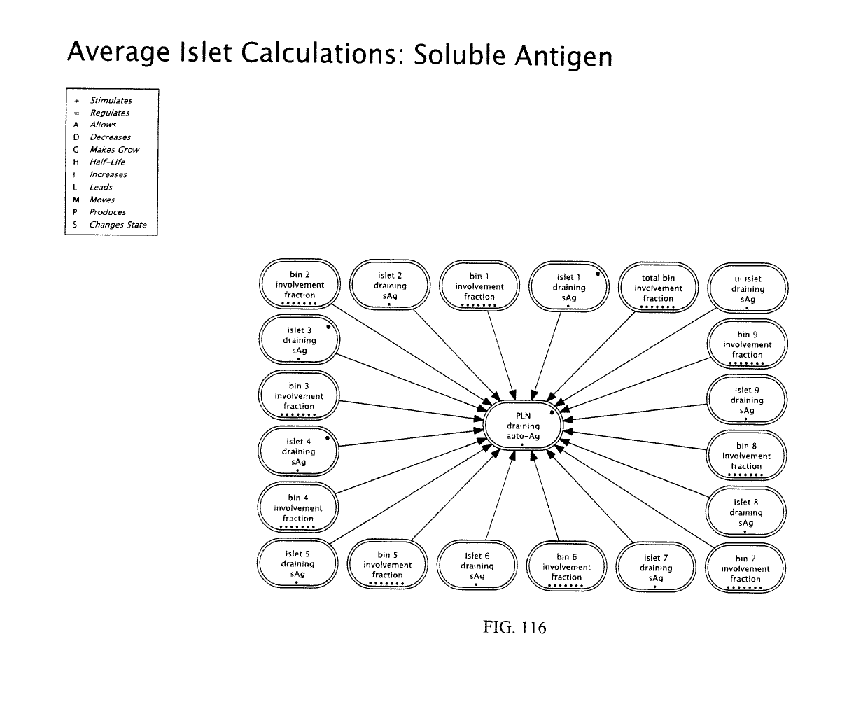

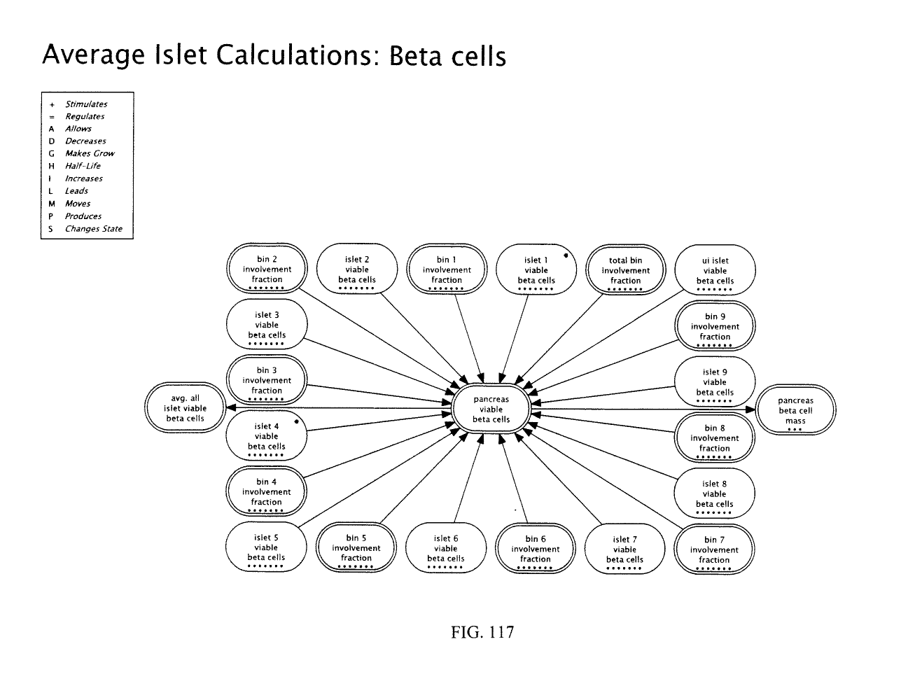

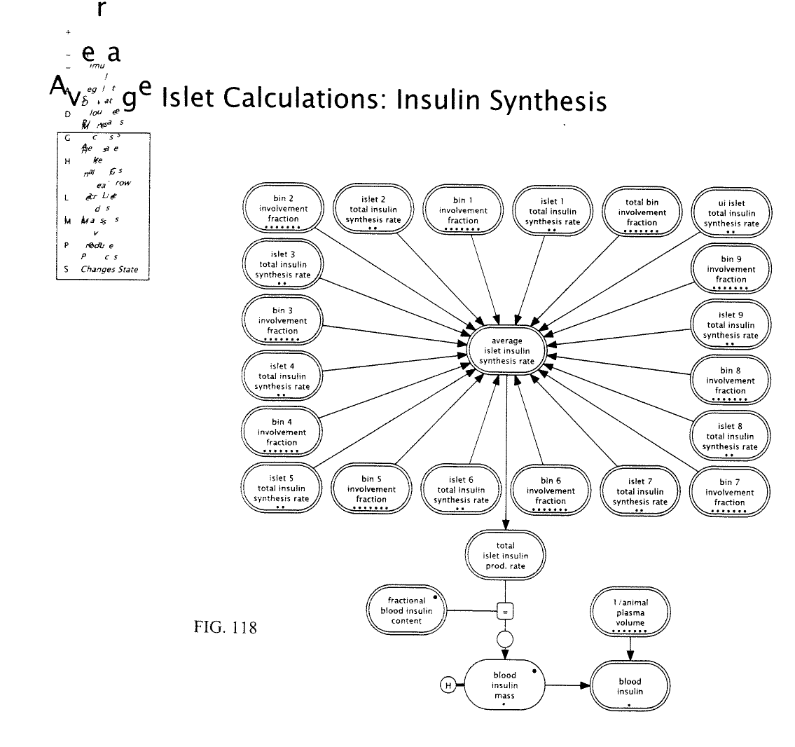

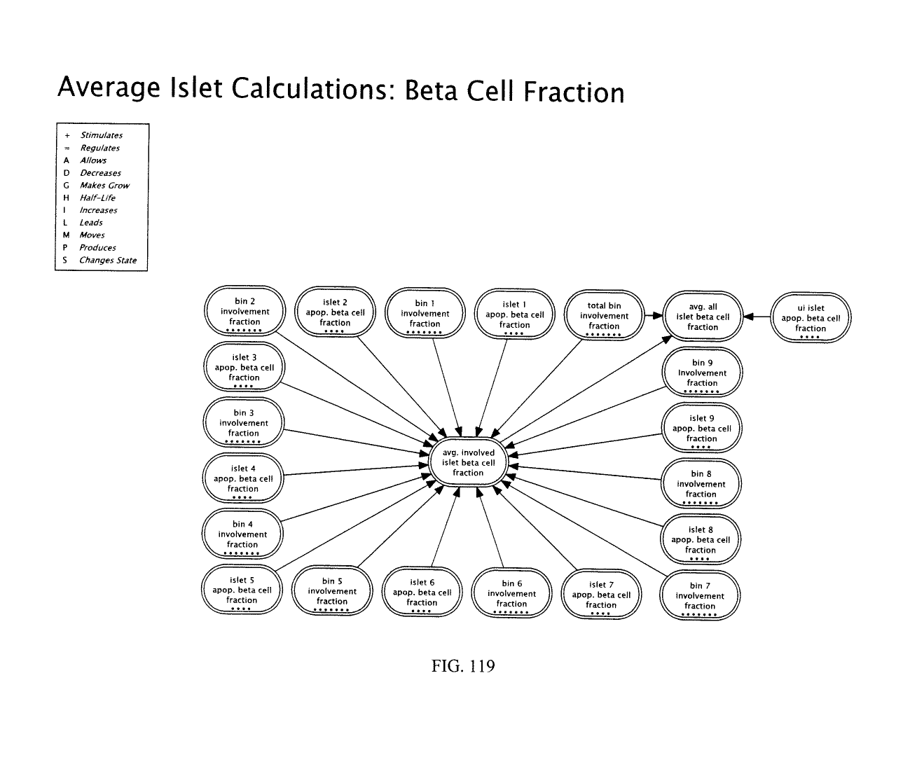

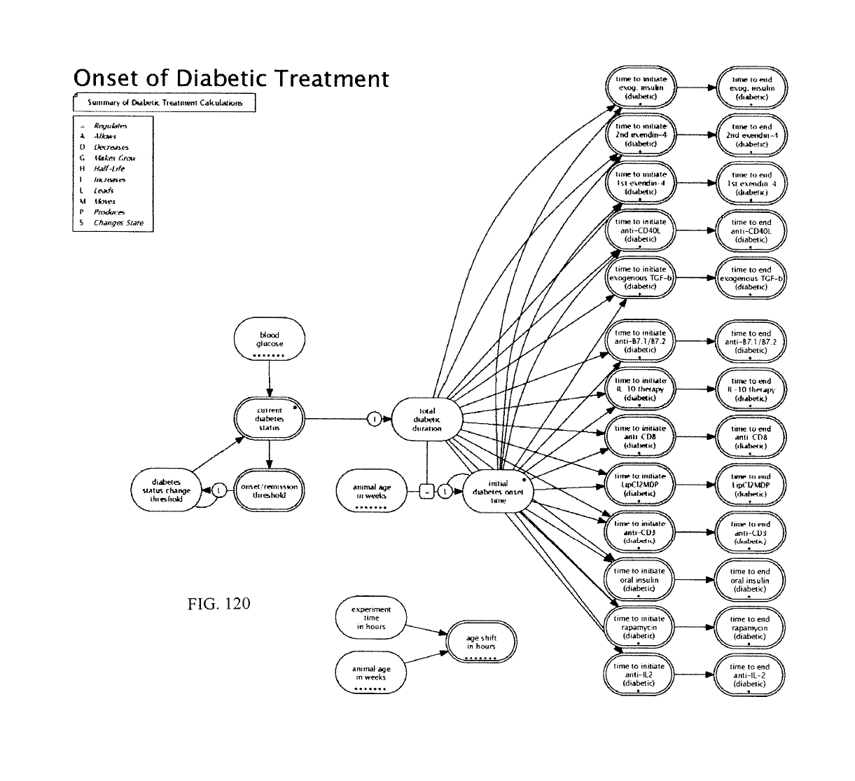

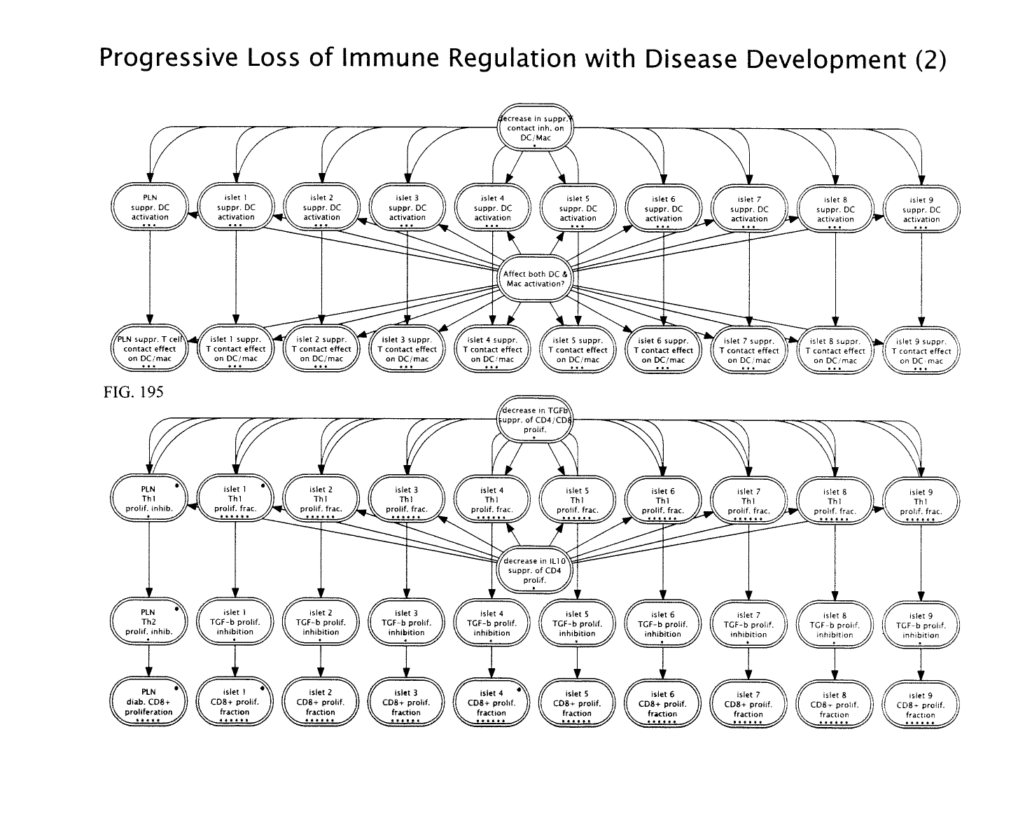

FIGS. 10-195 illustrate exemplary Effect Diagrams describing various aspects of the model of type 1 diabetes.
1. Tissue Compartments

The type 1 diabetes model includes representations of the pancreas and pancreatic lymph nodes. The various functions of β cells, macrophages and dendritic cells, CD4+ T lymphocytes, CD8+ T lymphocytes, regulatory T cells, B lymphocytes, endothelial cells, and NK cells are represented as appropriate in these two compartments. The model simulates the activation of autoreactive T and B lymphocytes in the pancreatic lymph nodes and insulitis progression in the pancreas (FIG. 4).

The contribution of the thymus to type 1 diabetes pathogenesis (Checkpoint 0) was represented by an influx of naïve CD4+, CD8+, and regulatory T cell populations into the circulation, whose numbers and character can be mathematically manipulated. The blood compartment is primarily represented by blood glucose and insulin to provide a clinical output.

The type 1 diabetes model also can include representations of the gut (intestinal) tissue and a prototypical gut-associated lymphoid tissue. The contributions of the gut and gut-associated lymphoid tissue to orally administered therapies are reproduced through the explicit representation of these tissues and the corresponding cellular populations. The various functions of macrophages, dendritic cells, CD4+ T lymphocytes, CD8+ T lymphocytes, regulatory T cells, B lymphocytes, and NK cells are represented as appropriate in these two compartments. The model simulates the distribution of orally administered therapy to the gut tissue as well as the blood. Therapy distributed to the gut can lead to activation of therapeutically-reactive T and B lymphocytes in the gut-associated lymphoid tissue. Therapy distributed to the blood can lead to activation of therapeutically-reactive T and B lymphocytes in the pancreatic lymph nodes and pancreas. The model simulates the activity of an orally administered therapy in multiple tissue compartments which can result in deletion and/or active suppression of therapeutically-reactive T and B lymphocytes in said compartments.
2. Macrophages and Dendritic Cells

Macrophages and dendritic cells are present early during the inflammatory infiltration of the pancreas and continue to accumulate throughout the destructive process. Phagocytic defects, synthesis of IL-12, and antigen presentation, particularly CD11c+CD11b+CD8α-dendritic cells, have all been implicated in the pathological priming and expansion of diabetogenic T lymphocytes in the pancreatic lymph nodes. In addition, macrophage and dendritic cell production of TNF-α, IL-1, and reactive nitrogen species can amplify an immune reaction as well as directly influence β cell function and survival in the islets.

In certain implementations, the computer model can comprise a representation of one or more biological processes associated with dendritic cells and macrophages including: (1) population dynamics in the pancreas, including recruitment, exit, and apoptosis; (2) population dynamics in the pancreatic lymph nodes, including influx and apoptosis; (3) activation in the pancreatic lymph nodes and pancreas, regulated by soluble factors and cell-contact with tissue lymphocytes; (4) phagocytosis and antigen uptake in the pancreas; (5) antigen presentation to and costimulation of CD4+ and CD8+ T lymphocytes and regulatory cells in the pancreatic lymph nodes and pancreas; (6) mediator secretion in the pancreatic lymph nodes and pancreas (e.g., TNF-α, IL-12, ROS/RNS); and (7) cell contact with tissue lymphocytes.

Dendritic cells and macrophages also have important differences that are critical to the immune response, including the occurrence of qualitatively distinct dendritic cell phenotypes and cross-presentation capabilities, as well as quantitative differences in phagocytosis, antigen uptake and presentation efficiencies, and cytokine synthesis rates. To address some of these significant differences, the particular biology associated with distinct functionalities can be modeled separately for the different cell populations (e.g., immature DCs developing into mature DCs with an inflammatory vs. suppressive phenotype). The contribution of dendritic cells and macrophages to the disease can be captured through their antigen-presenting function and through the secretion of soluble mediators that influence other immune cells as well as β and endothelial cells.

Dendritic cells have been identified as critical antigen presenting cells involved in an adaptive immune response. Specifically, dendritic cells serve as sentinels of the immune response, surveying peripheral tissues for potential antigens that may be “seen” by antigen-specific T and B lymphocytes. It has been increasingly appreciated that dendritic cells not only internalize, process, and present antigen, but also integrate various signals to provide additional information to antigen-specific T and B lymphocytes, where the additional information can profoundly influence the resulting response. These signals can include soluble mediators (e.g., TNF-alpha) or cell-contact (e.g., CD40-CD40L mediated cell-contact with T cells). The integration of these signals can alter the dendritic cell phenotype by for example, altering cell surface molecule expression, soluble mediator production, maturation state, and/or antigen presentation. Dendritic cell phenotypes may be characterized as inflammatory (i.e., inflammatory dendritic cells) or suppressive (i.e., suppressive dendritic cells). Inflammatory dendritic cells act to initiate or sustain an inflammatory immune response characterized by the expansion of effector T lymphocytes. Suppressive dendritic cells act to prevent or control an inflammatory immune response through the expansion and/or activation of regulatory T cell populations. The dendritic cell phenotypes are not exclusive as different phenotypes may be simultaneously generated over the course of a response with variation in temporal or spatial dominance. In this scenario, temporal or spatial variation in the balance of inflammatory vs. suppressive dendritic cells would subsequently influence the balance of effector vs. regulatory T cells. Further, the regulated determination of dendritic cell phenotypes can receive positive feedback as inflammatory effector T cells can further activate dendritic cells towards inflammatory activity, while regulatory T cells can induce suppressive dendritic cells, resulting in more regulatory T cells.

Representation of these functions can facilitate investigation of the following issues in type 1 diabetes pathogenesis: (1) the relative importance of antigen presentation and mediator secretion by macrophages and dendritic cells compared with other cell types; (2) the differential role of macrophages and/or dendritic cells during the initial inflammation vs. progression to β cell destruction; and (3) the impact of therapies targeting these populations, their trafficking, DC phenotype, or other specific functions.
3. CD4+ T Lymphocytes

CD4+ T lymphocytes play a central role in type 1 diabetes pathogenesis in the NOD mouse model. Several CD4+ clones can cause disease upon adoptive transfer to non-diabetic NOD mice, and in vivo depletion of CD4+ T lymphocytes in NOD mice can prevent the disease. CD4+ T lymphocytes start to infiltrate the pancreas at around three weeks of age and constitute a large portion of the infiltrate. Some of their key disease-promoting functions include providing help to other cells such as CD8+ T lymphocytes and macrophages/dendritic cells, secretion of inflammatory cytokines, and induction of pancreatic β cell death. However, CD4+ T lymphocytes may also play an important role in preventing or curing diabetes through their differentiation into adaptive regulatory T cells (aTregs) that secrete suppressive cytokines. In contrast, innate regulatory T cells (iTregs) (e.g., CD4+ CD25+, NKT) do not require additional peripheral differentiation for basic regulatory function and are described below.

In certain implementations, the computer model can comprise a representation of one or more biological processes associated with CD4+ T lymphocytes, including Th1, Th2, and adaptive regulatory T cell subsets. Preferred biological processes associated with CD4+ T lymphocytes that can be incorporated in the computer models of the invention include: (1) activation through antigen presentation and costimulation in the pancreatic lymph nodes and pancreas; (2) regulation of Th1, Th2, or adaptive regulatory subtype differentiation and activation, depending on antigen exposure, costimulation, dendritic cell phenotype, cytokines, and contact with regulatory T cells in pancreatic lymph nodes; (3) cross-regulation among CD4+ T cell subsets; (4) differential regulation of islet recruitment for CD4+ T cell subsets; (5) population dynamics in the pancreatic lymph nodes, including influx, proliferation, differentiation, apoptosis, and exit; (6) population dynamics in the pancreas, including recruitment, proliferation, and apoptosis; activation of antigen-presenting cells and potentiation of CD8+ T and B lymphocyte activation in the pancreatic lymph nodes and pancreas; (8) mediator secretion in the pancreatic lymph nodes and pancreas (e.g., IL-4, IL-10, IFN-γ); (9) killing of β cells by soluble mediators and cell contact; and (10) cell contact, for example, with other T and B lymphocytes, dendritic cells and macrophages.

Representation of these functions can facilitate investigation of the following issues in type 1 diabetes pathogenesis: (1) the relative importance of different CD4+ T lymphocyte functions to pathogenic events in the pancreatic lymph nodes as well as the pancreas; (2) the contribution of different CD4+ T cell cytokines to pathogenesis; (3) the relative contribution of different CD4+ T lymphocyte subtypes to disease initiation; (4) the role of regulatory T cells in the initiation/progression of disease; (5) the relative contribution of CD4+ T lymphocytes in β cell killing; and (6) the impact of therapies targeting CD4+ T lymphocyte proliferation, differentiation, apoptosis, and migration.
4. CD8+ T Lymphocytes

Similar to diabetogenic CD4+ T lymphocytes, the importance of CD8+ T lymphocytes in type 1 diabetes has been demonstrated through the identification of clones whose adoptive transfer induces disease and through prevention of diabetes by CD8+ T lymphocyte depletion. CD8+ T lymphocytes are present in pancreatic infiltrates and accumulate over time. Since their major function within the immune system is cytotoxic activity, CD8+ T lymphocytes are believed to play a central role in the killing of β cells. In addition to their cytotoxic potential, CD8+ T lymphocyte production of cytokines is believed to be important in disease progression.

In certain implementations, the computer model can comprise a representation of one or more biological processes associated with CD8+ T lymphocytes including: (1) activation through antigen exposure and cell contact in the pancreatic lymph nodes and pancreas; (2) population dynamics in the pancreatic lymph nodes, including influx, proliferation, apoptosis, and exit; (3) population dynamics in the pancreas, including recruitment, proliferation, and apoptosis; (4) mediator secretion in the pancreatic lymph nodes and pancreas (e.g., TNF-α, IFN-γ); (5) killing of β cells by soluble mediators and cell contact; and (6) cell contact, for example, with other T and B lymphocytes, dendritic cells and macrophages, and β cells.

Representation of these functions can facilitate investigation of the following issues in type 1 diabetes pathogenesis: (1) the relative importance of CD8+ T lymphocytes activities in the pancreatic lymph nodes and pancreas to disease progression; (2) the relative contribution of CD8+ T lymphocytes to β cell killing; and (3) the impact of therapies targeting CD8+ T lymphocyte proliferation, differentiation, apoptosis, and migration.
5. B Lymphocytes

Although autoantibody production is a hallmark in type 1 diabetes, the exact role of B lymphocytes in type 1 diabetes in the NOD mouse is still being elucidated. Disruption or genetic elimination of B lymphocytes appears to protect against disease, but the mechanism of this protection is not clear. B lymphocytes may contribute to disease through their role as antigen-presenting cells and the production of autoantibodies.

In certain implementations, the computer model can comprise a representation of one or more biological processes associated with B lymphocytes, including: (1) antigen presentation to and costimulation of CD4+ and CD8+ T lymphocytes in the pancreatic lymph nodes and pancreas; (2) activation of B lymphocytes through antigen exposure to surface immunoglobulin receptors and cell-contact in the pancreatic lymph nodes and pancreas; (3) activation of B lymphocytes through dendritic cell antigen transfer or cell-contact in the pancreatic lymph nodes; (4) activation of B lymphocytes through dendritic cell or islet β cell antigen transfer in the pancreas; (5) secretion of autoantibody and mediators in the pancreatic lymph nodes and pancreas; (6) population dynamics in the pancreatic lymph nodes and pancreas; and (7) cell contact, for example, with T lymphocytes.

Representation of these functions can facilitate investigation of: (1) the contribution of B lymphocyte antigen-presenting cell function and antibody production to disease progression; (2) the relative importance of B lymphocytes in disease initiation and. progression; (3) the contribution of DC:B lymphocyte interactions to the priming of B lymphocytes; and (4) the impact of therapeutic approaches targeting the B lymphocyte population or its functions.
6. Natural Killer (NK) Cells

There is a growing appreciation for the involvement of NK cells in the NOD mouse model of diabetes. However, seemingly conflicting evidence suggests that NK cells may either protect from autoimmune disease, or conversely, contribute to disease progression in NOD mice. Recent studies have suggested that NK cells play pivotal regulatory roles in adaptive immunity and may contribute to autoimmune disease. Several studies have confirmed that NK cells are functionally depressed in the NOD mouse. For instance, splenic NK cells from NOD mice aged 5-12 weeks were shown to have reduced lytic abilities, as well as reduced IFN-γ production compared to control mice. Additionally, several protective therapeutic interventions in the NOD mouse have been suggested to depend on NK cell activities. For example, the protection afforded by linomide and Complete Freund's Adjuvant (CFA) administration may depend on increased NK cell activities. Other protective agents, such as IFN-α and poly(I:C), while not specific, may also function partially through stimulation of NK cells. Based on these reports, a reasonable hypothesis is that NK cell dysfunction may contribute to disease pathogenesis in the NOD mouse, while enhancing their activity is protective, although other studies suggest that under certain conditions NK cell activity may contribute to the progression of the disease.

While NK cells appear to be playing an important role in type 1 diabetes, little data exist to constrain the in silico NK cell representation. For example, NK cell numbers in the pancreatic lymph nodes and islets during disease progression are not known in the NOD mouse. Moreover, while peripheral NK cells in the NOD mouse appear dysfunctional after β cell destruction begins, it is unclear what their activity is in the islets or if they are dysfunctional during early disease stages. In addition, the effect of NK cell depletion on spontaneous disease progression has not been demonstrated, although treatment with anti-Asialo GM1 in the cyclophosphamide model was protective. While the lack of constraining data reduce confidence in quantitative predictions for NK cells, some of the uncertainty outlined above can be explicitly analyzed and better understood through in silico hypothesis testing.

In certain implementations, the computer model can comprise a representation of one or more biological processes associated with NK cells, including: (1) constitutive NK cell traffic to the pancreatic lymph nodes; (2) regulated recruitment of NK cells to the pancreas; (3) regulation of NK cell activation by cell contact (e.g., DC and β cells) and cytokines (e.g., IL-12, TNF-α, TGF-β); (4) regulated proliferation and apoptosis of NK cells; and (5) NK effector functions, including cytokine production (e.g., IFN-γ, TNF-α, TGF-β) and cytotoxicity (e.g., impacting β cells and immature dendritic cells).

Due to the uncertainty regarding the role of NK cells during pathogenesis, several hypotheses can be considered in alternate virtual mice, which are used for assessing their consistency with reported data and impact on responses to currently implemented interventions.

Representation of these functions enables investigation of the following issues in type 1 diabetes pathogenesis: (1) the contribution of NK cell function to disease progression; (2) the relative importance of NK cells in disease initiation and progression; and (3) the impact of therapeutic approaches targeting the NK cell population or its functions.
7. Islet β Cells

Loss of pancreatic β cell mass is a pivotal event in the progression to type 1 diabetes. In non-autoimmune stains of mice, there is a balance between the growth and death of β cells during the life of the animal. In NOD mice, however, disease onset is characterized by a cascade of events that perturb this homeostasis, resulting in a significant loss of β cell mass. Although several mechanisms for this observation have been suggested in the literature, there is still debate concerning which of these mechanisms is the major contributor to disease outcome. Hence, the implementation of β cells in the model incorporates sufficient biological detail to allow exploration into various death-inducing mechanisms.

Net β cell mass is dependent on the balance between β cell replication and β cell death. Therefore, in certain implementations, the computer model can comprise a representation of one or more biological processes associated with β cell replication and/or cell death, including: (1) replication regulated by age and glucose exposure; and (2) death regulated by age, cell contact (e.g., Fas/FasL, perforin/granzyme), and soluble mediators (e.g., TNF-α, IFN-γ). Additional beta cell functions represented include (1) regulated expression of cell surface molecules (e.g., MHC class I, Fas); and (2) synthesis of soluble mediators (e.g., insulin, ROS/RNS).

Representation of these functions can facilitate investigation of the following issues in type 1 diabetes pathogenesis: (1) the relative role of different pathways that induce β cell death; (2) the impact of physiologic β cell turnover; and (3) the impact of therapies that inhibit β cell destruction.
8. Distinct Distributed Sites (Islet Bins)

Type 1 diabetes is a disease characterized by progressive destruction of islets within the pancreas. One major aspect of disease heterogeneity is the simultaneous presence of islets that are free of inflammatory infiltrates (uninvolved), as well as islets that are infiltrated (involved) to varying degrees and show varying degrees of β cell destruction. The representation of this heterogeneity and its dynamics were critical for the accurate reproduction of both untreated disease and treatment responses. Islet heterogeneity was reproduced through the explicit modeling of distinct distributed sites, representing islets demonstrating semi-independent disease involvement. More specifically, each distinct distributed site (or islet bin; FIG. 3) represents a fraction of the total pancreatic islets and can become infiltrated at distinct points in disease progression. Nine islet bins that have the potential to become infiltrated (i.e., involved islets) were explicitly represented; one islet bin which does not become infiltrated (i.e., uninvolved islets) was also represented. Over the course of simulated disease progression, islets that are infiltrated with autoreactive lymphocytes move from the uninvolved islet bin to an involved islet bin. These bins are progressively “filled” as more islets are infiltrated. For the purposes of brevity, the figures included herewith include representations of only one islet bin (islet 1). The remaining islet bins (2-9) can be represented in a similar manner.

While the mechanism(s) that determine when any particular islet or set of islets will become infiltrated (involved) is unknown, the whole pancreas dynamics of islet infiltration (involvement) have been characterized. The control of said dynamics are incompletely understood; however, CD4+ and CD8+ T lymphocytes are implicated by two indirect lines of evidence: (1) blockade of either cell population early in disease progression limits the extent of islet involvement; and (2) transfer of diabetogenic CD4+ or CD8+ T lymphocytes leads to islet involvement. Based on these data, the rate of islet involvement can be represented as a function of activated diabetes-antigen-specific CD4+ and CD8+ T lymphocytes in circulation and subsequently, the islet involvement curve can determine the timing with which the distinct distributed sites (islet bins) become infiltrated (involved).

Representation of distinct distributed sites enables the appropriate representation of heterogeneity in disease activity and progression and the effect of heterogeneity on therapeutic response. It further enables investigation of the relative effects of treatment timing on distinct islet bins.
9. Effective Antigen Pool

Several autoantigens have been identified in NOD mouse type 1 diabetes. For example, glutamate decarboxylase (GAD) and insulin are considered major autoantigens in the disease process based on autoantibody production and T lymphocyte responses. However, the endogenous antigens have not been identified for some isolated diabetogenic clones (e.g., BDC2.5). To facilitate use of these different data sources and representation of different potential autoantigens, the NOD mouse type 1 diabetes model includes a generalized, or effective, pancreatic antigen pool. The use of this effective antigen pool also facilitates the capture of the full diabetogenic T lymphocyte repertoire, as opposed to the response at the level of a single diabetogenic T lymphocyte clone. Where appropriate (e.g., implementation of a therapy targeting a specific antigen), specific antigens and antigen specific lymphocytes have been explicitly represented.

Where appropriate, the differential impact of soluble vs. cell-associated autoantigens on uptake and presentation was considered. In the model, the production of soluble autoantigens depends on β cell numbers and insulin synthesis activity, while the production of cell-associated autoantigens is related to the amount of apoptotic β cells and the rate of phagocytosis by dendritic cells and macrophages. To address alternative antigen source hypotheses, a β cell-independent source of autoantigen may be represented. Dendritic cells, macrophages, and B lymphocytes capture antigen and present MHC-antigen complexes with varying efficiencies depending on the antigen source and the presence of autoantibodies.

Representation of these functions enables investigation of the relative roles of soluble vs. cell-associated autoantigen in driving the autoimmune reaction, of the timing of autoantigen availability and its impact on disease progression, and of the therapeutic effect in targeting specific antigen.
10. Glucose Control

The model includes a minimal representation of insulin synthesis and secretion by β cells. The rate of insulin release depends on the glucose concentration. The impact of ROS/RNS on β cell insulin secretion was also included. The glucose concentration itself is regulated by insulin-dependent and independent mechanisms using an approach similar to the classical ‘minimal model’ representation, where parameters for glucose effectiveness and sensitivity to insulin are quantified. This implementation allows dynamic regulation of insulin by glucose during disease progression and enables the prediction of how changes in β cell mass and function affect circulating glucose and insulin concentrations.
11. Endothelial Cells

Endothelial cells regulate the entry of inflammatory cells into the pancreas, and some potential therapeutics have targeted leukocyte-endothelial interactions. One major function of the pancreatic endothelial cells is to regulate the influx of circulating leukocytes through adhesive interactions. In certain implementations, the computer model can comprise a representation of one or more biological processes associated with endothelial cells, including: (1) endothelial adhesion molecule expression, regulated by cytokines, in the pancreas; and (2) regulation of leukocyte recruitment to the pancreas by endothelial adhesion molecules. Representation of biological processes associated with endothelial cells allows assessment of the impact of modulating endothelial adhesion molecule expression levels on the efficacy of therapies.
12. Innate Regulatory T Cells (iTregs)

One hypothesized driver of type 1 diabetes is a deficiency in innate regulatory T cells. These cells may modulate the rate of disease progression in natural pathogenesis, limit progression in resistant phenotypes, and mediate the efficacy of some therapeutics. Numerical or functional defects in several populations of innate regulatory T cells (e.g., NKT, CD4⁺CD25⁺, DX5⁺) are thought to be important to type 1 diabetes pathogenesis in the NOD mouse. In addition, some therapeutics may act, at least in part, through stimulation of these populations. Two major mechanisms, cell contact and secretion of regulatory cytokines, appear to be common to multiple regulatory cell types. Thus, while some regulatory populations are insufficiently characterized to warrant modeling them individually, the functionality of different innate regulatory populations can be captured by modeling a common population that acts through cell contact and the secretion of regulatory cytokines. The population dynamics of CD4+ CD25+ T cells are the most well-characterized among the different subsets of innate regulatory T cells and was the focus of this population in the model. However, the NKT cell literature was also evaluated and used to represent functionality that is distinct from CD4+ CD25+ regulatory T cells.

In certain implementations, the computer model can comprise a representation of one or more biological processes associated with innate regulatory T cells, including: activation through antigen exposure and cell contact in the pancreatic lymph nodes and pancreas; (2) population dynamics in the pancreatic lymph nodes, including influx, proliferation, apoptosis, and exit; (3) population dynamics in the pancreas, including influx, proliferation, and apoptosis; (4) secretion of soluble mediators in the pancreatic lymph nodes and pancreas (e.g., TGF-β, IL 10, IL-4); (5) modulation of antigen-presenting cell function through cell contact in the pancreatic lymph nodes and pancreas; and (6) suppression of T cell, NK cell, and B lymphocyte functions through cell contact in the pancreatic lymph nodes and pancreas.

Modeling an innate regulatory T cell population (iTregs) was critical for the accurate representation of regulatory functions affecting disease progression and also for appropriate responses to some therapies. Specifically, the dynamic balance between effector and regulatory T cell populations was a significant determinant of the speed of disease progression, disease outcome (no diabetes vs. average diabetes vs. accelerated diabetes), and therapies with preferential effects on regulatory populations.

Representation of these functions enables investigation of the following issues in type 1 diabetes pathogenesis: (1) the relative contribution of suppression by cytokines versus cell contact; (2) the relative contribution of different suppressor cytokines to disease modulation; and (3) the impact and optimal administration of therapies targeting innate regulatory T cells.

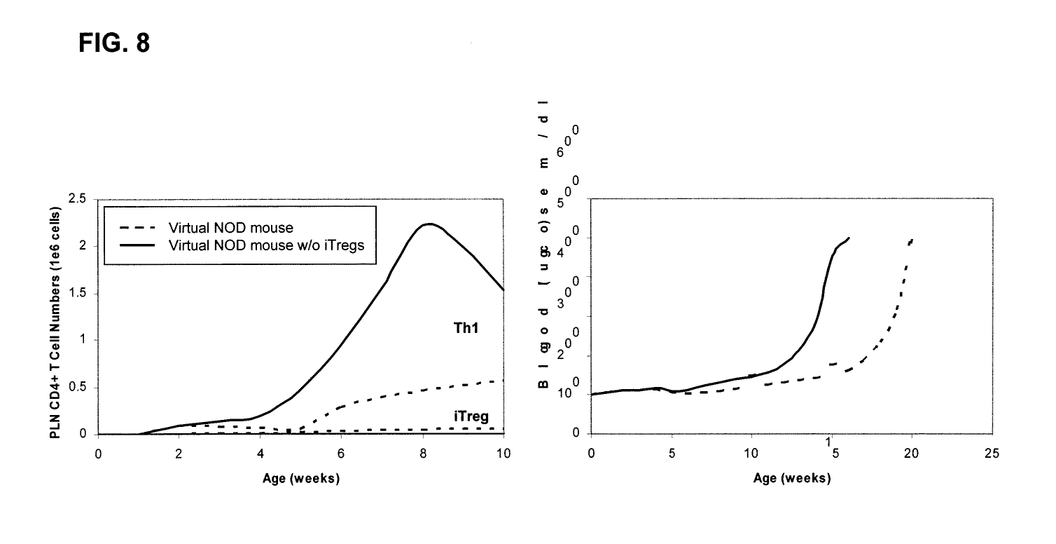

Simulated depletion of the innate regulatory T cell population can be used to demonstrate how the balance between effector vs. regulatory cells controls the rate of disease progression. Specifically, innate regulatory T cell depletion results in enhanced expansion of effector cell populations in the model, which ultimately accelerates disease progression. In FIG. 8, the innate regulatory T cell population has been set to 0 from the beginning of the simulation. Relative to the unmanipulated virtual mouse, the innate regulatory T cell deficient mouse demonstrates significantly elevated Th1 expansion in the PLN (FIG. 8A). The Th1 expansion is mirrored by other effector cell populations (data not shown). As a result of this shift in the balance of regulatory and effector cells, the virtual mouse demonstrates exacerbated disease, with frank diabetes manifesting at 15 weeks (FIG. 8B).

This invention can include a single computer model that serves a number of purposes. Alternatively, this layer can include a set of large-scale computer models covering a broad range of physiological systems. In addition to including a model of type 1 diabetes, the system can include complementary computer models, such as, for example, epidemiological computer models and pathogen computer models. For use in healthcare, computer models can be designed to analyze a large number of subjects and therapies. In some instances, the computer models can be used to create a large number of validated virtual patients and to simulate their responses to a large number of therapies.

The invention and all of the functional operations described in this specification can be implemented in digital electronic circuitry, or in computer software, firmware, or hardware, including the structural means disclosed in this specification and structural equivalents thereof, or in combinations of them. The invention can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple computers. A computer program (also known as a program, software, software application, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification, including the method steps of the invention, can be performed by one or more programmable processors executing one or more computer programs to perform functions of the invention by operating on input data and generating output. The processes and logic flows can also be performed by, and apparatus of the invention can be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. Information carriers suitable for embodying computer program instructions and data include all forms of non volatile memory, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The invention can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

E. Methods of Simulating Mammalian Type 1 Diabetes

One aspect of the invention provides computer-based mathematical representations of the biology related to type 1 diabetes. Each model includes a set of nonlinear, coupled, ordinary differential equations that describe the network of biological components and functions relevant to a disease; a graphical user interface that provides a visual display of the modeled biology; a reference and rationale documentation set that enables researchers to view the published literature and rationale that support modeling decisions; and a research architecture for conducting experiments in silico and managing the results. Development of computer-based models of metabolic diseases, such as diabetes, are described in detail in co-pending U.S. patent application Ser. No. 10/040,373, published as US 2003-0058245, incorporated herein by reference in its entirety.

The scope of each clinical model is defined by the system level (or clinical) behaviors that the model needs to reproduce. The underlying biology is then modeled in the detail needed to reproduce the desired behaviors and address specific research problems. If specific data are lacking, the model uses related data and general physiological and physical principles to produce a system level behavior consistent with the expected outcome.

Virtual patients, which are defined by the model equations and a specified set of associated parameter values, are developed to explore specific patient phenotypes and can reflect different clinical behaviors (e.g., disease severity, different rates of disease progression) or different disease pathophysiologies (e.g., B lymphocyte-dependent vs. B lymphocyte-independent inflammation). In the NOD mouse type 1 diabetes model, these representations are termed virtual mice. Just as virtual patients are developed as described in US 2003-0058245, different sets of parameters will be selected to represent virtual mice with different biological features, such as increased or decreased expression of a receptor, rate of cytokine production, or cell population numbers or function. The parameter sets defining these virtual mice will be stored in the model and used in many types of investigations, including investigations of disease mechanisms and development of potential therapeutics.

In certain implementations, alternate virtual NOD mice can be developed to represent and understand the processes that contribute to disease pathogenesis and heterogeneity. Alternate disease hypotheses that account for variability in the clinical manifestation of the disease or uncertainty regarding its underlying pathophysiology can be explicitly represented. Once implemented, the effect of each hypothesis on disease outcomes can be explored, and based on the results, experiments can be designed to distinguish between competing hypotheses, thereby providing in silico guidance for improved understanding of the underlying pathophysiology.

Additionally, in certain implementation, the colony of virtual mice can be expanded for use in research projects on NOD mouse biology and in the evaluation of novel therapeutic agents. For example, virtual NOD mice can be developed to represent different phenotypes and the diversity in mechanistic pathophysiology within a phenotype. The behaviors of these virtual NOD mice would be consistent with known pathogenesis and fall within the range of reported responses to the set of selected interventions tested in the reference NOD mouse. Representative examples of such alternative virtual mice include early disease onset, late disease onset, and resistance to disease over a reasonable lifespan of the mouse.

The invention also provides methods of simulating type 1 diabetes, said method comprises executing a computer model of type 1 diabetes as described above. Methods of simulating type 1 diabetes can further comprise applying a virtual protocol to the computer model to generate a set of outputs representing a phenotype of the biological system. The phenotype canF represent a normal state or a diseased state. In certain implementations, the methods can further include accepting user input specifying one or more parameters or variables associated with one or more mathematical representations prior to executing the computer model. Preferably, the user input comprises a definition of a virtual patient or a definition of the virtual protocol.

Running the computer model produces a set of outputs for a biological system represented by the computer model. The set of outputs represent one or more phenotypes of the biological system, i.e., the simulated subject, and includes values or other indicia associated with variables and parameters at a particular time and for a particular execution scenario. For example, a phenotype is represented by values at a particular time. The behavior of the variables is simulated by, for example, numerical or analytical integration of one or more mathematical relations to produce values for the variables at various times and hence the evolution of the phenotype over time.

The computer executable software code numerically solves the mathematical equations of the model(s) under various simulated experimental conditions. Furthermore, the computer executable software code can facilitate visualization and manipulation of the model equations and their associated parameters to simulate different patients subject to a variety of stimuli. See, e.g., U.S. Pat. No. 6,078,739, entitled “Managing objects and parameter values associated with the objects within a simulation model,” the disclosure of which is incorporated herein by reference. Thus, the computer model(s) can be used to rapidly test hypotheses and investigate potential drug targets or therapeutic strategies.

In one implementation, the computer model can represent a normal state as well as an abnormal (e.g., a diseased or toxic) state of a biological system. For example, the computer model includes parameters that are altered to simulate a diabetic state or a progression towards the diabetic state. The parameter changes to represent a disease state are typically modifications of the underlying biological processes involved in a disease state, for example, to represent the genetic or environmental effects of the disease on the underlying physiology. By selecting and altering one or more parameters, a user modifies a normal state and induces a disease state of interest. In one implementation, selecting or altering one or more parameters is performed automatically.

In the present embodiment of the invention, various mathematical relations of the computer model, along with a modification defined by the virtual stimulus, can be solved numerically by a computer using standard algorithms to produce values of variables at one or more times based on the modification. Such values of the variables can, in turn, be used to produce the first set of results of the first set of virtual measurements.

One or more virtual patients in conjunction with the computer model can be created based on an initial virtual patient that is associated with initial parameter values. A different virtual patient can be created based on the initial virtual patient by introducing a modification to the initial virtual patient. Such modification can include, for example, a parametric change (e.g., altering or specifying one or more initial parameter values), altering or specifying behavior of one or more variables, altering or specifying one or more functions representing interactions among variables, or a combination thereof. For instance, once the initial virtual patient is defined, other virtual patients may be created based on the initial virtual patient by starting with the initial parameter values and altering one or more of the initial parameter values. Alternative parameter values can be defined as, for example, disclosed in U.S. Pat. No. 6,078,739. These alternative parameter values can be grouped into different sets of parameter values that can be used to define different virtual patients of the computer model. For certain applications, the initial virtual patient itself can be created based on another virtual patient (e.g., a different initial virtual patient) in a manner as discussed above.

Alternatively, or in conjunction, one or more virtual patients in the computer model can be created based on an initial virtual patient using linked simulation operations as, for example, disclosed in the following publication: “Method and Apparatus for Conducting Linked Simulation Operations Utilizing A Computer-Based System Model”, (U.S. Application Publication No. 2001-0032068, published on Oct. 18, 2001). This publication discloses a method for performing additional simulation operations based on an initial simulation operation where, for example, a modification to the initial simulation operation at one or more times is introduced. In the present embodiment of the invention, such additional simulation operations can be used to create additional virtual patients in the computer model based on an initial virtual patient that is created using the initial simulation operation. In particular, a virtual patient can be customized to represent a particular subject. If desired, one or more simulation operations may be performed for a time sufficient to create one or more “stable” virtual patient of the computer model. Typically, a “stable” virtual patient is characterized by one or more variables under or substantially approaching equilibrium or steady-state condition.

Due to the observed heterogeneity in the clinical presentation of human type 1 diabetes and the limited availability of human data, creation of alternate virtual patients is a critical aspect of this plan. Representation of such patients allows the exploration of patient variability, as well as uncertainty in the underlying human pathogenesis and the resultant impact on therapeutic outcomes. Similar to alternate virtual NOD mice, alternate virtual patients may also show variations in therapeutic responsiveness that are consistent with the response heterogeneity observed in clinical trials. For instance, whereas the general clinical trial population responded poorly to insulin tolerization, better therapeutic responses were observed in patients characterized by high insulin autoantibody titers. Lastly, representation of alternate virtual patients enables the in silico evaluation of therapeutic efficacy in different clinical subpopulations and may aid in the design or optimization of new clinical trials. These alternate virtual patients will be developed using the same approach explained for developing alternate virtual mice. The resulting cohort of human virtual patients, together with the cohort of virtual NOD mice, provide the in silico resources to address translational efforts to develop therapies for type 1 diabetes. Thus, the model of type 1 diabetes can include representations of multiple virtual NOD mice and virtual human patients.

Various virtual patients of the computer model can represent variations of the biological system that are sufficiently different to evaluate the effect of such variations on how the biological system responds to a given therapy. In particular, one or more biological processes represented by the computer model can be identified as playing a role in modulating biological response to the therapy, and various virtual patients can be defined to represent different modifications of the one or more biological processes. The identification of the one or more biological processes can be based on, for example, experimental or clinical data, scientific literature, results of a computer model, or a combination of them. Once the one or more biological processes at issue have been identified, various virtual patients can be created by defining different modifications to one or more mathematical relations included in the computer model, which one or more mathematical relations represent the one or more biological processes. A modification to a mathematical relation can include, for example, a parametric change (e.g., altering or specifying one or more parameter values associated with the mathematical relation), altering or specifying behavior of one or more variables associated with the mathematical relation, altering or specifying one or more functions associated with the mathematical relation, or a combination of them. The computer model may be run based on a particular modification for a time sufficient to create a “stable” configuration of the computer model.

One aspect of the invention provides methods for developing a model of a non-insulin replacement treatment of type 1 diabetes said method comprising: identifying one or more biological processes associated with a β cell population in at least one of one or more pancreatic islets; identifying one or more biological processes associated with an effect of a non-insulin replacement treatment of type 1 diabetes; mathematically representing each biological process to generate one or more representations of a biological process associated with the β cell population and one or more representations of a biological process associated with an effect of the non-insulin replacement treatment of type 1 diabetes; and combining the representations of the biological processes to form the model of a non-insulin replacement treatment of type 1 diabetes. As used herein, the term “treatment of type 1 diabetes” refers to actual or contemplated regimens administered for the purpose of preventing, slowing or reversing the onset or progression of type 1 diabetes. The term “non-insulin,” as used herein, refers to regimens other than systemic, e.g. injected or inhaled, administration of insulin for the explicit purpose of controlling blood glucose levels. Regimens comprising the oral administration of insulin for the purpose of developing tolerance to insulin and/or insulin producing cells is excluded from the scope of “non-insulin replacement treatment.”

In certain implementations, the model of type 1 diabetes is executed while applying a virtual stimulus or protocol representing, e.g., administration of a drug. A virtual stimulus can be associated with a stimulus or perturbation that can be applied to a biological system. Different virtual stimuli can be associated with stimuli that differ in some manner from one another. Stimuli that can be applied to a biological system can include, for example, existing or hypothesized therapeutic agents, treatment regimens, and medical tests. Additional examples of stimuli include exposure to existing or hypothesized disease precursors. Further examples of stimuli include environmental changes such as those relating to changes in level of exposure to an environmental agent (e.g., an antigen).

A virtual protocol, e.g., a virtual therapy, representing an actual therapy can be applied to a virtual patient in an attempt to predict how a real-world equivalent of the virtual patient would respond to the therapy. Virtual protocols that can be applied to a biological system can include, for example, existing or hypothesized therapeutic agents and treatment regimens, mere passage of time, exposure to environmental toxins, increased exercise and the like. By applying a virtual protocol to a virtual patient, a set of results of the virtual protocol can be produced, which can be indicative of various effects of a therapy.

For certain applications, a virtual protocol can be created, for example, by defining a modification to one or more mathematical relations included in a model, which one or more mathematical relations can represent one or more biological processes affected by a condition or effect associated with the virtual protocol. A virtual protocol can define a modification that is to be introduced statically, dynamically, or a combination thereof, depending on the particular conditions and/or effects associated with the virtual protocol.

In certain implementations of the invention, the computer model is capable of simulating a perturbation (e.g., a therapy) or action of a perturbing agent selected from the group consisting of anti-CD3 monoclonal antibody, anti-CD8 monoclonal antibody, liposomal dichloromethylene diphosphonate (Lip-Cl₂MDP), exogenous IL-10, anti-B7.1/2 monoclonal antibodies, oral insulin, exendin-4, exogenous TGF-β, anti-CD40L monoclonal antibody, rapamycin, and anti-IL-2 monoclonal antibody.

Using an extensive analysis of interventions experimentally tested in the NOD mouse (Shoda, et al., Immunity 23(2): 115-26 (2005); incorporated herein by reference), a set of representative interventions was selected to satisfy multiple criteria, including agents with diverse, well characterized mechanisms of action; relevance to natural pathogenesis; differential efficacy; and application in human clinical trials. This set includes the following anti-CD3 monoclonal antibody, anti-CD8 monoclonal antibody, liposomal dichloromethylene diphosphonate (Lip-Cl₂MDP), exogenous IL-10, anti-B7.1/2 monoclonal antibodies, oral insulin, exendin-4, exogenous TGF-β, anti-CD40L monoclonal antibody, rapamycin, and anti-IL-2 monoclonal antibody.

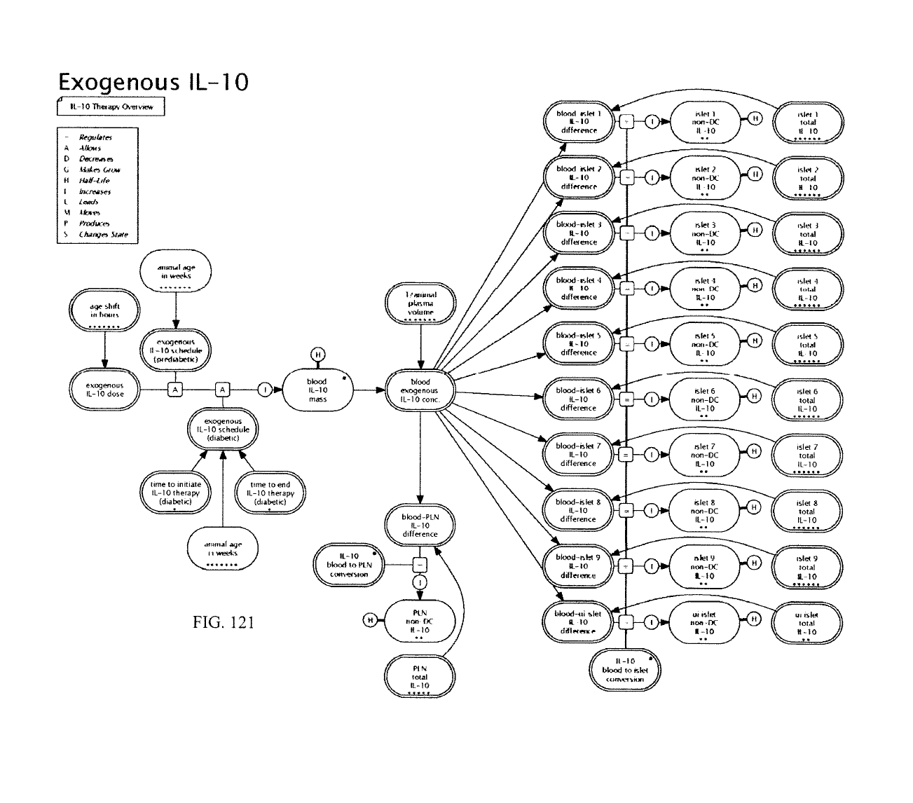

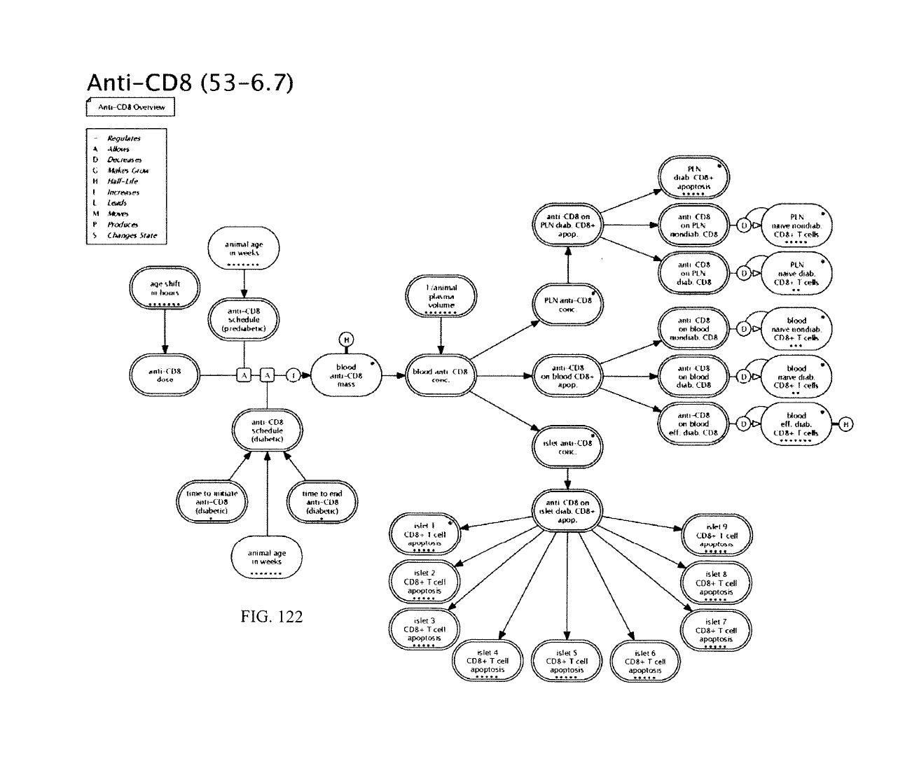

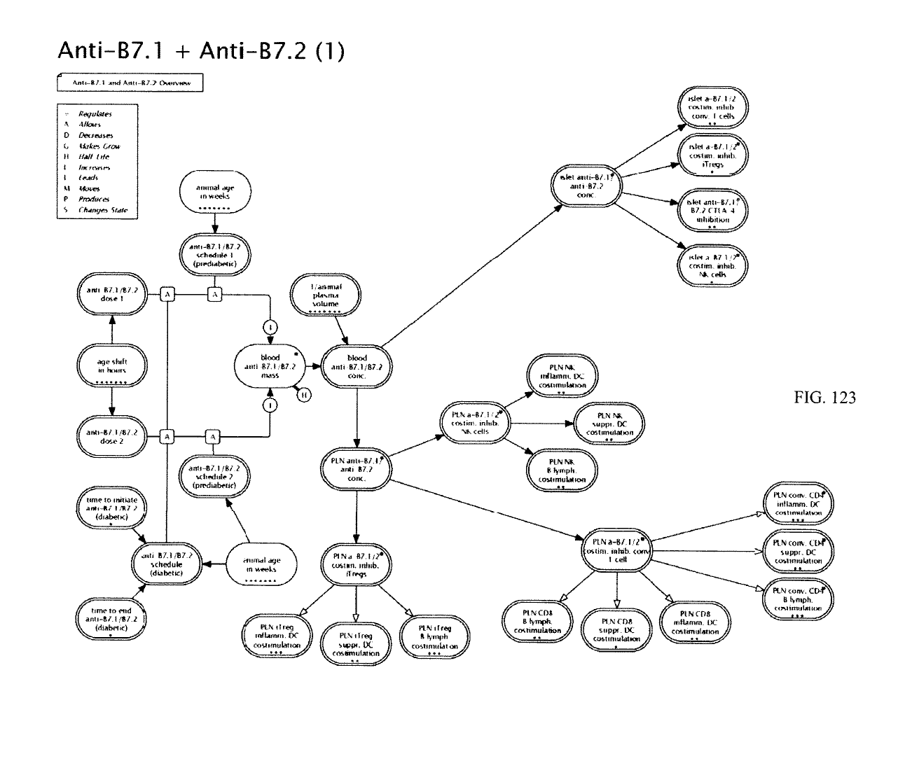

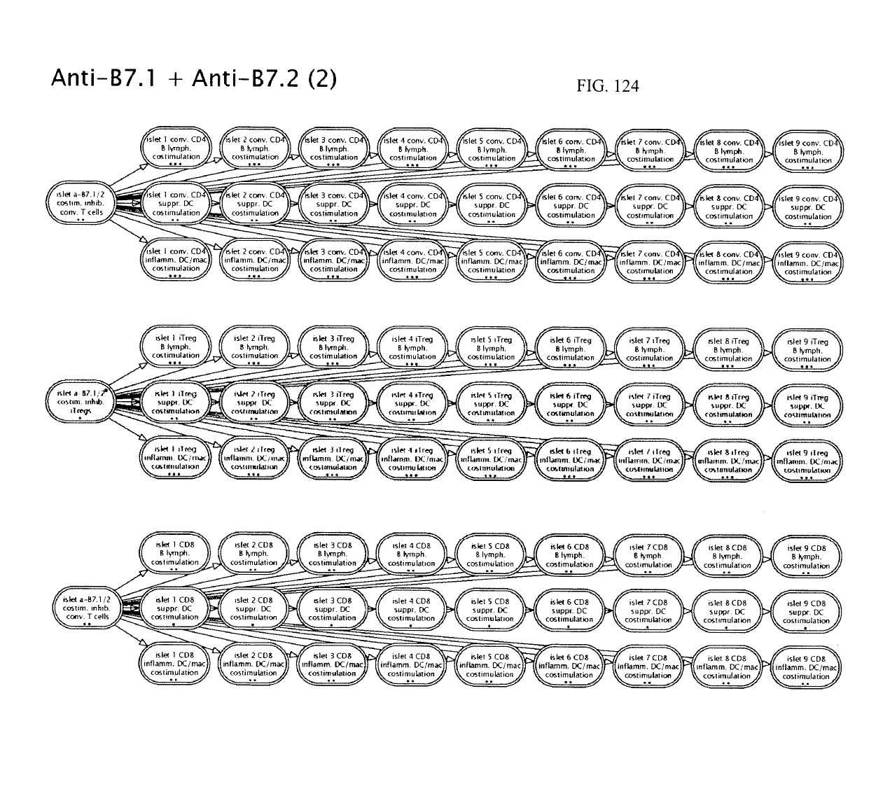

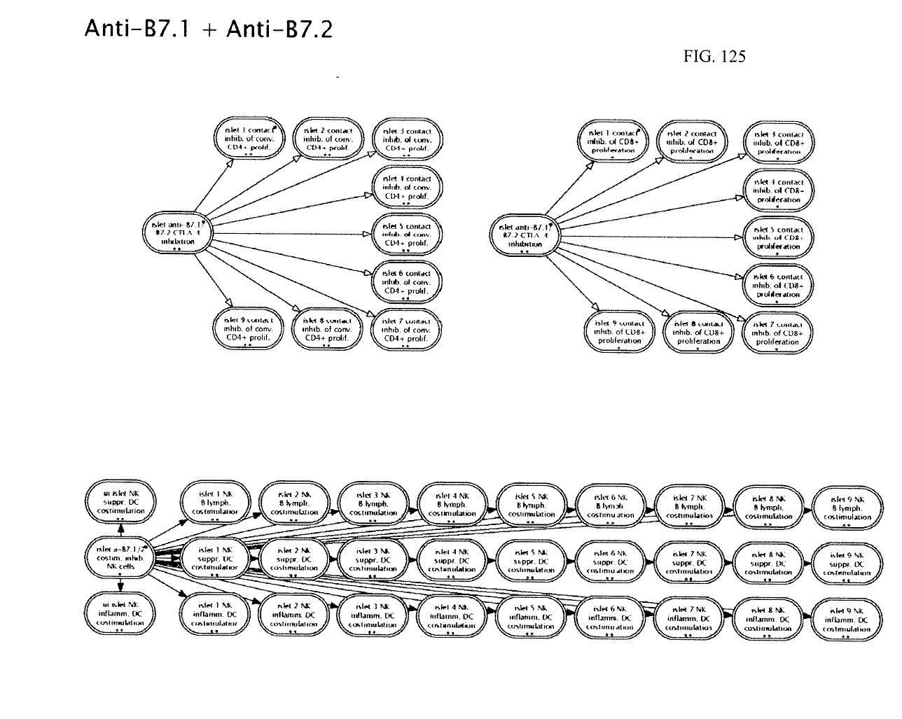

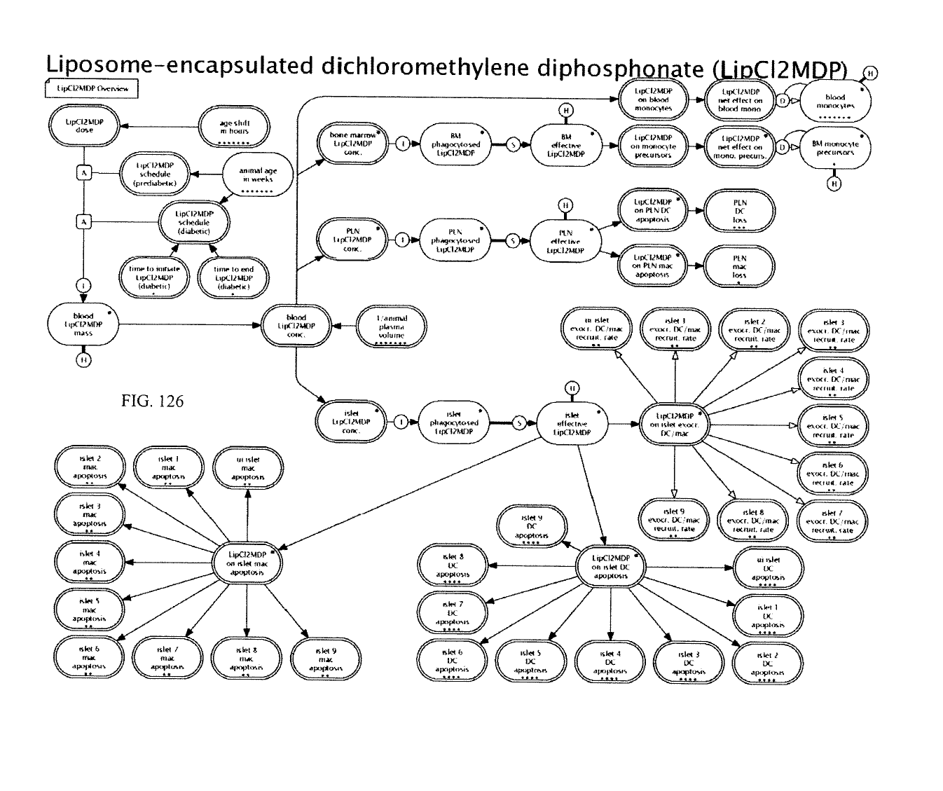

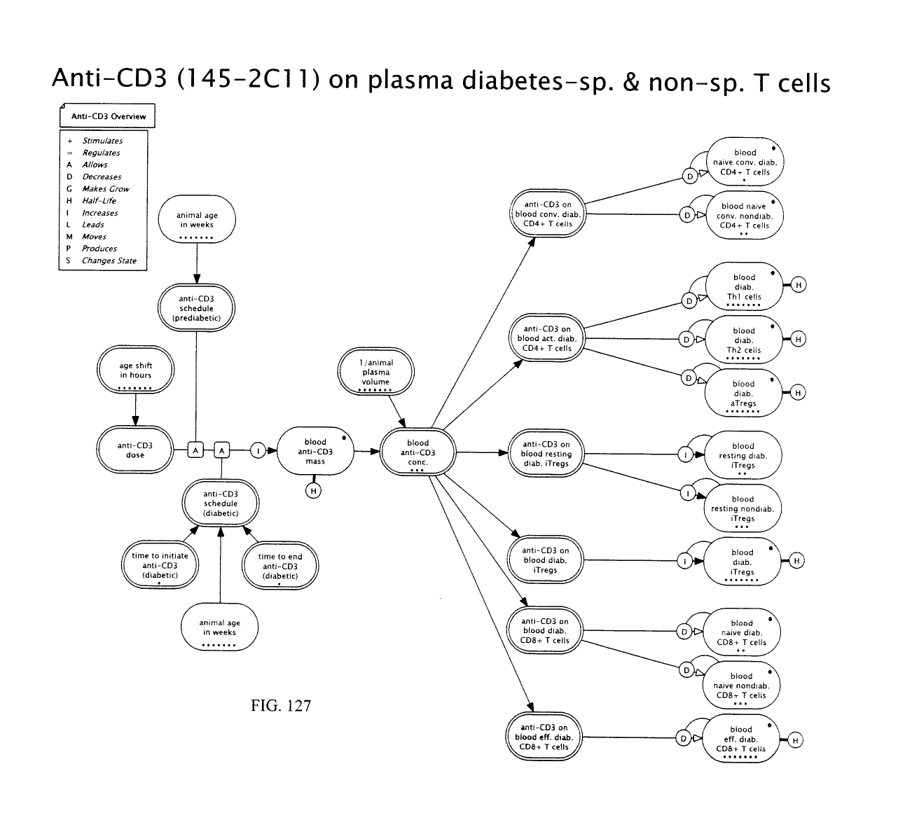

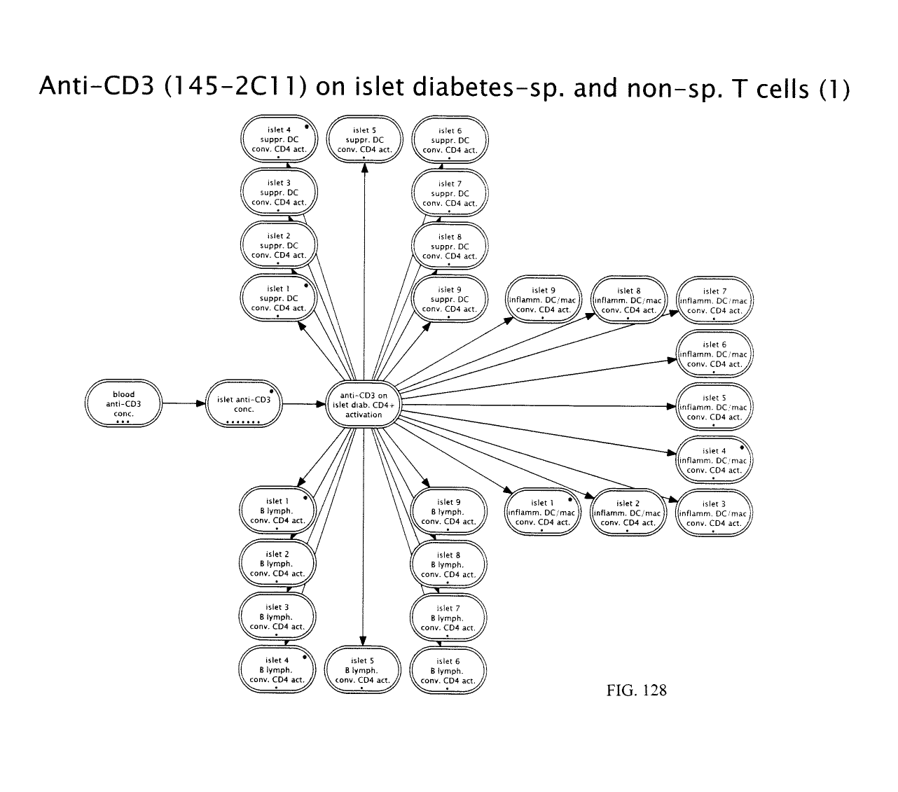

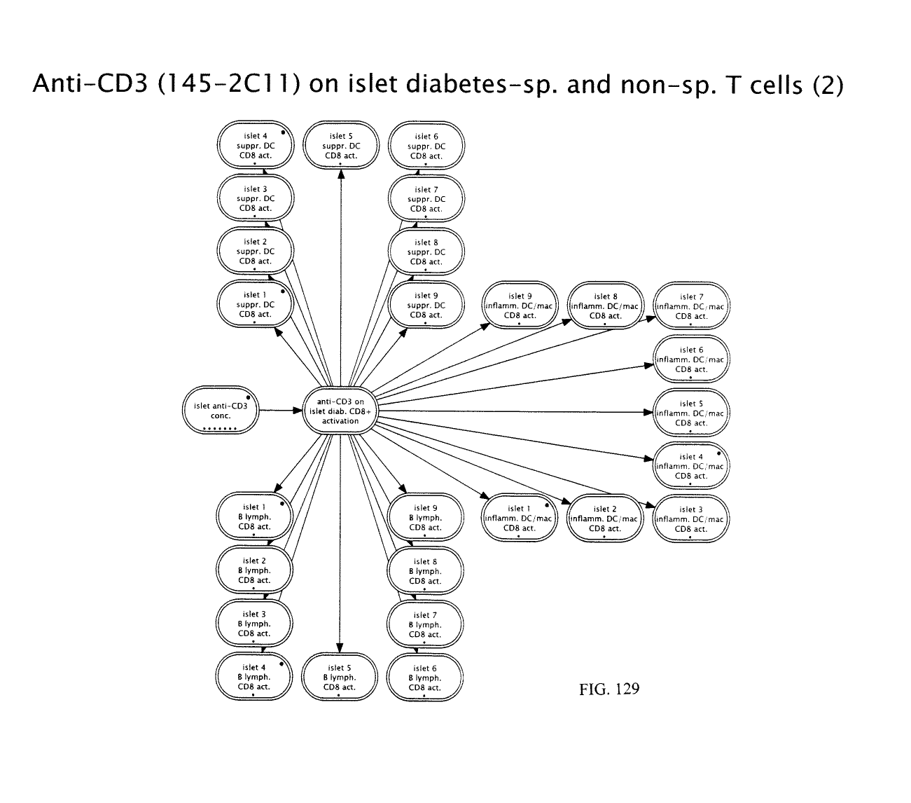

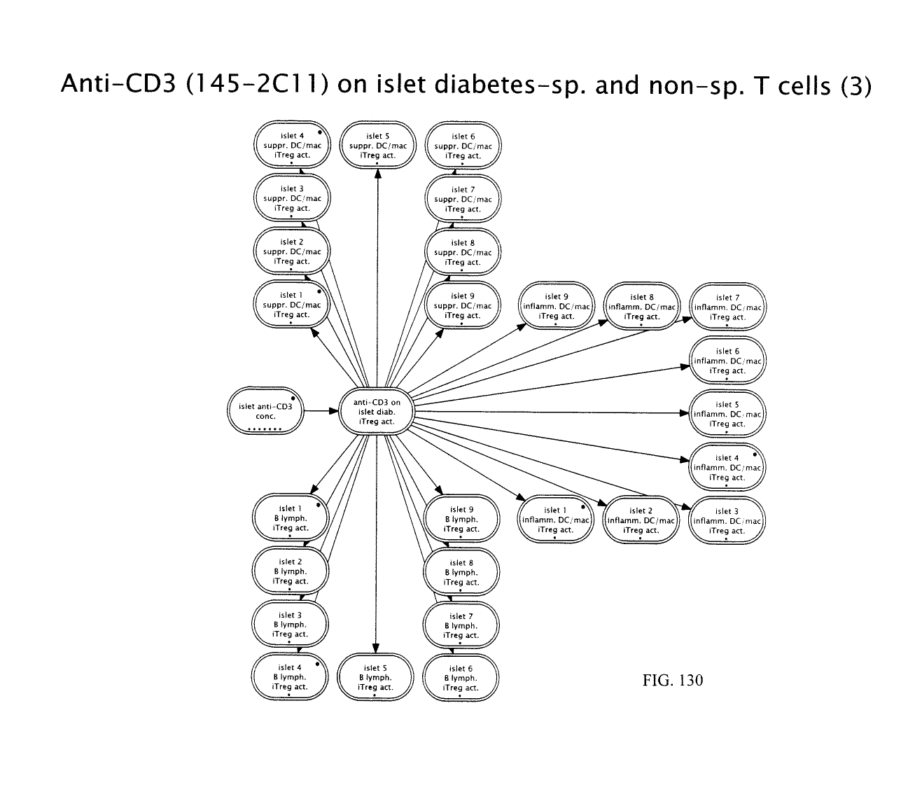

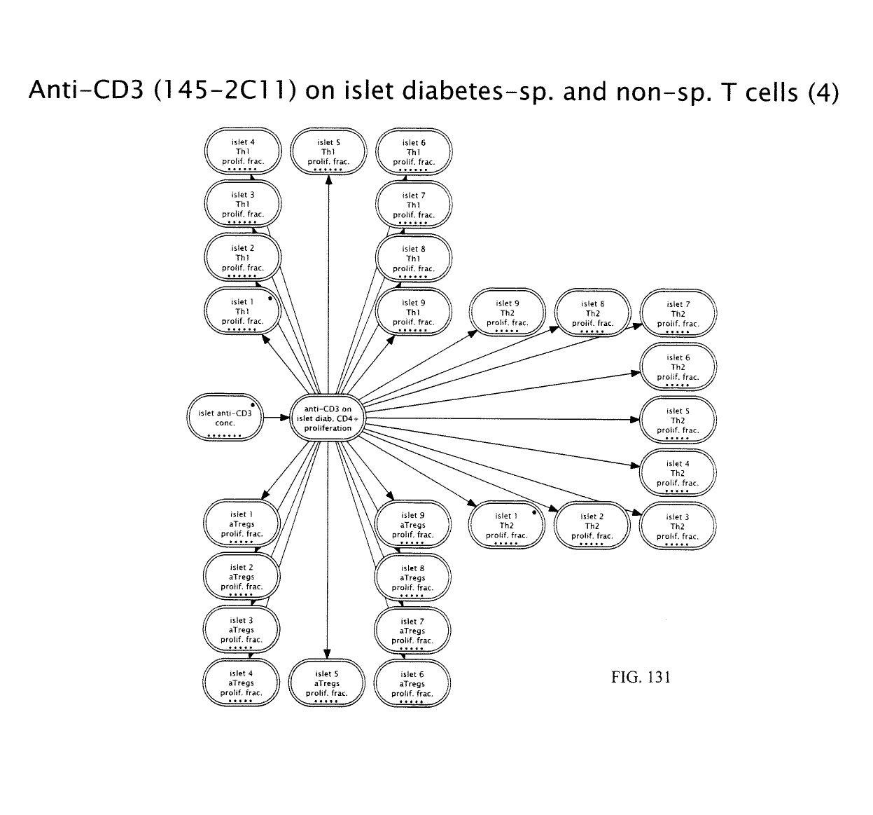

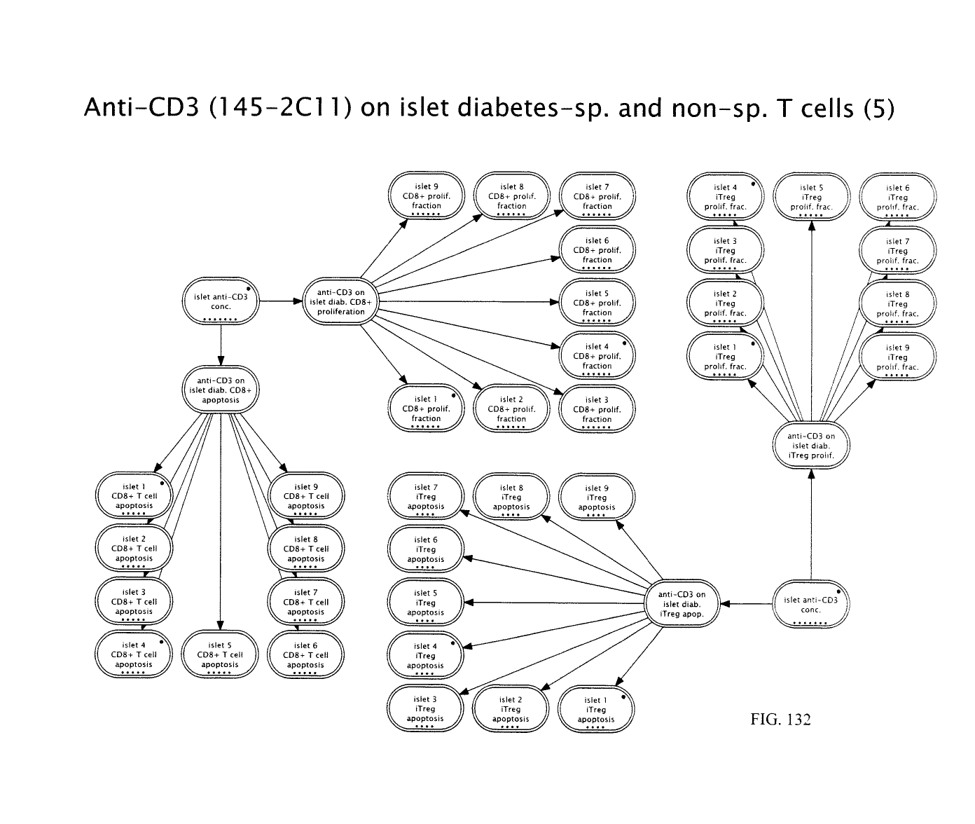

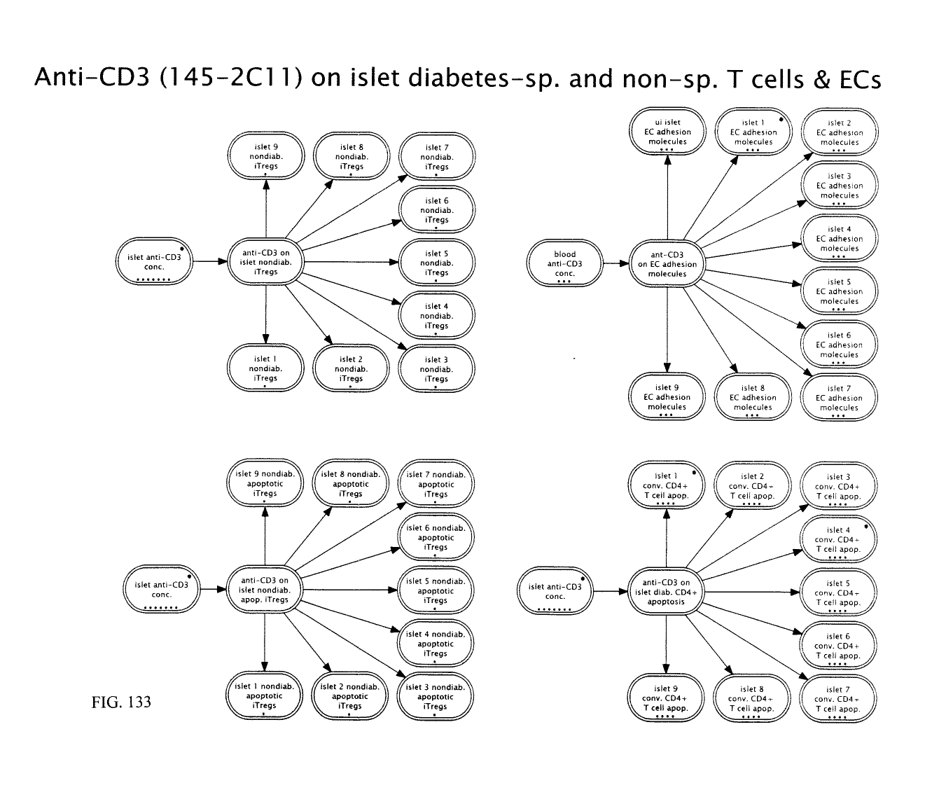

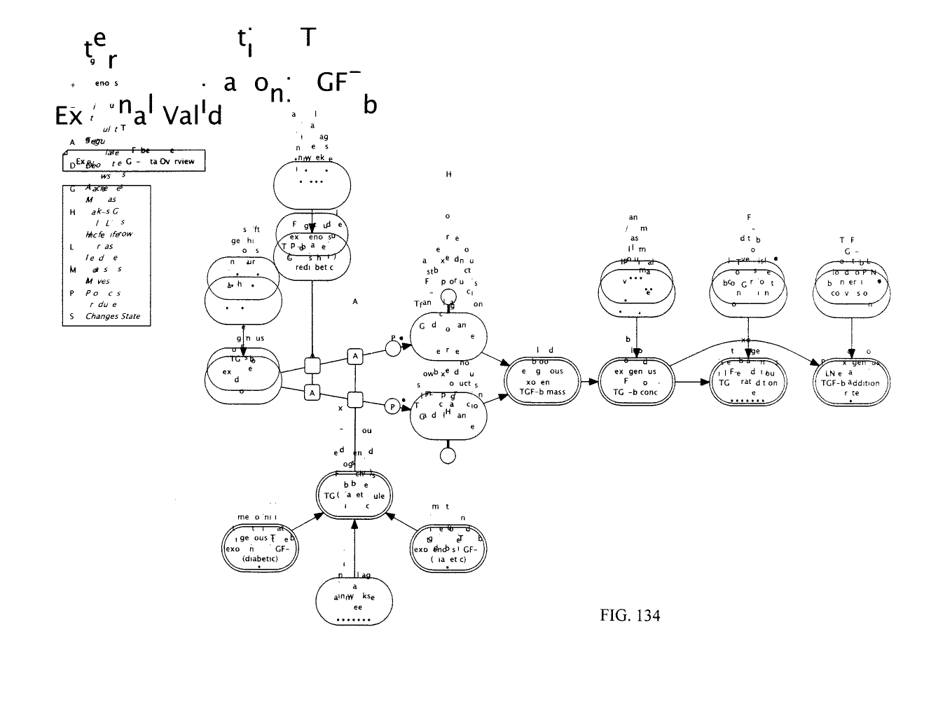

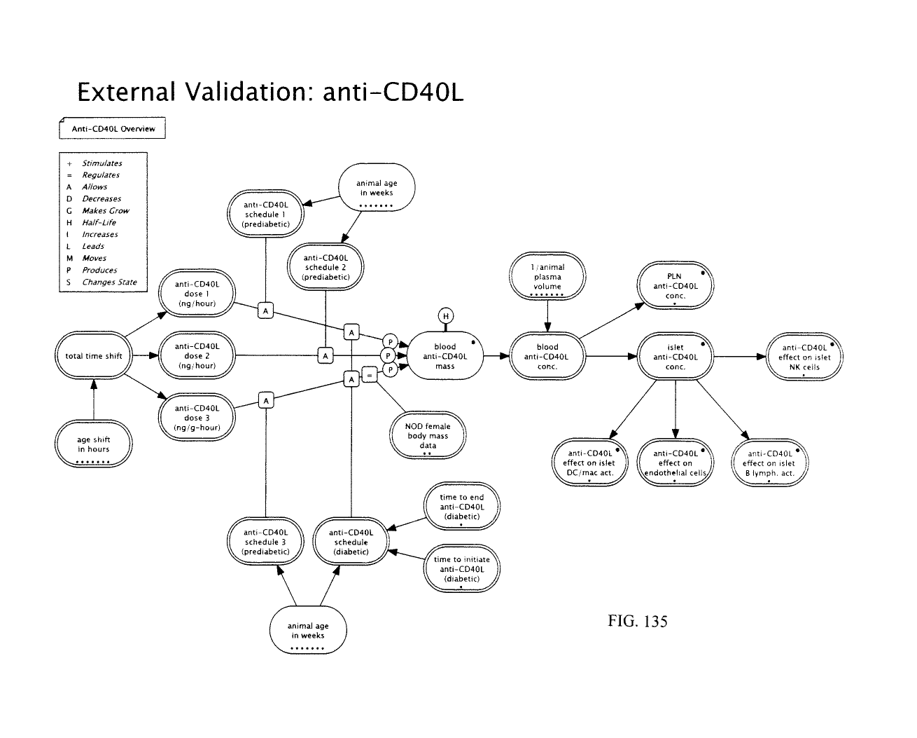

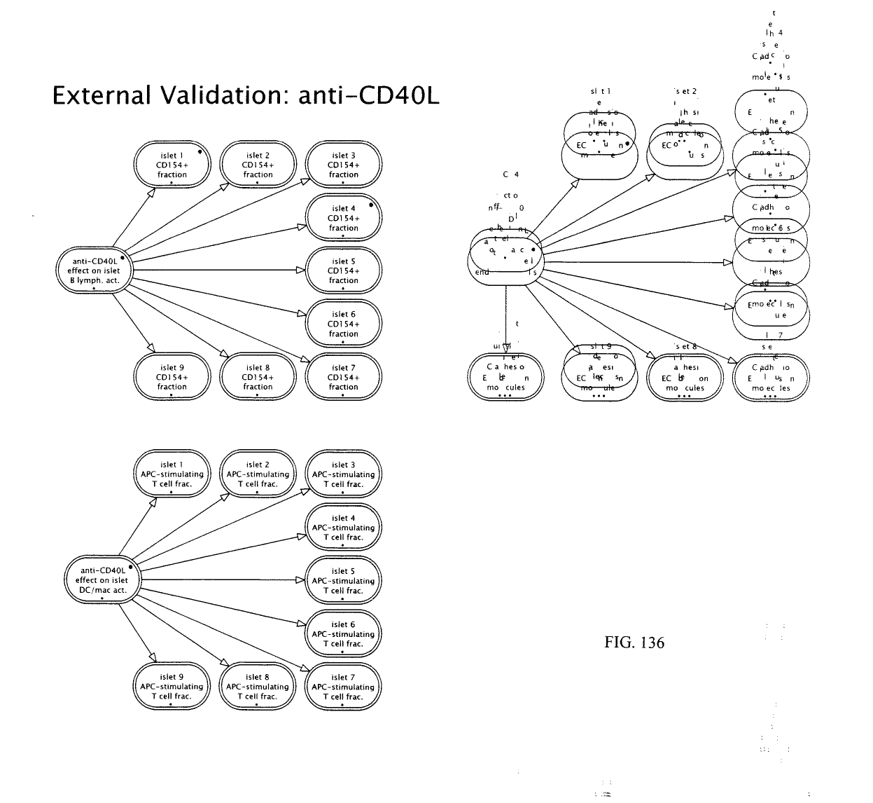

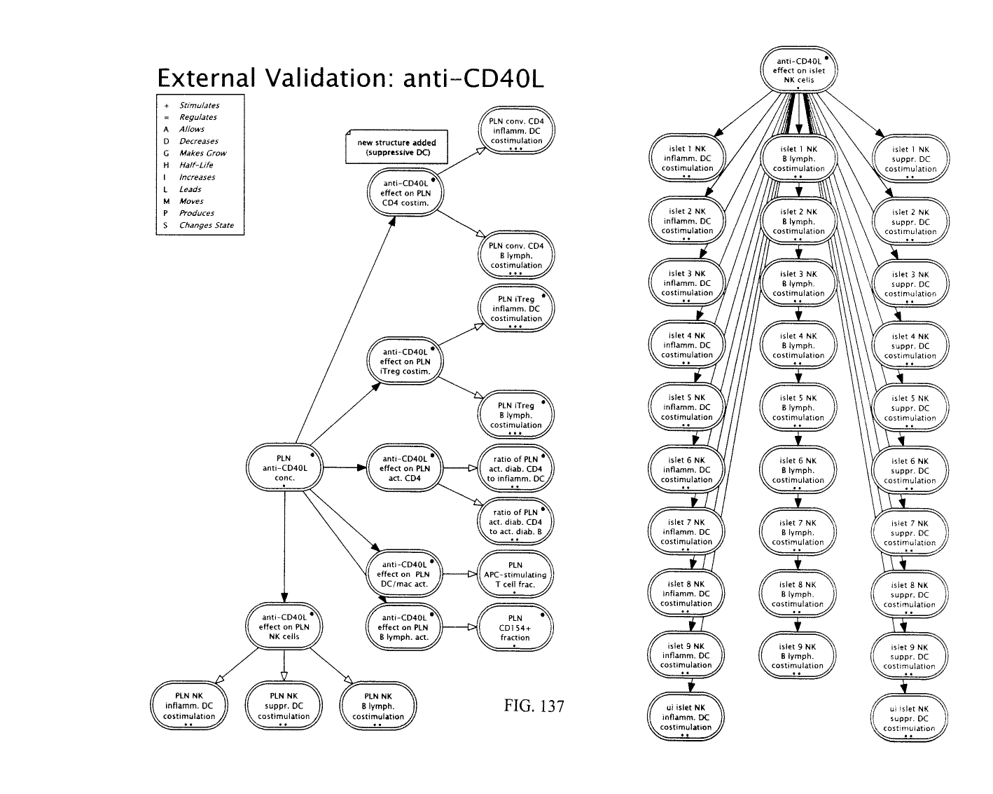

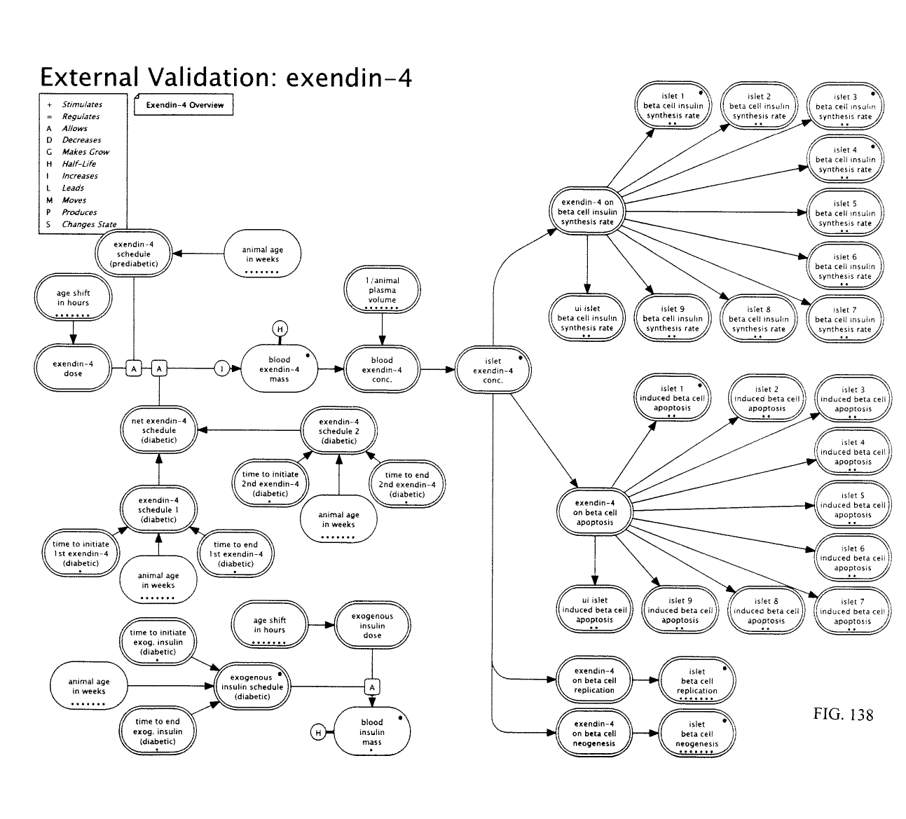

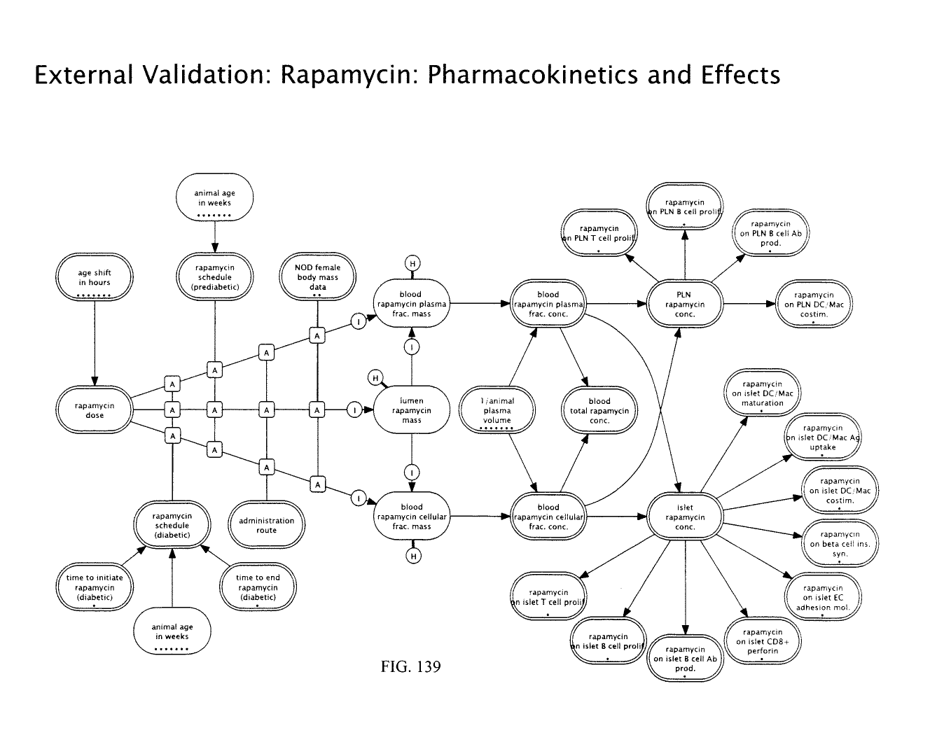

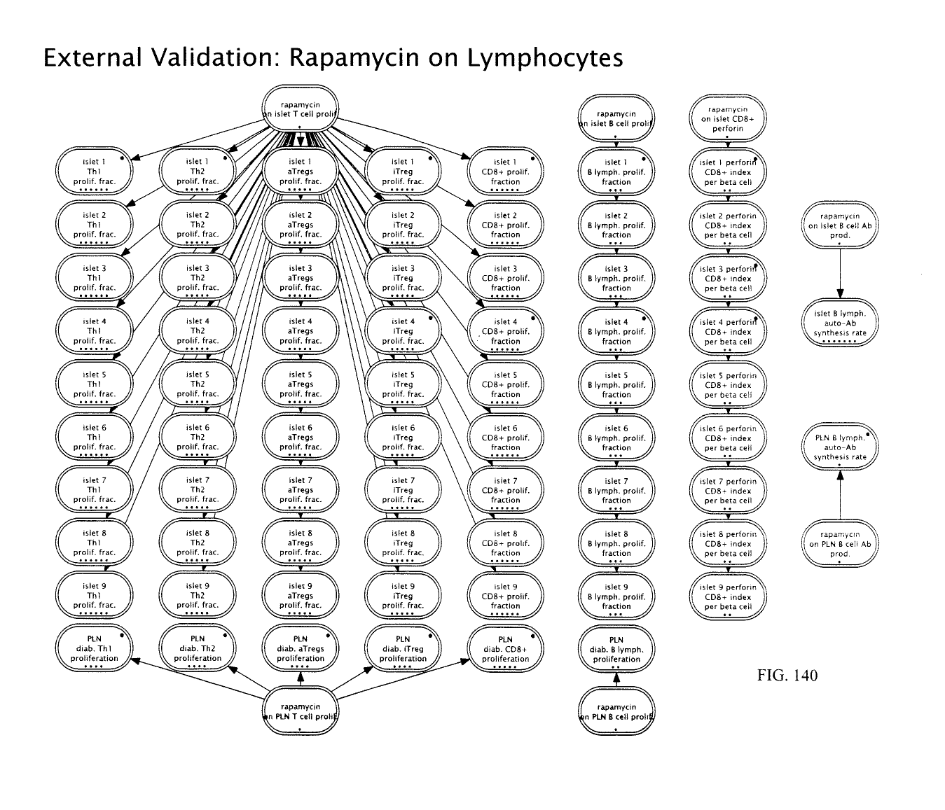

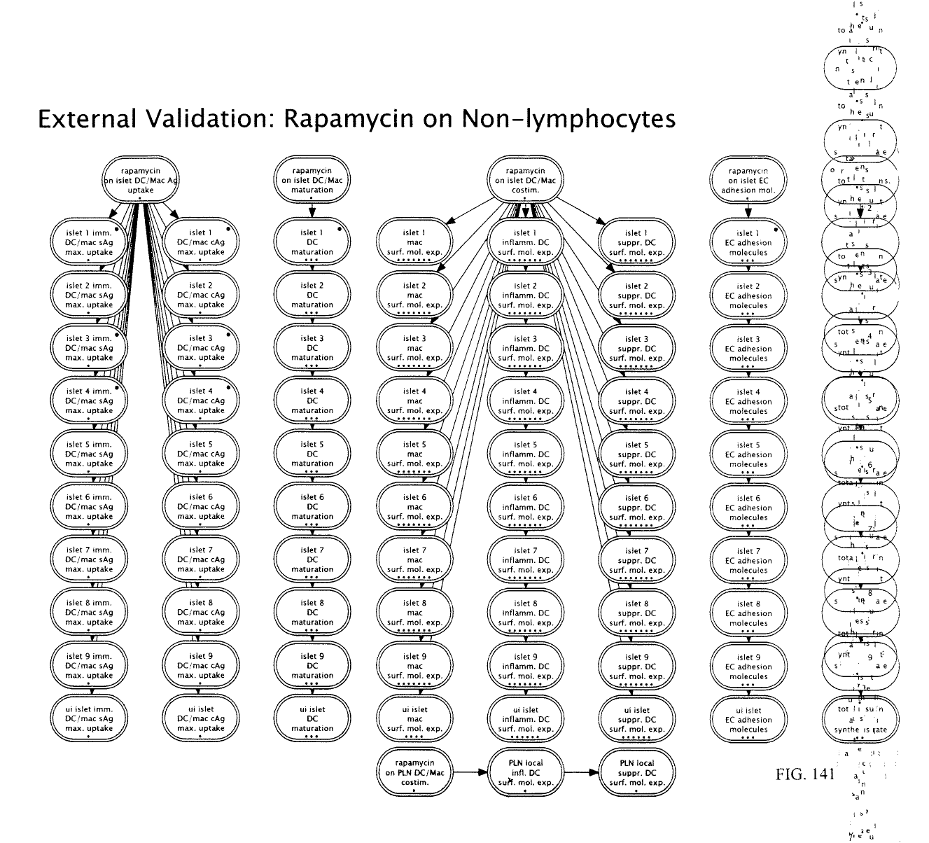

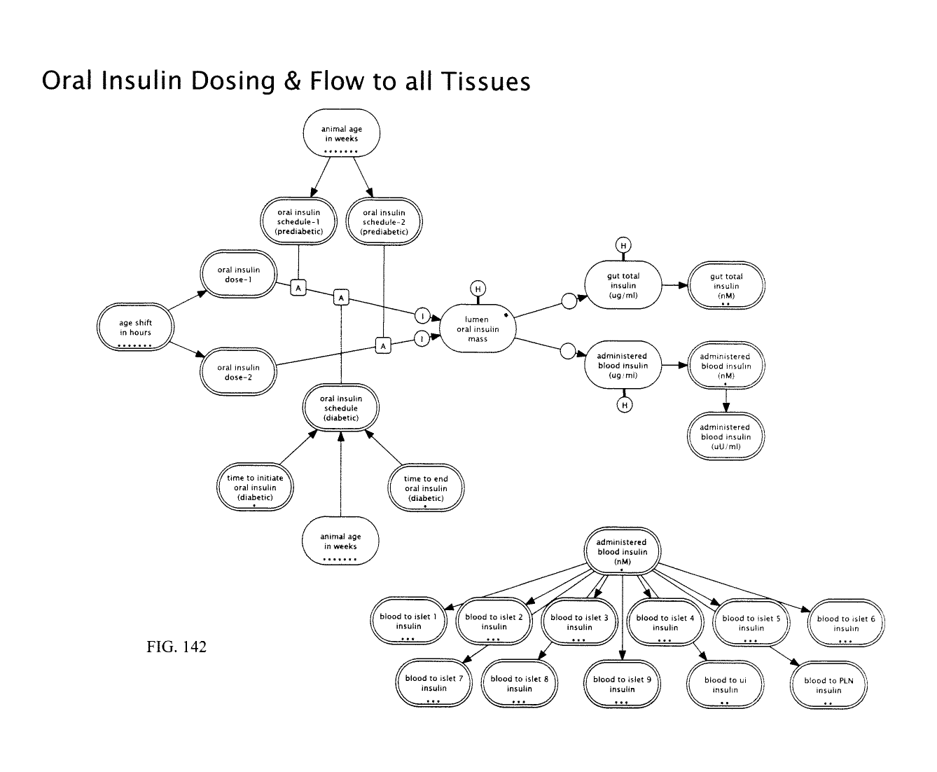

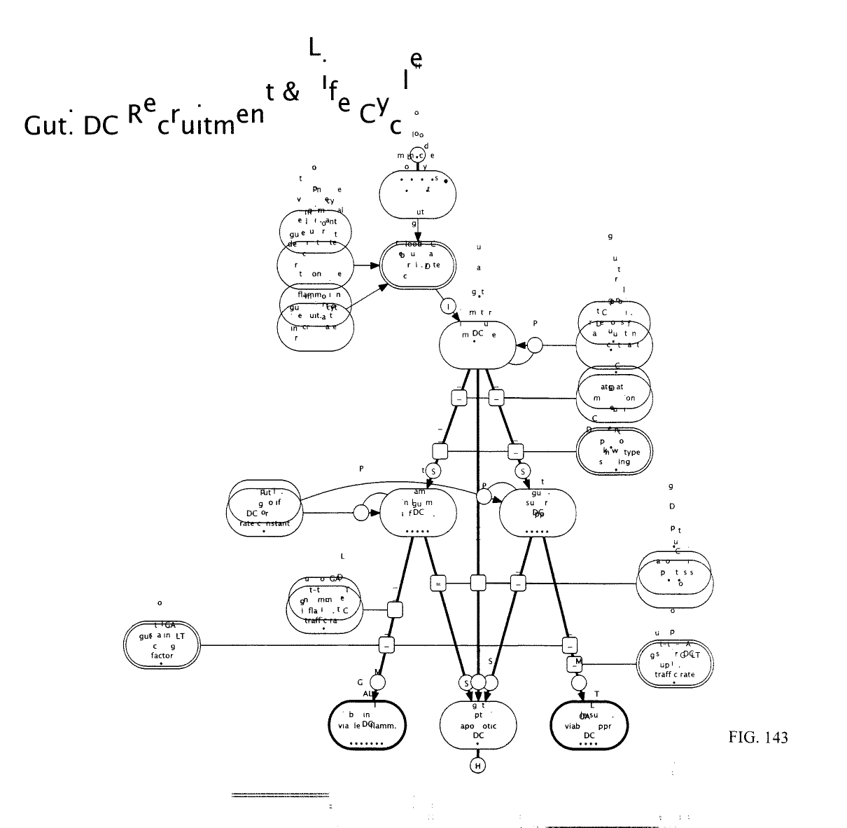

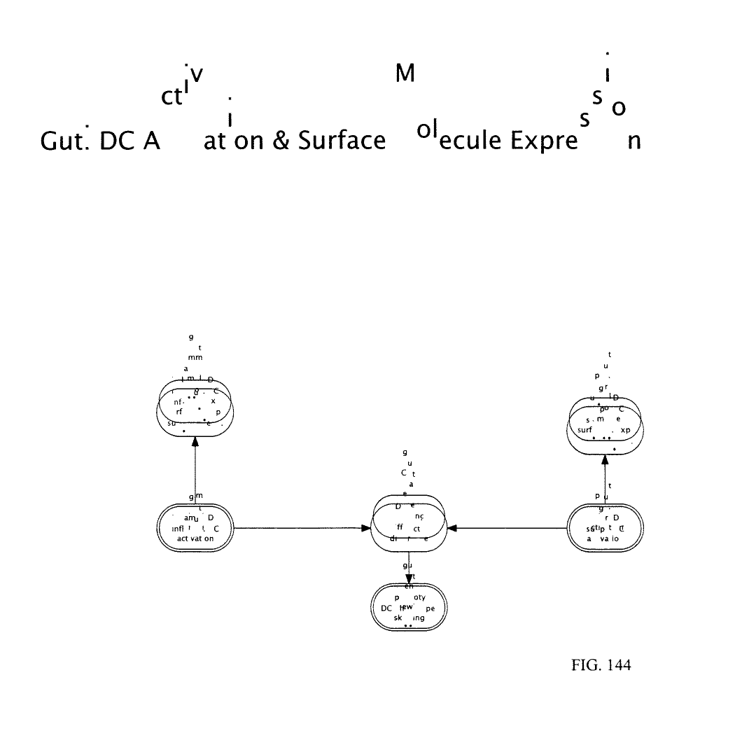

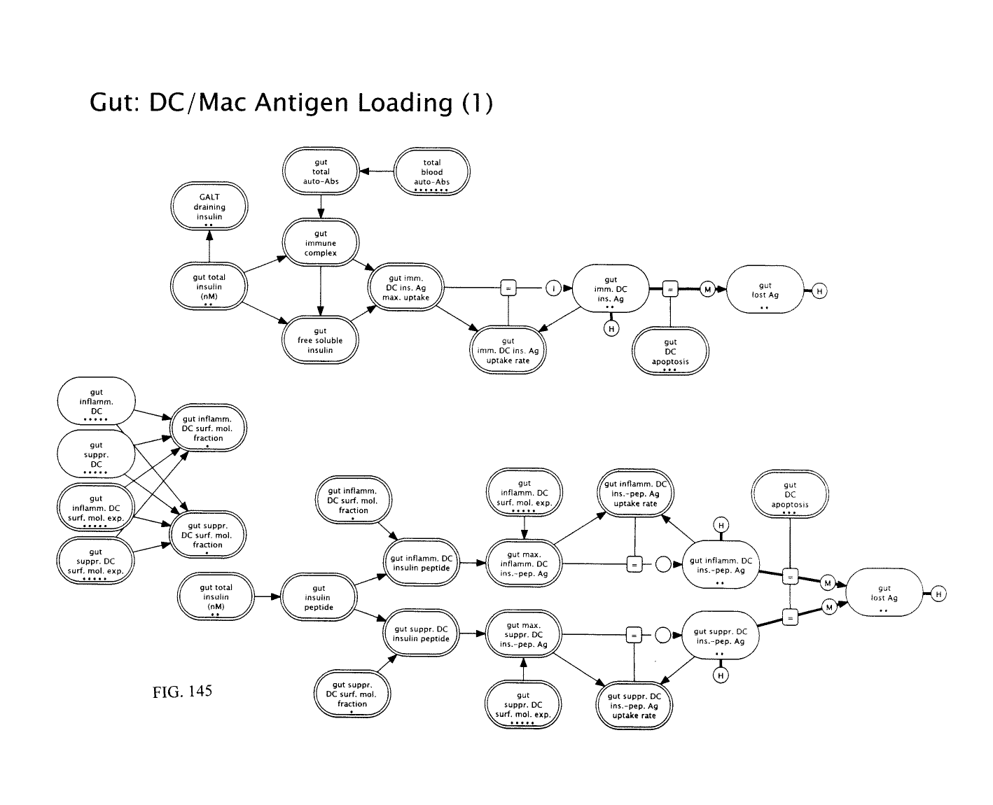

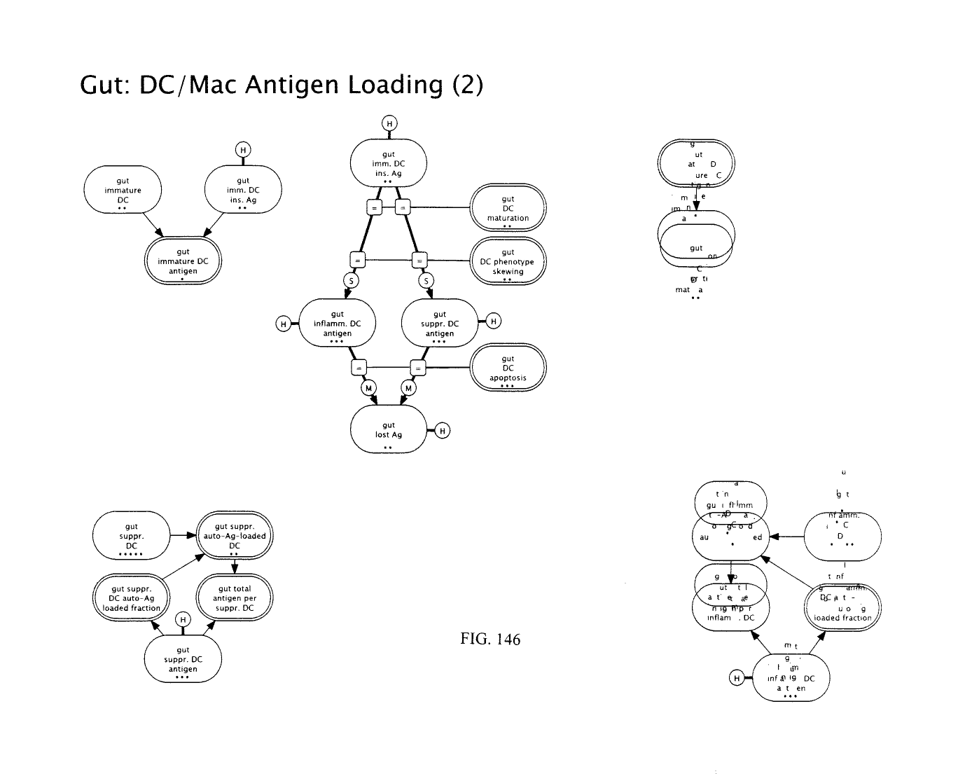

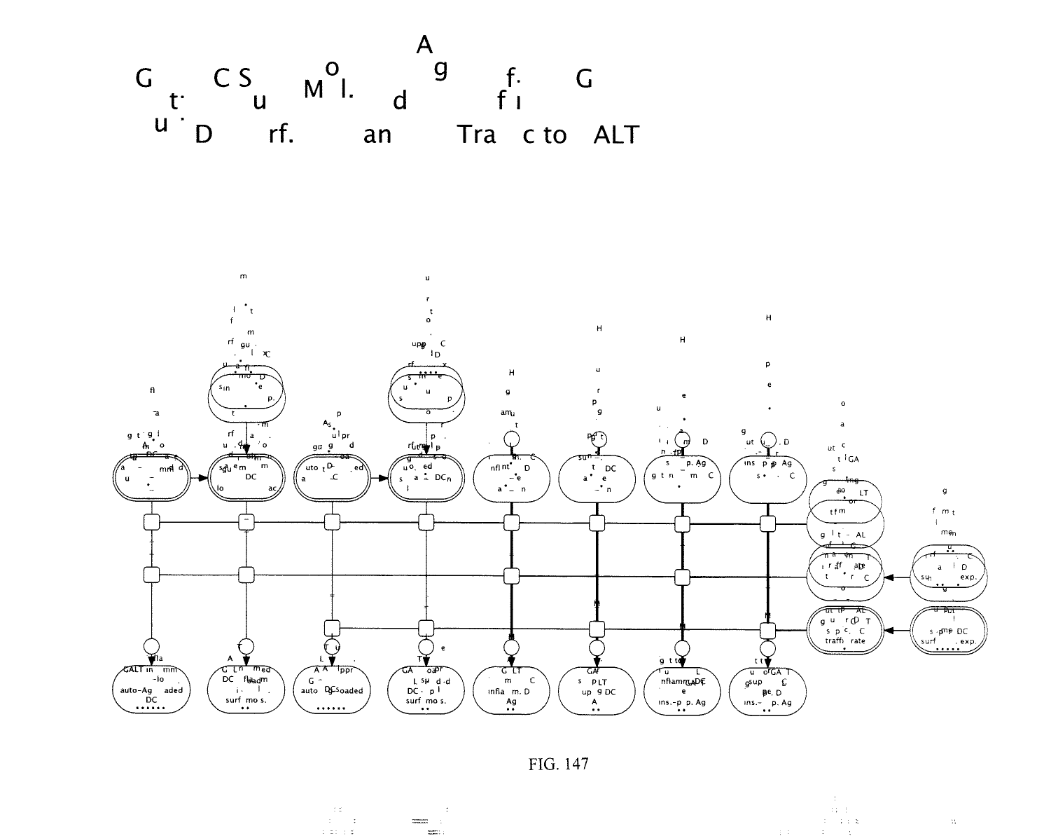

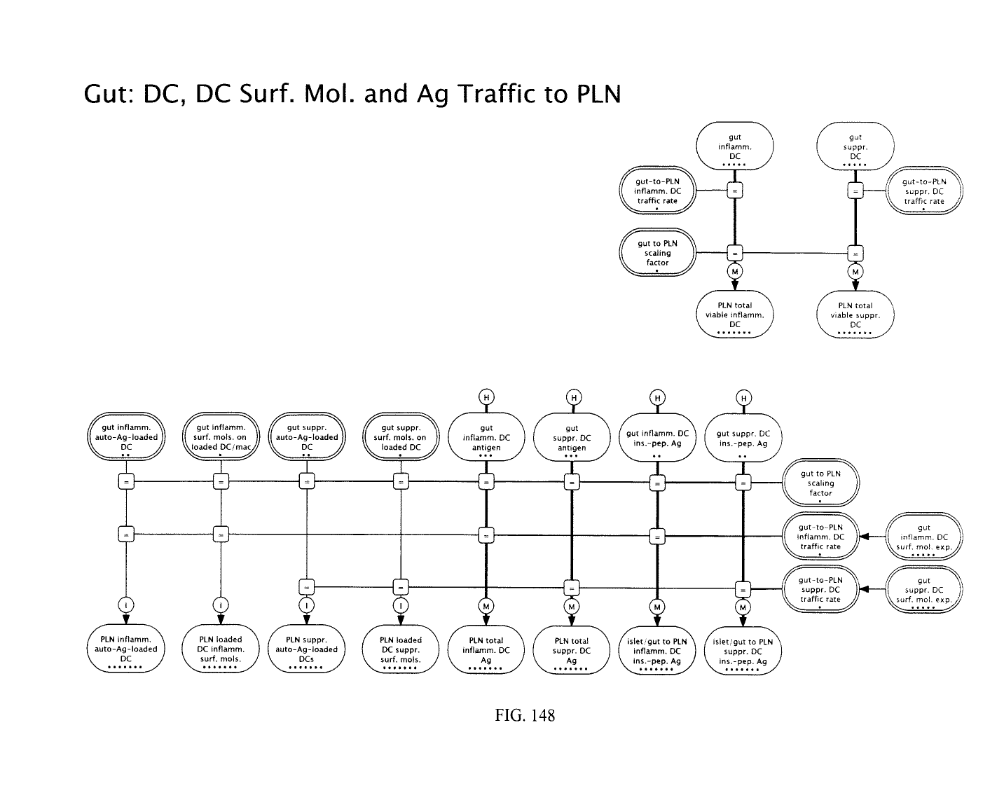

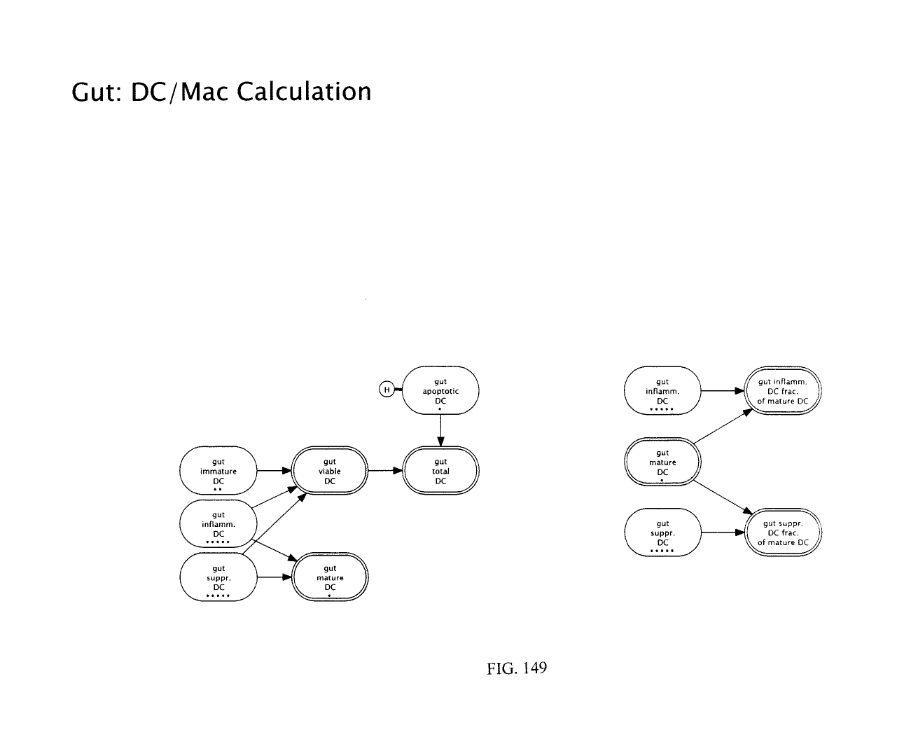

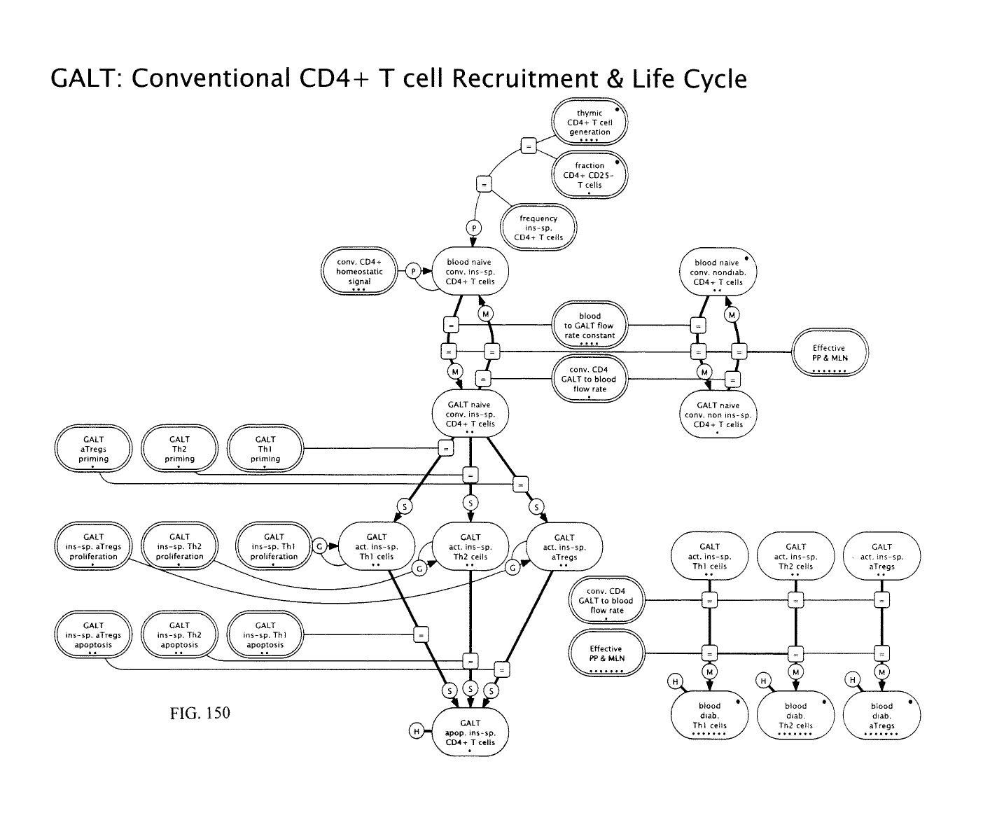

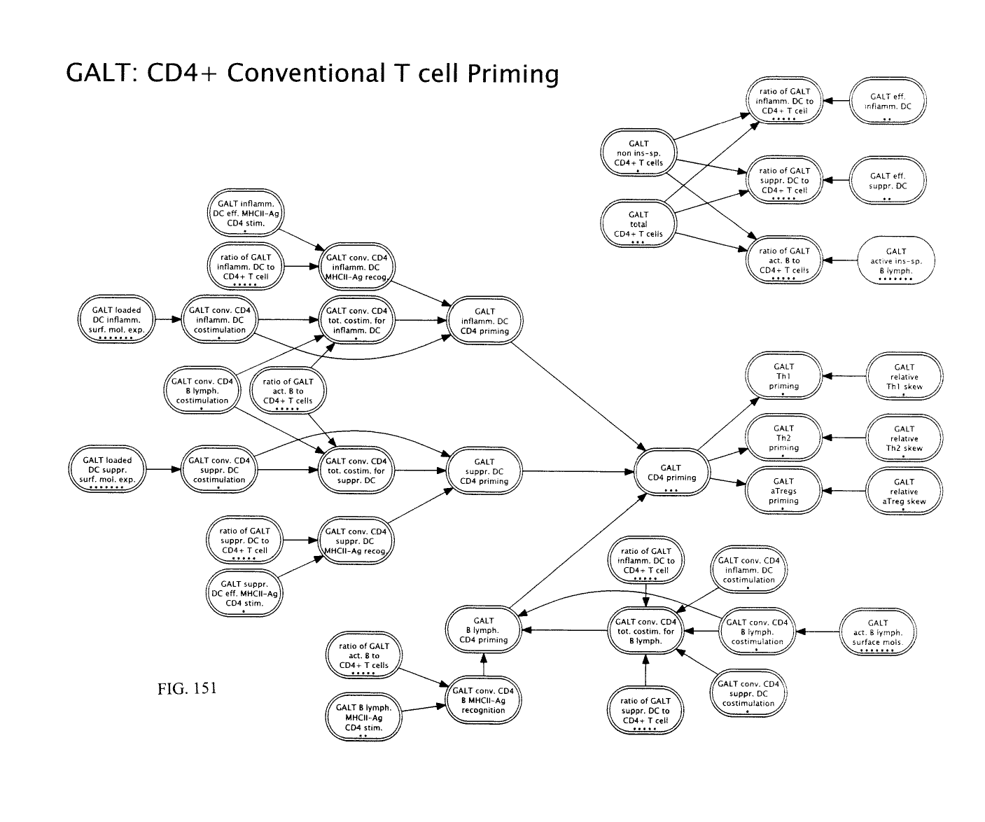

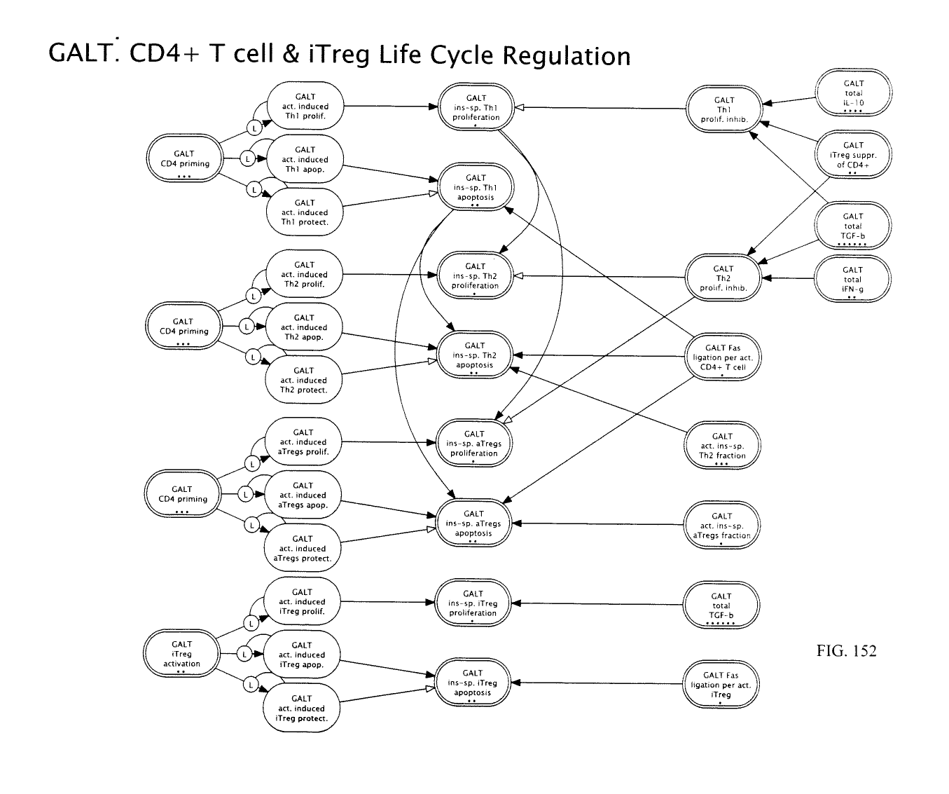

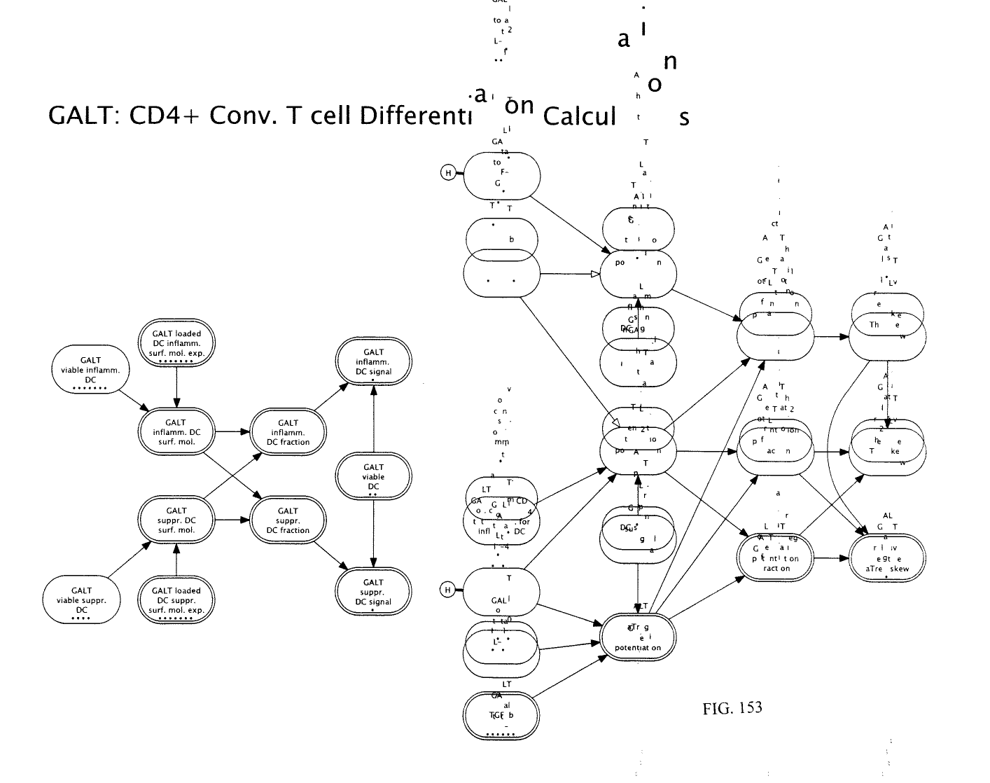

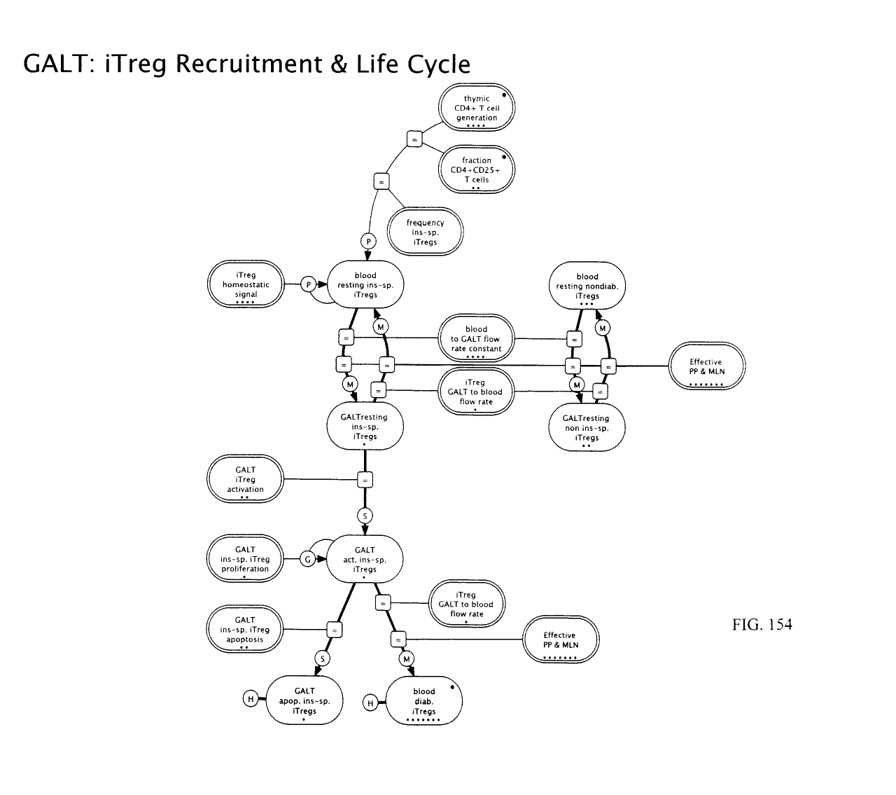

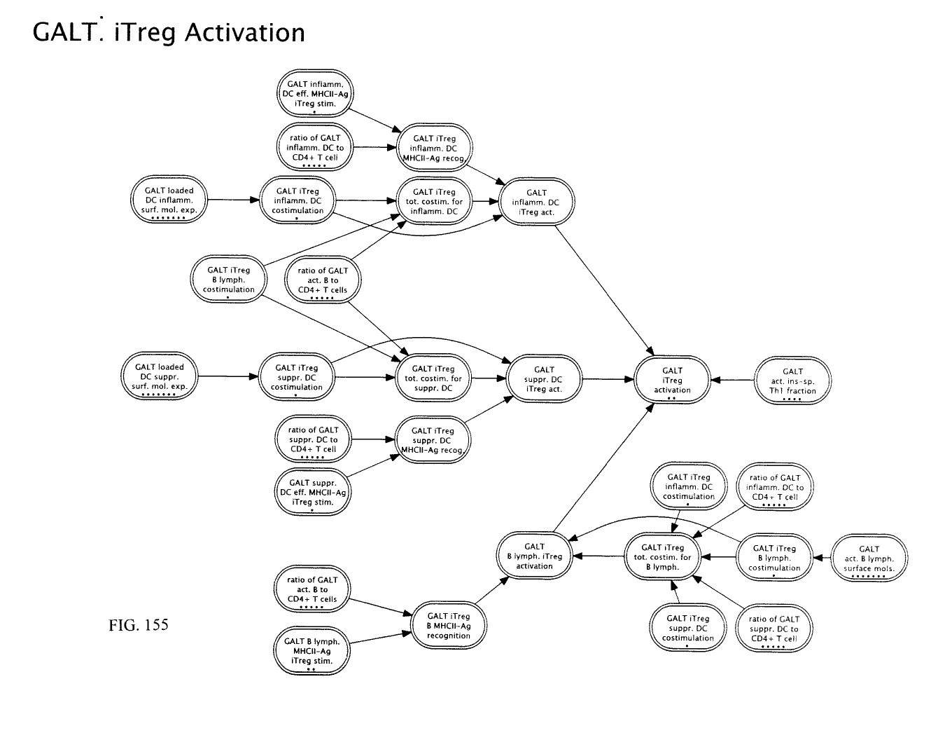

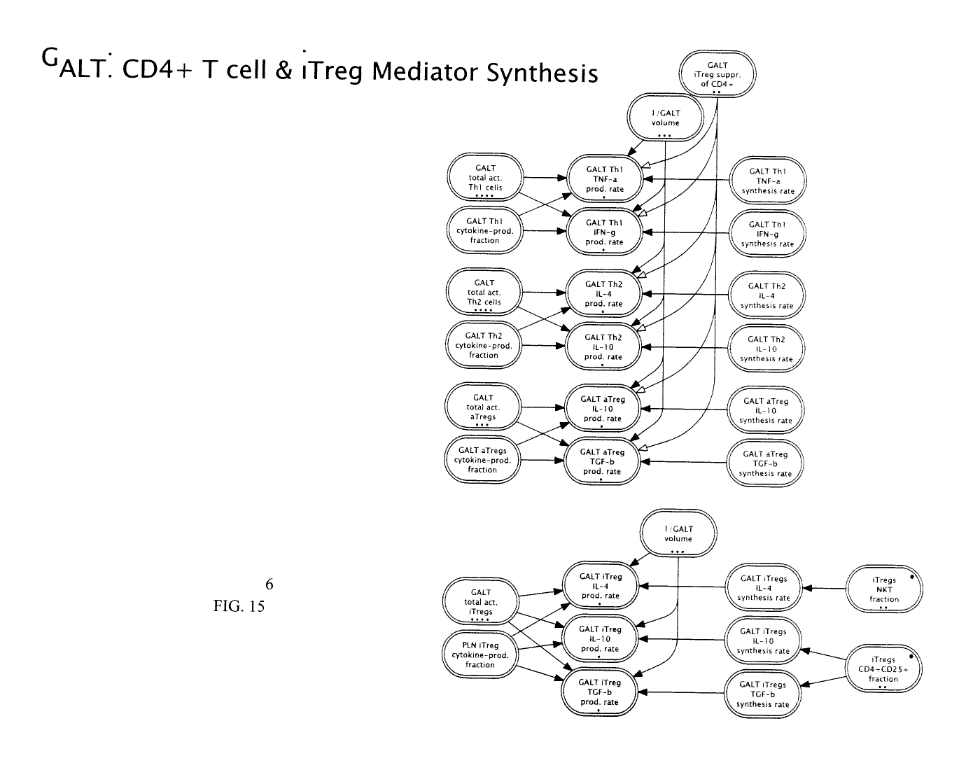

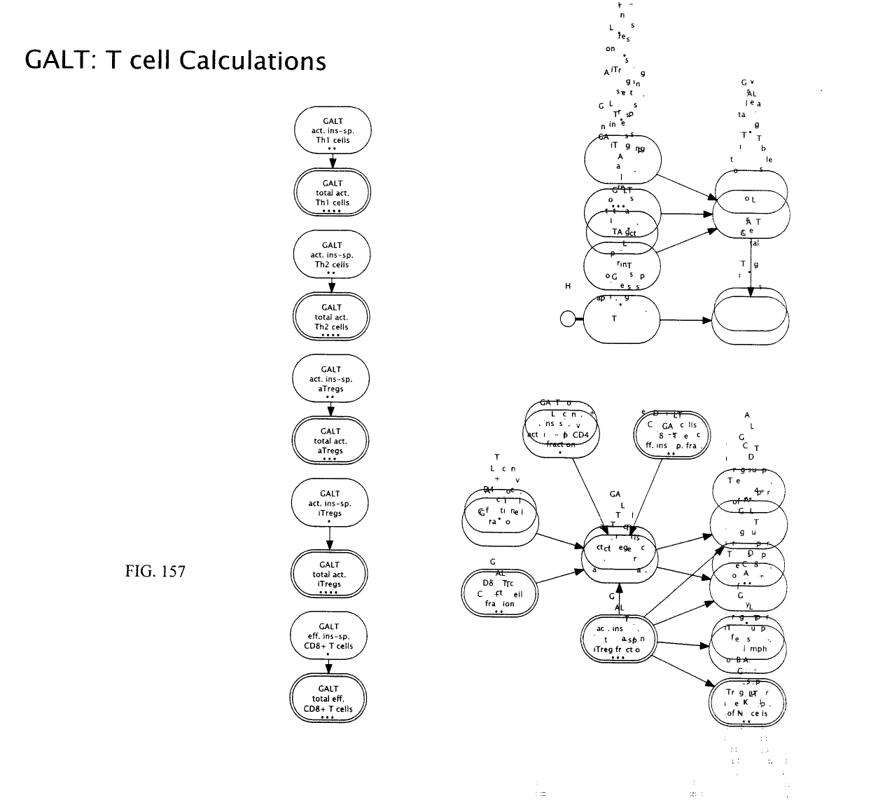

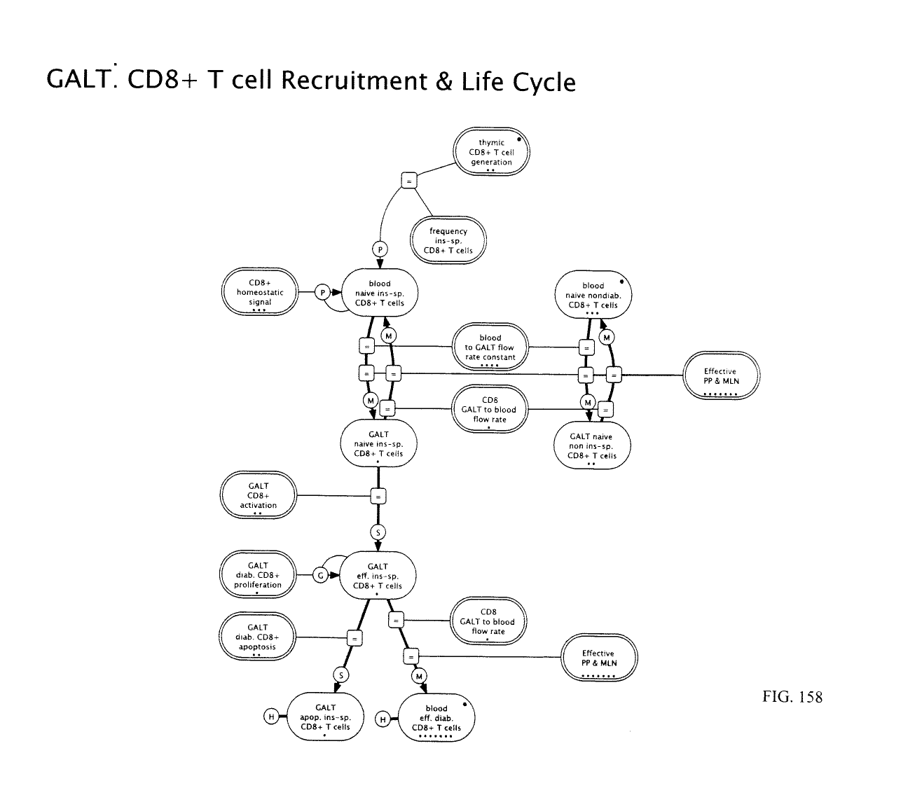

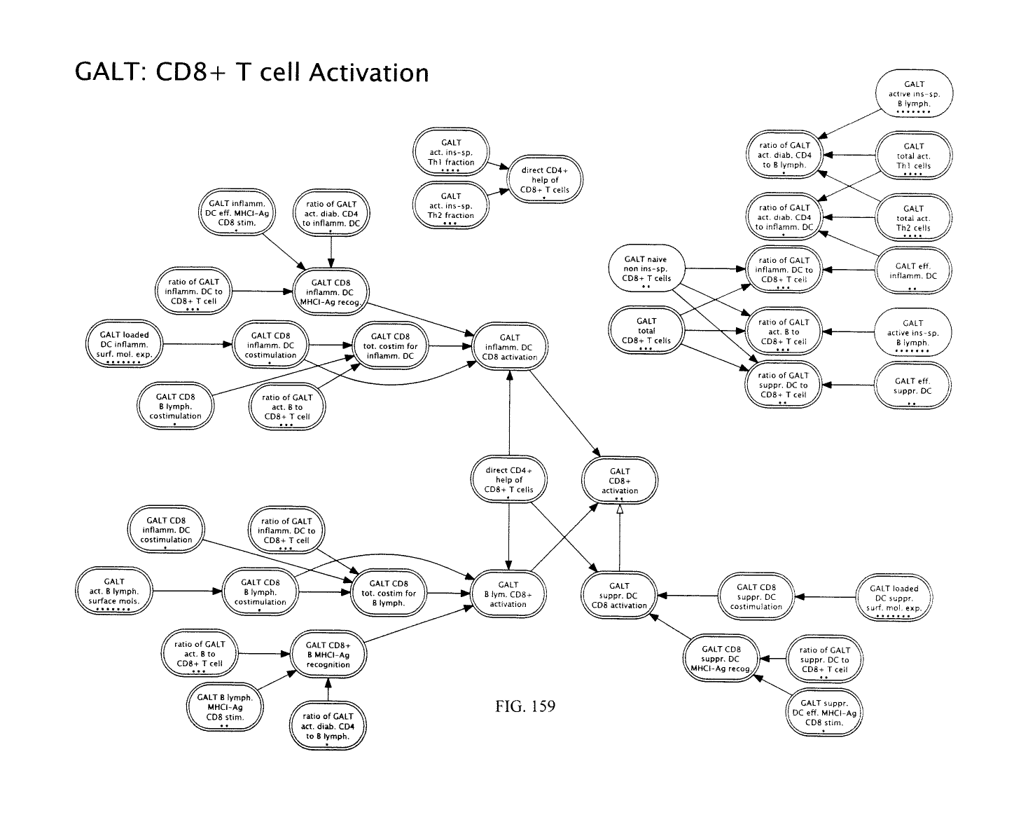

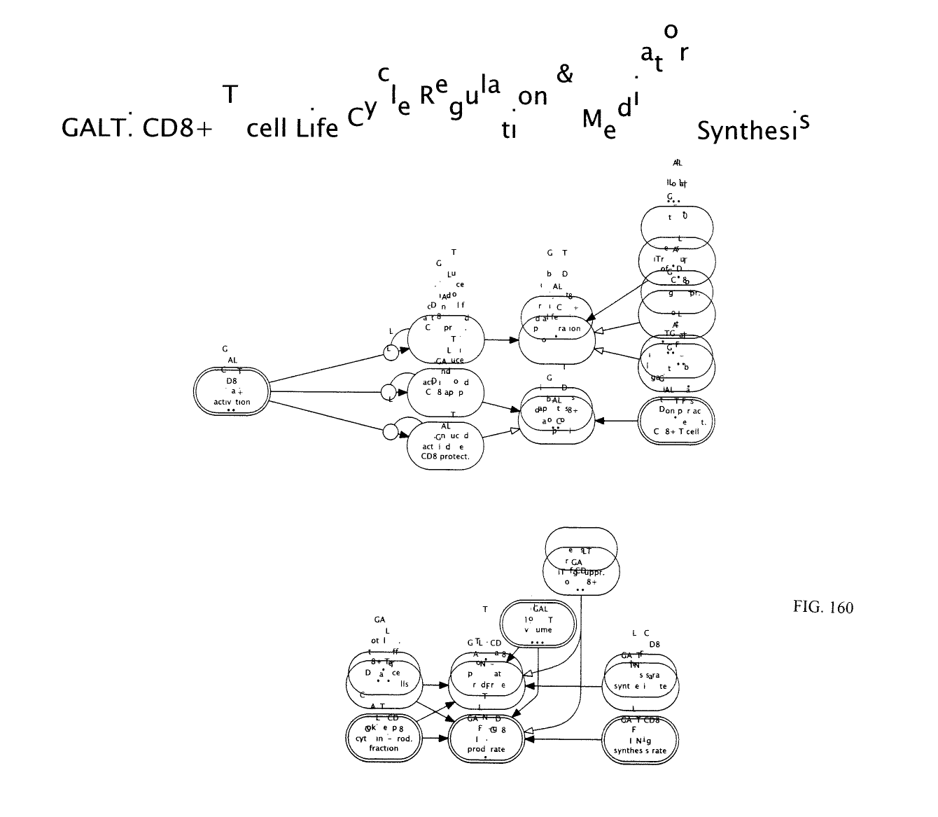

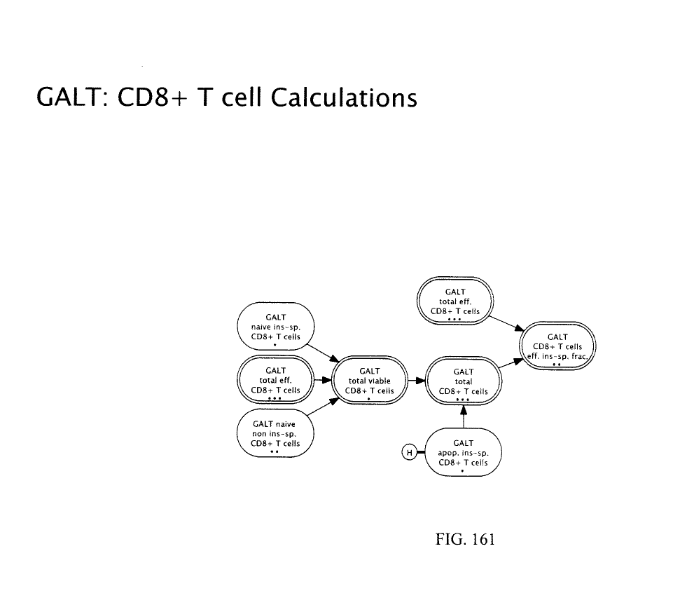

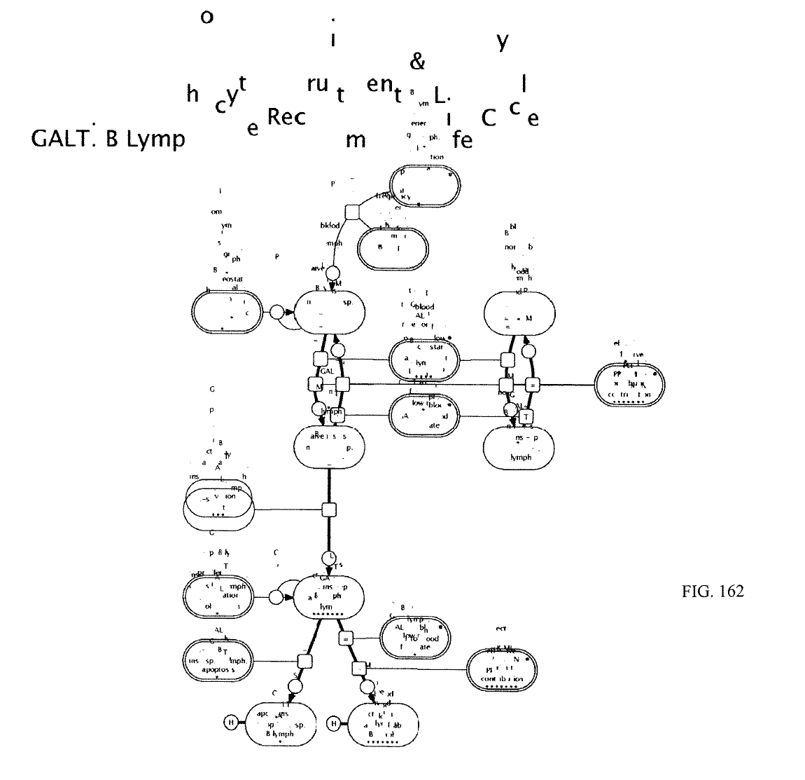

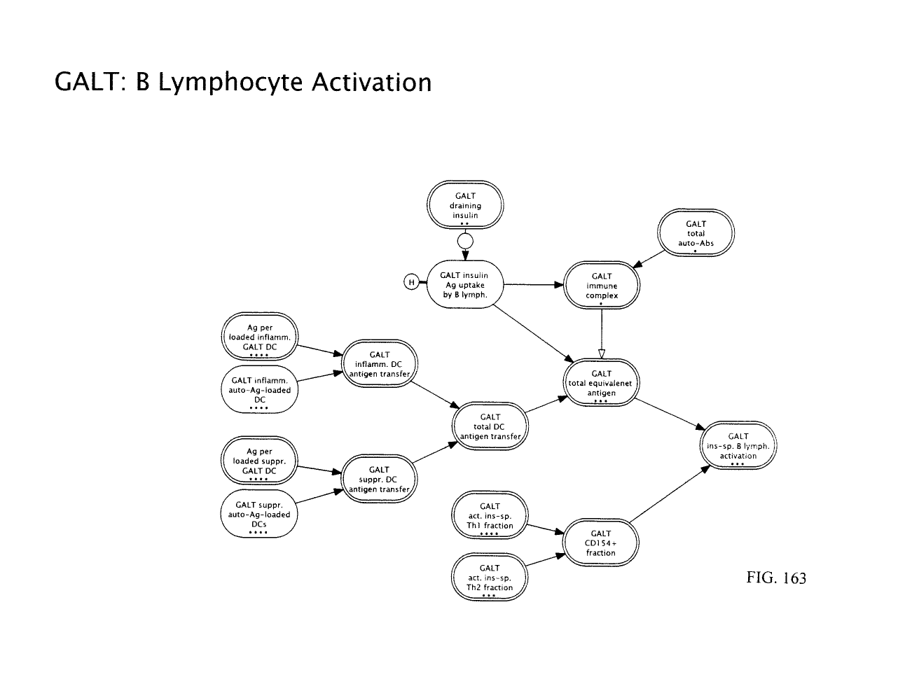

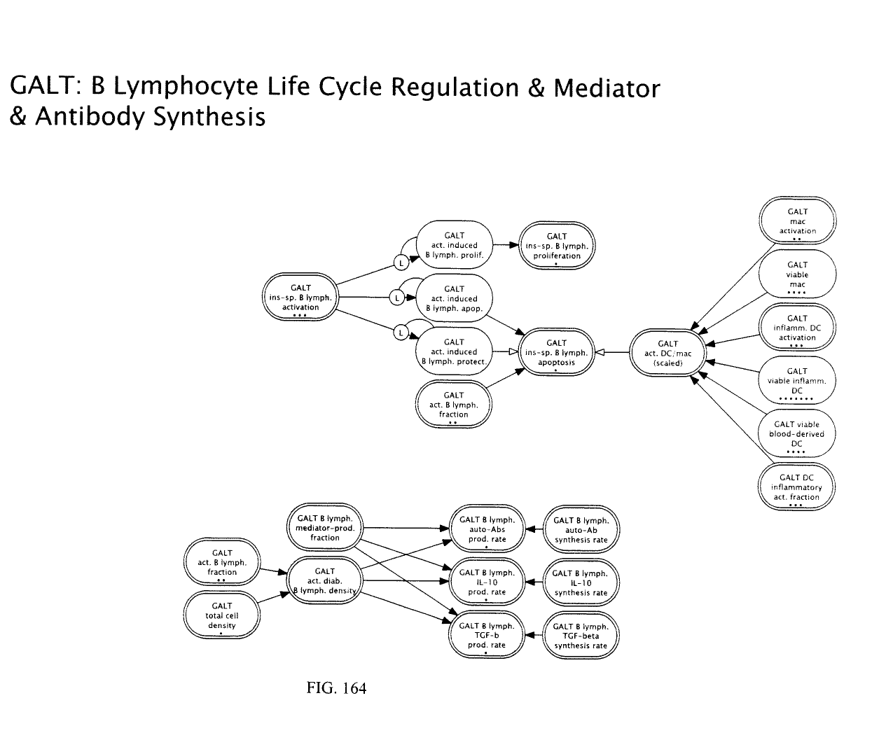

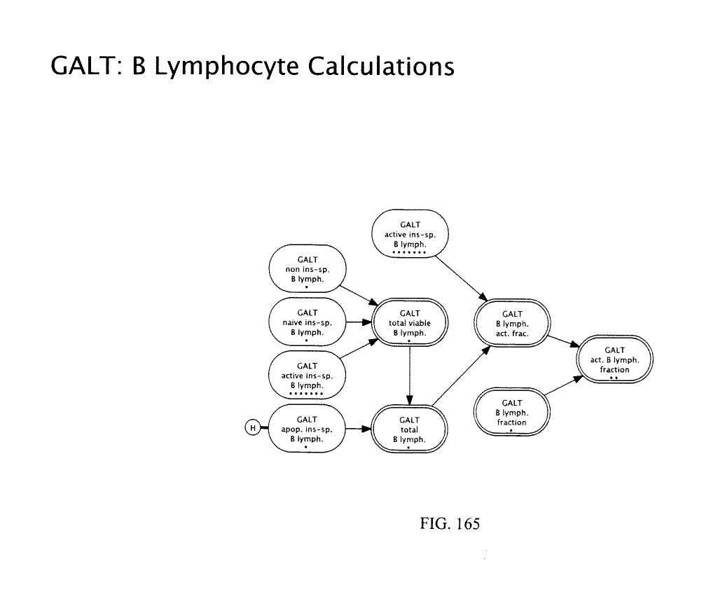

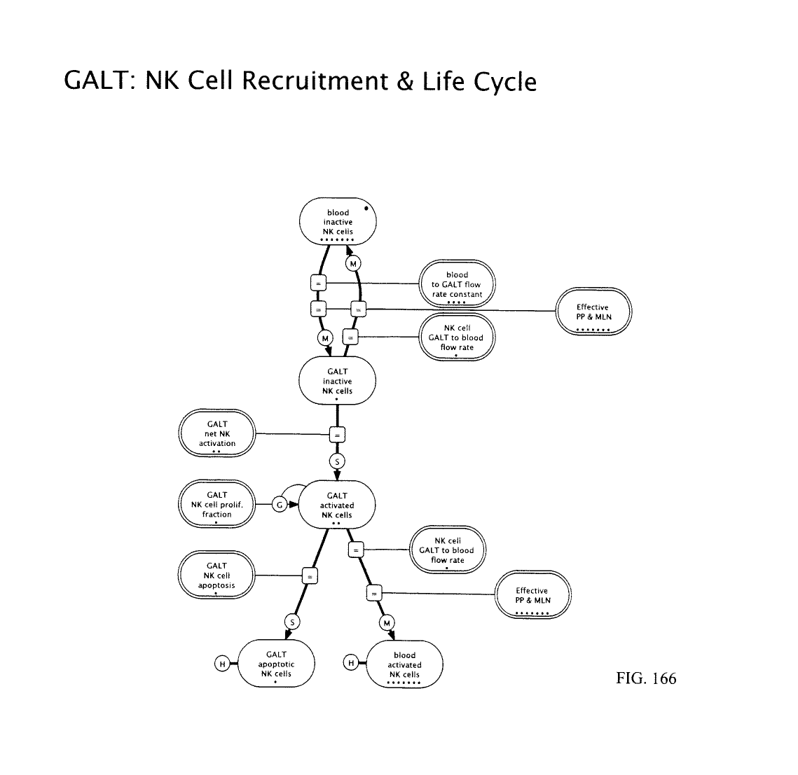

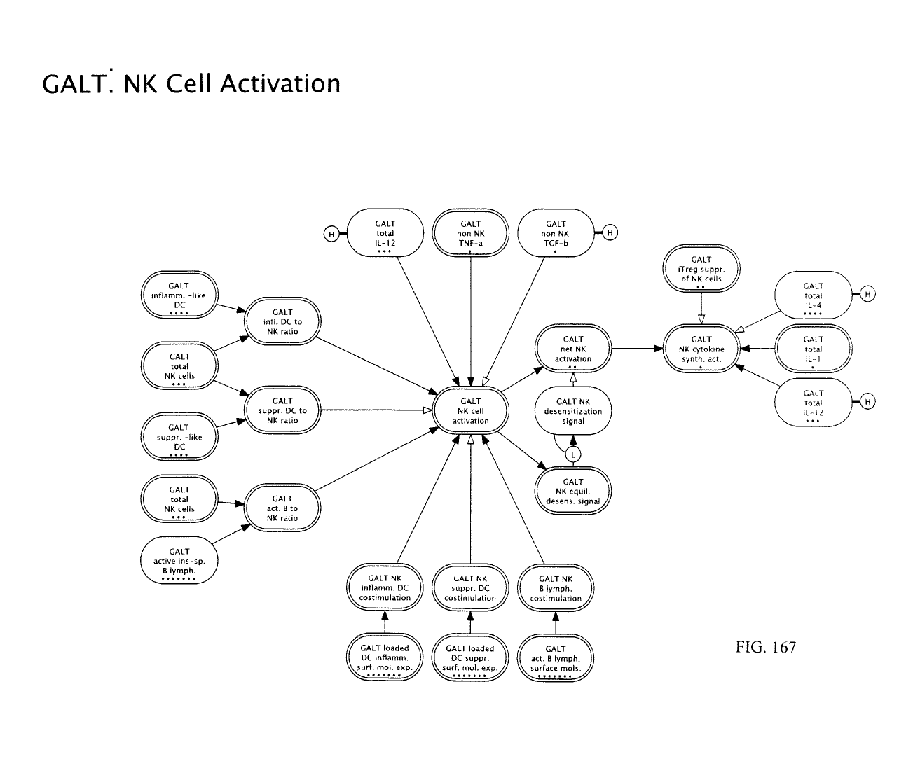

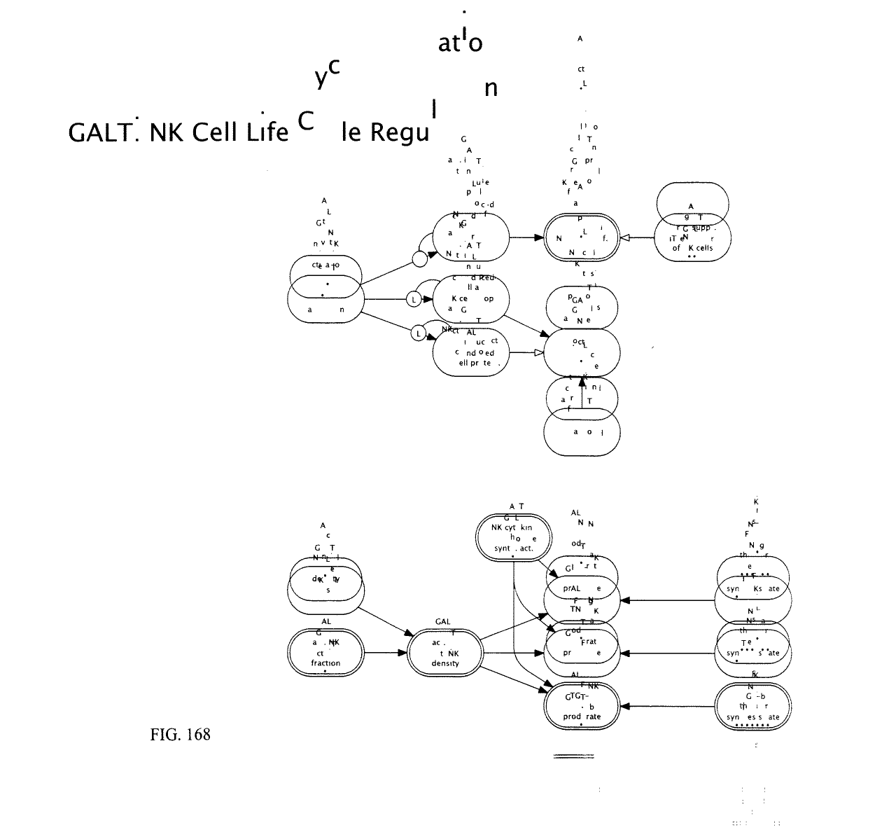

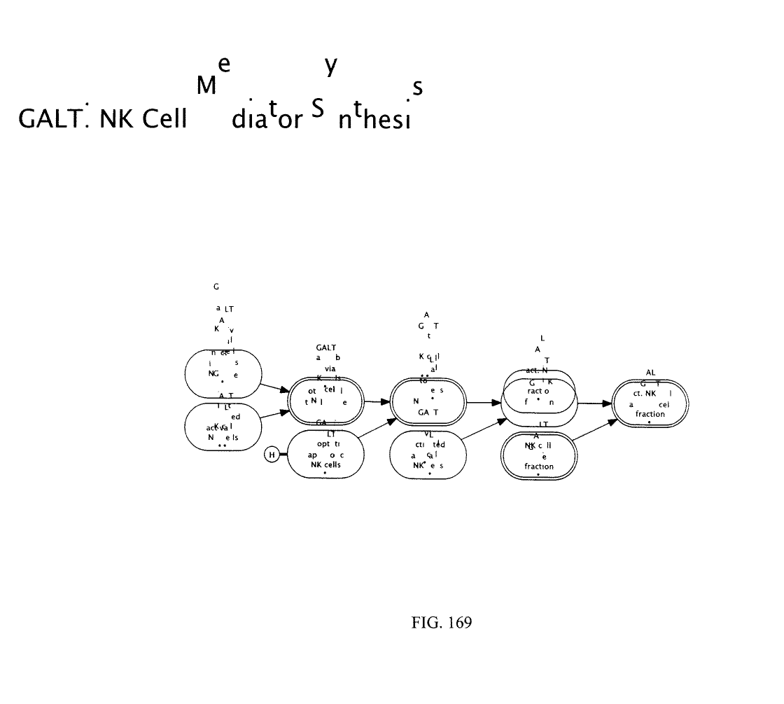

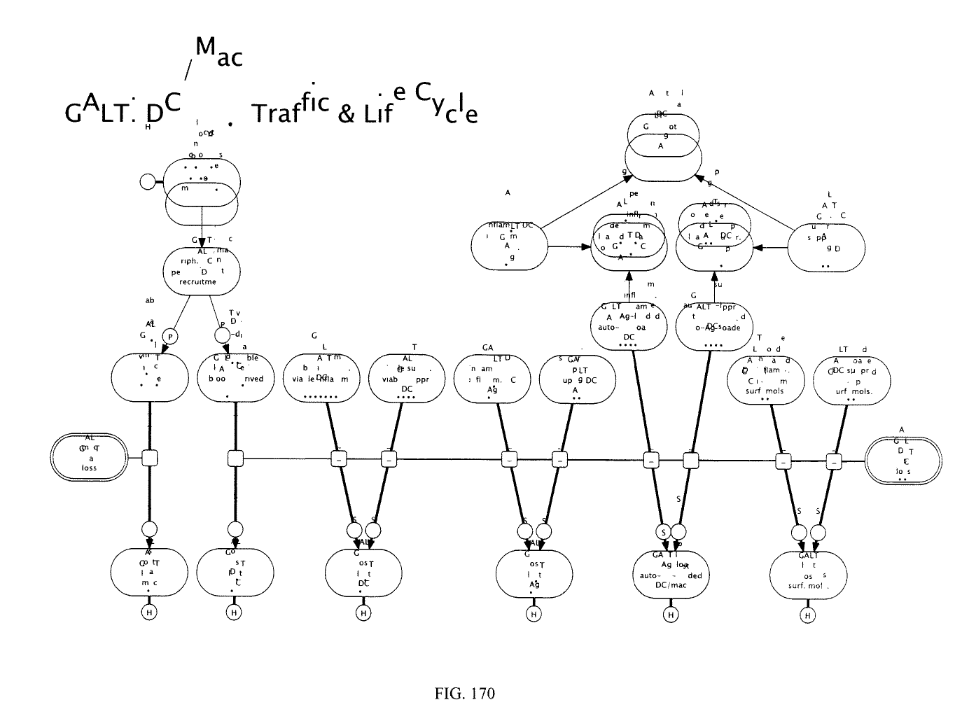

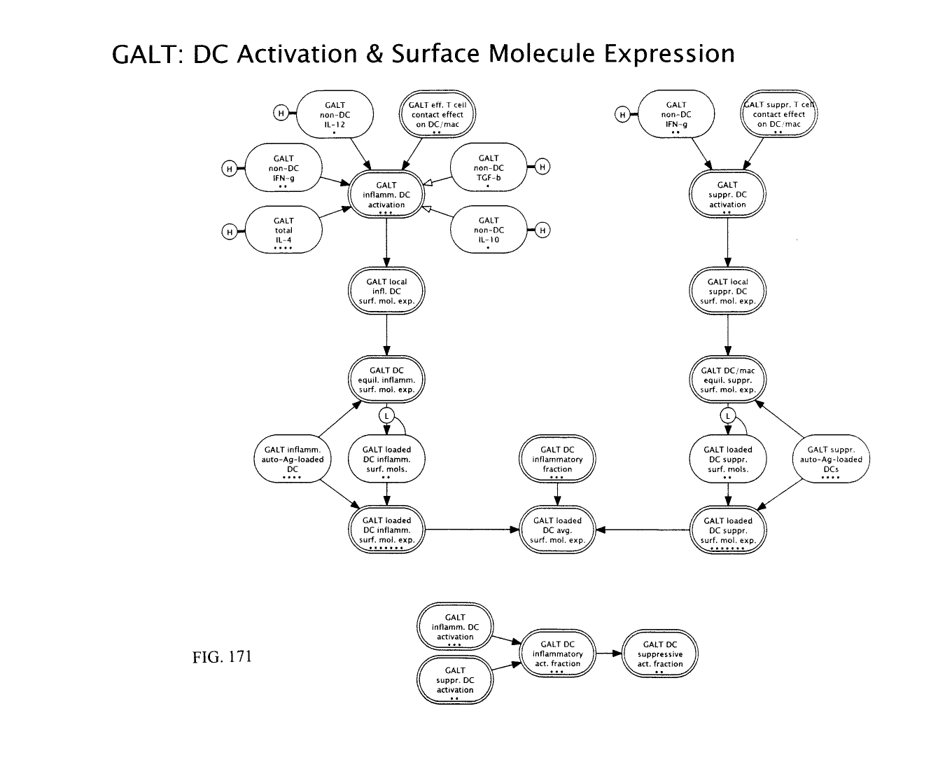

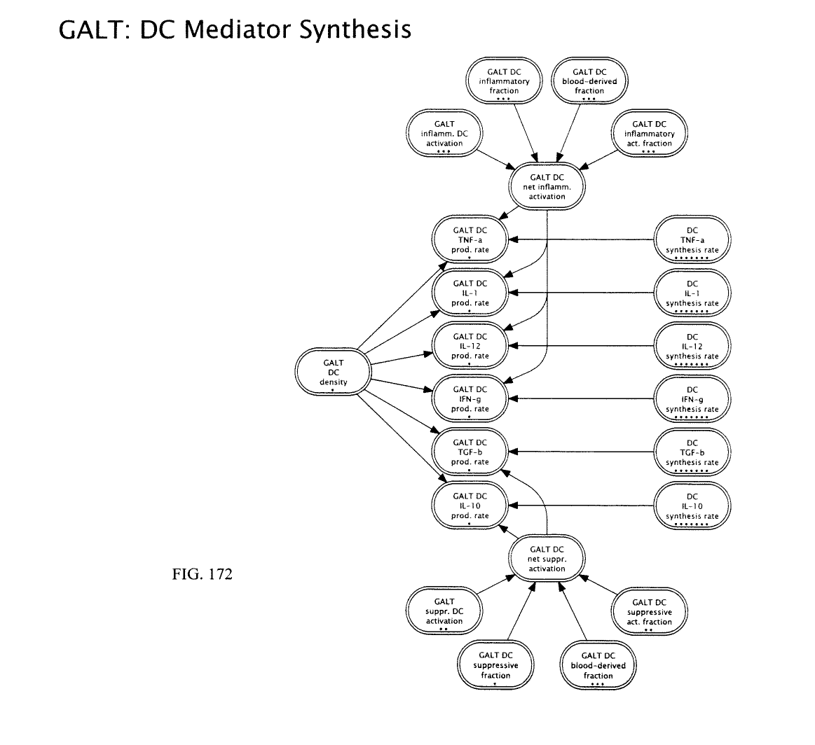

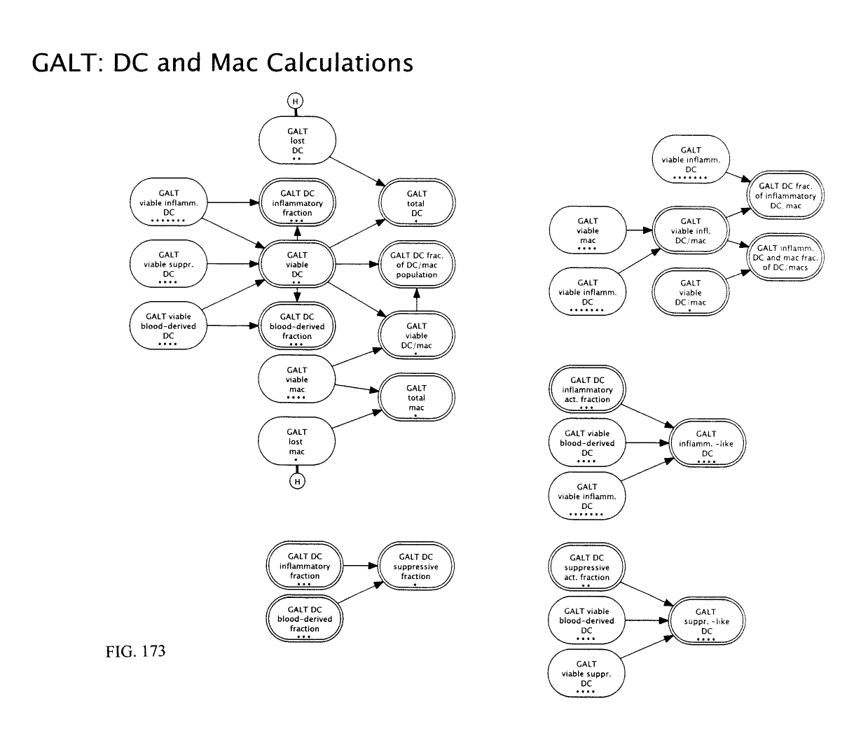

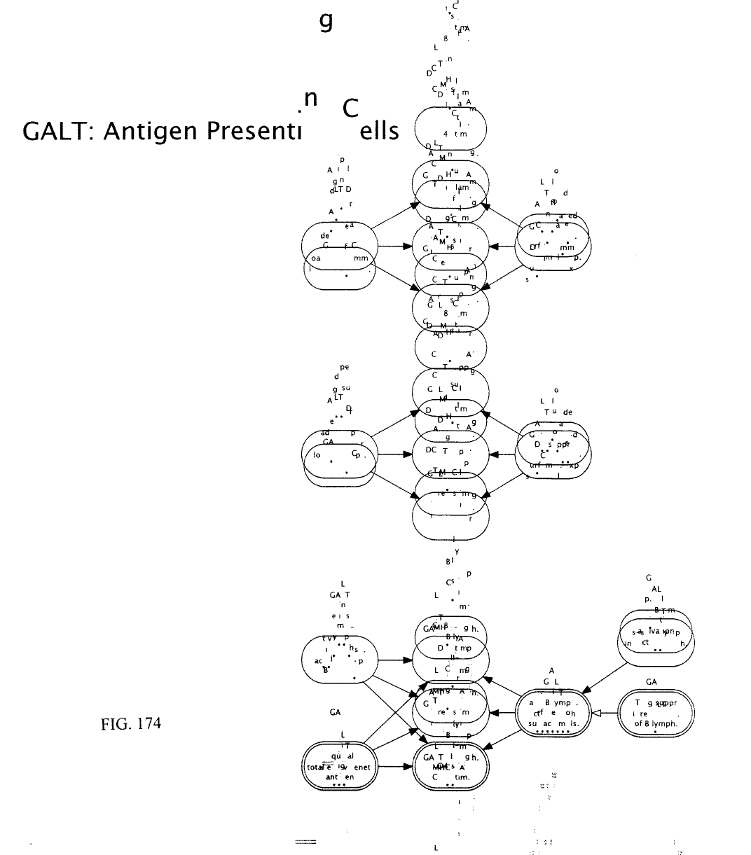

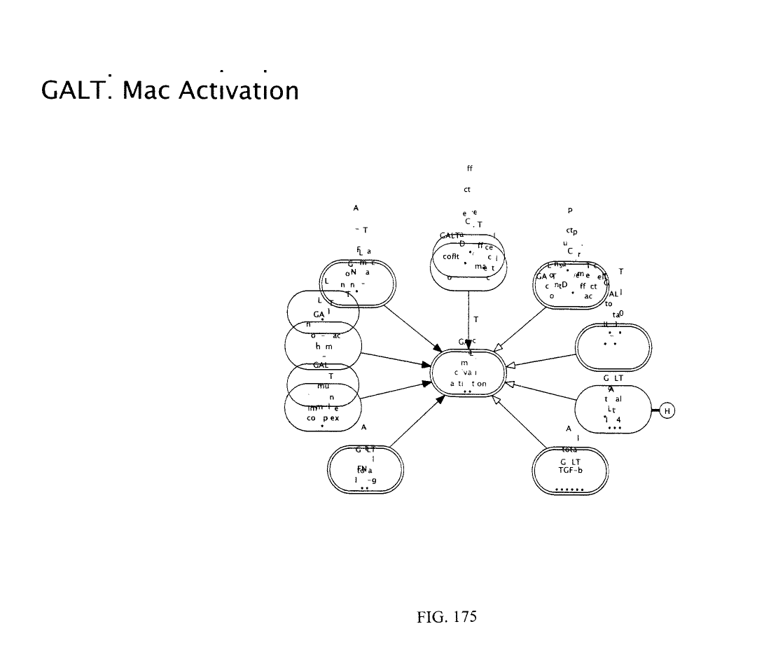

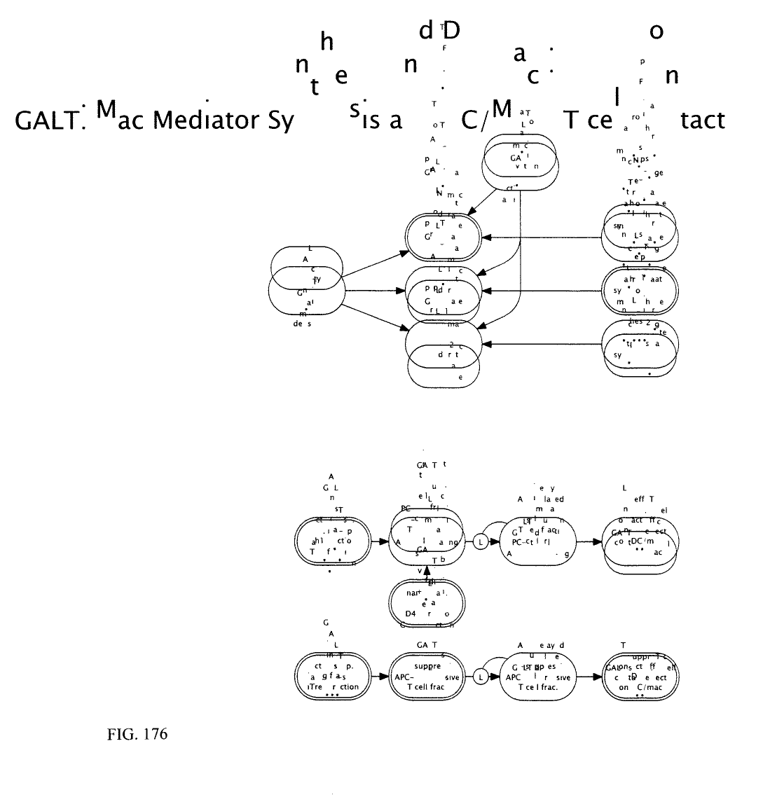

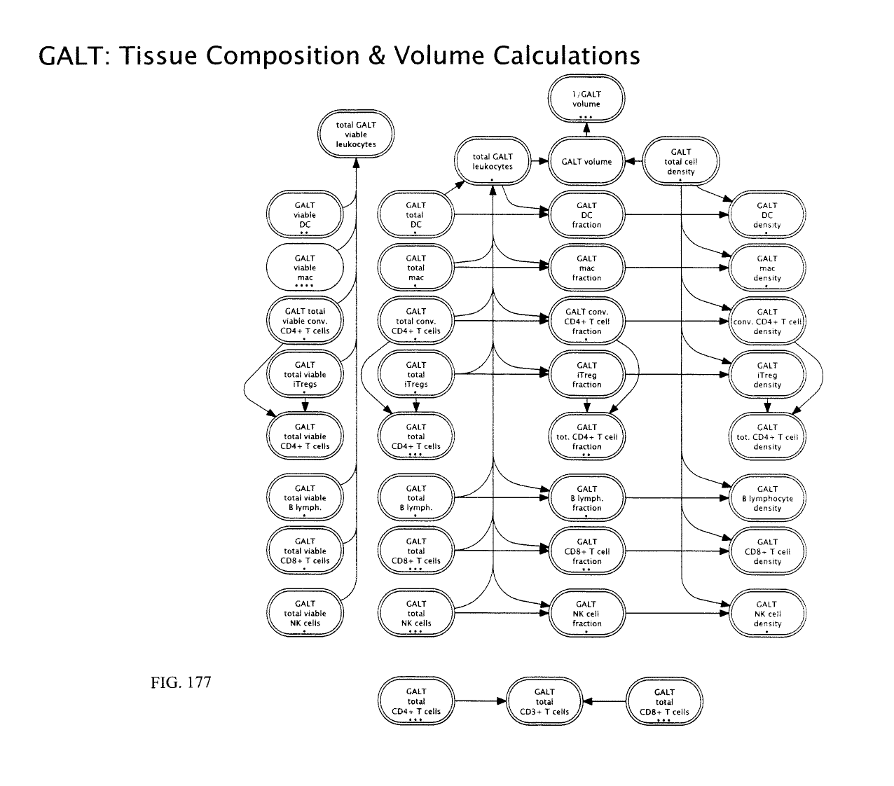

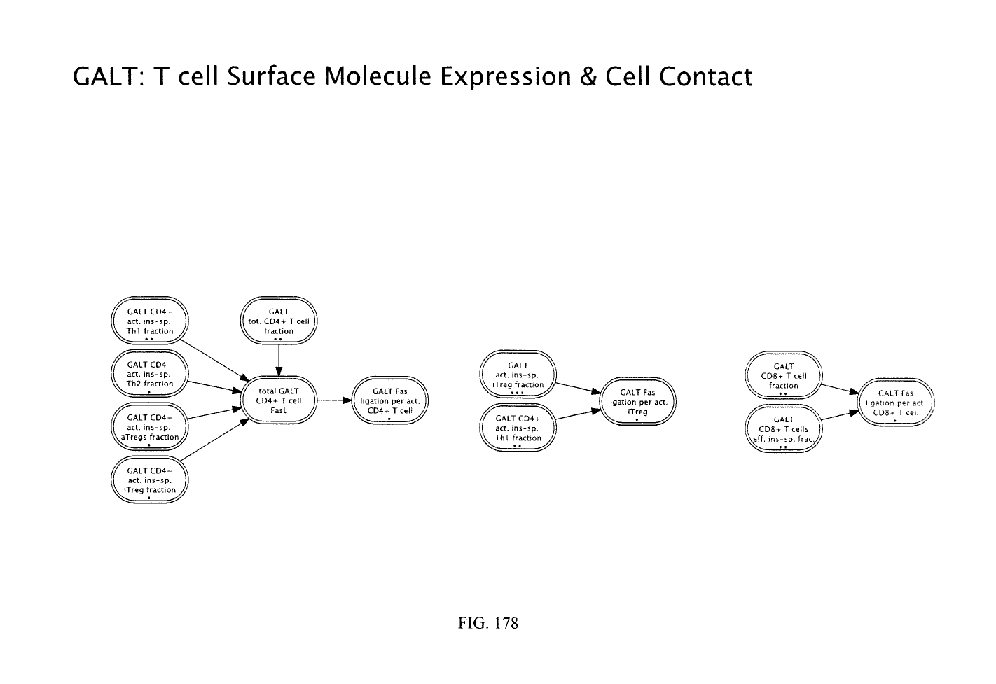

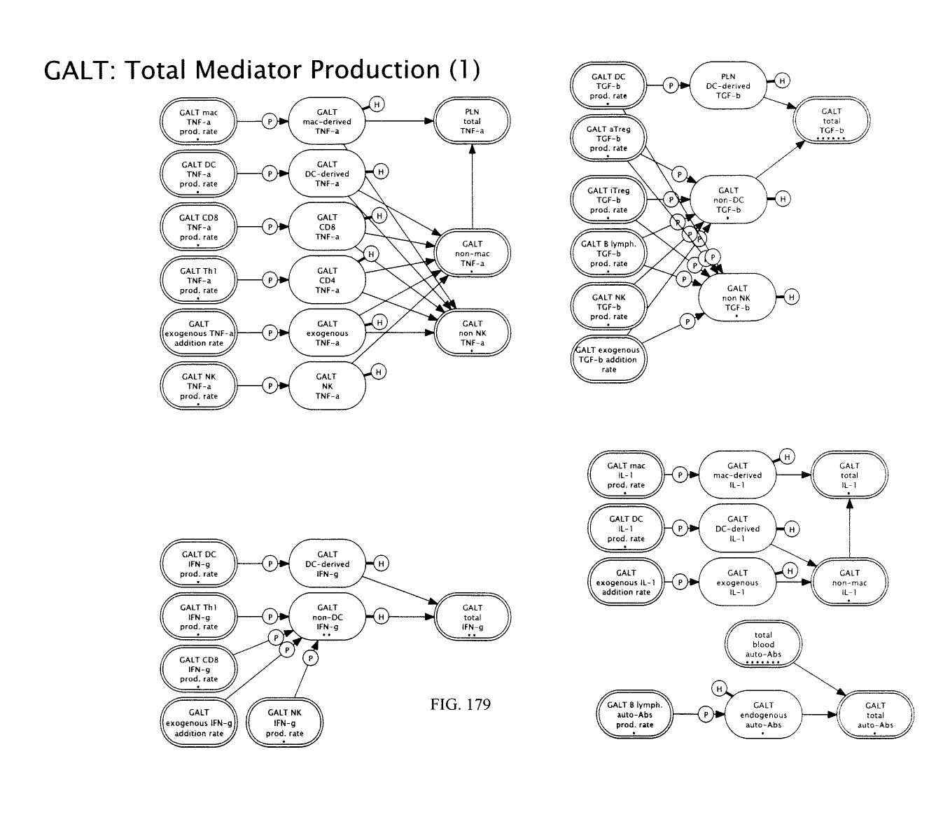

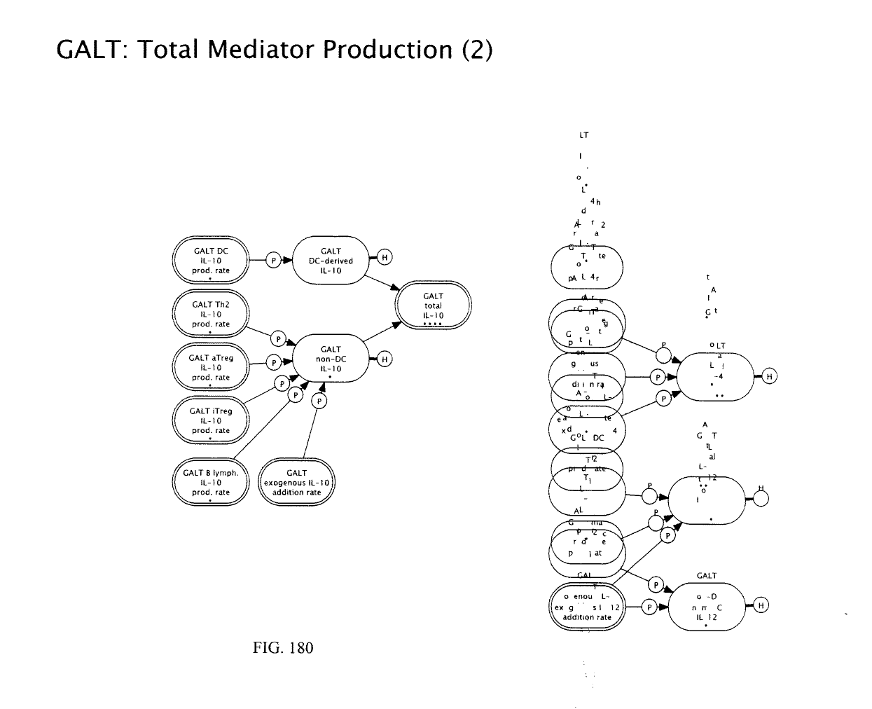

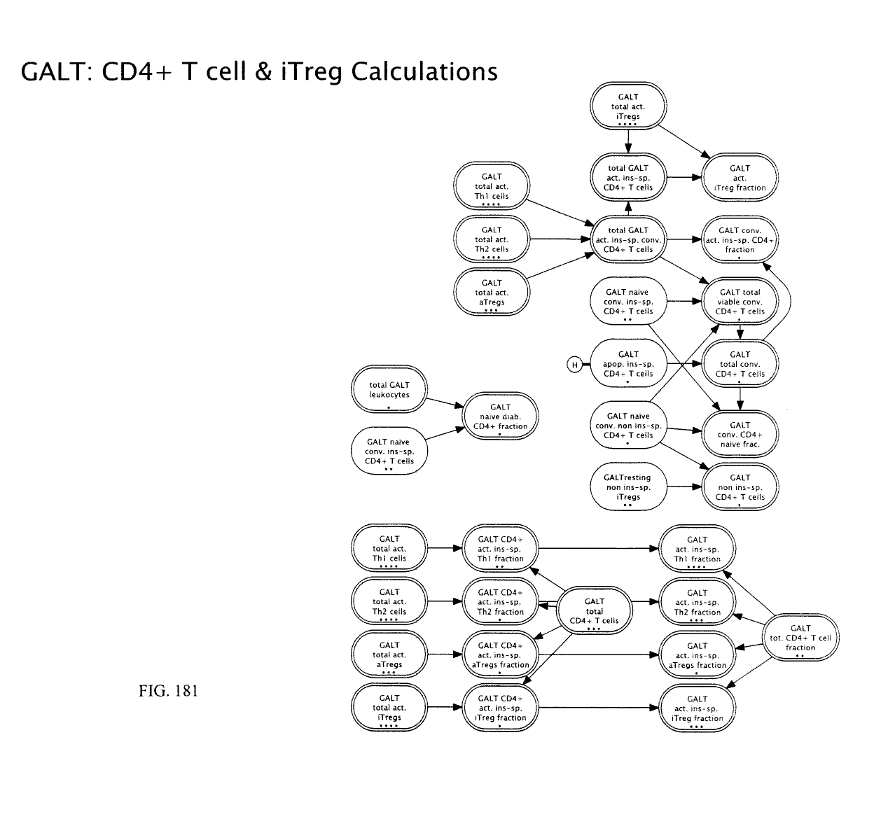

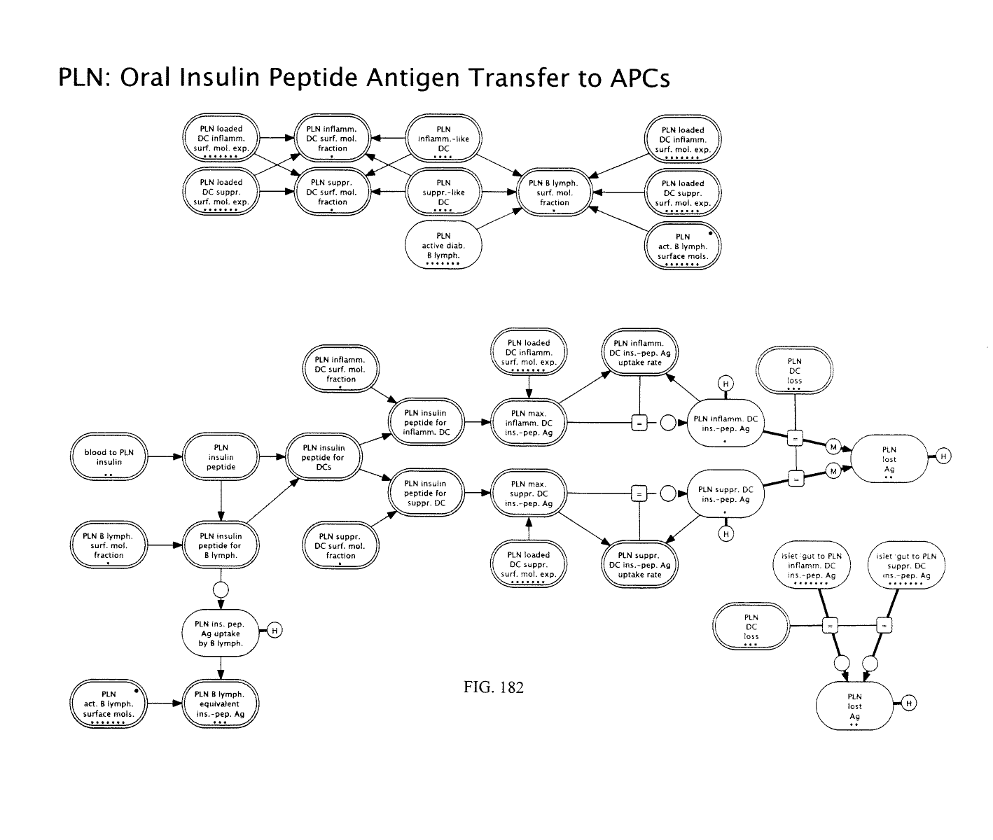

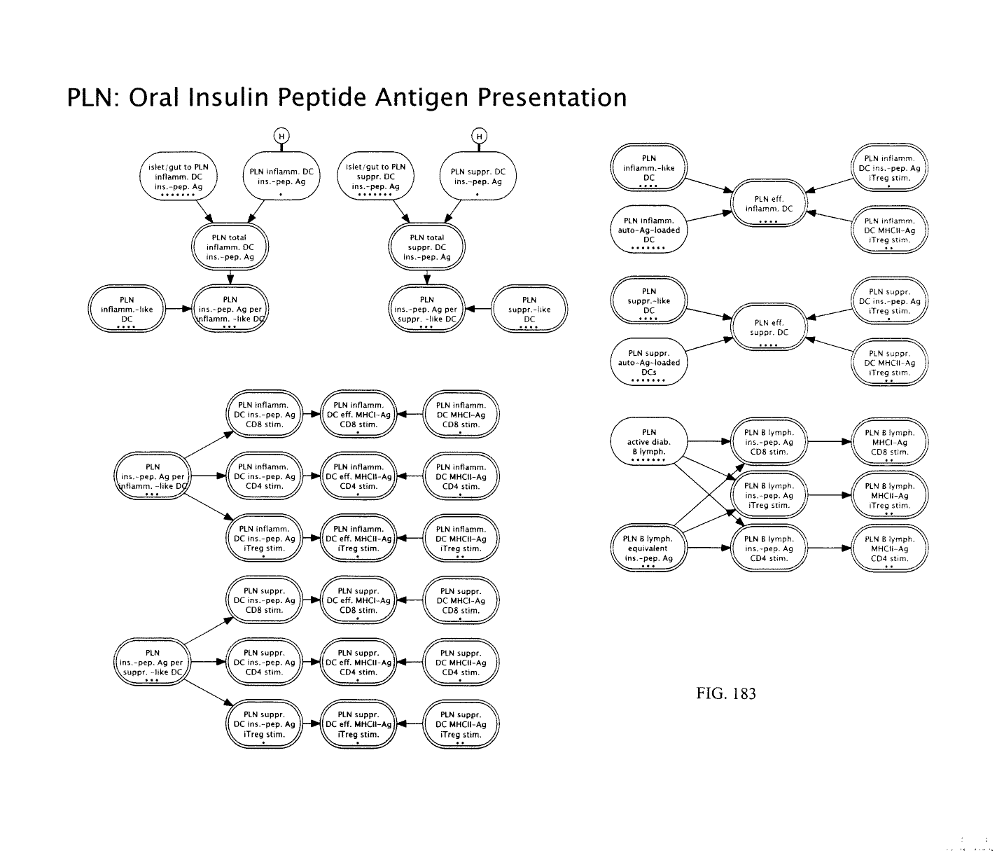

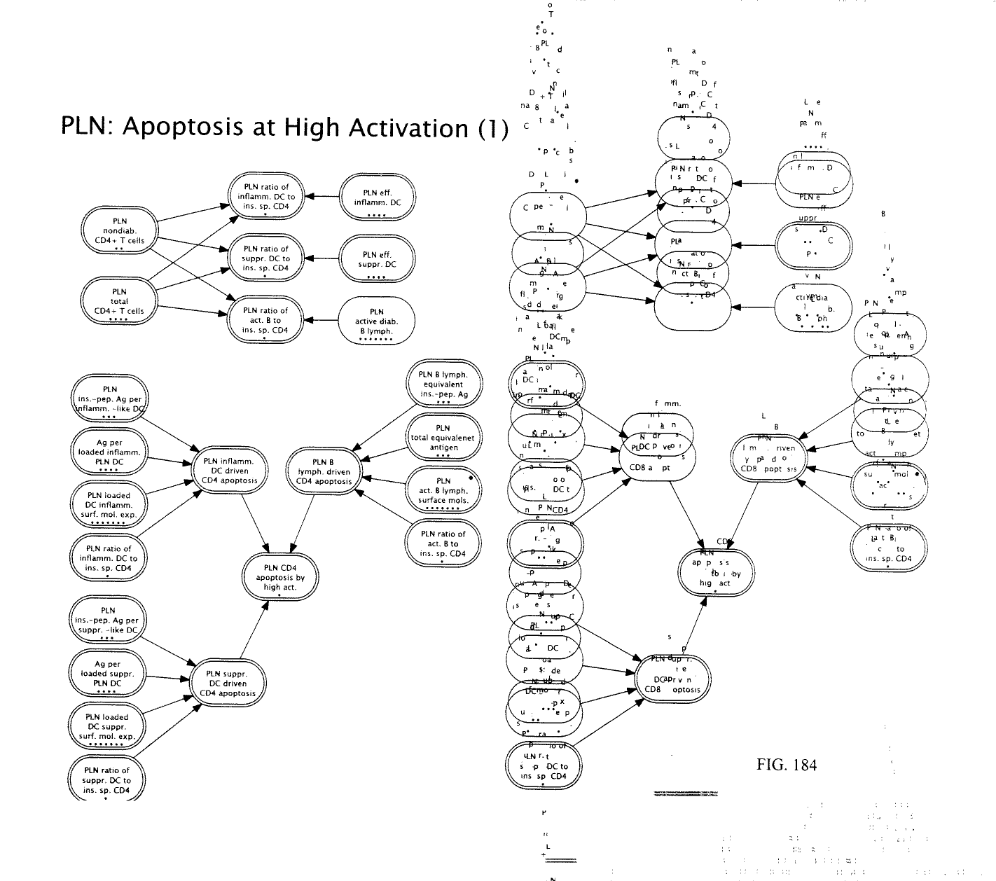

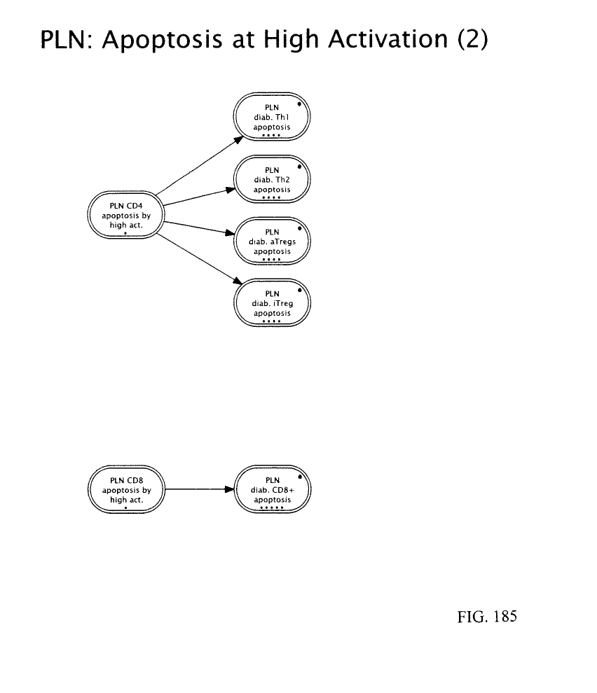

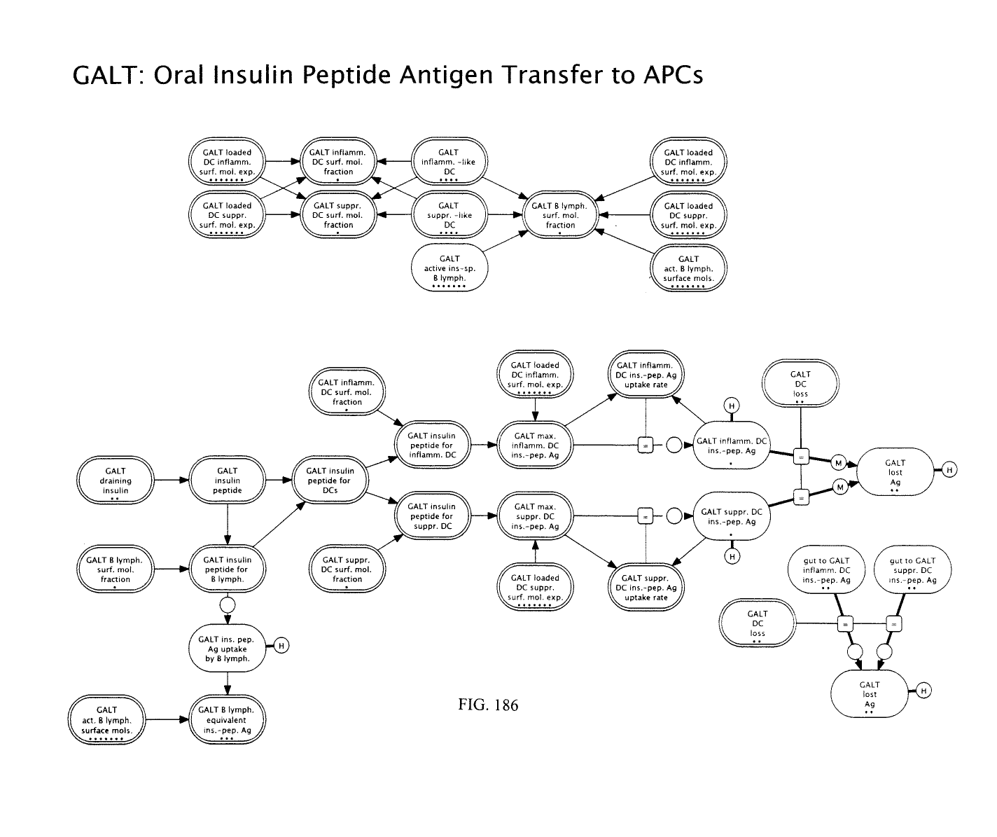

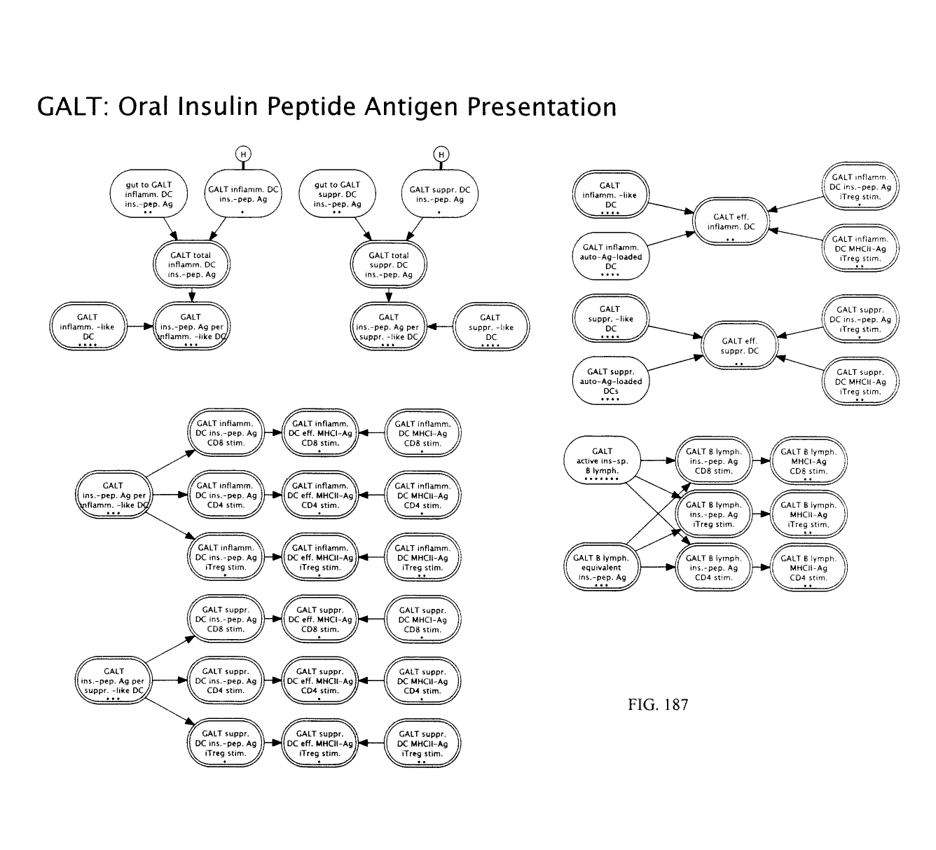

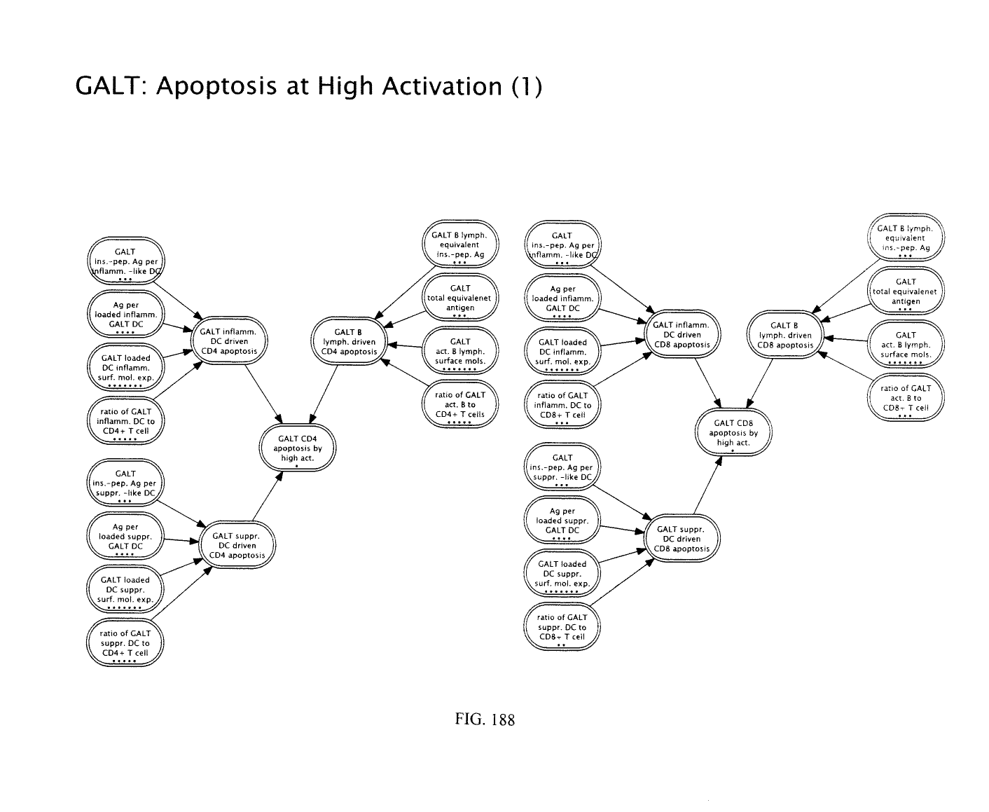

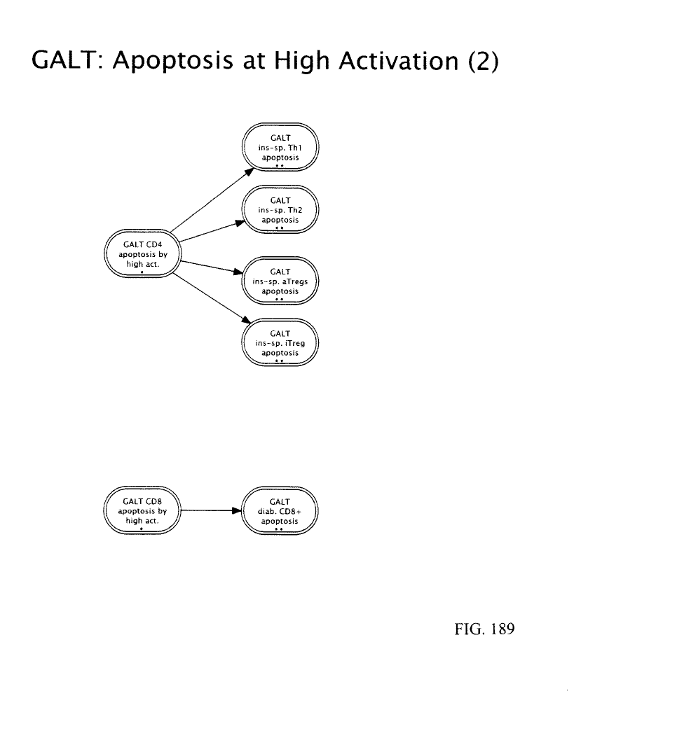

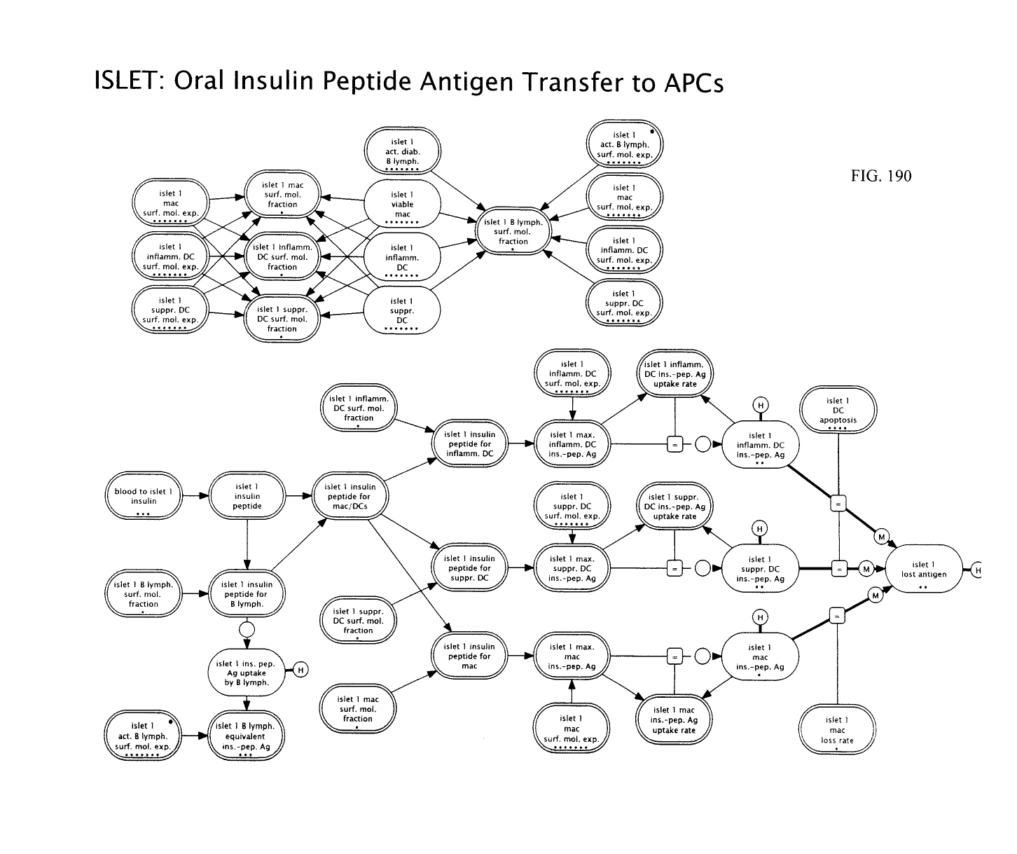

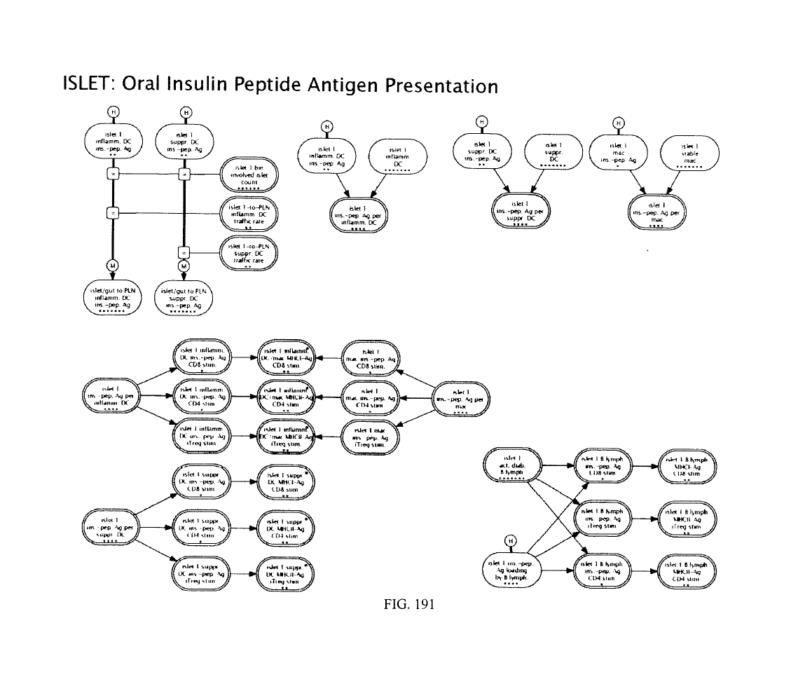

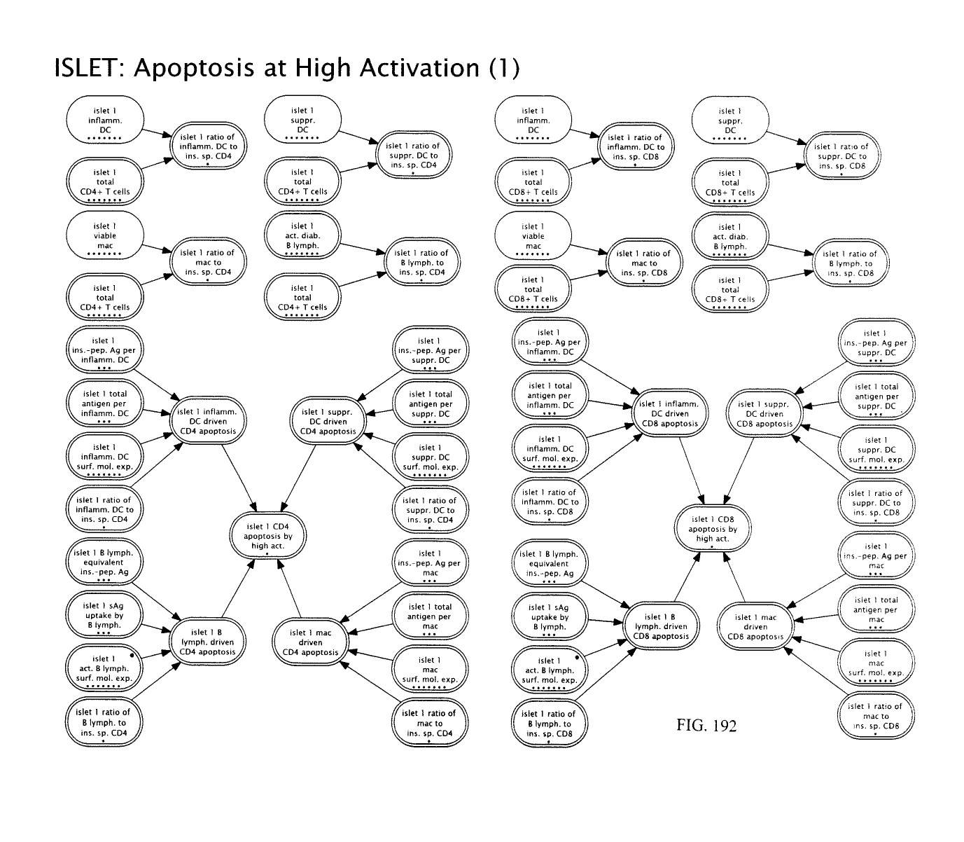

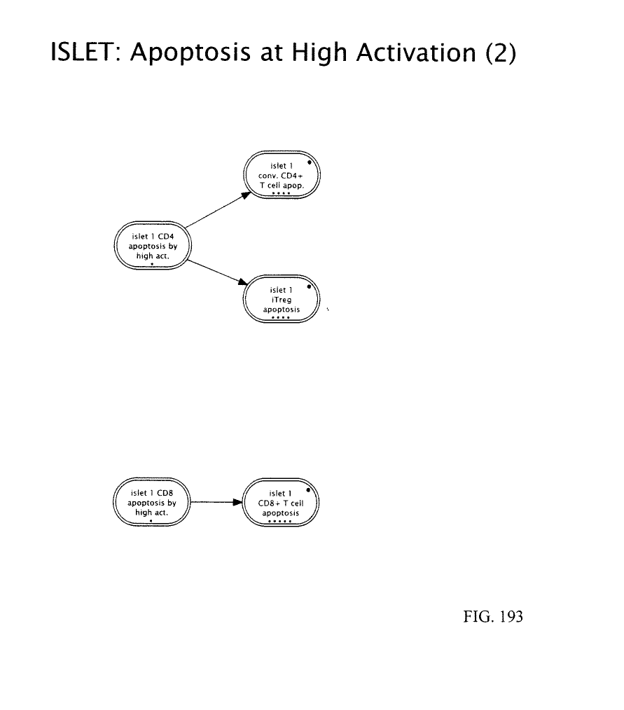

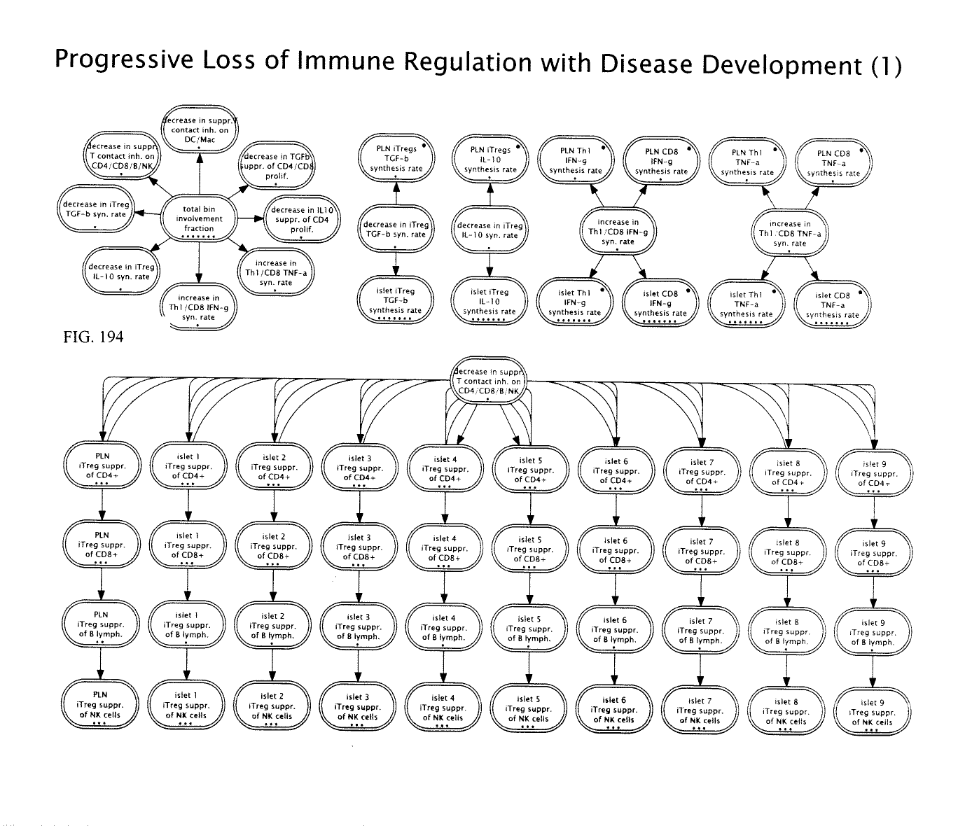

In one implementation, anti-CD8 therapy is simulated as described in FIG. 122. The antibody is administered in a manner consistent with the published experimental protocols (i.e., dose, schedule, timing), resulting in a blood anti-CD8 antibody concentration. The antibody acts to specifically deplete CD8+ T lymphocytes in the blood. In addition, some antibody is distributed to the pancreatic lymph nodes and pancreas. In these tissue environments, the antibody is similarly able to deplete CD8+ T lymphocytes. Implementations of other therapies are illustrated in FIGS. 121 and 123-142.

The computer models of the invention can be used to identify targets or pathways to which the biological system is particularly sensitive. Methods of identifying sensitive targets or pathways are described in detail in U.S. Patent Publication 2004-029639, entitled “Apparatus and Method for Identifying Therapeutic Targets Using a Computer Model,” incorporated herein by reference.

Key pathways in the virtual NOD mouse can be identified that, when modulated, cause significant changes in one or more disease outcomes. Sets of these pathways will be identified at different stages in disease progression. The results may explain the time-dependent efficacy of some therapies (i.e., identify therapeutic “windows”), suggest alternative timing/dose regimens that may increase the therapeutic efficacy of known agents and/or identify potential new therapeutic targets.

The identification of key disease-modulating pathways can be accomplished by conducting a systematic sensitivity analysis of the virtual NOD mouse or human patient. Selected biological pathways can be individually up- and down-modulated, and the consequent impact on disease outcomes determined via simulation. For example, the number of natural regulatory T lymphocytes in the neonatal virtual mouse can be decreased and increased by a factor of 10; simulations can then be performed using these values and the outcomes (e.g., rate of β cell destruction, age of diabetes onset) compared to those of the reference NOD mouse. To examine the importance of timing on such changes, the same change can be tested at different ages of the virtual mouse. In this way, sets of pathways or components whose modulation cause significant impact on disease outcomes can be identified at multiple stages of the disease (e.g., neonates, prediabetic, diabetic).

The computer models of the invention can be used to identify one or more biomarkers. A biomarker can refer to a biological characteristic that can be evaluated to infer or predict a particular result. For instance, biomarkers can be predictive of effectiveness, biological activity, safety, or side effects of a therapy. Biomarkers can be identified to select or create tests that can be used to differentiate subjects. Biomarkers that differentiate responders versus non-responders may be sufficient if the specific goal is to identify a recommended therapy for a subject. Similarly, biomarkers can be identified to diagnose or categorize subjects. Further, biomarkers can be identified to monitor the actual response of a subject to a therapy.

One aspect of the invention comprises identifying one or more biomarkers by executing a computer model of the invention absent a virtual protocol to produce a first set of results; executing the computer model based on the virtual protocol to produce a second set of results; comparing the first set of results with the second set of results; and identifying a correlation between one or more variables or parameters and a virtual measurement indicative of a pre-selected biological characteristic. Preferably the correlated variable(s) and/or parameter(s) is present in only one of the first or second set of results.

Results of two or more virtual measurements can be determined to be substantially correlated based on one or more standard statistical tests. Statistical tests that can be used to identify correlation can include, for example, linear regression analysis, nonlinear regression analysis, and rank correlation test. In accordance with a particular statistical test, a correlation coefficient can be determined, and correlation can be identified based on determining that the correlation coefficient falls within a particular range. Examples of correlation coefficients include goodness of fit statistical quantity, r², associated with linear regression analysis and Spearman Rank Correlation coefficient, rs, associated with rank correlation test.

A virtual patient in the computer model can be associated with a particular set of values for the parameters of the computer model. Thus, virtual patient A may include a first set of parameter values, and virtual patient B may include a second set of parameter values that differs in some fashion from the first set of parameter values. For instance, the second set of parameter values may include at least one parameter value differing from a corresponding parameter value included in the first set of parameter values. In a similar manner, virtual patient C may be associated with a third set of parameter values that differs in some fashion from the first and second set of parameter values.

A biological process that modulates biological response to the therapy can be associated with a knowledge gap or uncertainty, and various virtual patients of the computer model can be defined to represent different plausible hypotheses or resolutions of the knowledge gap. By way of example, biological processes associated with a pancreatic lymph node can be identified as playing a role in modulating biological response to a therapy for type 1 diabetes. While it may be understood that autoimmunity has an effect on type 1 diabetes, the relative effects of the different types of immune reactions against β cells as well as baseline concentrations of the different types of immune modulators may not be well understood. For such a scenario, various virtual patients can be defined to represent human subjects having different baseline concentrations of immune cells or immune modulators. Knowledge gaps can be identified and explored as described in co-pending Provisional U.S. Application No. 60/691,809, entitled “Hypothesis Sensitivity Analysis.”

### EXAMPLES

A. CD8+ T Cell Life Cycle

The following discussion provides an example of a process by which the modules of the above-described computer model can be developed. As discussed above, the various elements of the biological state are represented by the components shown in the Effect Diagram. These components are denoted by state and function nodes, which represent mathematical relationships that define the elements of the biological state. In general, these mathematical relationships are developed with the aid of appropriate publicly available information on the relevant biological variables and biological processes. The development of the mathematical relationships underlying the module diagram for the CD8+ T cell life cycle in the islet will be discussed here as an example.

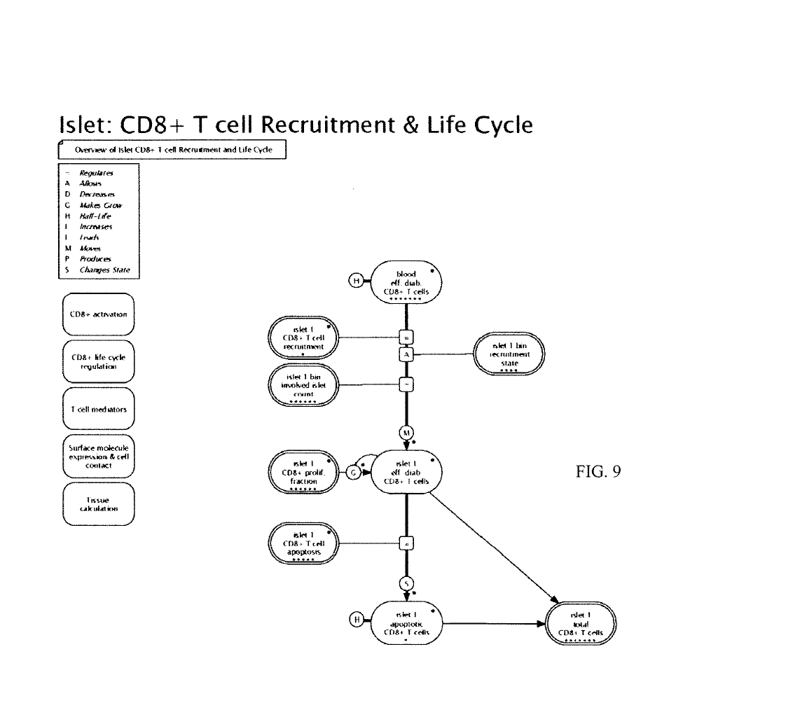

FIG. 9 shows an example of an effect diagram for the CD8+ T cell life cycle in the islet. As FIG. 9 illustrates the physiological components modeled for the life cycle of the islet T cells include: blood effector diabetes-antigen-specific CD8+ T cells; islet 1 CD8+ T cell recruitment rate; islet 1 bin involved islet count; islet 1 bin recruitment state; islet 1 CD8+ proliferative fraction; islet 1 effector diabetes-specific CD8+ T cells; islet 1 CD8+ T cell apoptosis; and islet 1 apoptotic CD8+ T cells.

In a pancreas affected by type 1 diabetes, CD8+ T cells accumulate in the pancreatic islets where they interact with other cell types via soluble mediators and direct cell-cell contact. These interactions are shaped by the number and activation state of the involved CD8+ T cells. FIG. 9 and the following description address only the calculation of the number of CD8+ T cells in the blood and an islet reference volume. The main processes of T cell turnover modeled in the islet are T cell recruitment, proliferation, and apoptosis. In the model, the numerical balance of these processes determines the number of viable islet CD8+ T cells, which modulate the net T cell activity in other parts of the model. Some of these processes and the role of T cells are reviewed in DiLorenzo and Serreze, Immunol Rev. 204:250-63 (2005). The number of apoptotic islet CD8+ T cells reflects the number of viable islet CD8+ T cells, the apoptosis rate, and clearance of apoptotic CD8+ T cells by phagocytosis or other means (as modeled by an apoptotic cell half-life).

FIG. 9 provides the graphical representation for the differential equations used to track the population of blood effector diabetes-antigen-specific, islet 1 viable and islet 1 apoptotic CD8+ T cells. The same processes govern CD8+ T cells in islets 2-9 (FIG. 3). As these differential equations depend on calculations of the recruitment, proliferation, and apoptosis rates, the latter are described first, followed by the description of the differential equations governing the population dynamics.

CD8+ T cell recruitment out of the blood and into islet 1 is governed by 3 nodes: islet 1 CD8+ T cell recruitment rate; islet 1 bin involved islet count; and islet 1 bin recruitment state.

The ability for CD8+ T cells to be recruited into islet 1 is controlled by islet 1 bin recruitment state. One aspect of type 1 diabetes disease heterogeneity is the simultaneous presence of islets at multiple stages of disease (i.e., uninfiltrated, mildly infiltrated, heavily infiltrated, destroyed). This heterogeneity is reproduced by representing the islets as distinct distributed sites, which become involved in the autoimmune destruction at different stages of the disease process. The islet 1 bin recruitment state specifies whether islet 1 may be infiltrated by autoimmune lymphocytes.

CD8+ T cell recruitment into islet 1 is further controlled by islet 1 bin involved islet count. Each distinct distributed sites (i.e., islet bin) represents a fraction of the total islets in the pancreas. Within each site, some of the islets are involved (infiltrated) while others are still free of lymphocytic infiltrates. The islet 1 bin involved islet count determines the number of involved islets within islet bin 1 as follows:
Islet 1 bin involved islet count=bin 1 involvement fraction*gl total islets

The bin 1 involvement fraction reflects the fraction of pancreatic islets that are involved and that are represented in islet bin 1. The parameter “gl total islets” specifies the total number of islets, 2000, in the pancreas as reported by Bock et al., Diabetes. 54(1):133-7 (2005).

The CD8+ T cell recruitment rate constant, which specifies the net influx rate of CD8+ T cells into the islet reference volume, is equal to the sum of two components: 1) a constitutive component independent of adhesion molecule upregulation by endothelial cells and 2) a regulated component that is dependent on the upregulation of adhesion molecules on endothelial cells in the islet, as shown below:
T cell recruitment rate constant=max recruitment rate constant*(1−constitutive fraction)*effect of islet EC adhesion molecules+max recruitment rate constant*constitutive fraction.

The constitutive fraction accounts for recruitment of activated cells at basal adhesion molecule expression, as has been shown by Savinov et al., J Exp Med 197:643-656 (2003). The regulated component accounts for the increased recruitment, over basal levels, as driven by increased adhesion molecule expression. The parameter “max recruitment rate constant” represents the maximum recruitment of circulating diabetes-antigen-specific CD8+ T cells recruited per hour. The “max recruitment rate constant” parameter subsumes other effects determining the maximum rate of CD8+ T cell recruitment including antigen presentation, T cell surface molecules, and chemotactic factors. This parameter has been adjusted to achieve the reported number of CD8+ T cells in the islet while maintaining a balance between the population's dependence of cell recruitment vs. cell proliferation.

The size of the population of blood effector diabetes-antigen-specific CD8+ T cells (T_{b}) is a major determinant of the islet CD8+ T cell population. The population of T_{b} is determined by using the values obtained from the evaluation of the T cell recruitment rate in each islet bin 1-9 (r_{bi}), the half life of effector CD8+ T cells in the blood (t_{b}½), and the rate of effector diabetes-antigen-specific CD8+ T cells entering the blood from the pancreatic lymph nodes (g). When the bin recruitment state for each islet bin is greater than 0, the rate of exit from T_{b} into that islet bin (r_{bi}) is proportional to the population of blood effector CD8+ T cells, that islet bin's involved islet count, and the recruitment rate in that islet bin. Otherwise, when the bin recruitment state for an islet bin is not greater than 0, there is no loss of T cells from the blood into that islet bin. The half life of effector CD8+ T cells in the blood was estimated based on the rate of apoptosis due to spontaneous death of activated T cells (e.g., 46 hour spontaneous half life for T cells, Kelly et al., J Immunol. 168 (2):597-603 (2002)). The rate of T_{b} exiting the pancreatic lymph node is calculated elsewhere in the model. Thus, the dynamics of the blood effector CD8+ T cell population are represented by the following equation:

$$\frac{ⅆ {T}_{b}}{ⅆ t} = - ln ( 2 ) / {t}_{b 1 / 2} * {T}_{b} - {\sum }_{i} {r}_{bi} + g$$

The proliferation of CD8+ T cells in the islet is determined from the fraction of cells entering mitosis at a specific moment (fₚ), as determined elsewhere in the model and represented by the node “islet 1 CD8+ proliferative fraction”. The proliferative fraction is driven by activation of CD8+ T cells by antigen presenting cells (APCs) and further regulated by both soluble mediators and cell-cell contact. The T cell proliferation rate constant (p) is set to ln(2)/8=0.0866 (1/hours), corresponding to a cycle time of 8 hours as reported by Swain, Curr Opin Immunol. 11 (2):180-185 (1999).

The apoptosis of CD8+ T cells is determined from the fraction of cells entering the apoptotic cascade at a given time (fₐ), as determined elsewhere in the model and represented by the node “islet 1 CD8+ T cell apoptosis”. The apoptotic fraction accounts for both death by neglect and activation induced cell death (AICD) and is regulated by soluble mediators. The T cell apoptosis rate constant (a) is set to ln(0.5)/5=0.14 (1/hours), corresponding to the observation that 52% of activated murine T cells were apoptotic after 5 hours (Zhang et al., J Exp Med 186:1677-1687 (1997)). These data subsumed both death by neglect and AICD.

The populations of viable CD8+ T cells (Tᵥ) and apoptotic CD8+ T cells (Tₐ) are determined using the values obtained from the evaluation of T cell recruitment rate (r), T cell proliferation rate constant (p), T cells proliferative fraction (fₚ), T cell apoptosis rate constant (a), and T cell apoptotic fraction (fₐ). When the islet 1 bin recruitment state is greater than 0, the recruitment rate (r) of cells into the viable cell population from the blood is proportional to the population of blood effector CD8+ T cells (T_{b}) and the T cell recruitment rate constant, otherwise there is no T cell recruitment into this islet. The viable CD8+ T cells proliferate at a rate proportional to the population of viable cells (Tᵥ), the fraction of viable CD8+ T cells proliferating (fₚ), and the proliferation rate constant (p). Thus, the dynamics of the viable CD8+ T cell population are represented by the following equation:

$$\frac{ⅆ {T}_{v}}{ⅆ t} = p * {f}_{p} * {T}_{v} + r - a * {f}_{a} * {T}_{v}$$

Subsequently, the viable cells enter apoptosis at a rate proportional to the population of viable cells (Tₐ), the T cell apoptotic fraction (fₐ) and the apoptosis rate constant (a), and exit the islet via phagocytosis/degradation characterized by the half-life (tₐ½). Thus, the population of apoptotic T cells is represented by the differential equation:

$$\frac{ⅆ {T}_{a}}{ⅆ t} = a * {f}_{a} * T - ln ( 2 ) / {t}_{a 1 / 2} * {T}_{a}$$

Together, these equations specify the population dynamics of blood CD8+ T cells and viable and apoptotic CD8+ T cells in the islet.

The values of the parameters used in the various functions within this module were determined so as to match experimental data and the guidelines described below. In one embodiment, these guidelines are manifested as the following constraints: (1) populations (Tᵥ, Tₐ) vary over time in the untreated reference mouse (reference mouse type definition); (2) a significant fraction of activated T cells can be recruited to a tissue, given basal levels of adhesion molecule expression (Savinov et al., 2003); (3) there are roughly 2000 islets in the NOD mouse (Bock et al., 2005), whose mass rather than number appears to change with growth of the animal; (4) at maximum leukocyte infiltration of the pancreas, the fraction of the T cell population that is apoptotic (Tₐ/(Tᵥ+Tₐ)) is less than 2%, based on calculations derived from data on total apoptotic events among islet leukocytes (Augstein et al., Diabetologia 41:1381-1388 (1998)); (5) the doubling time for viable CD8+ T cells is approximately 8 hours (Swain 1999), which yields a proliferative rate constant ln(2)/8=0.0866 (1/hours); and (6) the fraction of activated T cells that are apoptotic within 5 hours is 0.52, yielding a rate constant for apoptosis of activated T cells=lin(0.5)/5=0.14 (Zhang et al., . J Exp Med 186:1677-1687 (1997)), and apoptotic cells are phagocytosed within 4-8 hours of entry into the apoptotic cascade.

In keeping with these constraints, in one embodiment, the parameters are set as follows: “constitutive fraction” for recruitment=0.5; “gl total islets”=2000; “proliferative rate constant”=0.0866 (1/hours); “rate of apoptosis”=0.14 (1/hours); half-life for disappearance of apoptotic cells (tₐ½)=4 hours. These parameter values are not specifically reported in the public literature but have been determined to comply with constraints such as the ones above which in turn emerge from the public literature or clinical and laboratory experience. These parameter values do not necessarily have to uniquely satisfy the constraints, and can be changed in alternate embodiments with the same or different constraints, such as one describing a mouse with different proliferative or apoptotic fractions of T cells.

As this example of the life cycle of islet 1 CD8+ T cell model component generally illustrates, the components of the Effect Diagram, denoted by state and function nodes, represent mathematical relationships that define the elements of the biological state being modeled. These mathematical relationships can be developed with the aid of appropriate information on the relevant biological variables and biological processes. In other words, the Effect Diagram indicates the type of mathematical relationships that are modeled within a given model component. The information can then be put into a form that matches the structure of the Effect Diagram. In this way, the structure of the model was developed and other components of this model were developed in a similar fashion (FIGS. 10-195).

B. Simulation of Type 1 Diabetes Therapies

Numerous interventions have been tested in the NOD mouse. A major purpose of implementing selected interventions is to provide constraints for the virtual mouse, where constraints are defined as additional behavioral criteria that the virtual mouse should reproduce in a manner substantially consistent with published data. To serve this purpose, the interventions should be relevant to the modeled biology of the virtual mouse; act on different areas of the modeled biology to impose diverse constraints; and have well characterized mechanism(s) of action that can be directly implemented on the modeled biology. The impact of a variety of select, well characterized interventions on islet inflammation and glucose control was captured by modulating the biological pathways represented in the NOD mouse type 1 diabetes model and treating the virtual mouse. Based on these pathway effects, the simulation produces a resulting impact on glucose control and disease progression. Other systemic effects (e.g., kidney damage) were not represented.
1. Anti-CD3 Monoclonal Antibody (145.2C11)

Recent clinical studies on anti-CD3 have yielded promising results. In the NOD mouse, the effect of this therapy appears to depend on timing and dose. The role of anti-CD3 in T lymphocyte depletion, T lymphocyte activation, and regulatory cell generation/activity were included. The following effects of intervention were reproduced in the model: (1) disease protection when administered at birth; (2) no effect when administered during the prediabetic phase; and (3) restoration of euglycemia when administered to diabetic NOD mice.

The direct effects of a hamster anti-mouse antibody to CD3 (145-2C11) that were implemented included the following: (1) stimulates T cell activation, proliferation and apoptosis; (2) differential effects on conventional T cells versus innate regulatory T cells; (3) differential effect on naïve versus activated T cells; and (4) inhibition of endothelial cell adhesion molecule expression.

Several publications report treating NOD mice at different stages of disease and with different protocols. Three such publications that span the range of variation observed in the published literature are shown in Table 1.

TABLE 1
Representative Anti-CD3 protocols used to evaluate the average virtual
NOD mouse

| | Hayward 1992 | Chatenoud 1997 | Chatenoud 1994 |
|---|---|---|---|
| | (Neonatal) | (Intermediate) | (Diabetic) |
| Timing | 24 hrs | 4, 8, or 12 wks | Diabetic |
| of Rx | | | |
| Regimen | 200 μg ip | 5 μg iv/5 days | 5 μg iv/5 days |
| Incidence | 13% vs 75% | No effect at 30 wks | 64-80% remission |
| | control at 52 wks | | 12 wk follow-up |
| Insulitis | Reduced insulitis | No effect | Reduced insulitis |
| Expected | No diabetes | 4, 8 wks: no delay | Remission at 12 wk |
| virtual | at 52 wks | 12 wks: 4 wk delay | follow-up |
| mouse | | | |
| outcome | | | |

Simulation results from the neonatal treatment protocol showed sustained protection from the development of diabetes and no initial priming of CD4+ T lymphocytes, which are consistent with the published report. The simulation results of the intermediate dose initiation protocol were consistent with the published reports in that neither initiation of dosing at 4, 8 or 12 weeks protected from development of diabetes, although there was an increasing delay with later treatment. The simulation results from treating diabetic animals resulted in sustained remission, as reported in the published data. However, remission was dependent on rapid β cell re growth (in excess of normal homeostatic levels) following clearance of the inflammation.
2. Anti-CD8

The importance of CD8+ T lymphocytes to type 1 diabetes pathogenesis has been demonstrated through the effects of depleting antibodies and genetic manipulation. The role of anti-CD8 in eliminating CD8+ T lymphocytes was incorporated in the model. The following effects of intervention were reproduced in the model: (1) disease protection when administered to pre diabetic animals; (2) no effect when administered to diabetic animals; and (3) significant depletion of CD8+ T lymphocytes. The direct effects of antibody to CD8 that were implemented include depletion of CD8+ T lymphocytes.

Several publications report treating NOD mice at different stages of disease and with different protocols. Three such publications that span the range of variation observed in the published literature are shown in Table 2

TABLE 2
Representative Anti-CD8 protocols used to evaluate the average virtual NOD mouse

| | Chowdhury 2002 | Sempe 1993 | Mottram 2002 |
|---|---|---|---|
| | (Neonatal) | (Intermediate) | (Diabetic) |
| Timing of Rx | 2-4 wks | 12-32 wks | Diabetic |
| Regimen | 500 μg/2x week | 100 μg/weekly | 50 μg/daily for 5 days |
| Incidence | 87% vs. 0% treated at | 48% vs. 19% treated at | 33% remission at 7 wks |
| | 40 wks | 32 wks | follow-up |
| Insulitis | No insulitis | Insulitis | Not reported |
| Implementation | CD8+ T cell apoptosis | | |
| Expected virtual | No diabetes at 40 wks | No diabetes at 32 wks | No remission at 7 wks |
| mouse outcome | | | follow-up |

Simulation results from the neonatal treatment protocol showed sustained protection from the development of diabetes and no initial priming of CD8+ T lymphocytes, which are consistent with the published report. Simulation results from the intermediate dose initiation protocol were consistent with the published reports in that the average virtual NOD mouse was free of diabetes at 32 weeks. Finally, as expected, the diabetic treatment protocol did not lead to remission.
3. Lip-Cl₂MDP—liposomal dichloromethylene diphosphonate

The importance of macrophages (and dendritic cells) to type 1 diabetes pathogenesis has been demonstrated through therapeutics that target phagocytic cells. The effect of liposomal dichloromethylene diphosphonate (lip-Cl₂MDP) on apoptosis of phagocytic cells were included. The following effects of intervention were reproduced in the model: (1) disease protection when administered early; and (2) disease protection when administered late. The direct effects of lip-Cl₂MDP that were implemented included the following: (1) depletion of circulating monocytes; and (2) apoptosis of dendritic cells/macrophages in pancreatic lymph nodes and islets. Two published protocols were implemented, Table 3.

TABLE 3
LipCl₂MDP protocols used to evaluate the average virtual NOD mouse

| | June 1999 | Nikolic 2005 |
|---|---|---|
| | (early, prolonged) | (intermediate) |
| Timing of Rx | 3-20 wks | 8-8.6 wks |
| Regimen | 200 μl (1 mg) weekly | 200 μl (2 mg); |
| | | 2 injections separated by |
| | | 2 d |
| Incidence | 0% vs. 80% control at | 27% vs. 87.5% control at |
| | 35 wks | 35 wks |
| Insulitis | Reduced severity in | Normal to severe insulitis |
| | insulitis | |
| Expected virtual | No diabetes at 35 wks | No diabetes at 35 wks |
| mouse outcome | | |

Simulation results from the early protocol showed sustained protection from the development of diabetes, consistent with the published report. Simulation results from the intermediate protocol also showed protection from diabetes as expected.
4. Exogenous IL-10

IL-10, delivered in various ways, has been shown to protect against disease progression and may be of interest in future clinical trials. The effect of IL-10 on cellular activation, differentiation, and apoptosis were included. The following effects of intervention were reproduced in the model: (1) disease protection given recombinant human IL 10; and (2) reduced severity of insulitis.

Exogenous IL-10 was implemented, where the exogenous IL-10 combines with the pool of endogenous IL-10; thereby increasing the IL-10 concentration and effects on disease. The following protocol was implemented, Table 4.

TABLE 4
IL-10 protocol used to evaluate the average virtual NOD mouse

| | Pennline 1994 |
|---|---|
| Timing of Rx | 9-24 wks |
| Regimen | 1 μg recombinant human IL-10 daily, 15 wks |
| Incidence | 25% vs. 85% control at 28 wks |
| Insulitis | Reduced severity of insulitis |
| Implementation | Increase IL-10 conc. resulting in: |
| | Inhibition of CD4+ T cell |
| | proliferation |
| | Inhibition of DC/mac activation |
| | Potentiation of CD8+ T cell |
| | proliferation |
| | Potentiation of CD4+ aTreg |
| | differentiation |
| Expected virtual | No diabetes at 28 wks |
| mouse outcome | |

Simulation results of the protocol showed sustained protection from the development of diabetes, consistent with the published report. Further analysis of the simulation results indicated that IL-10 therapy reduces the priming of T cells in the pancreatic lymph nodes and this results in a reduction in inflammation of the pancreas.
5. Anti-B7.1 and Anti-B7.2

In initial testing, anti-B7.1 (16-10A1, hamster anti-mouse) and anti-B7.2 (GL1, rat anti-mouse) antibody treatment produced the surprising result of disease exacerbation rather than protection. The following effects of intervention were reproduced in the model: (1) exacerbation of disease and (2) reduction in innate regulatory T cell population.

The direct effect of these agents that were implemented included the following: (1) inhibition of innate regulatory T cell costimulation; (2) partial inhibition of conventional T cell costimulation; and (3) inhibition of contact-suppression of islet conventional T cells. The following protocol was implemented, Table 5.

TABLE 5
Anti-B7.1 plus anti-B7.2 antibody intervention protocol used to evaluate
the average virtual NOD mouse

| | Lenschow 1995 |
|---|---|
| Timing of Rx | 2-8 weeks |
| Regimen | 2-4 weeks: 50 μg |
| | each Ab every other |
| | day |
| | 6-8 weeks: 50 μg |
| | each Ab, 1x week |
| Incidence | 94% at 16 weeks vs. |
| | 70% control at 25 |
| | weeks |
| Expected virtual | 3-10 week |
| mouse outcome | exacerbation |

Simulation results of the protocol showed a 6.5 week acceleration in onset of frank diabetes, consistent with the published report. In response to the loss of innate regulatory T cells with this intervention, the simulation results suggested a significant over-expansion of pathogenic T cells in the islets.
6. Oral Insulin

Insulin is one of the autoantigens that has been identified in type 1 diabetes. Antigen specific therapies are of great interest as a treatment modality that might be expected to specifically down-modulate autoimmunity without affecting normal immune responses. Oral administration of insulin has been shown to protect against diabetes through the induction of oral tolerance. The following effect of intervention was reproduced in the model: disease protection following administration of porcine oral insulin to pre-diabetic NOD mice

Oral insulin administration was implemented as: (1) transferring across the gut wall, resulting in presentation of exogenous insulin antigen to insulin specific T cells in the local gut associated lymphoid tissues, requiring composition of a gut and gut associated lymphoid tissue; and (2) transferring across the gut wall, resulting in distribution through the blood and presentation in non local tissues including pancreatic lymph nodes and pancreas. Mechanisms of oral insulin induced tolerance, including suppression (via local induction of regulatory cells) and T cell deletion, were represented.

The following protocols including dose dependent efficacy were implemented, Table 6.

TABLE 6
Oral insulin protocol used to evaluate the average virtual NOD mouse

| | Zhang 1991 | Zhang 1991 | Homann 1999 |
|---|---|---|---|
| | (1 mg) | (0.1 mg) | (1 mg) |
| Timing of Rx | 5-52 weeks | 5-52 weeks | 5-45 weeks |
| Regimen | 5-10 weeks: 1 mg | 5-10 weeks: 0.1 mg | 5-10 weeks: 1 mg |
| | 2x/week | 2x/week | 2x/week |
| | 11-52 weeks: 1 mg | 11-52 weeks: 0.1 mg | 11-45 weeks: 1 mg |
| | 1x/week | 1x/week | 1x/week |
| Incidence | 26% treated vs. | 44% treated vs. | 26% treated vs. |
| | 49% control at 52 | 49% control at 52 | 63% control at 45 |
| | weeks | weeks | weeks |
| Expected virtual | No diabetes at 52 weeks | Diabetes similar to | No diabetes at 45 |
| mouse outcome | | control | weeks |

Simulation results showed disease protection while administering 1 mg, but not 0.1 mg oral insulin, consistent with the published data.
7. Exogenous TGF β

The direct effects of this agent that were implemented included the following: (1) inhibition of CD4+/CD8+ proliferation; (2) inhibition of CD4+/CD8+/iTreg apoptosis; (3) potentiation of iTreg proliferation; (4) inhibition of dendritic cell/macrophage and NK cell activation; (5) inhibition of Th1/Th2 T cell differentiation; (6) potentiation of adaptive regulatory T cell T cell differentiation; and (7) inhibition of endothelial cell adhesion molecules. The following protocol was implemented, Table 7.

TABLE 7
Exogenous TGF-β protocol used to
evaluate the average virtual NOD mouse

| | Piccirillo 1998 |
|---|---|
| Timing of treatment | 9-32 wks |
| Regimen | 200 μg of pCMV-TGF-β1 every 2 wks until |
| | 32 wks of age Implemented serum conc. reported |
| | Implemented TGF-β tissue partition |
| | (Rachmawati 2004) |
| Incidence | 42% vs. 100% control at 32 wks |
| Insulitis | Reduced severity of insulitis |
| Other observations | Reduced mRNA levels of islet IL-12 & IFN-γ |
| Expected virtual | No diabetes at 32 wks |
| mouse outcome | |

Simulation results of the protocol showed that TGF-β, when administered from 9-32 weeks protects the virtual mice from developing diabetes and reduces insulitis, which are consistent with the published report and several other published treatment protocols.
8. Exendin-4

The direct effects of this agent that were implemented included the following: (1) glucose-dependent enhancement of insulin secretion; (2) decrease in β cell apoptosis; (3) increased β cell proliferation; and (4) inhibition of endothelial cell adhesion molecules. The following protocol was implemented, Table 8.

TABLE 8
Protocol for Exendin-4 therapy used to evaluate
the average virtual NOD mouse

| | Ogawa 2004 |
|---|---|
| Timing of treatment | 7-17 days after diagnosis of diabetes |
| Regimen | 12 nmol/kg of exendin-4 for 4 consec. |
| | days twice (days 0-3 and 7-10) |
| | Dose: 0.3 nmol |
| | Effective dose: 0.003 nmol (Parkes 2001) |
| | Daily insulin until glycemic control |
| Incidence | No remission |
| Expected virtual mouse | No remission |
| outcome | |

Simulation results of Exendin-4 treatment were consistent with the published results that it had no effect on the incidence or development of diabetes, or preservation of β cell mass (data not shown).
9. Anti-CD40L

The direct effects of this agent that were implemented included the following: (1) inhibition of costimulation of conventional CD4+β cells; (2) CD8+ T cell activation by decreasing CD4+ T cell help; (3) B lymphocyte activation; (4) dendritic cell/macrophage activation; and (5) 50% inhibition of expression of adhesion molecules on endothelial cells. The following protocol was implemented, Table 9

TABLE 9
Protocol for anti-CD40L therapy used to evaluate
the average virtual NOD mouse

| | Balasa 1997 (early) |
|---|---|
| Timing of Rx | 3-12 wks |
| Regimen | 250 μg i.p. at day 0, 2, 4 |
| | and at wk 6, 9 and 12 |
| Incidence | 0% vs. 80% control at 31 wks |
| Insulitis | No insulitis |
| Expected virtual | No diabetes at 31 wks |
| mouse outcome | |

Simulation results of the protocol showed that anti-CD40L, when administered from 3-12 weeks protects the virtual mouse from developing diabetes and reduces insulitis, which is consistent with the published report and several other published treatment protocols.
10. Rapamycin

Rapamycin protects pre-diabetic NOD mice, but fails to induce remission in overtly diabetic NOD mice. This agent acts on the protein kinase mammalian target of rapamycin (mTOR), a key regulator of cell growth and proliferation with known effects on T and B lymphocytes
11. Anti-IL-2

Administration of an antibody against IL-2 to neonatal NOD mice exacerbates the disease, with treated animals developing diabetes earlier and at a higher incidence than control animals. The mechanism of action appears to be tied to selective reduction in the number of CD4+ CD25+ T regulatory cells over conventional CD4+ CD25− T cells.

The average virtual NOD mouse reproduces published responses for eleven different therapeutic agents, modulating a wide variety of pathways and treating different stages in disease progression. The ability of the average virtual NOD mouse to respond in a manner substantively consistent with these known patterns of response provides assurance that the virtual mouse is a high fidelity representation of the laboratory NOD mouse and establishes the mouse as validated.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for developing a model of type 1 diabetes said method comprising:
identifying one or more biological processes associated with a pancreatic lymph node;
identifying one or more biological processes associate with one or more pancreatic islets;
mathematically representing each biological process to generate one or more representations of a biological process associated with the pancreatic lymph node and one or more representations of a biological process associated with the one or more pancreatic islets; and
combining the representations of biological processes to form a model of type 1 diabetes.

2. The method of claim 1, further comprising:
identifying one or more biological processes associated with a gut and/or gut associated lymphoid tissue; and
mathematically representing each biological process associated with the gut and/or gut associated lymphoid tissue.

3. The method of claim 1, wherein the one or more pancreatic islets comprises at least two pancreatic islets.

4. The method of claim 1, wherein at least one of the one or more biological processes associated with a pancreatic lymph node is a biological process related to a balance of effector and regulatory cell populations.

5. The method of claim 4, wherein the regulatory cell population comprises cells of lymphoid lineage.

6. The method of claim 4, wherein the regulatory cell population comprises regulatory T cells.

7. The method of claim 6, wherein the regulatory T cells of the regulatory cell population do not express intrinsic effector cell activity.

8. The method of claim 6, wherein the regulatory T cells of the regulatory cell population include both innate regulatory T cells and adaptive regulatory T cells.

9. The method of claim 6, wherein the regulatory T cells of the regulatory cell population suppress effector T cell activity via direct and indirect mechanisms.

10. The method of claim 1, wherein at least one of the one or more biological processes associated with one or more pancreatic islets is a biological process related to a balance of effector and regulatory cell populations.

11. The method of claim 10, wherein the regulatory cell population comprises cells of lymphoid lineage.

12. The method of claim 10, wherein the regulatory cell population comprises regulatory T cells.

13. The method of claim 12, wherein the regulatory T cells of the regulatory cell population do not express intrinsic effector cell activity.

14. A computer-readable medium having computer-readable instructions stored thereon that, upon execution by a processor, cause the processor to simulate type 1 diabetes, and further wherein the instructions comprise:
a) mathematically representing one or more biological processes associated with a pancreatic lymph node;
b) mathematically representing one or more biological processes associated with one or more pancreatic islets;
c) defining a set of mathematical relationships between the representations of biological processes to form a model of type 1 diabetes.

15. The computer-readable medium of claim 14, wherein the instructions further comprise mathematically representing one or more biological processes associated with a gut and/or gut associated lymphoid tissue.

16. The computer-readable medium of claim 14, wherein the instructions further comprise accepting user input specifying one or more parameters associated with one or more of the mathematical representations.

17. The computer-readable medium of claim 14, wherein the instructions further comprise accepting user input specifying one or more variables associated with one or more of the mathematical representations.

18. The computer-readable medium of claim 14, wherein the instructions further comprise applying a virtual protocol to the model of type 1 diabetes.

19. The computer-readable medium of claim 14, wherein the instructions further comprise defining one or more virtual patients.

20. A system, comprising:
a) a processor including computer-readable instructions stored thereon that, upon execution by a processor, cause the processor to simulate type 1 diabetes in a mammal, the computer readable instructions comprising:
i) mathematically representing one or more biological processes associated with a pancreatic lymph node;
ii) mathematically representing one or more biological processes associated with one or more pancreatic islets;
iii) defining a set of mathematical relationships between the representations of biological processes associated with the pancreatic lymph node and representations of biological processes associated with the one or more pancreatic islets;
iv) applying a virtual protocol to the set of mathematical relationships to generate a set of outputs;
b) a first user terminal, the first user terminal operable to receive a user input specifying one or more parameters associated with one or more mathematical representations defined by the computer readable instructions; and
c) a second user terminal, the second user terminal operable to provide the set of outputs to a second user.

21. A method for developing a model of progression of type 1 diabetes said method comprising:
identifying one or more biological processes associated with development of each of at least two conditions selected from the group consisting of autoreactive T cell production, autoreactive T cell priming; insulitis and hyperglycemia;
mathematically representing each biological process to generate one or more representations of a biological process associated with each of the at least two conditions; and
combining the representations of biological processes to form a model of progression of type 1 diabetes.

22. The method of claim 21, wherein the at least two conditions comprise insulitis and hyperglycemia.

23. The method of claim 21, wherein the at least two conditions comprise autoreactive T cell priming.

24. The method of claim 21, wherein autoreactive T cell priming includes a biological process related to a balance of effector and regulatory cell populations.

25. A computer-readable medium having computer-readable instructions stored thereon that, upon execution by a processor, cause the processor to simulate progression of type 1 diabetes, and further wherein the instructions comprise:
a) mathematically representing one or more biological processes associated with development of each of at least two conditions selected from the group consisting of autoreactive T cell production, autoreactive T cell priming; insulitis and hyperglycemia;
b) defining a set of mathematical relationships between the representations of biological processes to form a model of progression of type 1 diabetes.

26. The computer-readable medium of claim 25, wherein the instructions further comprise accepting user input specifying one or more parameters associated with one or more of the mathematical representations.

27. The computer-readable medium of claim 25, wherein the instructions further comprise accepting user input specifying one or more variables associated with one or more of the mathematical representations.

28. The computer-readable medium of claim 25, wherein the instructions further comprise applying a virtual protocol to the model of type 1 diabetes.

29. The computer-readable medium of claim 25, wherein the instructions further comprise defining one or more virtual patients.

30. A system, comprising:
a) a processor including computer-readable instructions stored thereon that, upon execution by a processor, cause the processor to simulate progression of type 1 diabetes in a mammal, the computer readable instructions comprising:
i) mathematically representing one or more biological processes associated with development of each of at least two conditions selected from the group consisting of autoreactive T cell production, autoreactive T cell priming; insulitis and hyperglycemia;
ii) defining a set of mathematical relationships between the representations of biological processes associated with the at least two conditions;
iii) applying a virtual protocol to the set of mathematical relationships to generate a set of outputs;
b) a first user terminal, the first user terminal operable to receive a user input specifying one or more parameters associated with one or more mathematical representations defined by the computer readable instructions; and
c) a second user terminal, the second user terminal operable to provide the set of outputs to a second user.

31. A method for developing a model of a non-insulin replacement treatment of type 1 diabetes said method comprising:
identifying one or more biological processes associated with a β cell population in at least one of one or more pancreatic islets;
identifying one or more biological processes associated with an effect of the non-insulin replacement treatment of type 1 diabetes;
mathematically representing each biological process to generate one or more representations of a biological process associated with the β cell population and one or more representations of a biological process associated with an effect of the non-insulin replacement treatment of type 1 diabetes; and
combining the representations of biological processes to form a model of a non-insulin replacement treatment of type 1 diabetes.

32. The method of claim 31, further comprising the steps of:
identifying one or more biological processes associated with a pancreatic lymph node; and
mathematically representing each biological process to generate one or more representations of a biological process associated with the pancreatic lymph node.

33. The method of claim 31, wherein the one or more biological processes associated with the β cells comprises a biological process associated with an autoimmune response against the β cells.

34. The method of claim 31, wherein the one or more biological processes associated with increasing β cells comprises a biological process associated with resistance of the β cells to death.

35. The method of claim 31, wherein the one or more biological processes associated with increasing β cells comprises a biological process associated with β cell proliferation.

36. The method of claim 31, wherein the one or more biological processes associated with increasing β cells comprises a biological process associated with β cell neogenesis.

37. The method of claim 31, wherein at least one of the one or more biological processes associated with the β cell population is a biological process related to a balance of effector and regulatory cell populations.

38. The method of claim 37, wherein the regulatory cell population comprises cells of lymphoid lineage.

39. The method of claim 37, wherein the regulatory cell population comprises regulatory T cells.

40. The method of claim 39, wherein the regulatory T cells of the regulatory cell population do not express intrinsic effector cell activity.

41. A computer-readable medium having computer-readable instructions stored thereon that, upon execution by a processor, cause the processor to a non-insulin replacement treatment of type 1 diabetes and further wherein the instructions comprise:
a) mathematically representing one or more biological processes associated with a cell population in at least one of one or more pancreatic islets;
b) mathematically representing one or more biological processes associated with an effect of the non-insulin replacement treatment of type 1 diabetes;
c) defining a set of mathematical relationships between the representations of biological processes to form a model of the non-insulin replacement treatment of type 1 diabetes.

42. The computer-readable medium of claim 41, wherein the instructions further comprise mathematically representing one or more biological processes associated with a pancreatic lymph node.

43. The computer-readable medium of claim 41, wherein the instructions further comprise mathematically representing one or more biological processes associated with a gut and/or gut associated lymphoid tissue.

44. The computer-readable medium of claim 41, wherein the instructions further comprise accepting user input specifying one or more parameters associated with one or more of the mathematical representations.

45. The computer-readable medium of claim 41, wherein the instructions further comprise accepting user input specifying one or more variables associated with one or more of the mathematical representations.

46. The computer-readable medium of claim 41, wherein the instructions further comprise applying a virtual protocol to the model of type 1 diabetes.

47. The computer-readable medium of claim 41, wherein the instructions further comprise defining one or more virtual patients.

48. A system, comprising:
a) a processor including computer-readable instructions stored thereon that, upon execution by a processor, cause the processor to simulate progression of type 1 diabetes in a mammal, the computer readable instructions comprising:
i) mathematically representing one or more biological processes associated with one or more pancreatic islets;
ii) mathematically representing one or more biological processes associated with a β cell population in at least one of the one or more pancreatic islets;
iii) mathematically representing one or more biological processes associated with an effect of a non-insulin replacement treatment of type 1 diabetes;
iv) defining a set of mathematical relationships between the representations of biological processes associated with the one or more pancreatic islets and the representations of biological processes associated with the β cell population and the representations associated with an effect of the non-insulin replacement treatment of type 1 diabetes;
v) applying a virtual protocol to the set of mathematical relationships to generate a set of outputs;
b) a first user terminal, the first user terminal operable to receive a user input specifying one or more parameters associated with one or more mathematical representations defined by the computer readable instructions; and
c) a second user terminal, the second user terminal operable to provide the set of outputs to a second user.

49. A method of simulating type 1 diabetes, said method comprising executing a computer model of type 1 diabetes according to any one of claims 14, 25 and 41.

50. The method of claim 46, further comprising applying a virtual protocol to the computer model to generate set of outputs representing a phenotype of type 1 diabetes.

51. The method of claim 50, wherein the virtual protocol comprises a therapeutic regimen, a diagnostic procedure, passage of time, or exposure to environmental toxins.

52. The method of claim 50, wherein the phenotype represents a diseased state.

53. The method of claim 49, further comprising accepting user input specifying one or more parameters or variable associated with one or more mathematical representations prior to executing the computer model.

54. The method of claim 53, wherein the user input comprises a definition of a virtual patient.

55. A computer-based mathematical model of a biological system comprising a representation of a tissue, wherein the tissue comprises a plurality of distinct distributed sites and the representation of the tissue comprises a plurality of representations, wherein each of the plurality of representations associated with one of the plurality of distinct distributed sites.

56. The model of claim 55, wherein the tissue is selected from the group consisting of lung, brain, liver, joints, intestine and pancreas.

57. the model of claim 55, wherein the distinct distributed sites describe spatial heterogeneity within the tissue.

58. The model of claim 55, wherein the distinct distributed sites describe temporal heterogeneity within the tissue.

59. The model of claim 55, wherein the distinct distributed sites describe distinct stages in progression of a disorder within the tissue.
